SEQUENCE LISTING (163 sequences)

SEQ ID NO: 1 - protein, 23 aa, Artificial Sequence
  Description of Artificial Sequence Synthetic peptide
VRGIITSKTKSLDKGYNKALNDL

SEQ ID NO: 2 - protein, 23 aa, Artificial Sequence
  Description of Artificial Sequence Synthetic peptide
VRGIIPFKTKSLDEGYNKALNDL

SEQ ID NO: 3 - protein, 8 aa, Artificial Sequence
  Description of Artificial Sequence Synthetic peptide
KSVKAPGI

SEQ ID NO: 4 - protein, 15 aa, Artificial Sequence
  Description of Artificial Sequence Synthetic peptide
HKAIDGRSLYNKTLD

SEQ ID NO: 5 - protein, 12 aa, Artificial Sequence
  Description of Artificial Sequence Synthetic peptide
LRLTKNSRDDST

SEQ ID NO: 6 - protein, 13 aa, Artificial Sequence
  Description of Artificial Sequence Synthetic peptide
KNIVSVKGIRKSI

SEQ ID NO: 7 - protein, 15 aa, Artificial Sequence
  Description of Artificial Sequence Synthetic peptide
KSVIPRKGTKAPPRL

SEQ ID NO: 8 - protein, 13 aa, Artificial Sequence
  Description of Artificial Sequence Synthetic peptide
KPVMYKNTGKSEQ

SEQ ID NO: 9 - protein, 18 aa, Artificial Sequence
  Description of Artificial Sequence Synthetic peptide
EFVMNPANAQGHTPGTRL

SEQ ID NO: 10 - protein, 18 aa, Artificial Sequence
  Description of Artificial Sequence Synthetic peptide
EFVMNPANAQGHTAGTRL

SEQ ID NO: 11 - protein, 18 aa, Artificial Sequence
  Description of Artificial Sequence Synthetic peptide
EFVMNAANAQGHTPGTRL

SEQ ID NO: 12 - protein, 19 aa, Artificial Sequence
  Description of Artificial Sequence Synthetic peptide
EFVMNPANAQGRHTPGTRL

SEQ ID NO: 13 - protein, 21 aa, Artificial Sequence
  Description of Artificial Sequence Synthetic peptide
NPKEFVMNPANAQGHTPGTRL

SEQ ID NO: 14 - protein, 22 aa, Artificial Sequence
  Description of Artificial Sequence Synthetic peptide
NPKEFVMNPANAQGRHTPGTRL

SEQ ID NO: 15 - protein, 27 aa, Artificial Sequence
  Description of Artificial Sequence Synthetic peptide
KKIIPPTNIRENLYNRTASLTDLGGEL

SEQ ID NO: 16 - protein, 24 aa, Artificial Sequence
  Description of Artificial Sequence Synthetic peptide
CVRGIITSKTKSLDKGYNKALNDL

SEQ ID NO: 17 - protein, 24 aa, Artificial Sequence
  Description of Artificial Sequence Synthetic peptide
CVRGIIPFKTKSLDEGYNKALNDL

SEQ ID NO: 18 - protein, 9 aa, Artificial Sequence
  Description of Artificial Sequence Synthetic peptide
CKSVKAPGI

SEQ ID NO: 19 - protein, 16 aa, Artificial Sequence
  Description of Artificial Sequence Synthetic peptide
CHKAIDGRSLYNKTLD

SEQ ID NO: 20 - protein, 13 aa, Artificial Sequence
  Description of Artificial Sequence Synthetic peptide
CLRLTKNSRDDST

SEQ ID NO: 21 - protein, 14 aa, Artificial Sequence
  Description of Artificial Sequence Synthetic peptide
CKNIVSVKGIRKSI

SEQ ID NO: 22 - protein, 16 aa, Artificial Sequence
  Description of Artificial Sequence Synthetic peptide
CKSVIPRKGTKAPPRL

SEQ ID NO: 23 - protein, 14 aa, Artificial Sequence
  Description of Artificial Sequence Synthetic peptide
CKPVMYKNTGKSEQ

SEQ ID NO: 24 - protein, 19 aa, Artificial Sequence
  Description of Artificial Sequence Synthetic peptide
CEFVMNPANAQGHTPGTRL

SEQ ID NO: 25 - protein, 19 aa, Artificial Sequence
  Description of Artificial Sequence Synthetic peptide
CEFVMNPANAQGHTAGTRL

SEQ ID NO: 26 - protein, 19 aa, Artificial Sequence
  Description of Artificial Sequence Synthetic peptide
CEFVMNAANAQGHTPGTRL

SEQ ID NO: 27 - protein, 20 aa, Artificial Sequence
  Description of Artificial Sequence Synthetic peptide
CEFVMNPANAQGRHTPGTRL

SEQ ID NO: 28 - protein, 22 aa, Artificial Sequence
  Description of Artificial Sequence Synthetic peptide
CNPKEFVMNPANAQGHTPGTRL

SEQ ID NO: 29 - protein, 23 aa, Artificial Sequence
  Description of Artificial Sequence Synthetic peptide
CNPKEFVMNPANAQGRHTPGTRL

SEQ ID NO: 30 - protein, 28 aa, Artificial Sequence
  Description of Artificial Sequence Synthetic peptide
CKKIIPPTNIRENLYNRTASLTDLGGEL

SEQ ID NO: 31 - protein, 24 aa, Artificial Sequence
  Description of Artificial Sequence Synthetic peptide
GVRGIITSKTKSLDKGYNKALNDL

SEQ ID NO: 32 - protein, 24 aa, Artificial Sequence
  Description of Artificial Sequence Synthetic peptide
GVRGIIPFKTKSLDEGYNKALNDL

SEQ ID NO: 33 - protein, 9 aa, Artificial Sequence
  Description of Artificial Sequence Synthetic peptide
GKSVKAPGI

SEQ ID NO: 34 - protein, 16 aa, Artificial Sequence
  Description of Artificial Sequence Synthetic peptide
GHKAIDGRSLYNKTLD

SEQ ID NO: 35 - protein, 13 aa, Artificial Sequence
  Description of Artificial Sequence Synthetic peptide
GLRLTKNSRDDST

SEQ ID NO: 36 - protein, 14 aa, Artificial Sequence
  Description of Artificial Sequence Synthetic peptide
GKNIVSVKGIRKSI

SEQ ID NO: 37 - protein, 16 aa, Artificial Sequence
  Description of Artificial Sequence Synthetic peptide
GKSVIPRKGTKAPPRL

SEQ ID NO: 38 - protein, 14 aa, Artificial Sequence
  Description of Artificial Sequence Synthetic peptide
GKPVMYKNTGKSEQ

SEQ ID NO: 39 - protein, 19 aa, Artificial Sequence
  Description of Artificial Sequence Synthetic peptide
GEFVMNPANAQGHTPGTRL

SEQ ID NO: 40 - protein, 19 aa, Artificial Sequence
  Description of Artificial Sequence Synthetic peptide
GEFVMNPANAQGHTAGTRL

SEQ ID NO: 41 - protein, 19 aa, Artificial Sequence
  Description of Artificial Sequence Synthetic peptide
GEFVMNAANAQGHTPGTRL

SEQ ID NO: 42 - protein, 20 aa, Artificial Sequence
  Description of Artificial Sequence Synthetic peptide
GEFVMNPANAQGRHTPGTRL

SEQ ID NO: 43 - protein, 22 aa, Artificial Sequence
  Description of Artificial Sequence Synthetic peptide
GNPKEFVMNPANAQGHTPGTRL

SEQ ID NO: 44 - protein, 23 aa, Artificial Sequence
  Description of Artificial Sequence Synthetic peptide
GNPKEFVMNPANAQGRHTPGTRL

SEQ ID NO: 45 - protein, 28 aa, Artificial Sequence
  Description of Artificial Sequence Synthetic peptide
GKKIIPPTNIRENLYNRTASLTDLGGEL

SEQ ID NO: 46 - protein, 24 aa, Artificial Sequence
  Description of Artificial Sequence Synthetic peptide
VRGIITSKTKSLDKGYNKALNDLC

SEQ ID NO: 47 - protein, 24 aa, Artificial Sequence
  Description of Artificial Sequence Synthetic peptide
VRGIIPFKTKSLDEGYNKALNDLC

SEQ ID NO: 48 - protein, 9 aa, Artificial Sequence
  Description of Artificial Sequence Synthetic peptide
KSVKAPGIC

SEQ ID NO: 49 - protein, 16 aa, Artificial Sequence
  Description of Artificial Sequence Synthetic peptide
HKAIDGRSLYNKTLDC

SEQ ID NO: 50 - protein, 13 aa, Artificial Sequence
  Description of Artificial Sequence Synthetic peptide
LRLTKNSRDDSTC

SEQ ID NO: 51 - protein, 14 aa, Artificial Sequence
  Description of Artificial Sequence Synthetic peptide
KNIVSVKGIRKSIC

SEQ ID NO: 52 - protein, 16 aa, Artificial Sequence
  Description of Artificial Sequence Synthetic peptide
KSVIPRKGTKAPPRLC

SEQ ID NO: 53 - protein, 14 aa, Artificial Sequence
  Description of Artificial Sequence Synthetic peptide
KPVMYKNTGKSEQC

SEQ ID NO: 54 - protein, 19 aa, Artificial Sequence
  Description of Artificial Sequence Synthetic peptide
EFVMNPANAQGHTPGTRLC

SEQ ID NO: 55 - protein, 19 aa, Artificial Sequence
  Description of Artificial Sequence Synthetic peptide
EFVMNPANAQGHTAGTRLC

SEQ ID NO: 56 - protein, 19 aa, Artificial Sequence
  Description of Artificial Sequence Synthetic peptide
EFVMNAANAQGHTPGTRLC

SEQ ID NO: 57 - protein, 20 aa, Artificial Sequence
  Description of Artificial Sequence Synthetic peptide
EFVMNPANAQGRHTPGTRLC

SEQ ID NO: 58 - protein, 22 aa, Artificial Sequence
  Description of Artificial Sequence Synthetic peptide
NPKEFVMNPANAQGHTPGTRLC

SEQ ID NO: 59 - protein, 23 aa, Artificial Sequence
  Description of Artificial Sequence Synthetic peptide
NPKEFVMNPANAQGRHTPGTRLC

SEQ ID NO: 60 - protein, 28 aa, Artificial Sequence
  Description of Artificial Sequence Synthetic peptide
KKIIPPTNIRENLYNRTASLTDLGGELC

SEQ ID NO: 61 - protein, 18 aa, Artificial Sequence
  Description of Artificial Sequence Synthetic peptide
KSVKAPGIGGKSVKAPGI

SEQ ID NO: 62 - protein, 28 aa, Artificial Sequence
  Description of Artificial Sequence Synthetic peptide
KSVKAPGIGGKSVKAPGIGGKSVKAPGI

SEQ ID NO: 63 - protein, 10 aa, Artificial Sequence
  Description of Artificial Sequence Synthetic peptide
KSVKAPGIGG

SEQ ID NO: 64 - protein, 19 aa, Artificial Sequence
  Description of Artificial Sequence Synthetic peptide
CKSVKAPGIGGKSVKAPGI

SEQ ID NO: 65 - protein, 29 aa, Artificial Sequence
  Description of Artificial Sequence Synthetic peptide
CKSVKAPGIGGKSVKAPGIGGKSVKAPGI

SEQ ID NO: 66 - protein, 23 aa, Artificial Sequence
  Description of Artificial Sequence Synthetic peptide
GLFGAIAGFIENGWEGMIDGWYG

SEQ ID NO: 67 - protein, 24 aa, Artificial Sequence
  Description of Artificial Sequence Synthetic peptide
CGLFGAIAGFIENGWEGMIDGWYG

SEQ ID NO: 68 - protein, 24 aa, Artificial Sequence
  Description of Artificial Sequence Synthetic peptide
GLFGAIAGFIENGWEGMIDGWYGC

SEQ ID NO: 69 - protein, 6 aa, Artificial Sequence
  Description of Artificial Sequence Synthetic peptide
GRGDGG

SEQ ID NO: 70 - protein, 6 aa, Artificial Sequence
  Description of Artificial Sequence Synthetic peptide
CRGDGG

SEQ ID NO: 71 - protein, 6 aa, Artificial Sequence
  Description of Artificial Sequence Synthetic peptide
GRGDGC

SEQ ID NO: 72 - protein, 7 aa, Artificial Sequence
  Description of Artificial Sequence Synthetic peptide
THALWHT

SEQ ID NO: 73 - protein, 8 aa, Artificial Sequence
  Description of Artificial Sequence Synthetic peptide
GTHALWHT

SEQ ID NO: 74 - protein, 8 aa, Artificial Sequence
  Description of Artificial Sequence Synthetic peptide
THALWHTG

SEQ ID NO: 75 - protein, 8 aa, Artificial Sequence
  Description of Artificial Sequence Synthetic peptide
CTHALWHT

SEQ ID NO: 76 - protein, 8 aa, Artificial Sequence
  Description of Artificial Sequence Synthetic peptide
THALWHTC

SEQ ID NO: 77 - protein, 15 aa, Artificial Sequence
  Description of Artificial Sequence Synthetic peptide
QPFMQCLCLIYDASC

SEQ ID NO: 78 - protein, 16 aa, Artificial Sequence
  Description of Artificial Sequence Synthetic peptide
GQPFMQCLCLIYDASC

SEQ ID NO: 79 - protein, 16 aa, Artificial Sequence
  Description of Artificial Sequence Synthetic peptide
QPFMQCLCLIYDASCG

SEQ ID NO: 80 - protein, 15 aa, Artificial Sequence
  Description of Artificial Sequence Synthetic peptide
RNVPPIFNDVYWIAF

SEQ ID NO: 81 - protein, 16 aa, Artificial Sequence
  Description of Artificial Sequence Synthetic peptide
GRNVPPIFNDVYWIAF

SEQ ID NO: 82 - protein, 16 aa, Artificial Sequence
  Description of Artificial Sequence Synthetic peptide
RNVPPIFNDVYWIAFG

SEQ ID NO: 83 - protein, 16 aa, Artificial Sequence
  Description of Artificial Sequence Synthetic peptide
CRNVPPIFNDVYWIAF

SEQ ID NO: 84 - protein, 16 aa, Artificial Sequence
  Description of Artificial Sequence Synthetic peptide
RNVPPIFNDVYWIAFC

SEQ ID NO: 85 - protein, 14 aa, Artificial Sequence
  Description of Artificial Sequence Synthetic peptide
VFRVRPWYQSTSQS

SEQ ID NO: 86 - protein, 15 aa, Artificial Sequence
  Description of Artificial Sequence Synthetic peptide
GVFRVRPWYQSTSQS

SEQ ID NO: 87 - protein, 15 aa, Artificial Sequence
  Description of Artificial Sequence Synthetic peptide
VFRVRPWYQSTSQSG

SEQ ID NO: 88 - protein, 15 aa, Artificial Sequence
  Description of Artificial Sequence Synthetic peptide
CVFRVRPWYQSTSQS

SEQ ID NO: 89 - protein, 15 aa, Artificial Sequence
  Description of Artificial Sequence Synthetic peptide
VFRVRPWYQSTSQSC

SEQ ID NO: 90 - protein, 115 aa, Homo sapiens
MIPAKDMAKVMIVMLAICFLTKSDGKSVKKRSVSEIQLMHNLGKHLNSMERVEWLRKKLQDVHNFVALGAPLAPRDAGSQRPRKKEDNVLVESHEKSLGEADKADVNVLTKAKSQ

SEQ ID NO: 91 - protein, 138 aa, Homo sapiens
MTALFLMSMLFGLACGQAMSFCIPTEYTMHIERRECAYCLTINTTICAGYCMTRDINGKLFLPKYALSQDVCTYRDFIYRTVEIPGCPLHVAPYFSYPVALSCKCGKCNTDYSDCIHEAIKTNYCTKPQKSYLVGFSV

SEQ ID NO: 92 - protein, 242 aa, Homo sapiens
MPGPWLLLALALTLNLTGVPGGRAQPEAAQQEAVTAAEHPGLDDFLRQVERLLFLRENIQRLQGDQGEHSASQIFQSDWLSKRQHPGKREEEEEEGVEEEEEEEGGAVGPHKRQHPGRREDEASWSVDVTQHKRQHPGRRSPWLAYAVPKRQHPGRRLADPKAQRSWEEEEEEEEREEDLMPEKRQHPGKRALGGPCGPQGAYGQAGLLLGLLDDLSRSQGAEEKRQHPGRRAAWVREPLEE

SEQ ID NO: 93 - protein, 92 aa, Homo sapiens
MKPIQKLLAGLILLTWCVEGCSSQHWSYGLRPGGKRDAENLIDSFQEIVKEVGQLAETQRFECTTHQPRSPLRDLKGALESLIEEETGQKKI

SEQ ID NO: 94 - protein, 120 aa, Homo sapiens
MASSRRGLLLLLLLTAHLGPSEAQHWSHGWYPGGKRALSSAQDPQNALRPPGRALDTAAGSPVQTAHGLPSDALAPLDDSMPWEGRTTAQWSLHRKRHLARTLLTAAREPRPAPPSSNKV

SEQ ID NO: 95 - protein, 196 aa, Homo sapiens
MRLPLLVSAGVLLVALLPCPPCRALLSRGPVPGARQAPQHPQPLDFFQPPPQSEQPQQPQARPVLLRMGEEYFLRLGNLNKSPAAPLSPASSLLAGGSGSRPSPEQATANFFRVLLQQLLLPRRSLDSPAALAERGARNALGGHQEAPERERRSEEPPISLDLTFHLLREVLEMARAEQLAQQAHSNRKLMEIIGK

SEQ ID NO: 96 - protein, 267 aa, Homo sapiens
MPRSCCSRSGALLLALLLQASMEVRGWCLESSQCQDLTTESNLLECIRACKPDLSAETPMFPGNGDEQPLTENPRKYVMGHFRWDRFGRRNSSSSGSSGAGQKREDVSAGEDCGPLPEGGPEPRSDGAKPGPREGKRSYSMEHFRWGKPVGKKRRPVKVYPNGAEDESAEAFPLEFKRELTGQRLREGDGPDGPADDGAGAQADLEHSLLVAAEKKDEGPYRMEHFRWGSPPKDKRYGGFMTSEKSQTPLVTLFKNAIIKNAYKKGE

SEQ ID NO: 97 - protein, 622 aa, Homo sapiens
MAHVRGLQLPGCLALAALCSLVHSQHVFLAPQQARSLLQRVRRANTFLEEVRKGNLERECVEETCSYEEAFEALESSTATDVFWAKYTACETARTPRDKLAACLEGNCAEGLGTNYRGHVNITRSGIECQLWRSRYPHKPEINSTTHPGADLQENFCRNPDSSTTGPWCYTTDPTVRRQECSIPVCGQDQVTVAMTPRSEGSSVNLSPPLEQCVPDRGQQYQGRLAVTTHGLPCLAWASAQAKALSKHQDFNSAVQLVENFCRNPDGDEEGVWCYVAGKPGDFGYCDLNYCEEAVEEETGDGLDEDSDRAIEGRTATSEYQTFFNPRTFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

SEQ ID NO: 98 - protein, 1663 aa, Homo sapiens
MGPTSGPSLLLLLLTHLPLALGSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNPRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAARRRRSVQLTEKRMDKVGKYPKELRKCCEDGMRENPMRFSCQRRTRFISLGEACKKVFLDCCNYITELRRQHARASHLGLARSNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 99 - protein, 1744 aa, Homo sapiens
MRLLWGLIWASSFFTLSLQKPRLLLFSPSVVHLGVPLSVGVQLQDVPRGQVVKGSVFLRNPSRNNVPCSPKVDFTLSSERDFALLSLQVPLKDAKSCGLHQLLRGPEVQLVAHSPWLKDSLSRTTNIQGINLLFSSRRGHLFLQTDQPIYNPGQRVRYRVFALDQKMRPSTDTITVMVENSHGLRVRKKEVYMPSSIFQDDFVIPDISEPGTWKISARFSDGLESNSSTQFEVKKYVLPNFEVKITPGKPYILTVPGHLDEMQLDIQARYIYGKPVQGVAYVRFGLLDEDGKKTFFRGLESQTKLVNGQSHISLSKAEFQDALEKLNMGITDLQGLRLYVAAAIIESPGGEMEEAELTSWYFVSSPFSLDLSKTKRHLVPGAPFLLQALVREMSGSPASGIPVKVSATVSSPGSVPEVQDIQQNTDGSGQVSIPIIIPQTISELQLSVSAGSPHPAIARLTVAAPPSGGPGFLSIERPDSRPPRVGDTLNLNLRAVGSGATFSHYYYMILSRGQIVFMNREPKRTLTSVSVFVDHHLAPSFYFVAFYYHGDHPVANSLRVDVQAGACEGKLELSVDGAKQYRNGESVKLHLETDSLALVALGALDTALYAAGSKSHKPLNMGKVFEAMNSYDLGCGPGGGDSALQVFQAAGLAFSDGDQWTLSRKRLSCPKEKTTRKKRNVNFQKAINEKLGQYASPTAKRCCQDGVTRLPMMRSCEQRAARVQQPDCREPFLSCCQFAESLRKKSRDKGQAGLQRALEILQEEDLIDEDDIPVRSFFPENWLWRVETVDRFQILTLWLPDSLTTWEIHGLSLSKTKGLCVATPVQLRVFREFHLHLRLPMSVRRFEQLELRPVLYNYLDKNLTVSVHVSPVEGLCLAGGGGLAQQVLVPAGSARPVAFSVVPTAAAAVSLKVVARGSFEFPVGDAVSKVLQIEKEGAIHREELVYELNPLDHRGRTLEIPGNSDPNMIPDGDFNSYVRVTASDPLDTLGSEGALSPGGVASLLRLPRGCGEQTMIYLAPTLAASRYLDKTEQWSTLPPETKDHAVDLIQKGYMRIQQFRKADGSYAAWLSRDSSTWLTAFVLKVLSLAQEQVGGSPEKLQETSNWLLSQQQADGSFQDLSPVIHRSMQGGLVGNDETVALTAFVTIALHHGLAVFQDEGAEPLKQRVEASISKANSFLGEKASAGLLGAHAAAITAYALSLTKAPVDLLGVAHNNLMAMAQETGDNLYWGSVTGSQSNAVSPTPAPRNPSDPMPQAPALWIETTAYALLHLLLHEGKAEMADQASAWLTRQGSFQGGFRSTQDTVIALDALSAYWIASHTTEERGLNVTLSSTGRNGFKSHALQLNNRQIRGLEEELQFSLGSKINVKVGGNSKGTLKVLRTYNVLDMKNTTCQDLQIEVTVKGHVEYTMEANEDYEDYEYDELPAKDDPDAPLQPVTPLQLFEGRRNRRRREAPKVVEEQESRVHYTVCIWRNGKVGLSGMAIADVTLLSGFHALRADLEKLTSLSDRYVSHFETEGPHVLLYFDSVPTSRECVGFEAVQEVPVGLVQPASATLYDYYNPERRCSVFYGAPSKSRLLATLCSAEVCQCAEGKCPRQRRALERGLQDEDGYRMKFACYYPRVEYGFQVKVLREDSRAAFRLFETKITQVLHFTKDVKAAANQMRNFLVRASCRLRLEPGKEYLIMGLDGATYDLEGHPQYLLDSNSWIEEMPSERLCRSTRQRAACAQLNDFLQEYGTQGCQV

SEQ ID NO: 100 - protein, 770 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 101 - protein, 217 aa, Homo sapiens
MATGSRTSLLLAFGLLCLPWLQEGSAFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 102 - protein, 117 aa, Homo sapiens
MPSPGTVCSLLLLGMLWLDLAMAGSSFLSPEHQRVQQRKESKKPPAKLQPRALAGWLRPEDGGQAEGAEDELEVRFNAPFDVGIKLSGVQYQQHSQALGKFLQDILWEEAKEAPADK

SEQ ID NO: 103 - protein, 390 aa, Homo sapiens
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 104 - protein, 99 aa, Homo sapiens
MKVSAALLCLLLIAATFIPQGLAQPDAINAPVTCCYNFTNRKISVQRLASYRRITSSKCPKEAVIFKTIVAKEICADPKQKWVQDSMDHLDKQTQTPKT

SEQ ID NO: 105 - protein, 798 aa, Homo sapiens
MNLQPIFWIGLISSVCCVFAQTDENRCLKANAKSCGECIQAGPNCGWCTNSTFLQEGMPTSARCDDLEALKKKGCPPDDIENPRGSKDIKKNKNVTNRSKGTAEKLKPEDITQIQPQQLVLRLRSGEPQTFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSAVGTLSANSSNVIQLIIDAYNSLSSEVILENGKLSEGVTISYKSYCKNGVNGTGENGRKCSNISIGDEVQFEISITSNKCPKKDSDSFKIRPLGFTEEVEVILQYICECECQSEGIPESPKCHEGNGTFECGACRCNEGRVGRHCECSTDEVNSEDMDAYCRKENSSEICSNNGECVCGQCVCRKRDNTNEIYSGKFCECDNFNCDRSNGLICGGNGVCKCRVCECNPNYTGSACDCSLDTSTCEASNGQICNGRGICECGVCKCTDPKFQGQTCEMCQTCLGVCAEHKECVQCRAFNKGEKKDTCTQECSYFNITKVESRDKLPQPVQPDPVSHCKEKDVDDCWFYFTYSVNGNNEVMVHVVENPECPTGPDIIPIVAGVVAGIVLIGLALLLIWKLLMIIHDRREFAKFEKEKMNAKWDTGENPIYKSAVTTVVNPKYEGK

SEQ ID NO: 106 - protein, 156 aa, Homo sapiens
MYRMQLLSCIALILALVTNSAPTSSSTKKTKKTQLQLEHLLLDLQMILNGINNYKNPKLTRMLTFKFYMPKKATELKQLQCLEEELKPLEEVLNLAQSKNFHLRPRDLISNINVIVLELKGSETTFMCEYADETATIVEFLNRWITFCQSIISTLT

SEQ ID NO: 107 - protein, 986 aa, Homo sapiens
MPGVARLPLLLGLLLLPRPGRPLDLADYTYDLAEEDDSEPLNYKDPCKAAAFLGDIALDEEDLRAFQVQQAVDLRRHTARKSSIKAAVPGNTSTPSCQSTNGQPQRGACGRWRGRSRSRRAATSRPERVWPDGVIPFVIGGNFTGSQRAVFRQAMRHWEKHTCVTFLERTDEDSYIVFTYRPCGCCSYVGRRGGGPQAISIGKNCDKFGIVVHELGHVVGFWHEHTRPDRDRHVSIVRENIQPGQEYNFLKMEPQEVESLGETYDFDSIMHYARNTFSRGIFLDTIVPKYEVNGVKPPIGQRTRLSKGDIAQARKLYKCPACGETLQDSTGNFSSPEYPNGYSAHMHCVWRISVTPGEKIILNFTSLDLYRSRLCWYDYVEVRDGFWRKAPLRGRFCGSKLPEPIVSTDSRLWVEFRSSSNWVGKGFFAVYEAICGGDVKKDYGHIQSPNYPDDYRPSKVCIWRIQVSEGFHVGLTFQSFEIERHDSCAYDYLEVRDGHSESSTLIGRYCGYEKPDDIKSTSSRLWLKFVSDGSINKAGFAVNFFKEVDECSRPNRGGCEQRCLNTLGSYKCSCDPGYELAPDKRRCEAACGGFLTKLNGSITSPGWPKEYPPNKNCIWQLVAPTQYRISLQFDFFETEGNDVCKYDFVEVRSGLTADSKLHGKFCGSEKPEVITSQYNNMRVEFKSDNTVSKKGFKAHFFSDKDECSKDNGGCQQDCVNTFGSYECQCRSGFVLHDNKHDCKEAGCDHKVTSTSGTITSPNWPDKYPSKKECTWAISSTPGHRVKLTFMEMDIESQPECAYDHLEVFDGRDAKAPVLGRFCGSKKPEPVLATGSRMFLRFYSDNSVQRKGFQASHATECGGQVRADVKTKDLYSHAQFGDNNYPGGVDCEWVIVAEEGYGVELVFQTFEVEEETDCGYDYMELFDGYDSTAPRLGRYCGSGPPEEVYSAGDSVLVKFHSDDTITKKGFHLRYTSTKFQDTLHSRK

SEQ ID NO: 108 - protein, 148 aa, Homo sapiens
MRGSELPLVLLALVLCLAPRGRAVPLPAGGGTVLTKMYPRGNHWAVGHLMGKKSTGESSSVSERGSLKQQLREYIRWEEAARNLLGLIEAKENRNHQPPQPKALGNQQPSWDSEDSSNFKDVGSKGKVGRLSAPGSQREGRNPQLNQQ

SEQ ID NO: 109 - protein, 170 aa, Homo sapiens
MDTRNKAQLLVLLTLLSVLFSQTSAWPLYRAPSALRLGDRIPFEGANEPDQVSLKEDIDMLQNALAENDTPYYDVSRNARHADGVFTSDFSKLLGQLSAKKYLESLMGKRVSSNISEDPVPVKRHSDAVFTDNYTRLRKQMAVKKYLNSILNGKRSSEGESPDFPEELEK

SEQ ID NO: 110 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 111 - protein, 153 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKEVHLKNASRGSAGNKNYRM

SEQ ID NO: 112 - protein, 195 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKYQPPSTNKNTKSQRRKGWPKTHPGGEQKEGTEASLQIRGKKKEQRREIGSRNAECRGKKGK

SEQ ID NO: 113 - protein, 141 aa, Homo sapiens
MGFQKFSPFLALSILVLLQAGSLHAAPFRSALESSPADPATLSEDEARLLLAALVQDYVQMKASELEQEQEREGSSLDSPRSKRCGNLSTCMLGTYTQDFNKFHTFPQTAIGVGAPGKKRDMSSDLERDHRPHVSMPQNAN

SEQ ID NO: 114 - protein, 554 aa, Homo sapiens
MTAPGAAGRCPPTTWLGSLLLLVCLLASRSITEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQDVVTKPDCNCLYPKAIPSSDPASVSPHQPLAPSMAPVAGLTWEDSEGTEGSSLLPGEQPLHTVDPGSAKQRPPRSTCQSFEPPETPVVKDSTIGGSPQPRPSVGAFNPGMEDILDSAMGTNWVPEEASGEASEIPVPQGTELSPSRPGGGSMQTEPARPSNFLSASSPLPASAKGQQPADVTGTALPRVGPVRPTGQDWNHTPQKTDHPSALLRDPPEPGSPRISSLRPQGLSNPSTLSAQPQLSRSHSSGSVLPLGELEGRRSTRDRRSPAEPEGGPASEGAARPLPRFNSVPLTDTGHERQSEGSSSPQLQESVFHLLVPSVILVLLAVGGLLFYRWRRRSHQEPQRADSPLEQPEGSPLTQDDRQVELPV

SEQ ID NO: 115 - protein, 20 aa, Artificial Sequence
  Description of Artificial Sequence Synthetic peptide
GTFVYGGCRAKRNNFKSAED

SEQ ID NO: 116 - protein, 20 aa, Artificial Sequence
  Description of Artificial Sequence Synthetic peptide
GPFFYGGCGGNRNNFDTEEY

SEQ ID NO: 117 - protein, 20 aa, Artificial Sequence
  Description of Artificial Sequence Synthetic peptide
GTFFYGGCRGKRNNFKTEEY

SEQ ID NO: 118 - protein, 20 aa, Artificial Sequence
  Description of Artificial Sequence Synthetic peptide
GTFFYGGSRGKRNNFKTEEY

SEQ ID NO: 119 - protein, 20 aa, Artificial Sequence
  Description of Artificial Sequence Synthetic peptide
GRFFYGGSRGKRNNFRTEEY

SEQ ID NO: 120 - protein, 20 aa, Artificial Sequence
  Description of Artificial Sequence Synthetic peptide
GTFFYGGSRGRRNNFRTEEY

SEQ ID NO: 121 - protein, 20 aa, Artificial Sequence
  Description of Artificial Sequence Synthetic peptide
CTFVYGGCRAKRNNFKSAED

SEQ ID NO: 122 - protein, 20 aa, Artificial Sequence
  Description of Artificial Sequence Synthetic peptide
CPFFYGGCGGNRNNFDTEEY

SEQ ID NO: 123 - protein, 20 aa, Artificial Sequence
  Description of Artificial Sequence Synthetic peptide
CTFFYGGCRGKRNNFKTEEY

SEQ ID NO: 124 - protein, 20 aa, Artificial Sequence
  Description of Artificial Sequence Synthetic peptide
CTFFYGGSRGKRNNFKTEEY

SEQ ID NO: 125 - protein, 20 aa, Artificial Sequence
  Description of Artificial Sequence Synthetic peptide
CRFFYGGSRGKRNNFRTEEY

SEQ ID NO: 126 - protein, 20 aa, Artificial Sequence
  Description of Artificial Sequence Synthetic peptide
CTFFYGGSRGRRNNFRTEEY

SEQ ID NO: 127 - protein, 20 aa, Artificial Sequence
  Description of Artificial Sequence Synthetic peptide
TFVYGGCRAKRNNFKSAEDG

SEQ ID NO: 128 - protein, 20 aa, Artificial Sequence
  Description of Artificial Sequence Synthetic peptide
PFFYGGCGGNRNNFDTEEYG

SEQ ID NO: 129 - protein, 20 aa, Artificial Sequence
  Description of Artificial Sequence Synthetic peptide
TFFYGGCRGKRNNFKTEEYG

SEQ ID NO: 130 - protein, 20 aa, Artificial Sequence
  Description of Artificial Sequence Synthetic peptide
TFFYGGSRGKRNNFKTEEYG

SEQ ID NO: 131 - protein, 20 aa, Artificial Sequence
  Description of Artificial Sequence Synthetic peptide
RFFYGGSRGKRNNFRTEEYG

SEQ ID NO: 132 - protein, 20 aa, Artificial Sequence
  Description of Artificial Sequence Synthetic peptide
TFFYGGSRGRRNNFRTEEYG

SEQ ID NO: 133 - protein, 20 aa, Artificial Sequence
  Description of Artificial Sequence Synthetic peptide
TFVYGGCRAKRNNFKSAEDC

SEQ ID NO: 134 - protein, 20 aa, Artificial Sequence
  Description of Artificial Sequence Synthetic peptide
PFFYGGCGGNRNNFDTEEYC

SEQ ID NO: 135 - protein, 20 aa, Artificial Sequence
  Description of Artificial Sequence Synthetic peptide
TFFYGGCRGKRNNFKTEEYC

SEQ ID NO: 136 - protein, 20 aa, Artificial Sequence
  Description of Artificial Sequence Synthetic peptide
TFFYGGSRGKRNNFKTEEYC

SEQ ID NO: 137 - protein, 20 aa, Artificial Sequence
  Description of Artificial Sequence Synthetic peptide
RFFYGGSRGKRNNFRTEEYC

SEQ ID NO: 138 - protein, 20 aa, Artificial Sequence
  Description of Artificial Sequence Synthetic peptide
TFFYGGSRGRRNNFRTEEYC

SEQ ID NO: 139 - protein, 1296 aa, Clostridium botulinum [sequence omitted]

SEQ ID NO: 140 - protein, 1291 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 141 - protein, 1291 aa, Clostridium botulinum
MPITINNFNYSDPVDNKNILYLDTHLNTLANEPEKAFRITGNIWVIPDRFSRNSNPNLNKPPRVTSPKSGYYDPNYLSTDSDKDPFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 142 - protein, 1276 aa, Clostridium botulinum
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTKNSRDDSTCIKVKNNRLPYVADKDSISQEIFENKIITDETNVQNYSDKFSLDESILDGQVPINPEIVDPLLPNVNMEPLNLPGEEIVFYDDITKYVDYLNSYYYLESQKLSNNVENITLTTSVEEALGYSNKIYTFLPSLAEKVNKGVQAGLFLNWANEVVEDFTTNIMKKDTLDKISDVSVIIPYIGPALNIGNSALRGNFNQAFATAGVAFLLEGFPEFTIPALGVFTFYSSIQEREKIIKTIENCLEQRVKRWKDSYQWMVSNWLSRITTQFNHINYQMYDSLSYQADAIKAKIDLEYKKYSGSDKENIKSQVENLKNSLDVKISEAMNNINKFIRECSVTYLFKNMLPKVIDELNKFDLRTKTELINLIDSHNIILVGEVDRLKAKVNESFENTMPFNIFSYTNNSLLKDIINEYFNSINDSKILSLQNKKNALVDTSGYNAEVRVGDNVQLNTIYTNDFKLSSSGDKIIVNLNNNILYSAIYENSSVSFWIKISKDLTNSHNEYTIINSIEQNSGWKLCIRNGNIEWILQDVNRKYKSLIFDYSESLSHTGYTNKWFFVTITNNIMGYMKLYINGELKQSQKIEDLDEVKLDKTIVFGIDENIDENQMLWIRDFNIFSKELSNEDINIVYEGQILRNVIKDYWGNPLKFDTEYYIINDNYIDRYIAPESNVLVLVQYPDRSKLYTGNPITIKSVSDKNPYSRILNGDNIILHMLYNSRKYMIIRDTDTIYATQGGECSQNCVYALKLQSNLGNYGIGIFSIKNIVSKNKYCSQIFSSFRENTMLLADIYKPWRFSFKNAYTPVAVTNYETKLLSTSSFWKFISRDPGWVE

SEQ ID NO: 143 - protein, 1251 aa, Clostridium botulinum
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHRFGSIAIVTFSPEYSFRFNDNCMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLRTKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKIINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTFEDNRGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNCTMNFKNNNGNNIGLLGFKADTVVASTWYYTHMRDHTNSNGCFWNFISEEHGWQEK

SEQ ID NO: 144 - protein, 1274 aa, Clostridium botulinum
MPVAINSFNYNDPVNDDTILYMQIPYEEKSKKYYKAFEIMRNVWIIPERNTIGTNPSDFDPPASLKNGSSAYYDPNYLTTDAEKDRYLKTTIKLFKRINSNPAGKVLLQEISYAKPYLGNDHTPIDEFSPVTRTTSVNIKLSTNVESSMLLNLLVLGAGPDIFESCCYPVRKLIDPDVVYDPSNYGFGSINIVTFSPEYEYTFNDISGGHNSSTESFIADPAISLAHELIHALHGLYGARGVTYEETIEVKQAPLMIAEKPIRLEEFLTFGGQDLNIITSAMKEKIYNNLLANYEKIATRLSEVNSAPPEYDINEYKDYFQWKYGLDKNADGSYTVNENKFNEIYKKLYSFTESDLANKFKVKCRNTYFIKYEFLKVPNLLDDDIYTVSEGFNIGNLAVNNRGQSIKLNPKIIDSIPDKGLVEKIVKFCKSVIPRKGTKAPPRLCIRVNNSELFFVASESSYNENDINTPKEIDDTTNLNNNYRNNLDEVILDYNSQTIPQISNRTLNTLVQDNSYVPRYDSNGTSEIEEYDVVDFNVFFYLHAQKVPEGETNISLTSSIDTALLEESKDIFFSSEFIDTINKPVNAALFIDWISKVIRDFTTEATQKSTVDKIADISLIVPYVGLALNIIIEAEKGNFEEAFELLGVGILLEFVPELTIPVILVFTIKSYIDSYENKNKAIKAINNSLIEREAKWKEIYSWIVSNWLTRINTQFNKRKEQMYQALQNQVDAIKTAIEYKYNNYTSDEKNRLESEYNINNIEEELNKKVSLAMKNIERFMTESSISYLMKLINEAKVGKLKKYDNHVKSDLLNYILDHRSILGEQTNELSDLVTSTLNSSIPFELSSYTNDKILIIYFNRLYKKIKDSSILDMRYENNKFIDISGYGSNISINGNVYIYSTNRNQFGIYNSRLSEVNIAQNNDIIYNSRYQNFSISFWVRIPKHYKPMNHNREYTIINCMGNNNSGWKISLRTVRDCEIIWTLQDTSGNKENLIFRYEELNRISNYINKWIFVTITNNRLGNSRIYINGNLIVEKSISNLGDIHVSDNILFKIVGCDDETYVGIRYFKVFNTELDKTEIETLYSNEPDPSILKNYWGNYLLYNKKYYLFNLLRKDKYITLNSGILNINQQRGVTEGSVFLNYKLYEGVEVIIRKNGPIDISNTDNFVRKNDLAYINVVDRGVEYRLYADTKSEKEKIIRTSNLNDSLGQIIVMDSIGNNCTMNFQNNNGSNIGLLGFHSNNLVASSWYYNNIRRNTSSNGCFWSSISKENGWKE

SEQ ID NO: 145 - protein, 1297 aa, Clostridium botulinum
  MOD_RES (7)..(7): Any amino acid
MPVNIKXFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYKNTGKSEQCIIVNNEDLFFIANKDSFSKDLAKAETIAYNTQNNTIENNFSIDQLILDNDLSSGIDLPNENTEPFTNFDDIDIPVYIKQSALKKIFVDGDSLFEYLHAQTFPSNIENLQLTNSLNDALRNNNKVYTFFSTNLVEKANTVVGASLFVNWVKGVIDDFTSESTQKSTIDKVSDVSIIIPYIGPALNVGNETAKENFKNAFEIGGAAILMEFIPELIVPIVGFFTLESYVGNKGHIIMTISNALKKRDQKWTDMYGLIVSQWLSTVNTQFYTIKERMYNALNNQSQAIEKIIEDQYNRYSEEDKMNINIDFNDIDFKLNQSINLAINNIDDFINQCSISYLMNRMIPLAVKKLKDFDDNLKRDLLEYIDTNELYLLDEVNILKSKVNRHLKDSIPFDLSLYTKDTILIQVFNNYISNISSNAILSLSYRGGRLIDSSGYGATMNVGSDVIFNDIGNGQFKLNNSENSNITAHQSKFVVYDSMFDNFSINFWVRTPKYNNNDIQTYLQNEYTIISCIKNDSGWKVSIKGNRIIWTLIDVNAKSKSIFFEYSIKDNISDYINKWFSITITNDRLGNANIYINGSLKKSEKILNLDRINSSNDIDFKLINCTDTTKFVWIKDFNIFGRELNATEVSSLYWIQSSTNTLKDFWGNPLRYDTQYYLFNQGMQNIYIKYFSKASMGETAPRTNFNNAAINYQNLYLGLRFIIKKASNSRNINNDNIVREGDYIYLNIDNISDESYRVYVLVNSKEIQTQLFLAPINDDPTFYDVLQIKKYYEKTTYNCQILCEKDTKTFGLFGIGKFVKDYGYVWDTYDNYFCISQWYLRRISENINKLRLGCNWQFIPVDEGWTE

SEQ ID NO: 146 - protein, 1315 aa, Clostridium tetani
MPITINNFRYSDPVNNDTIIMMEPPYCKGLDIYYKAFKITDRIWIVPERYEFGTKPEDFNPPSSLIEGASEYYDPNYLRTDSDKDRFLQTMVKLFNRIKNNVAGEALLDKIINAIPYLGNSYSLLDKFDTNSNSVSFNLLEQDPSGATTKSAMLTNLIIFGPGPVLNKNEVRGIVLRVDNKNYFPCRDGFGSIMQMAFCPEYVPTFDNVIENITSLTIGKSKYFQDPALLLMHELIHVLHGLYGMQVSSHEIIPSKQEIYMQHTYPISAEELFTFGGQDANLISIDIKNDLYEKTLNDYKAIANKLSQVTSCNDPNIDIDSYKQIYQQKYQFDKDSNGQYIVNEDKFQILYNSIMYGFTEIELGKKFNIKTRLSYFSMNHDPVKIPNLLDDTIYNDTEGFNIESKDLKSEYKGQNMRVNTNAFRNVDGSGLVSKLIGLCKKIIPPTNIRENLYNRTASLTDLGGELCIKIKNEDLTFIAEKNSFSEEPFQDEIVSYNTKNKPLNFNYSLDKIIVDYNLQSKITLPNDRTTPVTKGIPYAPEYKSNAASTIEIHNIDDNTIYQYLYAQKSPTTLQRITMTNSVDDALINSTKIYSYFPSVISKVNQGAQGILFLQWVRDIIDDFTNESSQKTTIDKISDVSTIVPYIGPALNIVKQGYEGNFIGALETTGVVLLLEYIPEITLPVIAALSIAESSTQKEKIIKTIDNFLEKRYEKWIEVYKLVKAKWLGTVNTQFQKRSYQMYRSLEYQVDAIKKIIDYEYKIYSGPDKEQIADEINNLKNKLEEKANKAMININIFMRESSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 147 - protein, 567 aa, Corynephage beta
MLVRGYVVSRKLFASILIGALLGIGAPPSAHAGADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 148 - protein, 638 aa, Pseudomonas aeruginosa
MHLTPHWIPLVASLGLLAGGSFASAAEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGKIYRVLAGNPAKHDLDIKPTVISHRLHFPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAAGECAGPADSGDALLERNYPTGAEFLGDGGDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 149 - protein, 21 aa, Artificial Sequence
  Description of Artificial Sequence Synthetic peptide
STEELRVRLASHLRKLRKRLL

SEQ ID NO: 150 - protein, 39 aa, Artificial Sequence
  Description of Artificial Sequence Synthetic polypeptide
SSVIDALQYKLEGTTRLTRKRGLKLATALSLSNKFVEGS

SEQ ID NO: 151 - protein, 5 aa, Artificial Sequence
  Description of Artificial Sequence Synthetic peptide
CSKCG

SEQ ID NO: 152 - protein, 8 aa, Artificial Sequence
  Description of Artificial Sequence Synthetic peptide
CFTKWFFC

SEQ ID NO: 153 - protein, 8 aa, Artificial Sequence
  Description of Artificial Sequence Synthetic peptide
TFTKWFFF

SEQ ID NO: 154 - protein, 27 aa, Artificial Sequence
  Description of Artificial Sequence Synthetic peptide
EELRVRLASHLRKLRKRLLRDADDLQK

SEQ ID NO: 155 - protein, 21 aa, Artificial Sequence
  Description of Artificial Sequence Synthetic peptide
GQSTEELRARLASHLRKLRKR

SEQ ID NO: 156 - protein, 16 aa, Artificial Sequence
  Description of Artificial Sequence Synthetic peptide
RLASHLRKLRKRLLRD

SEQ ID NO: 157 - DNA, 25 bp, Artificial Sequence
  Description of Artificial Sequence Synthetic oligonucleotide
gccagacuuuguuggauuugaaatt

SEQ ID NO: 158 - RNA, 27 nt, Artificial Sequence
  Description of Artificial Sequence Synthetic oligonucleotide
aauuucaaauccaacaaagucuggcuu

SEQ ID NO: 159 - RNA, 25 nt, Artificial Sequence
  Description of Artificial Sequence Synthetic oligonucleotide
ggagggcuuucuuuguguauuugcc

SEQ ID NO: 160 - RNA, 27 nt, Artificial Sequence
  Description of Artificial Sequence Synthetic oligonucleotide
ggcaaauacacaaagaaagcccucccc

SEQ ID NO: 161 - protein, 8 aa, Artificial Sequence
  Description of Artificial Sequence Synthetic peptide
RRRRRRRR

SEQ ID NO: 162 - protein, 9 aa, Artificial Sequence
  Description of Artificial Sequence Synthetic peptide
RRRRRRRRR

SEQ ID NO: 163 - protein, 85 aa, Artificial Sequence
  Description of Artificial Sequence Synthetic polypeptide
XXXXXXXXXXXXXXXXXXXXXXXXXXXXXXXXXXXXXXXXFXYGGXXXXXXXXXXXXXXXXXXXXXXXXXXXXXXXXXXXXXXXX

(19) United States
(12) Patent Application Publication
(10) Pub. No.: US 2011/0111056 A1
(43) Pub. Date: May 12, 2011

(54) PEPTIDE DICER SUBSTRATE AGENTS AND METHODS FOR THEIR SPECIFIC INHIBITION OF GENE EXPRESSION

(75) Inventors: Sujit K. Basu, Newton, MA (US); Bob D. Brown, Millington, NJ (US)

(73) Assignee: Dicerna Pharmaceuticals, Inc., Watertown, MA (US)

(21) Appl. No.: 12/793,442

(22) Filed: Jun. 3, 2010

Related U.S. Application Data
(60) Provisional application No. 61/183,818, filed on Jun. 3, 2009.
(60) Provisional application No. 61/183,815, filed on Jun. 3, 2009.

(51) Int. Cl. A61K 33/24; C07K 2/00; C12N 5/00; C12P 21/00; A61K 38/14; A61P 35/04; A61P 31/12; A61P 31/04; A61P 31/10; A61P 33/02; A61P 33/10; A61P 33/12

(52) U.S. Cl. 424/649; 530/322; 435/375; 435/68.1; 514/20.9

(57) ABSTRACT

This invention relates to compounds, compositions, and methods useful for reducing a target RNA and protein levels via use of Dicer substrate siRNA (DsiRNA)-peptide conjugates.

### CROSS-REFERENCE TO RELATED APPLICATIONS

The present application is related to and claims priority under 35 U.S.C. §119(e) to U.S. provisional patent application No. 61/183,815, filed Jun. 3, 2009, and to U.S. provisional patent application No. 61/183,818, filed Jun. 3, 2009. The entire teachings of these applications are incorporated herein by reference.

### FIELD OF THE INVENTION

The invention relates to peptide-dicer substrate conjugates and their method of use.

### BACKGROUND OF THE INVENTION

Identification of peptide aptamers is important in view of a need for safe, efficient delivery of therapeutic molecules. Peptide aptamers have been described (reviewed in Beldhoen 2008 Int. J. Mol. Sci. 9:1276-1320; Moschos et al. 2007 Biochemical Society Transaction Vol. 35, pt 4: 807-810).

### BRIEF SUMMARY OF THE INVENTION

The present invention is directed to compositions that contain double stranded RNA (“dsRNA”) conjugated to peptides and methods for preparing them. The dsRNAs of the invention are double stranded RNA, small interfering RNA (siRNA) and Dicer substrate siRNAs (“DsiRNAs”) with structures that are optimized, by conjugation to a peptide, for efficient delivery and/targeting and to act as effective and highly potent inhibitory agents, optionally possessing extended duration of inhibitory effect.

The invention provides dsRNA-peptide conjugates for improved delivery and targeting.

In one embodiment, the invention provides an isolated double stranded ribonucleic acid (dsRNA) composition comprising a first oligonucleotide strand having a 5′ terminus and a 3′ terminus and a second oligonucleotide strand having a 5′ terminus and a 3′ terminus, wherein the first strand and the second strand have a length that is at least 16 and at most 50 nucleotides in length, wherein the peptide is conjugated to the dsRNA and wherein the dsRNA-peptide conjugate binds to a target.

In another embodiment, the invention provides an isolated double stranded ribonucleic acid (dsRNA) composition comprising a first oligonucleotide strand having a 5′ terminus and a 3′ terminus and a second oligonucleotide strand having a 5′ terminus and a 3′ terminus, wherein the first strand and the second strand have a length that is at least 16 and at most 50 nucleotides in length, wherein the peptide is conjugated to the dsRNA and wherein the dsRNA-peptide conjugate is internalized by a target cell.

In one aspect, the first strand and the second strand have a length that is at least 25 and at most 35 nucleotides, at least 19 and at most 35 nucleotides, at least 19 and at most 24 nucleotides, at least 25 and at most 30 nucleotides, at least 26 and at most 30 nucleotides or at least 21 and a most 23 nucleotides.

In another aspect, the second strand comprises an overhang at the 3′ terminus.

In another aspect the first strand comprises an overhang at the 3′ terminus.

In another aspect at least one of the second strand and the first strand comprises an overhang at the 3′ terminus.

In another aspect the nucleotides of the 3′ overhang of the first and/or second strand comprise a modified nucleotide.

In another aspect the 3′ overhang(s) is/are 1-5 nucleotides in length.

In another aspect each of the first and second strands consists of the same number of nucleotide residues.

In another aspect the ultimate residue of the 5′ terminus of the first strand and the ultimate residue of the 3′ terminus of the second strand form a mismatched base pair.

In another aspect the ultimate residue of the 3′ terminus of the first strand and the ultimate residue of the 5′ terminus of the second strand form a mismatched base pair.

In another aspect the ultimate and penultimate residues of the 5′ terminus of the first strand and the ultimate and penultimate residues of the 3′ terminus of the second strand form two mismatched base pairs.

In an other aspect the ultimate and penultimate residues of the 3′ terminus of the first strand and the ultimate and penultimate residues of the 5′ terminus of the second strand form two mismatched base pairs.

In another aspect the peptide comprises 6-100 amino acids.

In another aspect the peptide comprises 10-50 amino acids.

In another aspect the peptide comprises 15-30 amino acids.

In another aspect the peptide comprises 10 amino acids.

In another aspect the dsRNA-peptide conjugate binds to a receptor.

In another aspect the dsRNA-peptide conjugate binds at least one member of the LDL receptor family.

In another aspect the peptide is a PAR ligand.

In another aspect the peptide is a PAR1 ligand.

In another aspect the peptide is a growth factor ligand.

In another aspect the peptide is an insulin or insulin-like growth factor ligand.

In another aspect the peptide is an IGF-1 ligand.

In another aspect the peptide is a hormone ligand.

In another aspect the peptide is a PTH ligand.

In another aspect the peptide is a PTH-1 ligand.

In another aspect the dsRNA-peptide conjugate binds to a receptor binding protein.

In another aspect the peptide is conjugated to the dsRNA with a stable linker.

In another aspect the stable linker comprises a homobifunctional crosslinker.

In another aspect the stable linker comprises a hetero-bifunctional crosslinker.

In another aspect the stable linker comprises a trifunctional crosslinker.

In another aspect the peptide is conjugated to the dsRNA with a cleavable linker.

In another aspect the cleavable linker comprises a disulfide linker.

In another aspect the peptide is conjugated to the dsRNA with a carbon linker.

In another aspect the carbon linker comprises no more than eighteen carbons

In another aspect the carbon linker comprises 6 carbons.

In another aspect the peptide and the dsRNA are conjugated without a linker.

In another aspect the peptide is conjugated to the 3′ end of the first strand of the dsRNA.

In another aspect the peptide is conjugated to the 3′ end of the second strand of the dsRNA.

In another aspect the peptide is conjugated to the 5′ end of the first strand of the dsRNA.

In another aspect the peptide is conjugated to the 5′ end of the second strand of the dsRNA.

In another aspect the peptide is conjugated to the 5′ end of the first strand and the 5′ end of the second strand of the dsRNA.

In another aspect the peptide is conjugated to the 5′ end of the first strand and the 3′ end of the second strand of the dsRNA.

In another aspect the peptide is conjugated to the 3′ end of the first strand and the 3′ end of the second strand of the dsRNA.

In another aspect the peptide is conjugated to the 3′ end of the first strand and the 5′ end of the second strand of the dsRNA.

In another aspect at least one peptide is conjugated internally to the first strand of the dsRNA.

In another aspect at least one peptide is conjugated internally to the second strand of the dsRNA.

In another aspect at least one peptide is conjugated internally to the first strand and at least one peptide is conjugated internally to the second strand of the dsRNA.

In another aspect at least two peptides are conjugated to the dsRNA.

In another aspect the at least two peptides are identical.

In another aspect the at least two peptides are not identical.

In another aspect the composition further comprises at least one dye molecule, and the dye molecule is conjugated to at least one of the dsRNA and the peptide.

In another aspect the dye molecule is polyaromatic.

In another aspect the dye is a fluorescent dye.

In another aspect the composition further comprises a therapeutic agent.

In another aspect the therapeutic agent is an anticancer drug.

In another aspect the anticancer drug is selected from the group consisting of: paclitaxel, tamoxifen, cisplatin, doxorubicin and vinblastine.

In another aspect the therapeutic agent is a drug to treat a metabolic disease or disorder.

In another aspect the peptide comprises a portion of a targeting moiety of a toxin.

In another aspect the neurotoxin is a clostridial neurotoxin.

In another aspect the composition further comprises at least one delivery peptide.

In another aspect starting from the first nucleotide (position 1) at the 3′ terminus of the first oligonucleotide strand of the dsRNA, position 1, 2 and/or 3 is/are substituted with a modified nucleotide.

In another aspect the modified nucleotide is a deoxyribonucleotide.

In another aspect one or both of the first and second oligonucleotide strands comprises a 5′ phosphate.

In another aspect at least one nucleotide of the first or second strand is modified.

In another aspect the modified nucleotide residues are selected from the group consisting of 2′-O-methyl, 2′-methoxyethoxy, 2′-fluoro, 2′-allyl, 2′-O-[2-(methylamino)-2-oxoethyl], 4′-thio, 4′-CH₂—O-2′-bridge, 4′-(CH₂)₂—O-2′-bridge, 2′-LNA, 2′-amino and 2′-O—(N-methlycarbamate).

In another aspect the dsRNA is cleaved endogenously in the cell by Dicer.

In another aspect the amount of the isolated double stranded nucleic acid sufficient to reduce expression of the target gene is selected from the group consisting of 1 nanomolar or less, 200 picomolar or less, 100 picomolar or less, 50 picomolar or less, 20 picomolar or less and 10 picomolar or less in the environment of the cell.

In another aspect the first and second strands are joined by a chemical linker.

In another aspect the 3′ terminus of the first strand and the 5′ terminus of the second strand are joined by a chemical linker.

In another aspect a nucleotide of the second or first strand is substituted with a modified nucleotide that directs the orientation of Dicer cleavage.

In another aspect the isolated composition comprises a modified nucleotide selected from the group consisting of a deoxyribonucleotide, a dideoxyribonucleotide, an acyclonucleotide, a 3′-deoxyadenosine (cordycepin), a 3′-azido-3′-deoxythymidine (AZT), a 2′,3′-dideoxyinosine (ddI), a 2′,3′-dideoxy-3′-thiacytidine (3TC), a 2′,3′-didehydro-2′,3′-dideoxythymidine (d4T), a monophosphate nucleotide of 3′-azido-3′-deoxythymidine (AZT), a 2′,3′-dideoxy-3′-thiacytidine (3TC) and a monophosphate nucleotide of 2′,3′-didehydro-2′,3′-dideoxythymidine (d4T), a 4-thiouracil, a 5-bromouracil, a 5-iodouracil, a 5-(3-aminoallyl)-uracil, a 2′-O-alkyl ribonucleotide, a 2′-O-methyl ribonucleotide, a 2′-amino ribonucleotide, a 2′-fluoro ribonucleotide, and a locked nucleic acid.

In another embodiment, the isolated composition comprises a phosphate backbone modification selected from the group consisting of a phosphonate, a phosphorothioate and a phosphotriester.

In another aspect the modified nucleotide residue of the 3′ terminus of the first strand is selected from the group consisting of a deoxyribonucleotide, an acyclonucleotide and a fluorescent molecule.

In another aspect at least one of the nucleotides of the first strand and at least one of the nucleotides of the second strand form a mismatched base pair.

In another aspect the delivery peptide has an amino acid sequence selected from the group consisting of SEQ ID NO: 1-89.

In another aspect the composition is a pharmaceutical composition.

In another embodiment, the invention provides for a method for reducing expression of a target gene in a cell, comprising: contacting a cell with the isolated compositions of the invention, in an amount effective to reduce expression of a target gene in a cell in comparison to a reference dsRNA.

In another embodiment, the invention provides for a method for selectively inhibiting the growth of a cell comprising contacting a cell with an amount of the isolated compositions of the invention sufficient to inhibit the growth of the cell.

In another embodiment, the invention provides for a method for reducing expression of a target gene in an animal, comprising: treating an animal with the isolated compositions of the invention in an amount effective to reduce expression of a target gene in a cell of the animal in comparison to a reference dsRNA.

In one aspect the isolated composition possesses enhanced pharmacokinetics when compared to an appropriate control dsRNA.

In another aspect the dsRNA possesses enhanced pharmacodynamics when compared to an appropriate control dsRNA.

In another aspect the dsRNA possesses reduced toxicity when compared to an appropriate control dsRNA.

In another aspect the dsRNA possesses enhanced intracellular uptake when compared to an appropriate control dsRNA.

In another embodiment, the invention provides for a pharmaceutical composition for reducing expression of a target gene in a cell of a subject comprising the isolated compositions of the invention in an amount effective to reduce expression of a target gene in a cell in comparison to a reference dsRNA and a pharmaceutically acceptable carrier.

In another embodiment, the invention provides for a method of synthesizing a dsRNA-peptide conjugate of the invention, comprising chemically or enzymatically synthesizing the dsRNA.

In another embodiment, the invention provides for a kit comprising the dsRNA-peptide conjugate of the invention and instructions for its use.

### BRIEF DESCRIPTION OF THE DRAWINGS

FIG. 1 (A-G) presents exemplary structures of dsRNA-peptide conjugates useful according to the invention. “P”=a peptide according to the invention (A-blunt-blunt), (B and C-blunt-overhang), (D and E-asymmetric) and (F and G-mismatched ends).

FIG. 2 shows exemplary sequences of HPRT1- and KRAS-targeting dsRNAs of the invention. Underlined residues indicate positions of 2′-O-methyl modifications. Arrows indicate projected sites of dicer enzyme cleavage within the dsRNAs, while dashed lines indicate the projected position of Argonaute2-mediated cleavage within a corresponding target RNA sequence.

FIG. 3 shows exemplary peptide sequences of the peptide-conjugated dsRNAs of the invention. Targets bound by peptides are also listed.

FIG. 4 schematically depicts exemplary DsiRNA-peptide conjugates of the invention, with size shifts of properly conjugated molecules shown in lanes 2, 3, 5 and 6 for respective DsiRNA-peptide conjugates numbered 2, 3, 5 and 6. Arrowheads in schematics indicate projected dicer enzyme cleavage sites within the DsiRNA and DsiRNA-peptide conjugates.

FIG. 5 schematically depicts additional exemplary DsiRNA-peptide conjugates of the invention, with size shifts indicating properly conjugated molecules shown in lanes 2 and 3 for the DsiRNA-peptide conjugates numbered 2 and 3. Arrowheads in schematics indicate projected dicer enzyme cleavage sites within the DsiRNA and DsiRNA-peptide conjugates.

FIG. 6 schematically depicts further exemplary DsiRNA-peptide conjugates of the invention, with size shifts of properly conjugated molecules shown in lanes 2, 3, 4 and 5 for respective DsiRNA-peptide conjugates numbered 2, 3, 4 and 5. Arrowheads in schematics indicate projected dicer enzyme cleavage sites within the DsiRNA and DsiRNA-peptide conjugates.

FIG. 7 schematically depicts exemplary DsiRNA-peptide conjugates, including cleavable peptide conjugates, of the invention, with size shifts of properly conjugated molecules shown in lanes 2, 3, 4 and 5 for respective DsiRNA-peptide conjugates numbered 2, 3, 4 and 5. Arrowheads in schematics indicate projected dicer enzyme cleavage sites within the DsiRNA and DsiRNA-peptide conjugates.

FIG. 8 schematically depicts an exemplary DsiRNA-cyclic peptide conjugate of the invention, with a size shift indicating a properly conjugated molecule shown in lane 2 for the DsiRNA-peptide conjugate numbered 2. Arrowheads in schematics indicate projected dicer enzyme cleavage sites within the DsiRNA and DsiRNA-peptide conjugates.

FIG. 9 schematically depicts an exemplary DsiRNA-peptide conjugate of the invention, with results of Dicer processing assays shown for both DsiRNA and DsiRNA-peptide conjugates.

FIG. 10 shows histogram data demonstrating that a transfected DsiRNA-peptide conjugate was an effective gene silencing agent that retained potency in vitro. Transfection assays were performed in HeLa cells.

FIG. 11 demonstrates serum stability of exemplary DsiRNA-peptide conjugates, with half-lives indicated.

FIG. 12 shows histogram data demonstrating that exemplary DsiRNA-peptide conjugates showed target gene silencing efficacy in vitro in the absence of transfection vehicle, with improved delivery observed with increasing DsiRNA-peptide conjugate concentration. Assays were performed in HeLa cells.

FIG. 13 shows histogram data demonstrating that exemplary DsiRNAs and DsiRNA-peptide conjugates knocked down target gene in HepG2 cells in vitro, in the absence of transfection vehicle. DsiRNA, DsiRNA-peptides and peptides were administered at 5 μM concentrations.

FIG. 14 shows IC₅₀ curve data demonstrating that exemplary DsiRNAs and DsiRNA-peptide conjugates knocked down target gene in HepG2 cells in vitro, in the absence of transfection vehicle. Schematics of tested agents are also shown.

### DETAILED DESCRIPTION OF THE INVENTION

The present invention is directed to compositions that contain double stranded RNA (“dsRNA”) comprising a peptide capable of enhancing the delivery and/or biodistribution or targeting of a dsRNA to a target and adding further functionality and/or enhancing, e.g. pharmacokinetics or pharmacodynamics of such agents as compared to dsRNA molecules that do not comprise a peptide as described herein. The present invention is also directed to methods of preparing dsRNAs comprising a peptide that are capable of reducing the level and/or expression of genes in vivo or in vitro.

The invention provides for novel dsRNA peptide conjugates.

The invention also provides for novel dsRNA-peptide conjugates for targeting dsRNA to a specific tissue. The peptide based targeting described herein occurs via highly specific binding of the targeting peptide to a surface marker on a tissue or tumor of interest. This specificity of peptide binding provides the dsRNA-peptide conjugates of the invention with an increased ability to target the dsRNA to a target in a highly specific, selective and efficient manner that is advantageous to dsRNA targeting methods or agents known in the art.

The invention provides the following advantages. The invention provides for delivery peptides that enhance delivery of a dsRNA of the invention. The invention provides for delivery peptides that are close to neutral or are neutral. Nucleic acids conjugated to cationic peptides, for example. TAT (Tat⁴⁸⁻⁶⁰), penetratin (Antp⁴³⁻⁵⁸, oligoarginine (R8, R9), etc.) are known in the art. Unlike the peptides of the invention which are neutral or close to neutral, cationic peptide conjugation is especially disadvantageous for dsRNA conjugation due to the polyanionic nature of nucleic acids.

The peptides of the invention are also advantageous over the peptides known in the art because the peptides described herein, do not need to be linked to the dsRNA via a cleavable linker but can be conjugated to a dsRNA via a stable linker, since dicer enzyme will process the dsRNA-peptides of the invention to produce the siRNA molecule suitable for processing in the RISC pathway. This is especially advantageous for pharmaceutical compositions due to improved stability of stable linkers (cleavable linkers may cleave during manufacturing and/or shelf storage thereby losing their functionality).

### DEFINITIONS

The invention provides improved compositions and methods for reducing expression of a target gene in a cell, involving contacting a target, with an isolated dsRNA in an amount effective to reduce expression of a target gene in a cell. The dsRNA molecules of the invention comprise a peptide, as defined herein to provide a dsRNA-peptide conjugate. The peptide enhances the delivery and/or biodistribution or targeting of a dsRNA to a target RNA and add further functionality, e.g. pharmacokinetics or pharmacodynamics as compared to dsRNA agents of corresponding length that do not contain a pattern of modified nucleotides.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The present invention features one or more dsRNA molecules conjugated to one or more peptides according to the invention and methods of using these dsRNA molecules to modulate the levels of an RNA or encoded protein of interest.

A dsRNA-peptide of the invention can be cleaved by dicer and can inhibit expression of a target RNA.

A “peptide” as used herein includes a “delivery peptide” and a “targeting peptide.”

A “peptide” as used herein means a linear peptide, a branched peptide or a cyclic peptide.

The present invention further relates to the use of a peptide for transporting a dsRNA to a desired target, for example a cell or a receptor on or internal to a cell, a desired target tissue or a desired target cell.

In accordance with the present invention, the desired site may be, for example and without limitation, the brain, the adrenal or other sites outside the brain (e.g., an extracranial site) such as for example, the kidney, the liver, the pancreas, the heart, the spleen, the gastrointestinal (GI) tract (e.g., stomach, intestine, colon), the eyes, the lungs, skin, adipose, muscle, lymph nodes, bone marrow, the urinary and reproductive systems (ovary, breasts, testis, prostrate), placenta, blood cells and combination thereof. Therefore, the desired target site may be one or more site selected from the group consisting of the brain, the adrenal or other sites outside the brain (e.g., an extracranial site) such as for example, the kidney, the liver, the pancreas, the heart, the spleen, the gastrointestinal (GI) tract (e.g., stomach, intestine, colon), the eyes, the lungs, skin, adipose, muscle, lymph nodes, bone marrow, the urinary and reproductive systems (ovary, breasts, testis, prostrate), placenta, blood cells and combination thereof.

A “target cell” means any cell as defined herein, for example a cell derived from or present in any organ including but not limited to the brain, the adrenal or other sites outside the brain (e.g., an extracranial site) such as for example, the kidney, the liver, the pancreas, the heart, the spleen, the gastrointestinal (GI) tract (e.g., stomach, intestine, colon), the eyes, the lungs, skin, adipose, muscle, lymph nodes, bone marrow, the urinary and reproductive systems (ovary, breasts, testis, prostrate), placenta, blood cells and a combination thereof.

As used herein, a “delivery peptide” means a peptide that is neutral or essentially neutral. “Essentially neutral” means having a net charge of +5 or less, for example, +5, +4, +3, +2, +1 or zero.

A “net charge” according to the invention is determined according to methods known in the art. For example, the net charge as defined herein is determined by obtaining the net charge of the total number of cationic amino acids (lysine, arginine, histidine) and the total number of anionic amino acids (aspartic acid and glutamic acid.)

As used herein, “delivery peptide” means at least 6 amino acids wherein the peptide has a net charge of about +5 or less (for example, +5, +4, +3, +2, +1 or zero). In one aspect, a peptide is 6-100 amino acids, for example, 6, 7, 8, 9, 10, 15, 20, 25, 30, 35, 40, 45, 50, 55, 60, 65, 70, 75, 80, 85, 90, 95 or 100, and has a net charge of about +5 or less. In another embodiment, a peptide is 10-50 amino acids (for example, 10, 15, 20, 25, 30, 35, 40, 45 or 50 amino acids) and has a net charge of about +5 or less. In another embodiment, a peptide is 15-30 amino acids (for example, 15, 16, 17, 18, 19, 20, 21, 22, 23, 24, 25, 26, 27, 28, 29 or 30 amino acids) and has a net charge of about +5 or less.

A “delivery peptide” according to the invention includes a peptide that is at least 6 amino acids and is a neutral peptide. In one aspect, a peptide is 6-100 amino acids, for example, 6, 7, 8, 9, 10, 15, 20, 25, 30, 35, 40, 45, 50, 55, 60, 65, 70, 75, 80, 85, 90, 95 or 100, and has no net charge. In another embodiment, a peptide is 10-50 amino acids (for example, 10, 11, 12, 13, 14, 15, 20, 25, 30, 35, 40, 45 or 50 amino acids) and has no net charge. In another embodiment, a peptide is 15-30 amino acids (for example, 15, 16, 17, 18, 19, 20, 21, 22, 23, 24, 25, 26, 27, 28, 29 or 30 amino acids) and has no net charge.

A “delivery peptide” according to the invention also means a peptide that is at least 6 and no more than 19 amino acids, wherein the peptide has a net charge of about +5 or less (for example, +5, +4, +3, +2, +1, or zero).

As used herein, “delivery peptide” means at least 6 amino acids wherein the peptide has a net charge of about +5 or less (for example, +5, +4, +3, +2, +1 or zero) and wherein the peptide has at least one anionic amino acid. In one aspect, a peptide is 6-100 amino acids, for example, 6, 7, 8, 9, 10, 15, 20, 25, 30, 35, 40, 45, 50, 55, 60, 65, 70, 75, 80, 85, 90, 95 or 100, and has a net charge of about +4 or less. In another embodiment, a peptide is 10-50 amino acids (for example, 10, 15, 20, 25, 30, 35, 40, 45 or 50 amino acids) and has a net charge of about +5 or less. In another embodiment, a peptide is 15-30 amino acids (for example, 15, 16, 17, 18, 19, 20, 21, 22, 23, 24, 25, 26, 27, 28, 29 or 30 amino acids) and has a net charge of about +5 or less.

As used herein, “at least one anionic amino acid” means at least one of glutamic acid (E) or aspartic acid (D). For example, XXXEXX or XXXDXX or XXDXEXX or XXXEDXX wherein X is any amino acid, wherein the peptide has a net charge of +5 or less.

A peptide that has no net charge means a “neutral peptide.”

As used herein, a “neutral peptide” has a net charge that is approximately zero at neutral pH (for example pH 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4 or 8.5).

A “neutral peptide” also includes a peptide that has a net charge that is approximately zero at neutral pH and/or has an isoelectric point (pI) of about pH 7 (for example pH 6. 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4 or 8.5).

Positively charged amino acids are Lysine (Lys, K), Arginine (Arg, R) and Histidine (His, H). Negatively charged amino acids are Aspartic acid or aspartate (Asp, D), Glutamic acid or glutamate (Glu, E). (Reference: Lehninger Principles of Biochemistry, 3^{rd} Ed., 2000. Edited by David L. Nelson and Michael M. Cox, Worth Publishers, New York, N.Y.)

A “delivery peptide” according to the invention is an amino acid sequence that can deliver a dsRNA to the appropriate target RNA when conjugated to a dsRNA of the invention.

A “delivery peptide” also means an amino acid sequence that can transport a dsRNA across a cell membrane when the dsRNA is conjugated to the peptide.

A “delivery peptide” that is useful according to the invention increases the internalization of a dsRNA to a target cell when the peptide is conjugated to the dsRNA, as compared to a dsRNA that is not conjugated to a peptide.

A “delivery peptide” that is useful according to the invention increases the delivery of a dsRNA to a target RNA when the peptide is conjugated to the dsRNA, as compared to a dsRNA that is not conjugated to a peptide.

As used herein, “increases” means delivery of a peptide-dsRNA to a target RNA is 1, 2, 3, 4, 5, 10, 15, 20, 25, 40, 35, 40, 45, 50, 100, 1000 or 10,000-fold or more greater than delivery of a dsRNA that is not conjugated to a peptide.

As used herein, “increases” means delivery of a peptide-dsRNA conjugate to a target is 1, 5, 10, 15, 20, 25, 30, 35, 40, 45, 50, 55, 60, 65, 70, 75, 80, 85, 90, 95, 99 or 100% greater than delivery of a dsRNA that is not conjugated to a peptide.

“Delivery” of a dsRNA, a peptide or a dsRNA-peptide conjugate is assessed by internalization or uptake assays described hereinbelow.

In another embodiment a “peptide” as used herein means a “targeting peptide” that is 6-100 amino acids, for example, 6, 7, 8, 9, 10, 15, 20, 25, 30, 35, 40, 45, 50, 55, 60, 65, 70, 75, 80, 85, 90, 95 or 100, and that binds to a target cell.

A “targeting peptide” according to the invention binds specifically to a target or a binding site when conjugated to a dsRNA as defined herein.

As used herein, “specifically binds” means via hydrogen bonding or electrostatic attraction to a receptor of interest.

In one aspect, the target is a receptor or a receptor binding protein.

In one aspect, the target or binding site or receptor is on the surface of a cell.

In another aspect, the target or binding site or receptor is internal, for example, in a cell, (for example in the cytoplasm, in the nucleus or on the surface of the nucleus.)

In another aspect, the target or binding site or receptor is naked in solution.

“Specific binding” is determined by a binding assay known in the art and as defined herein (See for example US20080064092 and US2009004174). In one embodiment, specific binding is determined by comparing the binding of a dsRNA-delivery peptide to the stated, corresponding receptor to the binding of the dsRNA-peptide to other receptors, wherein all receptors are present in a mixture. An increase, as defined herein, in binding to the stated receptor, as compared to other receptors, is indicative of specific binding.

In one embodiment, specific binding is determined by comparing the binding of a dsRNA-delivery peptide to the stated cell to the binding of the dsRNA-peptide to other cells, wherein all cells are present in a mixture. An increase, as defined herein, in binding to the stated cell, as compared to other cells, is indicative of specific binding.

“Specific binding” is determined in vitro by determining the binding of a dsRNA-peptide to a naked receptor in solution or in vivo by determining the binding of a dsRNA-peptide to a cell.

As used herein, a “receptor” includes cell surface receptors, naked receptors in solution and receptors that are internal to a cell, for example in the cytoplasm, the nucleus or on the surface of the nucleus.

As used herein, a “receptor binding protein” means

A “targeting peptide” as used herein, can do at least one of cross a cell membrane when conjugated to a dsRNA, transport a dsRNA across a cell membrane when conjugated to a dsRNA according to the invention and bind a receptor for the ligand, for example a cell surface receptor, when conjugated to a dsRNA.

In one aspect, a “targeting peptide” is conjugated to a translocation domain or a portion thereof, for example a translocation domain of a neurotoxin.

As used herein, a translocation domain refers to an amino acid sequence that facilitates penetration and/or internalization of a protein.

As used herein, a “portion thereof” means an amino acid sequence that is sufficient to maintain the stated function, for example directing cell entry or facilitating cell surface binding, for example cell surface receptor binding, as defined herein. A “portion thereof” also means 1% or more, for example, 1, 5, 10, 20, 25, 30, 35, 40, 45, 50, 55, 60, 65, 70, 75, 80, 85, 90, 95 or 99% of the complete amino acid sequence.

In one aspect, the targeting peptide is capable of internalization (e.g. by direct penetration or by and endocytic pathway that requires endosome formation and is also referred to as receptor-mediated endocytosis.)

“Binding” of a dsRNA, a peptide or a dsRNA-peptide conjugate is assessed by a ligand binding assay.

In one embodiment the binding affinity of the peptide or dsRNA-peptide conjugate for the corresponding receptor is about 100 μM. In another embodiment the binding affinity of the peptide or dsRNA-peptide conjugate for the corresponding receptor is about 1 μM. In another embodiment the binding affinity of the peptide or dsRNA-peptide conjugates for the corresponding receptor is about 100 nM. In another embodiment the binding affinity of the peptide or dsRNA-peptide conjugate for the corresponding receptor is about 10 nM. In another embodiment the binding affinity of the peptide or dsRNA-peptide conjugate for the corresponding receptor is about 5 nM. In another embodiment the binding affinity of the peptide or dsRNA-peptide conjugate for the corresponding receptor is about 1 nM. In another embodiment the binding affinity of the peptide or dsRNA-peptide conjugate for the corresponding receptor is about 0.1 nM or less (Gauguin et al., J Biol. Chem. 2008; 283:2604-2613; Grupping et al., Endocrinology 1997; 138(10):4064-4068; and Stone, Chervin and Kranz, Immunology. 2009; 126(2):165-76.)

In one embodiment, a “targeting peptide” means 6-100 amino acids, for example, 6, 7, 8, 9, 10, 15, 20, 25, 30, 35, 40, 45, 50, 55, 60, 65, 70, 75, 80, 85, 90, 95 or 100 amino acids, that bind to a target cell and that comprises at least a portion of an amino acid sequence of interest, for example, the amino acid sequence of a target peptide.

A peptide that is useful according to the invention increases the targeting of a dsRNA to a cell when the peptide is conjugated to the dsRNA as compared to a dsRNA that is not conjugated to a peptide.

As used herein, “increases” means targeting of a peptide-dsRNA conjugate to a cell is 1, 2, 3, 4, 5, 10, 15, 20, 25, 40, 35, 40, 45, 50, 100, 1000 or 10,000-fold or more greater than targeting of a dsRNA that is not conjugated to a peptide.

As used herein, “increases” means targeting of a peptide-dsRNA conjugate to a cell is 1, 5, 10, 15, 20, 25, 30, 35, 40, 45, 50, 55, 60, 65, 70, 75, 80, 85, 90, 95, 99 or 100% greater than targeting of a dsRNA that is not conjugated to a peptide.

As used herein, “increases” means targeting, as defined hereinbelow, of a peptide-dsRNA conjugate to a cell requires less dsRNA (a lower dose of dsRNA) as compared to the amount or dose of an identical dsRNA that is not conjugated to a peptide and that is required to achieve an equivalent level of binding, association or internalization, as determined by the IC₅₀, in the assays described hereinbelow. For example, the IC₅₀ for a dsRNA-peptide conjugate that is required to achieve a 50% reduction in RNA/gene expression is decreased as compared to the IC₅₀ for an identical dsRNA that is not conjugated to a peptide, as measured in vivo or in vitro (see for example Hefner et al. J Biomol Tech. 2008 September: 19(4) 231-237; Zimmermann et al. Nature. 2006 May 4: 441(7089):111-114; Durcan et al. Mol. Pharm. 2008 July-August; 5(4):559-566; Heidel et al. Proc Natl Acad Sci USA. 2007 Apr. 3: 104(14):5715-5721).

As used herein, “decreased” means that the IC₅₀ for a dsRNA-peptide conjugate is 1, 2, 3, 4, 5, 10, 15, 20, 25, 40, 35, 40, 45, 50, 100, 1000 or 10,000-fold or more less than the IC₅₀ for an identical dsRNA that is not conjugated to a peptide.

As used herein, “decreased” means that the IC₅₀ for a dsRNA-peptide conjugate is 1, 5, 10, 15, 20, 25, 30, 35, 40, 45, 50, 55, 60, 65, 70, 75, 80, 85, 90, 95, 99 or 100% less than the IC₅₀ for an identical dsRNA that is not conjugated to a peptide.

In one embodiment increased targeting of a dsRNA-peptide conjugate as compared to dsRNA alone as expressed by a binding coefficient, K_{d}, is about 25%. In another embodiment the increased targeting of a dsRNA-peptide conjugate as compared to a dsRNA alone is about 100%, i.e., the dsRNA-peptide conjugate exhibits about a 2-fold increase in binding affinity (i.e., decreased K_{d}) compared to dsRNA alone. In another embodiment the dsRNA-peptide conjugate exhibits about a 5-fold increase in binding affinity compared to dsRNA alone. In another embodiment the dsRNA-peptide conjugate exhibits about a 10-fold increase in binding affinity compared to dsiRNA alone. In another embodiment the dsRNA-peptide conjugate exhibits about a 100-fold increase in binding affinity compared to dsRNA alone. In another embodiment the dsRNA-peptide conjugate exhibits about a 1,000-fold or more increase in binding affinity compared to dsRNA alone.

“Binding” is determined by a binding assay known in the art and as defined herein. In one embodiment, binding is determined by determining the binding of a dsRNA-delivery peptide to the stated receptor.

In another embodiment, binding is determined by determining the binding of a dsRNA-delivery peptide to the stated cell wherein all cells are present in a mixture.

“Binding” is determined in vitro by determining the binding of a dsRNA-peptide to a naked receptor in solution or in vivo by determining the binding of a dsRNA-peptide to a cell.

As used herein, “targeting” means preferential or specific binding or association or internalization of a dsRNA peptide conjugate to a receptor of interest, as compared to another receptor in a mixture of receptors. As used herein “targeting” encompasses preferential or specific binding or association or internalization of a dsRNA peptide conjugate to a receptor of interest on a cell, as compared to another receptor on a cell, in a mixture of cells. As used herein “targeting” encompasses preferential or specific binding or association or internalization of a dsRNA peptide conjugate to a cell, as compared to another cell, in a mixture of cells. That is, “targeting” according to the invention, is determined or measured both in vitro and in vivo.

“Targeting” also means transport or delivery of a “peptide” of the invention to the appropriate binding site on a cell, for example, if the peptide is a ligand, targeting means delivery of the peptide to the appropriate receptor, binding or adhesion protein for the ligand.

A peptide according to the invention can be attached to the 5′ or 3′ end of the first strand or the 5′ or 3′ end of the second strand or to the 5′ end of the first strand and the 5′ end of the second strand, to the 5′ end of the first strand and the 3′ end of the second strand, to the 3′ end of the first strand and the 5′ end of the second strand or to the 3′ end of the first strand and the 3′ end of the second strand of a dsRNA of the invention.

A peptide according to the invention can also be attached internally, for example via a specific functional group on the amino acid residue (e.g., —SH group on Cys or amino group of Lys), to the first and/or second strand.

In one aspect, more than one peptide, for example a dimer, a trimer or a multitude or peptides are attached to a dsRNA.

As used herein, a “dimer” means two peptides that are conjugated to each other, in any structural orientation, e.g., linearly via carboxy and amino termini, or in parallel via a covalent bond between an amino acid of each peptide, and wherein one of the two peptides is also conjugated to a dsRNA. A dimer also means two peptides wherein each peptide is conjugated to a unique site on a dsRNA.

As used herein, a “trimer” means three peptides that are conjugated to each other and wherein one of the three peptides is conjugated to a dsRNA. A trimer also means three peptides wherein each peptide is conjugated to a unique site on a dsRNA. A trimer also means three peptides wherein two of the three peptides are conjugated to each other and wherein one of the two peptides is also conjugated to a dsRNA and a third peptide is conjugated to a unique site on a dsRNA.

As used herein, a “multitude” means more than 1 peptide, for example 2, 3, 4, 5, 6, 7, 8, 9, 10 or more. The invention provides for a dsRNA that is conjugated to multiple peptides wherein the peptides are of the same or different sequences. In one embodiment, a multitude of peptides means one or more delivery peptide and one or more targeting peptide.

The term “peptide” embraces a limited number of contiguous amino acids that are peptide bonded together and comprises a targeting or delivery peptide as defined herein, whether the peptide is a naturally occurring molecule or synthetic. (i.e. a naturally occurring molecule, or a chemically/physically modified variant thereof) that is capable of delivering a dsRNA and/or binding to a peptide target, for example, a cell or a receptor on a cell.

A “peptide” as used herein can originate from a naturally occurring protein.

A “peptide” as used herein can comprise different protein domains (for example a chimeric peptide).

A “peptide” as used herein can be a synthetic peptide that is designed based on a structure-function relationship for a particular amino acid sequence and does not necessarily have homology to a natural sequence.

A peptide of the invention is conjugated to a dsRNA of the invention.

As used herein, conjugated means attached via any covalent or non-covalent association known in the art.

A peptide of the invention can be conjugated to a dsRNA of the invention via any amino acid residue in the peptide, e.g., the C-terminal amino acid of the C-terminus via the carboxyl group of the C-terminal amino acid or the N-terminal amino acid of the N-termimus via the α-amino group of the N-terminal amino acid or to a specific functional group on the amino acid residue (e.g., —SH group on Cys or amino group of Lys).

A peptide of the invention can be conjugated to a dsRNA of the invention via any amino acid residue internal in the peptide sequence, e.g., via the amino group of Lysine residues in the middle of the peptide sequence.

A peptide according to the invention can be conjugated to a dsRNA of the invention via a stable covalent linkage including but not limited to a zero-length linker, homobifunctional linker, heterobifunctional linker or a trifunctional linker (References: Bioconjugate Techniques, 1996. Greg T. Hermanson, Academic Press, San Diego, Calif.; Chemistry of Protein Conjugation and Cross-linking, 1991. Shan S. Wong, CRC Press, Boca Raton, Fla.).

As used herein, a “zero-length linker” means conjugation via a reaction where the reactants (e.g., the reactive groups on the dsRNAs and the functional groups on the peptides, such as reactive groups on the amino acid side chains, free amino and carboxyl groups of the terminal amino acid residues, etc.) are condensed to form a conjugated molecule without a linker. A “zero-length linker” is formed, for example, by reacting a terminal reactant of a peptide with the terminal reactant of a dsRNA. Examples of zero-length linking includes but are not limited to disulfides, amides, esters, thioesters, etc.

As used herein, a “homobifunctional linker” means conjugation with a linker having two similar functional groups. Examples of homobifunctional linkers include but are not limited to amino directed, carboxyl directed, sulfhydryl directed, etc.

As used herein, a “heterobifunctional linker” means conjugation with a linker having two dissimilar functional groups of different specificities. Examples of heterobifunctional linkers include but are not limited to combinations of amino and sulfhydryl directed, amino and carboxyl directed, carboxyl and sulfhydryl directed, etc.

As used herein, a “trifunctional linker” means conjugation with a linker having three reactive functional groups. Examples of trifunctional linkers include but are not limited to 4-azido-2-nitrophenylbiocytin-4-nitrophenyl ester (ABNP), sulfosuccinimidyl-2-[6-(biotinamido)-2-(p-azidobenzamido)hexanoamido]ethyl-1,3′-dithiopropionate (sulfo-SBED), other biocytin based molecules, etc.

A peptide according to the invention can also be conjugated to a dsRNA via a cleavable linker including but not limited to a disulfide, an ester, a glycol, a diazo, and a sulfone linker.

A peptide according to the invention can be conjugated to a dsRNA by a carbon linker, for example a carbon linker that is 1 or more carbons, for example, 1, 2, 3, 4, 5, 6, 7, 8, 9, 10, 11, 12, 13, 14, 15, 16, 17, 18, 19, 20, 21, 22, 23, 24, 25 or more carbons.

A peptide according to the invention can be conjugated to a dsRNA using a prosthetic group. Prosthetic groups include but are not limited to metal ions, porphyrin groups, coenzymes and other nonpeptidyl moieties, e.g., carbohydrates or oligosaccharides (Wong, S. S. (1991), Chemistry of protein conjugation and cross-linking, CRC Press).

In one embodiment, a peptide and a dsRNA are conjugated by expression as a fusion construct.

A “peptide” may be attached to a dsRNA by any conventional chemical conjugation techniques, which are well known to a skilled person. In this regard, reference is made to Hermanson, G. T. (1996), Bioconjugate techniques, Academic Press, and to Wong, S. S. (1991), Chemistry of protein conjugation and cross-linking, CRC Press.

A “peptide” may be conjugated to a dsRNA non-covalently via ionic interactions.

As used herein, a peptide-dsRNA conjugate” means a peptide that is conjugated to a dsRNA by a method including but not limited to the methods of attachment/conjugation described herein.

In one aspect a peptide-dsRNA conjugate further comprises one or more dye molecules.

As used herein, a “dye molecule” includes but is not limited to a polyaromatic dye or a fluorescent dye, for example Cy3, Cy5, Cy5.5, Alexa Fluor® (e.g, Alexa Fluor 488, Alexa Fluor 555, Alexa Fluor 647, etc.)

In one aspect, a peptide-dsRNA conjugate further comprises a delivery peptide, as defined herein.

In one aspect, a peptide-dsRNA conjugate further comprises a therapeutic agent, for example, an anticancer agent or an agent that treats a metabolic disease or disorder. Anticancer agents include but are not limited to antiviral agents (Fiume et al. FEBS Lett. 1983; 153(1):6-10), cisplatin (Mukhopadhyay S et al., Bioconjug Chem. 2008; 19(1):39-49), doxorubicin (Guan H et al., Bioconjug Chem. 2008; 19(9):1813-21), paclitaxel (Dubikovskaya E A et al., Proc Natl Acad Sci USA. 2008; 105(34):12128-33, Regina A et al., Br J. Pharmacol. 2008; 155(2):185-97), tamoxifen (Rickert et al. Biomacromolecules. 2007; 8(11):3608-3612) and vinblastine (DeFeo-Jones D et al., Mol Cancer Ther. 2002; 1(7):451-459). A composition that includes a therapeutic agent in combination with the peptide-dsRNA conjugate can include a ratio of agent/conjugate from 1:10,000 to 1:1 to 10,000:1, respectively; for example, 1:5000 to 5000:1, 1:1000 to 1000:1, 1:100-100:1, 1:10 to 10:1, on a molecular weight, molar basis, or on an actual weight basis.

A “peptide-dsRNA conjugate” refers to a molecule wherein both of said peptide and said dsRNA retain their function.

As used herein, “decreased” for example, a decrease in the onset of action of a dsRNA-peptide conjugate, or a decrease in the speed of delivery of a dsRNA-peptide conjugate, means 1, 2, 3, 4, 5, 10, 15, 20, 25, 40, 35, 40, 45, 50, 100, 1000 or 10,000-fold or more less than the onset of action or speed of delivery of an identical dsRNA that is not conjugated to a peptide.

As used herein, “decreased” for example, a decrease in the onset of action of a dsRNA-peptide conjugate, or a decrease in the speed of delivery of a dsRNA-peptide conjugate, means 1, 5, 10, 15, 20, 25, 30, 35, 40, 45, 50, 55, 60, 65, 70, 75, 80, 85, 90, 95, 99 or 100% less than an the onset of action or speed of delivery of identical dsRNA that is not conjugated to a peptide.

As used herein, “onset of action” means the time period between the administration of a dsRNA in vitro (for example to a cell or to tissue culture medium) or in vivo (for example to a human or animal (e.g. mouse or rat) subject) and the arrival of the dsRNA at the target RNA.

As used herein, “speed of delivery” means the time required for a dsRNA to reach a target RNA following administration of a dsRNA.

As used herein, “duration of action” means the time period during which dsRNA inhibits expression of a target RNA.

As used herein, a “control” or a “reference”, for example a control dsRNA, means a dsRNA that is comparable in length to the dsRNA that is specific for a particular target RNA (the test dsRNA), but that is not specific for a particular target RNA. A control RNA has a nucleotide sequence that is not identical to the dsRNA that is specific for a target of interest. A control, for example a control peptide means a peptide that is comparable in one or more of length and charge but has an amino acid sequence that is different from the amino acid sequence of the peptide that is conjugated to a dsRNA that is specific for a target RNA (the test peptide). A control, for example a control dsRNA-peptide conjugate means a dsRNA-peptide conjugate wherein the dsRNA is comparable in length to the dsRNA that is specific for a particular target RNA, but is not specific for a particular target RNA. A control dsRNA-peptide conjugate also means a dsRNA-peptide conjugate wherein the peptide is comparable in one or more of length and charge but has an amino acid sequence that is different from the amino acid sequence of the peptide that is conjugated to a dsRNA that is specific for a target RNA. A control dsRNA-peptide conjugate also means a dsRNA-peptide conjugate wherein the peptide is comparable in one or more of length and charge but has an amino acid sequence that is different from the amino acid sequence of the peptide that is conjugated to a dsRNA that is specific for a target RNA and wherein the dsRNA is comparable in length to the dsRNA that is specific for a particular target RNA, but that is not specific for a particular target RNA.

As used herein, a test peptide or a test dsRNA means a peptide or dsRNA that comprises a conjugate that decreases the expression of a target RNA according to the invention. A test dsRNA means a dsRNA that decreases the expression of a target RNA according to the invention. A “test” dsRNA-peptide conjugate comprises a test dsRNA conjugated to a test peptide.

As used herein, the term “nucleic acid” refers to deoxyribonucleotides, ribonucleotides, or modified nucleotides, and polymers thereof in single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

As used herein, “nucleotide” is used as recognized in the art to include those with natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see, e.g., Usman and McSwiggen, supra; Eckstein, et al., International PCT Publication No. WO 92/07065; Usman et al, International PCT Publication No. WO 93/15187; Uhlman & Peyman, supra, all are hereby incorporated by reference herein). There are several examples of modified nucleic acid bases known in the art as summarized by Limbach, et al, Nucleic Acids Res. 22:2183, 1994. Some of the non-limiting examples of base modifications that can be introduced into nucleic acid molecules include, hypoxanthine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, and others (Burgin, et al., Biochemistry 35:14090, 1996; Uhlman & Peyman, supra). By “modified bases” in this aspect is meant nucleotide bases other than adenine, guanine, cytosine and uracil at 1′ position or their equivalents.

As used herein, “modified nucleotide” refers to a nucleotide that has one or more modifications to the nucleoside, the nucleobase, pentose ring, or phosphate group. For example, modified nucleotides exclude ribonucleotides containing adenosine monophosphate, guanosine monophosphate, uridine monophosphate, and cytidine monophosphate and deoxyribonucleotides containing deoxyadenosine monophosphate, deoxyguanosine monophosphate, deoxythymidine monophosphate, and deoxycytidine monophosphate. Modifications include those naturally occurring that result from modification by enzymes that modify nucleotides, such as methyltransferases. Modified nucleotides also include synthetic or non-naturally occurring nucleotides. Synthetic or non-naturally occurring modifications in nucleotides include those with 2′ modifications, e.g., 2′-O-methyl, 2′-methoxyethoxy, 2′-fluoro, 2′-allyl, 2′-O-[2-(methylamino)-2-oxoethyl], 4′-thio, 4′-CH₂—O-2′-bridge, 4′-(CH₂)₂—O-2′-bridge, 2′-LNA, and 2′-O-(N-methylcarbamate) or those comprising base analogs. In connection with 2′-modified nucleotides as described for the present disclosure, by “amino” is meant 2′-NH₂ or 2′-O—NH₂, which can be modified or unmodified. Such modified groups are described, e.g., in Eckstein et al., U.S. Pat. No. 5,672,695 and Matulic-Adamic et al., U.S. Pat. No. 6,248,878.

In reference to the nucleic acid molecules of the present disclosure, modifications may exist upon these agents in patterns on one or both strands of the dsRNA). As used herein, “alternating positions” refers to a pattern where every other nucleotide is a modified nucleotide or there is an unmodified nucleotide (e.g., an unmodified ribonucleotide) between every modified nucleotide over a defined length of a strand of the dsRNA (e.g., 5′-MNMNMN-3′; 3′-MNMNMN-5′; where M is a modified nucleotide and N is an unmodified nucleotide). In certain embodiments, the modification pattern starts from the first nucleotide position at either the 5′ or 3′ terminus according to any of the position numbering conventions described herein (in certain embodiments, position 1 is designated in reference to the terminal residue of a strand following a projected Dicer cleavage event of a DsiRNA agent of the invention; thus, position 1 does not always constitute a 3′ terminal or 5′ terminal residue of a pre-processed agent of the invention). In other embodiments, position 1 is designated in reference to the nucleotide residue of a first or second strand that is complementary to the 5′ or 3′ end of the opposite strand. For example, in certain embodiments, position 1 is the nucleotide residue of the second strand that is complementary to the 5′ terminal nucleotide residue of the first oligonucleotide strand. The invention encompasses dsRNAs wherein the modification pattern starts at any one of positions 1, 2, 3, 4, 5, 6, 7, 8, 9, 10, 11, 12, 13, 14, 15, 16, 12, 18, 19, 20, 21, 22, 23 or 24 from the 5′ or 3′ terminus according to any of the position numbering conventions described herein. The invention also encompasses dsRNAs wherein the modification patterns starts at any position that is at least one nucleotide from the 5′ or 3′ terminal residue.

The pattern of modified nucleotides at alternating positions may run the full length of the strand, but in certain embodiments includes at least 2, 3, 4, 5, 6, 7, 8, 9, 10, 11, 12, 13, 14 15, 16, 17, 18, 19, 20, 21, 22, 23, 24, 25, 26, 27, 28 or more nucleotides containing at least 2, 3, 4, 5, 6, 7, 8, 9, 10, 11, 12, 13, 14, 15, 16, 17, 18, 19, 20, 21, 22, 23, 24 or more modified nucleotides, respectively.

As used herein, “alternating pairs of positions” refers to a pattern where two consecutive modified nucleotides are separated by two consecutive unmodified nucleotides over a defined length of a strand of the dsRNA (e.g., 5′-MMNNMMNNMMNN-3′; 3′-MMNNMMNNMMNN-5′; where M is a modified nucleotide and N is an unmodified nucleotide). In one embodiment, the modification pattern starts from the first nucleotide position at either the 5′ or 3′ terminus according to any of the position numbering conventions described herein. The pattern of modified nucleotides at alternating positions may run the full length of the strand, but preferably includes at least 2, 3, 4, 5, 6, 7, 8, 9, 10, 11, 12, 13, 14 15, 16, 17, 18, 19, 20, 21, 22, 23, 24, 25, 26, 27, 28 nucleotides containing at least 2, 3, 4, 5, 6, 7, 8, 9, 10, 11, 12, 13, 14, 15, 16, 17, 18, 19, 20, 21, 22, 23, 24 modified nucleotides, respectively. It is emphasized that the above modification patterns are exemplary and are not intended as limitations on the scope of the invention.

As used herein, “base analog” refers to a heterocyclic moiety which is located at the 1′ position of a nucleotide sugar moiety in a modified nucleotide that can be incorporated into a nucleic acid duplex (or the equivalent position in a nucleotide sugar moiety substitution that can be incorporated into a nucleic acid duplex). In the dsNAs of the invention, a base analog is generally either a purine or pyrimidine base excluding the common bases guanine (G), cytosine (C), adenine (A), thymine (T), and uracil (U). Base analogs can duplex with other bases or base analogs in dsRNAs. Base analogs include those useful in the compounds and methods of the invention, e.g., those disclosed in U.S. Pat. Nos. 5,432,272 and 6,001,983 to Benner and US Patent Publication No. 20080213891 to Manoharan, which are herein incorporated by reference. Non-limiting examples of bases include hypoxanthine (I), xanthine (X), 3β-D-ribofuranosyl-(2,6-diaminopyrimidine) (K), 3-β-D-ribofuranosyl-(1-methyl-pyrazolo[4,3-d]pyrimidine-5,7(4H,6H)-dione) (P), iso-cytosine (iso-C), iso-guanine (iso-G), 1-β-D-ribofuranosyl-(5-nitroindole), 1-β-D-ribofuranosyl-(3-nitropyrrole), 5-bromouracil, 2-aminopurine, 4-thio-dT, 7-(2-thienyl)-imidazo[4,5-b]pyridine (Ds) and pyrrole-2-carbaldehyde (Pa), 2-amino-6-(2-thienyl)purine (S), 2-oxopyridine (Y), difluorotolyl, 4-fluoro-6-methylbenzimidazole, 4-methylbenzimidazole, 3-methyl isocarbostyrilyl, 5-methyl isocarbostyrilyl, and 3-methyl-7-propynyl isocarbostyrilyl, 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylindolyl, 4,6-dimethylindolyl, phenyl, napthalenyl, anthracenyl, phenanthracenyl, pyrenyl, stilbenzyl, tetracenyl, pentacenyl, and structural derivates thereof (Schweitzer et al., J. Org. Chem., 59:7238-7242 (1994); Berger et al., Nucleic Acids Research, 28(15):2911-2914 (2000); Moran et al., J. Am. Chem. Soc., 119:2056-2057 (1997); Morales et al., J. Am. Chem. Soc., 121:2323-2324 (1999); Guckian et al., J. Am. Chem. Soc., 118:8182-8183 (1996); Morales et al., J. Am. Chem. Soc., 122(6):1001-1007 (2000); McMinn et al., J. Am. Chem. Soc., 121:11585-11586 (1999); Guckian et al., J. Org. Chem., 63:9652-9656 (1998); Moran et al., Proc. Natl. Acad. Sci., 94:10506-10511 (1997); Das et al., J. Chem. Soc., Perkin Trans., 1:197-206 (2002); Shibata et al., J. Chem. Soc., Perkin Trans., 1: 1605-1611 (2001); Wu et al., J. Am. Chem. Soc., 122(32):7621-7632 (2000); O'Neill et al., J. Org. Chem., 67:5869-5875 (2002); Chaudhuri et al., J. Am. Chem. Soc., 117:10434-10442 (1995); and U.S. Pat. No. 6,218,108.). Base analogs may also be a universal base.

As used herein, “universal base” refers to a heterocyclic moiety located at the 1′ position of a nucleotide sugar moiety in a modified nucleotide, or the equivalent position in a nucleotide sugar moiety substitution, that, when present in a nucleic acid duplex, can be positioned opposite more than one type of base without altering the double helical structure (e.g., the structure of the phosphate backbone). Additionally, the universal base does not destroy the ability of the single stranded nucleic acid in which it resides to duplex to a target nucleic acid. The ability of a single stranded nucleic acid containing a universal base to duplex a target nucleic can be assayed by methods apparent to one in the art (e.g., UV absorbance, circular dichroism, gel shift, single stranded nuclease sensitivity, etc.). Additionally, conditions under which duplex formation is observed may be varied to determine duplex stability or formation, e.g., temperature, as melting temperature (Tm) correlates with the stability of nucleic acid duplexes. Compared to a reference single stranded nucleic acid that is exactly complementary to a target nucleic acid, the single stranded nucleic acid containing a universal base forms a duplex with the target nucleic acid that has a lower Tm than a duplex formed with the complementary nucleic acid. However, compared to a reference single stranded nucleic acid in which the universal base has been replaced with a base to generate a single mismatch, the single stranded nucleic acid containing the universal base forms a duplex with the target nucleic acid that has a higher Tm than a duplex formed with the nucleic acid having the mismatched base.

Some universal bases are capable of base pairing by forming hydrogen bonds between the universal base and all of the bases guanine (G), cytosine (C), adenine (A), thymine (T), and uracil (U) under base pair forming conditions. A universal base is not a base that forms a base pair with only one single complementary base. In a duplex, a universal base may form no hydrogen bonds, one hydrogen bond, or more than one hydrogen bond with each of G, C, A, T, and U opposite to it on the opposite strand of a duplex. Preferably, the universal bases does not interact with the base opposite to it on the opposite strand of a duplex. In a duplex, base pairing between a universal base occurs without altering the double helical structure of the phosphate backbone. A universal base may also interact with bases in adjacent nucleotides on the same nucleic acid strand by stacking interactions. Such stacking interactions stabilize the duplex, especially in situations where the universal base does not form any hydrogen bonds with the base positioned opposite to it on the opposite strand of the duplex. Non-limiting examples of universal-binding nucleotides include inosine, 1-β-D-ribofuranosyl-5-nitroindole, and/or 1-β-D-ribofuranosyl-3-nitropyrrole (US Pat. Appl. Publ. No. 20070254362 to Quay et al.; Van Aerschot et al., An acyclic 5-nitroindazole nucleoside analogue as ambiguous nucleoside. Nucleic Acids Res. 1995 Nov. 11; 23(21):4363-70; Loakes et al., 3-Nitropyrrole and 5-nitroindole as universal bases in primers for DNA sequencing and PCR. Nucleic Acids Res. 1995 Jul. 11; 23(13):2361-6; Loakes and Brown, 5-Nitroindole as an universal base analogue. Nucleic Acids Res. 1994 Oct. 11; 22(20):4039-43).

As used herein, “loop” refers to a structure formed by a single strand of a nucleic acid, in which complementary regions that flank a particular single stranded nucleotide region hybridize in a way that the single stranded nucleotide region between the complementary regions is excluded from duplex formation or Watson-Crick base pairing. A loop is a single stranded nucleotide region of any length. Examples of loops include the unpaired nucleotides present in such structures as hairpins, stem loops, or extended loops.

As used herein, “extended loop” in the context of a dsRNA refers to a single stranded loop and in addition 1, 2, 3, 4, 5, 6 or up to 20 base pairs or duplexes flanking the loop. In an extended loop, nucleotides that flank the loop on the 5′ side form a duplex with nucleotides that flank the loop on the 3′ side. An extended loop may form a hairpin or stem loop.

As used herein, “tetraloop” in the context of a dsRNA refers to a loop (a single stranded region) consisting of four nucleotides that forms a stable secondary structure that contributes to the stability of an adjacent Watson-Crick hybridized nucleotides. Without being limited to theory, a tetraloop may stabilize an adjacent Watson-Crick base pair by stacking interactions. In addition, interactions among the four nucleotides in a tetraloop include but are not limited to non-Watson-Crick base pairing, stacking interactions, hydrogen bonding, and contact interactions (Cheong et al., Nature 1990 Aug. 16; 346(6285):680-2; Heus and Pardi, Science 1991 Jul. 12; 253(5016):191-4). A tetraloop confers an increase in the melting temperature (Tm) of an adjacent duplex that is higher than expected from a simple model loop sequence consisting of four random bases. For example, a tetraloop can confer a melting temperature of at least 55° C. in 10 mM NaHPO₄ to a hairpin comprising a duplex of at least 2 base pairs in length. A tetraloop may contain ribonucleotides, deoxyribonucleotides, modified nucleotides, and combinations thereof. Examples of RNA tetraloops include the UNCG family of tetraloops (e.g., UUCG), the GNRA family of tetraloops (e.g., GAAA), and the CUUG tetraloop. (Woese et al., Proc Natl Acad Sci USA. 1990 November; 87(21):8467-71; Antao et al., Nucleic Acids Res. 1991 Nov. 11; 19(21):5901-5). Examples of DNA tetraloops include the d(GNNA) family of tetraloops (e.g., d(GTTA), the d(GNRA)) family of tetraloops, the d(GNAB) family of tetraloops, the d(CNNG) family of tetraloops, the d(TNCG) family of tetraloops (e.g., d(TTCG)). (Nakano et al. Biochemistry, 41 (48), 14281-14292, 2002; SHINJI et al. Nippon Kagakkai Koen Yokoshu VOL. 78th; NO. 2; PAGE. 731 (2000).)

The dsRNA compositions of the invention, because they are modeled to enter the RNAi pathway as substrates of the Dicer enzyme, at least in part due the strand lengths of such compositions, are also referred to as Dicer substrate siRNA (“DsiRNA”) agents herein. The “DsiRNA agent” compositions of the instant invention comprise dsRNA which is a precursor molecule for Dicer enzyme processing, i.e., the DsiRNA of the present invention is processed in vivo to produce an active siRNA. Specifically, the DsiRNA is processed by Dicer to an active siRNA which is incorporated into RISC. This precursor molecule, primarily referred to as a “DsiRNA agent” or “DsiRNA molecule” herein, can also be referred to as a precursor RNAi molecule herein. As used herein, the term “active siRNA” refers to a double stranded nucleic acid in which each strand comprises RNA, RNA analog(s) or RNA and DNA. The siRNA comprises between 19 and 23 nucleotides or comprises 21 nucleotides. The active siRNA typically has 2 bp overhangs on the 3′ ends of each strand such that the duplex region in the siRNA comprises 17-21 nucleotides, or 19 nucleotides.

In certain embodiments, dsRNAs of the invention include but are not limited to dsRNAs comprising first and second strands comprising between 16 and 50, 19 and 35, 19 and 24, 25 and 30, 25 and 35, 26 and 30, 21 and 23 nucleotides in length.

A DsiRNA agent of the instant invention has a length sufficient such that it is processed by Dicer to produce an siRNA. Accordingly, a suitable DsiRNA agent contains one oligonucleotide sequence, a first sequence, that is at least 25 nucleotides in length and no longer than about 35 nucleotides. This sequence of RNA can be between about 26 and 35, 26 and 34, 26 and 33, 26 and 32, 26 and 31, 26 and 30, and 26 and 29 nucleotides in length. This sequence can be about 27 or 28 nucleotides in length or 27 nucleotides in length. The second sequence of the DsiRNA agent can be any sequence that anneals to the first sequence under biological conditions, such as within the cytoplasm of a eukaryotic cell. Generally, the second oligonucleotide sequence will have at least 19 complementary base pairs with the first oligonucleotide sequence, more typically the second oligonucleotides sequence will have about 21 or more complementary base pairs, or about 25 or more complementary base pairs with the first oligonucleotide sequence. In one embodiment, the second sequence is the same length as the first sequence, and the DsiRNA agent is blunt ended. In another embodiment, the ends of the DsiRNA agent have one or more overhangs. In certain embodiments, wherein the second sequence is the same length as the first sequence, the ultimate residue of said 3′ terminus of said first strand and the ultimate residue of the said 5′ terminus of the second strand form a mismatched base pair. In other embodiments, wherein the second sequence is the same length as the first sequence, the ultimate residue of the 5′ terminus of said first strand and the ultimate residue of the 3′ terminus of the second strand form a mismatched base pair. In other embodiments, wherein the second sequence is the same length as the first sequence, the ultimate and penultimate residues of the 3′ terminus of the first strand and the ultimate and penultimate residues of the 5′ terminus of the second strand form two mismatched base pairs. In still other embodiments, wherein the second sequence is the same length as the first sequence, the ultimate and penultimate residues of the 5′ terminus of the first strand and the ultimate and penultimate residues of the 3′ terminus of the second strand form two mismatched base pairs.

In certain embodiments, the first and second oligonucleotide sequences of the DsiRNA agent exist on separate oligonucleotide strands that can be and typically are chemically synthesized. In some embodiments, both strands are between 26 and 35 nucleotides in length. In other embodiments, both strands are between 25 and 30 or 26 and 30 nucleotides in length. In one embodiment, both strands are 27 nucleotides in length, are completely complementary and have blunt ends. In one embodiment, one or both oligonucleotide strands are capable of serving as a substrate for Dicer. In other embodiments, at least one modification is present that promotes Dicer to bind to the double-stranded RNA structure in an orientation that maximizes the double-stranded RNA structure's effectiveness in inhibiting gene expression. In certain embodiments of the instant invention, the DsiRNA agent is comprised of two oligonucleotide strands of differing lengths, with the DsiRNA possessing a blunt end at the 3′ terminus of a first strand (sense strand) and a 3′ overhang at the 3′ terminus of a second strand (antisense strand). The DsiRNA can also contain one or more deoxyribonucleic acid (DNA) base substitutions.

Suitable DsiRNA compositions that contain two separate oligonucleotides can be chemically linked outside their annealing region by chemical linking groups. Many suitable chemical linking groups are known in the art and can be used. Suitable groups will not block Dicer activity on the DsiRNA and will not interfere with the directed destruction of the RNA transcribed from the target gene. Alternatively, the two separate oligonucleotides can be linked by a third oligonucleotide such that a hairpin structure is produced upon annealing of the two oligonucleotides making up the DsiRNA composition. The hairpin structure will not block Dicer activity on the DsiRNA and will not interfere with the directed destruction of the target RNA.

As used herein, a dsRNA, e.g., DsiRNA or siRNA, having a sequence “sufficiently complementary” to a target RNA or cDNA sequence means that the dsRNA has a sequence sufficient to trigger the destruction of the target RNA (where a cDNA sequence is recited, the RNA sequence corresponding to the recited cDNA sequence) by the RNAi machinery (e.g., the RISC complex) or process. The dsRNA molecule can be designed such that every residue of the antisense strand is complementary to a residue in the target molecule. Alternatively, substitutions can be made within the molecule to increase stability and/or enhance processing activity of said molecule. Substitutions can be made within the strand or can be made to residues at the ends of the strand. In certain embodiments, substitutions and/or modifications are made at specific residues within a DsiRNA agent. Such substitutions and/or modifications can include, e.g., deoxy-modifications at one or more residues of positions 1, 2 and 3 when numbering from the 3′ terminal position of the sense strand of a DsiRNA agent; deoxy-modifications at one or more residues of positions 1, 2, 3 or 4 when numbering from the 5′ terminal position of the antisense strand of a DsiRNA agent and introduction of 2′-O-alkyl (e.g., 2′-O-methyl) modifications at the 3′ terminal residue of the antisense strand of DsiRNA agents, with such modifications also or alternatively being present at overhang positions of the 3′ portion of the antisense strand and/or throughout the DsiRNA agent, for example at alternating residues or in pairs of residues of the antisense strand of the DsiRNA that are included within the region of a DsiRNA agent that is processed to form an active siRNA agent. The preceding modifications are offered as exemplary, and are not intended to be limiting in any manner. Further consideration of the structure of preferred DsiRNA agents, including further description of the modifications and substitutions that can be performed upon the DsiRNA agents of the instant invention, can be found below.

By “complementarity” is meant that a nucleic acid can form hydrogen bond(s) with another nucleic acid sequence by either traditional Watson-Crick or other non-traditional types. In reference to the nucleic molecules of the present invention, the binding free energy for a nucleic acid molecule with its complementary sequence is sufficient to allow the relevant function of the nucleic acid to proceed, e.g., RNAi activity. Determination of binding free energies for nucleic acid molecules is well known in the art (see, e.g., Turner et al., 1987, CSH Symp. Quant. Biol. LII pp. 123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, or 10 nucleotides out of a total of 10 nucleotides in the first oligonucleotide being based paired to a second nucleic acid sequence having 10 nucleotides represents 50%, 60%, 70%, 80%, 90%, and 100% complementary respectively). “Perfectly complementary” means that all the contiguous residues of a nucleic acid sequence will hydrogen bond with the same number of contiguous residues in a second nucleic acid sequence. In one embodiment, a DsiRNA molecule of the invention comprises about 19 to about 30 (e.g., about 19, 20, 21, 22, 23, 24, 25, 26, 27, 28, 29, or or more) nucleotides that are complementary to one or more target nucleic acid molecules or a portion thereof.

The phrase “duplex region” refers to the region in two complementary or substantially complementary oligonucleotides that form base pairs with one another, either by Watson-Crick base pairing or any other manner that allows for a duplex between oligonucleotide strands that are complementary or substantially complementary. For example, an oligonucleotide strand having 21 nucleotide units can base pair with another oligonucleotide of 21 nucleotide units, yet only 19 bases on each strand are complementary or substantially complementary, such that the “duplex region” consists of 19 base pairs. The remaining base pairs may, for example, exist as 5′ and 3′ overhangs. Further, within the duplex region, 100% complementarity is not required; substantial complementarity is allowable within a duplex region.

Substantial complementarity refers to complementarity between the strands such that they are capable of annealing under biological conditions. Techniques to empirically determine if two strands are capable of annealing under biological conditions are well know in the art. Alternatively, two strands can be synthesized and added together under biological conditions to determine if they anneal to one another.

Single-stranded nucleic acids that base pair over a number of bases are said to “hybridize.” Hybridization is typically determined under physiological or biologically relevant conditions (e.g., intracellular: pH 7.2, 140 mM potassium ion; extracellular pH 7.4, 145 mM sodium ion). Hybridization conditions generally contain a monovalent cation and biologically acceptable buffer and may or may not contain a divalent cation, complex anions, e.g. gluconate from potassium gluconate, uncharged species such as sucrose, and inert polymers to reduce the activity of water in the sample, e.g. PEG. Such conditions include conditions under which base pairs can form.

Hybridization is measured by the temperature required to dissociate single stranded nucleic acids forming a duplex, i.e., (the melting temperature; Tm). Hybridization conditions are also conditions under which base pairs can form. Various conditions of stringency can be used to determine hybridization (see, e.g., Wahl, G. M. and S. L. Berger (1987) Methods Enzymol. 152:399; Kimmel, A. R. (1987) Methods Enzymol. 152:507). Stringent temperature conditions will ordinarily include temperatures of at least about 30° C., more preferably of at least about 37° C., and most preferably of at least about 42° C. The hybridization temperature for hybrids anticipated to be less than 50 base pairs in length should be 5-10° C. less than the melting temperature (Tm) of the hybrid, where Tm is determined according to the following equations. For hybrids less than 18 base pairs in length, Tm(° C.)=2(# of A+T bases)+4(# of G+C bases). For hybrids between 18 and 49 base pairs in length, Tm(° C.)=81.5+16.6(log 10[Na+])+0.41 (% G+C)−(600/N), where N is the number of bases in the hybrid, and [Na+] is the concentration of sodium ions in the hybridization buffer ([Na+] for 1×SSC=0.165 M). For example, a hybridization determination buffer is shown in Table 1.

TABLE 1

| | | | | | | | To make 50 mL | |
|---|---|---|---|---|---|---|---|---|
| | final conc. | | Vender | Cat# | Lot# | m.w./Stock | solution | |
| NaCl | 100 | mM | Sigma | S-5150 | 41K8934 | 5M | 1 | mL |
| KCl | 80 | mM | Sigma | P-9541 | 70K0002 | 74.55 | 0.298 | g |
| MgCl₂ | 8 | mM | Sigma | M-1028 | 120K8933 | 1M | 0.4 | mL |
| sucrose | 2% | w/v | Fisher | BP220- | 907105 | 342.3 | 1 | g |
| | | | | 212 | | | | |
| Tris-HCl | 16 | mM | Fisher | BP1757- | 12419 | 1 | 0.8 | mL |
| | | | | 500 | | | | |
| NaH₂PO₄ | 1 | mM | Sigma | S-3193 | 52H- | 120.0 | 0.006 | g |
| | | | | | 029515 | | | |
| EDTA | 0.02 | mM | Sigma | E-7889 | 110K89271 | 0.5M | 2 | μL |

| | | | | | |
|---|---|---|---|---|---|
| H₂O | Sigma | W-4502 | 51K2359 | to 50 | mL |

| | |
|---|---|
| pH = 7.0 | adjust with |
| at 20° C. | HCl |

Useful variations on hybridization conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Antisense to Molecular Cloning Techniques, 1987, Academic Press, New York); and Sambrook et al., Molecular Cloning: A Laboratory Manual, Cold Spring Harbor Laboratory Press, New York.

As used herein, “oligonucleotide strand” is a single stranded nucleic acid molecule. An oligonucleotide may comprise ribonucleotides, deoxyribonucleotides, modified nucleotides (e.g., nucleotides with 2′ modifications, synthetic base analogs, etc.) or combinations thereof. Such modified oligonucleotides can be preferred over native forms because of properties such as, for example, enhanced cellular uptake and increased stability in the presence of nucleases.

Certain dsRNAs of this invention can be chimeric double-stranded ribonucleic acids (dsRNAs). “Chimeric dsRNAs” or “chimeras”, in the context of this invention, are dsRNAs which contain two or more chemically distinct regions, each made up of at least one nucleotide. These dsRNAs typically contain at least one region primarily comprising ribonucleotides (optionally including modified ribonucleotides) that form a Dicer substrate siRNA (“DsiRNA”) molecule. This DsiRNA region can be covalently attached to a second region comprising base paired deoxyribonucleotides (a “dsDNA region”) on either flank of the ribonucleotide duplex region, which can confer one or more beneficial properties (such as, for example, increased efficacy, e.g., increased potency and/or duration of DsiRNA activity, function as a recognition domain or means of targeting a chimeric dsNA to a specific location, for example, when administered to cells in culture or to a subject, functioning as an extended region for improved attachment of functional groups, payloads, detection/detectable moieties, functioning as an extended region that allows for more desirable modifications and/or improved spacing of such modifications, etc.). This second region, e.g., comprising base paired deoxyribonucleotides may also include modified or synthetic nucleotides and/or modified or synthetic deoxyribonucleotides.

As used herein, the term “ribonucleotide” encompasses natural and synthetic, unmodified and modified ribonucleotides. Modifications include changes to the sugar moiety, to the base moiety and/or to the linkages between ribonucleotides in the oligonucleotide. As used herein, the term “ribonucleotide” specifically excludes a deoxyribonucleotide, which is a nucleotide possessing a single proton group at the 2′ ribose ring position.

As used herein, the term “deoxyribonucleotide” encompasses natural and synthetic, unmodified and modified deoxyribonucleotides. Modifications include changes to the sugar moiety, to the base moiety and/or to the linkages between deoxyribonucleotide in the oligonucleotide. As used herein, the term “deoxyribonucleotide” also includes a modified ribonucleotide that does not permit Dicer cleavage of a dsRNA agent, e.g., a 2′-O-methyl ribonucleotide, a phosphorothioate-modified ribonucleotide residue, etc., that does not permit Dicer cleavage to occur at a bond of such a residue.

As used herein, the term “PS-NA” refers to a phosphorothioate-modified nucleotide residue. The term “PS-NA” therefore encompasses both phosphorothioate-modified ribonucleotides (“PS-RNAs”) and phosphorothioate-modified deoxyribonucleotides (“PS-DNAs”).

As used herein, “Dicer” refers to an endoribonuclease in the RNase III family that cleaves a dsRNA or dsRNA-containing molecule, e.g., double-stranded RNA (dsRNA) or pre-microRNA (miRNA), into double-stranded nucleic acid fragments about 19-25 nucleotides long, usually with a two-base overhang on the 3′ end. With respect to the dsRNAs of the invention, the duplex formed by a dsRNA region of a dsRNA of the invention is recognized by Dicer and is a Dicer substrate on at least one strand of the duplex. Dicer catalyzes the first step in the RNA interference pathway, which consequently results in the degradation of a target RNA. The protein sequence of human Dicer is provided at the NCBI database under accession number NP_{—}085124, hereby incorporated by reference.

Dicer “cleavage” is determined as follows (e.g., see Collingwood et al., Oligonucleotides 18:187-200 (2008)). In a Dicer cleavage assay, RNA duplexes (100 pmol) are incubated in 20 μL of 20 mM Tris pH 8.0, 200 mM NaCl, 2.5 mM MgCl2 with or without 1 unit of recombinant human Dicer (Stratagene, La Jolla, Calif.) at 37° C. for 18-24 hours. Samples are desalted using a Performa SR 96-well plate (Edge Biosystems, Gaithersburg, Md.). Electrospray-ionization liquid chromatography mass spectroscopy (ESI-LCMS) of duplex RNAs pre- and post-treatment with Dicer is done using an Oligo HTCS system (Novatia, Princeton, N.J.; Hail et al., 2004), which consists of a ThermoFinnigan TSQ7000, Xcalibur data system, ProMass data processing software and Paradigm MS4 HPLC (Michrom BioResources, Auburn, Calif.). In this assay, Dicer cleavage occurs where at least 5%, 10%, 20%, 30%, 40%, 50%, 60%, 70%, 80%, 90%, 95%, or even 100% of the Dicer substrate dsRNA, (i.e., 25-30 bp, dsRNA, preferably 26-30 bp dsRNA) is cleaved to a shorter dsRNA (e.g., 19-23 bp dsRNA, preferably, 21-23 bp dsRNA).

As used herein, “Dicer cleavage site” refers to the sites at which Dicer cleaves a dsRNA (e.g., the dsRNA region of a dsRNA of the invention). Dicer contains two RNase III domains which typically cleave both the sense and antisense strands of a dsRNA. The average distance between the RNase III domains and the PAZ domain determines the length of the short double-stranded nucleic acid fragments it produces and this distance can vary (Macrae I, et al. (2006). “Structural basis for double-stranded RNA processing by Dicer”. Science 311 (5758): 195-8.). Dicer is projected to cleave certain double-stranded nucleic acids of the instant invention that possess an antisense strand having a 2 nucleotide 3′ overhang at a site between the 21^{st} and 22^{nd} nucleotides removed from the 3′ terminus of the antisense strand, and at a corresponding site between the 21^{st} and 22^{nd} nucleotides removed from the 5′ terminus of the sense strand. The projected and/or prevalent Dicer cleavage site(s) for dsRNA molecules distinct from those are known in the art or may be similarly identified via art-recognized methods, including those described in Macrae et al. Dicer cleavage of a dsRNA (e.g., DsiRNA) can result in generation of Dicer-processed siRNA lengths of 19 to 23 nucleotides in length. Indeed, in one aspect of the invention that is described in greater detail below, a double stranded DNA region is included within a dsRNA for purpose of directing prevalent Dicer excision of a typically non-preferred 19mer siRNA.

As used herein, “overhang” refers to unpaired nucleotides, in the context of a duplex having one, two, three, four or five free ends at either the 5′ terminus or 3′ terminus of a dsRNA. In certain embodiments, the overhang is a 3′ or 5′ overhang on the antisense strand or sense strand.

As used herein, the term “DmiRNA” refers to a species of Dicer substrate siRNA (“DsiRNA”) that possesses at least one mismatch nucleotide within the antisense (guide) strand of the DmiRNA agent, specifically within the region of the antisense strand that functions as an RNA interference agent and is believed to hybridize with the sequence of a target RNA. Such mismatch nucleotide can exist either with respect to the sense (passenger) strand, with respect to the target RNA sequence to which the antisense strand of the DmiRNA is believed to hybridize, or with respect to both.

As used herein, the term “RNA processing” refers to processing activities performed by components of the siRNA, miRNA or RNase H pathways (e.g., Drosha, Dicer, Argonaute2 or other RISC endoribonucleases, and RNaseH), which are described in greater detail below (see “RNA Processing” section below). The term is explicitly distinguished from the post-transcriptional processes of 5′ capping of RNA and degradation of RNA via non-RISC- or non-RNase H-mediated processes. Such “degradation” of an RNA can take several forms, e.g. deadenylation (removal of a 3′ poly(A) tail), and/or nuclease digestion of part or all of the body of the RNA by any of several endo- or exo-nucleases (e.g., RNase III, RNase P, RNase T1, RNase A (1, 2, 3, 4/5), oligonucleotidase, etc.).

By “homologous sequence” is meant, a nucleotide sequence that is shared by one or more polynucleotide sequences, such as genes, gene transcripts and/or non-coding polynucleotides. For example, a homologous sequence can be a nucleotide sequence that is shared by two or more genes encoding related but different proteins, such as different members of a gene family, different protein epitopes, different protein isoforms or completely divergent genes, such as a cytokine and its corresponding receptors. A homologous sequence can be a nucleotide sequence that is shared by two or more non-coding polynucleotides, such as noncoding DNA or RNA, regulatory sequences, introns, and sites of transcriptional control or regulation. Homologous sequences can also include conserved sequence regions shared by more than one polynucleotide sequence. Homology does not need to be perfect homology (e.g., 100%), as partially homologous sequences are also contemplated by the instant invention (e.g., 99%, 98%, 97%, 96%, 95%, 94%, 93%, 92%, 91%, 90%, 89%, 88%, 87%, 86%, 85%, 84%, 83%, 82%, 81%, 80% etc.). Indeed, design and use of the DsiRNA agents of the instant invention contemplates the possibility of using such DsiRNA agents not only against target RNAs of interest possessing perfect complementarity with the presently described DsiRNA agents, but also against target RNAs of interest possessing sequences that are, e.g., only 99%, 98%, 97%, 96%, 95%, 94%, 93%, 92%, 91%, 90%, 89%, 88%, 87%, 86%, 85%, 84%, 83%, 82%, 81%, 80% etc. complementary to said DsiRNA agents. Similarly, it is contemplated that the presently described DsiRNA agents of the instant invention might be readily altered by the skilled artisan to enhance the extent of complementarity between said DsiRNA agents and a target RNA of interest, e.g., of a specific allelic variant (e.g., an allele of enhanced therapeutic interest). Indeed, DsiRNA agent sequences with insertions, deletions, and single point mutations relative to the target sequence of interest can also be effective for inhibition (possibly believed to act via microRNA-like translational inhibition, rather than destruction, of targeted transcripts; accordingly, such DsiRNA agents can be termed “DmiRNAs”). Alternatively, DsiRNA agent sequences with nucleotide analog substitutions or insertions can be effective for inhibition.

Sequence identity may be determined by sequence comparison and alignment algorithms known in the art. To determine the percent identity of two nucleic acid sequences (or of two amino acid sequences), the sequences are aligned for optimal comparison purposes (e.g., gaps can be introduced in the first sequence or second sequence for optimal alignment). The nucleotides (or amino acid residues) at corresponding nucleotide (or amino acid) positions are then compared. When a position in the first sequence is occupied by the same residue as the corresponding position in the second sequence, then the molecules are identical at that position. The percent identity between the two sequences is a function of the number of identical positions shared by the sequences (i.e., % homology=# of identical positions/total # of positions.times.100), optionally penalizing the score for the number of gaps introduced and/or length of gaps introduced.

The comparison of sequences and determination of percent identity between two sequences can be accomplished using a mathematical algorithm. In one embodiment, the alignment generated over a certain portion of the sequence aligned having sufficient identity but not over portions having low degree of identity (i.e., a local alignment). A preferred, non-limiting example of a local alignment algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the BLAST programs (version 2.0) of Altschul, et al. (1990) J. Mol. Biol. 215:403-10.

In another embodiment, the alignment is optimized by introducing appropriate gaps and percent identity is determined over the length of the aligned sequences (i.e., a gapped alignment). To obtain gapped alignments for comparison purposes, Gapped BLAST can be utilized as described in Altschul et al., (1997) Nucleic Acids Res. 25(17):3389-3402. In another embodiment, the alignment is optimized by introducing appropriate gaps and percent identity is determined over the entire length of the sequences aligned (i.e., a global alignment). A preferred, non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

Greater than 80% sequence identity, e.g., 80%, 81%, 82%, 83%, 84%, 85%, 86%, 87%, 88%, 89%, 90%, 91%, 92%, 93%, 94%, 95%, 96%, 97%, 98%, 99% or even 100% sequence identity, between the DsiRNA antisense strand and a portion of the RNA sequence of interest is preferred. Alternatively, the DsiRNA may be defined functionally as a nucleotide sequence (or oligonucleotide sequence) that is capable of hybridizing with a portion of the RNA of interest (e.g., 400 mM NaCl, 40 mM PIPES pH 6.4, 1 mM EDTA, 50° C. or 70° C. hybridization for 12-16 hours; followed by washing). Additional preferred hybridization conditions include hybridization at 70° C. in 1×SSC or 50° C. in 1×SSC, 50% formamide followed by washing at 70° C. in 0.3×SSC or hybridization at 70° C. in 4×SSC or 50° C. in 4×SSC, 50% formamide followed by washing at 67° C. in 1×SSC. The hybridization temperature for hybrids anticipated to be less than 50 base pairs in length should be 5-10° C. less than the melting temperature (Tm) of the hybrid, where Tm is determined according to the following equations. For hybrids less than 18 base pairs in length, Tm(° C.)=2(# of A+T bases)+4(# of G+C bases). For hybrids between 18 and 49 base pairs in length, Tm(° C.)=81.5+16.6(log 10[Na+])+0.41 (% G+C)−(600/N), where N is the number of bases in the hybrid, and [Na+] is the concentration of sodium ions in the hybridization buffer ([Na+] for 1×SSC=0.165 M). Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4. The length of the identical nucleotide sequences may be at least about 10, 12, 15, 17, 20, 22, 25, 27 or 30 bases.

By “conserved sequence region” is meant, a nucleotide sequence of one or more regions in a polynucleotide does not vary significantly between generations or from one biological system, subject, or organism to another biological system, subject, or organism. The polynucleotide can include both coding and non-coding DNA and RNA.

By “sense region” is meant a nucleotide sequence of a DsiRNA molecule having complementarity to an antisense region of the DsiRNA molecule. In addition, the sense region of a DsiRNA molecule can comprise a nucleic acid sequence having homology with a target nucleic acid sequence.

By “antisense region” is meant a nucleotide sequence of a DsiRNA molecule having complementarity to a target nucleic acid sequence. In addition, the antisense region of a DsiRNA molecule comprises a nucleic acid sequence having complementarity to a sense region of the DsiRNA molecule.

As used herein, “antisense strand” refers to a single stranded nucleic acid molecule which has a sequence complementary to that of a target RNA. When the antisense strand contains modified nucleotides with base analogs, it is not necessarily complementary over its entire length, but must at least hybridize with a target RNA.

As used herein, “sense strand” refers to a single stranded nucleic acid molecule which has a sequence complementary to that of an antisense strand. When the antisense strand contains modified nucleotides with base analogs, the sense strand need not be complementary over the entire length of the antisense strand, but must at least duplex with the antisense strand.

As used herein, “guide strand” refers to a single stranded nucleic acid molecule of a dsRNA or dsRNA-containing molecule, which has a sequence sufficiently complementary to that of a target RNA to result in RNA interference. After cleavage of the dsRNA or dsRNA-containing molecule by Dicer, a fragment of the guide strand remains associated with RISC, binds a target RNA as a component of the RISC complex, and promotes cleavage of a target RNA by RISC. As used herein, the guide strand does not necessarily refer to a continuous single stranded nucleic acid and may comprise a discontinuity, preferably at a site that is cleaved by Dicer. A guide strand is an antisense strand.

As used herein, “passenger strand” refers to an oligonucleotide strand of a dsRNA or dsRNA-containing molecule, which has a sequence that is complementary to that of the guide strand. As used herein, the passenger strand does not necessarily refer to a continuous single stranded nucleic acid and may comprise a discontinuity, preferably at a site that is cleaved by Dicer. A passenger strand is a sense strand.

By “target nucleic acid” is meant any nucleic acid sequence whose expression, level or activity is to be modulated. The target nucleic acid can be DNA or RNA. Levels of expression may also be targeted via targeting of upstream effectors of the target of interest, or the effects of a modulated or misregulated target may also be modulated by targeting molecules downstream of, for example, the signaling pathway of a target of interest.

As is known, RNAi methods are applicable to a wide variety of genes in a wide variety of organisms and the disclosed compositions and methods can be utilized in each of these contexts. Examples of genes which can be targeted by the disclosed compositions and methods include endogenous genes which are genes that are native to the cell or to genes that are not normally native to the cell. Without limitation these genes include oncogenes, cytokine genes, idiotype (Id) protein genes, prion genes, genes that expresses molecules that induce angiogenesis, genes for adhesion molecules, cell surface receptors, proteins involved in metastasis, proteases, apoptosis genes, cell cycle control genes, genes that express EGF and the EGF receptor, multi-drug resistance genes, such as the MDR1 gene.

More specifically, the target mRNA of the invention specifies the amino acid sequence of a cellular protein (e.g., a nuclear, cytoplasmic, transmembrane, or membrane-associated protein). In another embodiment, the target mRNA of the invention specifies the amino acid sequence of an extracellular protein (e.g., an extracellular matrix protein or secreted protein). As used herein, the phrase “specifies the amino acid sequence” of a protein means that the mRNA sequence is translated into the amino acid sequence according to the rules of the genetic code. The following classes of proteins are listed for illustrative purposes: developmental proteins (e.g., adhesion molecules, cyclin kinase inhibitors, Wnt family members, Pax family members, Winged helix family members, Hox family members, cytokines/lymphokines and their receptors, growth/differentiation factors and their receptors, neurotransmitters and their receptors); oncogene-encoded proteins (e.g., ABLI, BCLI, BCL2, BCL6, CBFA2, CBL, CSFIR, ERBA, ERBB, EBRB2, ETSI, ETSI, ETV6, FGR, FOS, FYN, HCR, HRAS, JUN, KRAS, LCK, LYN, MDM2, MLL, MYB, MYC, MYCLI, MYCN, NRAS, PIM I, PML, RET, SRC, TALI, TCL3, and YES); tumor suppressor proteins (e.g., BRCA1, BRCA2, MADH4, MCC, NF I, NF2, RB I, TP53, and WTI); and enzymes (e.g., ACC synthases and oxidases, ACP desaturases and hydroxylases, ADP-glucose pyrophorylases, ATPases, alcohol dehydrogenases, amylases, amyloglucosidases, catalases, cellulases, chalcone synthases, chitinases, cyclooxygenases, decarboxylases, dextriinases, DNA and RNA polymerases, galactosidases, glucanases, glucose oxidases, granule-bound starch synthases, GTPases, helicases, hernicellulases, integrases, inulinases, invertases, isomerases, kinases, lactases, lipases, lipoxygenases, lysozymes, nopaline synthases, octopine synthases, pectinesterases, peroxidases, phosphatases, phospholipases, phosphorylases, phytases, plant growth regulator synthases, polygalacturonases, proteinases and peptidases, pullanases, recombinases, reverse transcriptases, RUBISCOs, topoisomerases, and xylanases), ApoB100 and HPRT1.

In one aspect, the target mRNA molecule of the invention specifies the amino acid sequence of a protein associated with a pathological condition. For example, the protein may be a pathogen-associated protein (e.g., a viral protein involved in immunosuppression of the host, replication of the pathogen, transmission of the pathogen, or maintenance of the infection), or a host protein which facilitates entry of the pathogen into the host, drug metabolism by the pathogen or host, replication or integration of the pathogen's genome, establishment or spread of infection in the host, or assembly of the next generation of pathogen. Pathogens include RNA viruses such as flaviviruses, picornaviruses, rhabdoviruses, filoviruses, retroviruses, including lentiviruses, or DNA viruses such as adenoviruses, poxviruses, herpes viruses, cytomegaloviruses, hepadnaviruses or others. Additional pathogens include bacteria, fungi, helminths, schistosomes and trypanosomes. Other kinds of pathogens can include mammalian transposable elements. Alternatively, the protein may be a tumor-associated protein or an autoimmune disease-associated protein.

The target gene may be derived from or contained in any organism. The organism may be a plant, animal, protozoa, bacterium, virus or fungus. See e.g., U.S. Pat. No. 6,506,559, incorporated herein by reference.

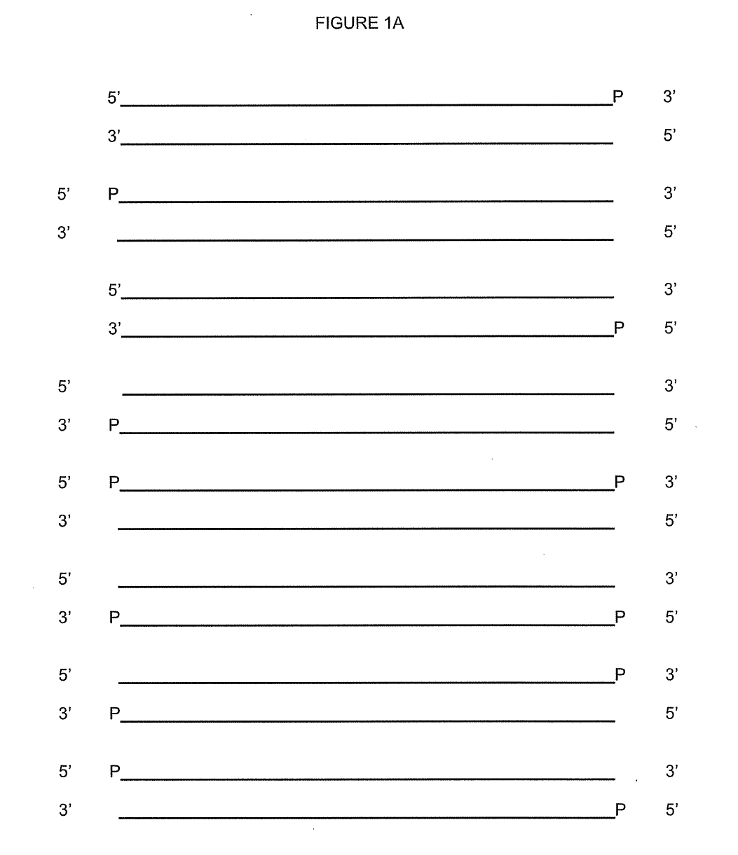

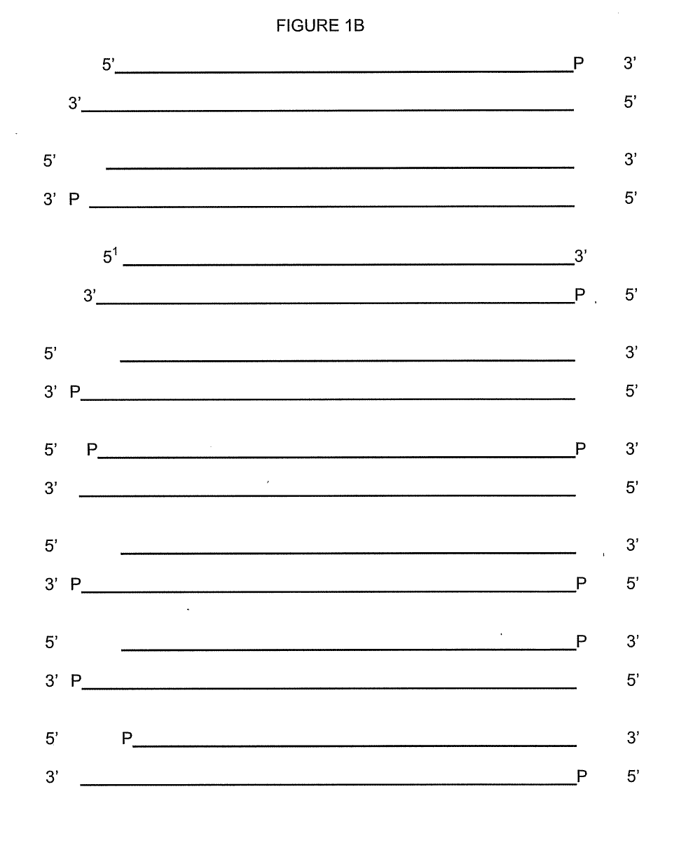

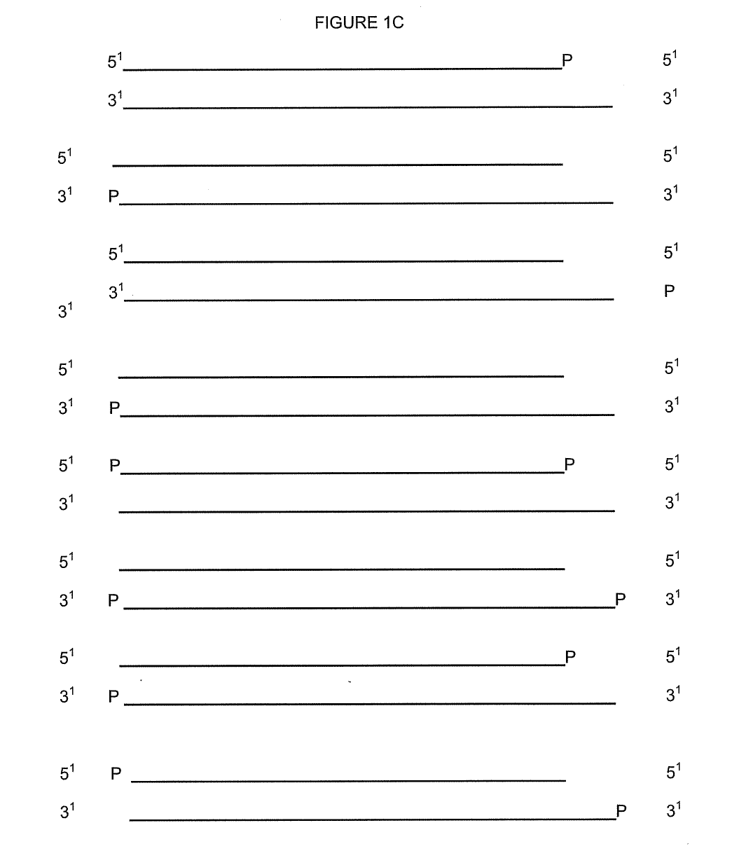

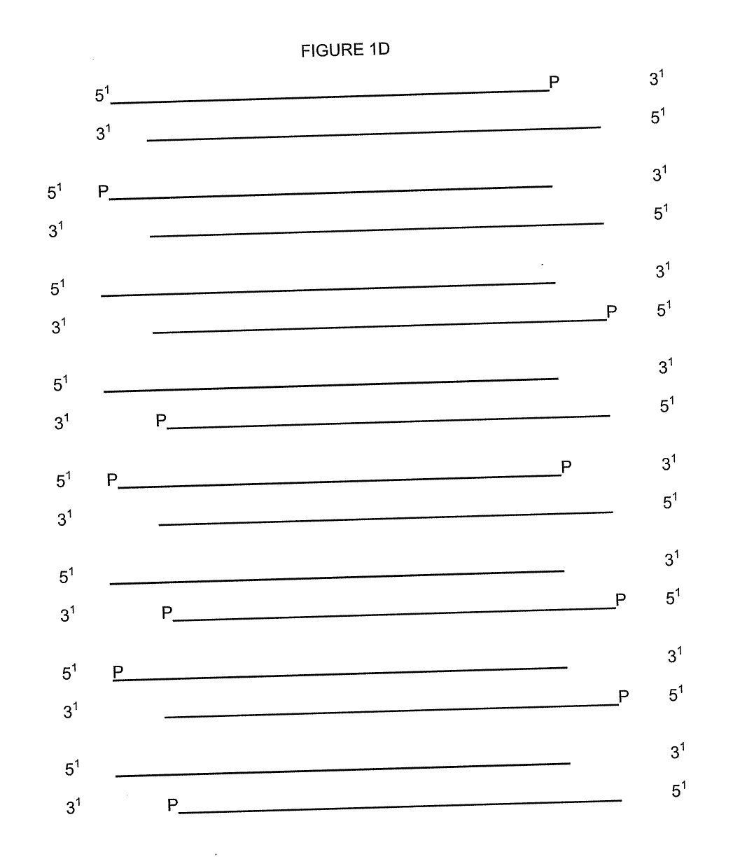

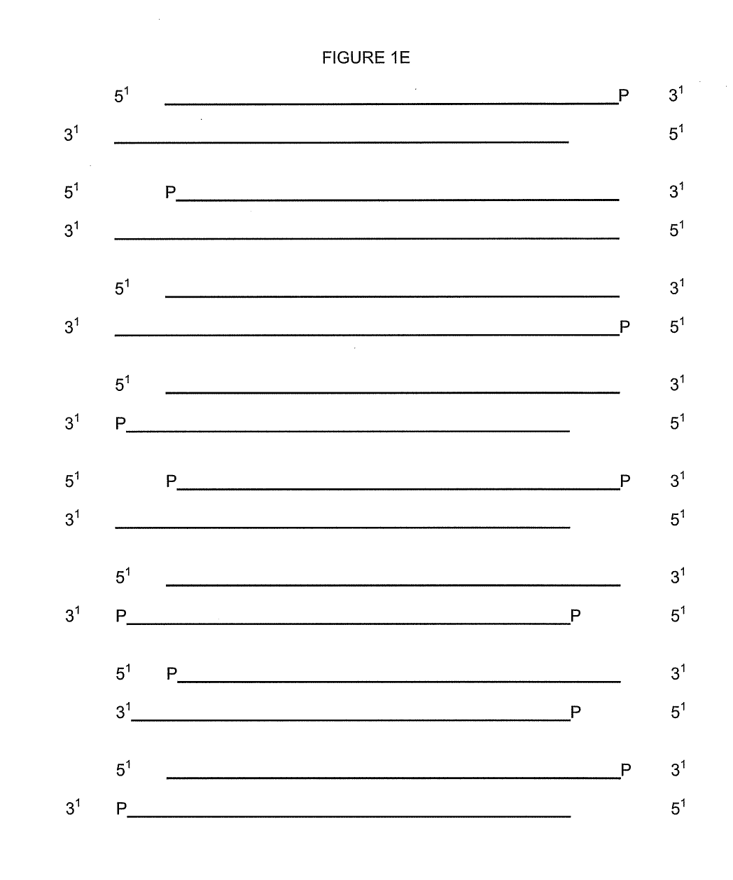

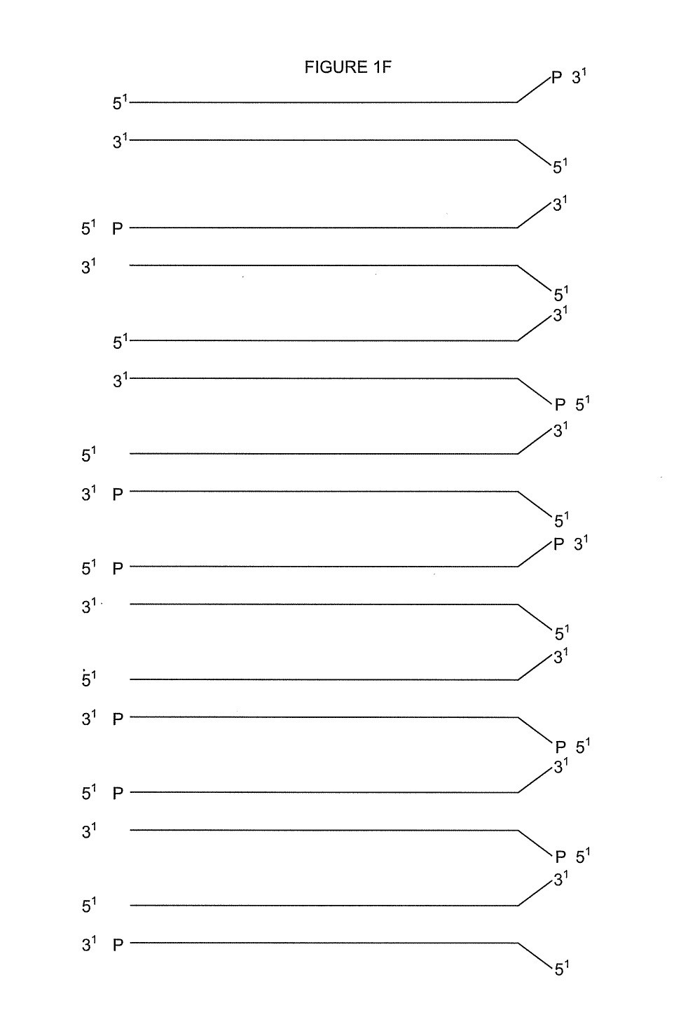

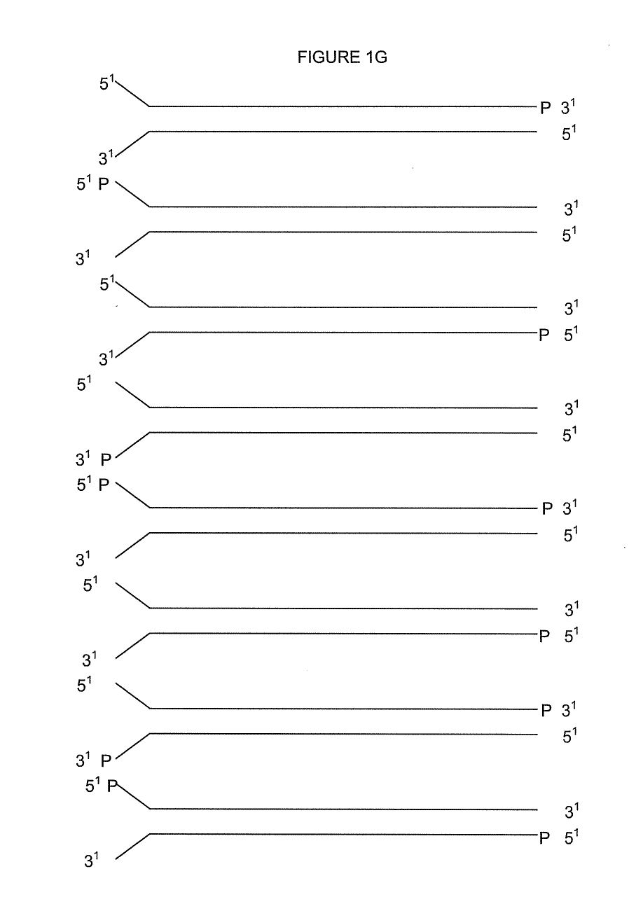

In one embodiment of the present invention, each sequence of a DsiRNA molecule of the invention is independently about 25 to about 35 nucleotides in length, in specific embodiments about 25, 26, 27, 28, 29, 30, 31, 32, 33, 34 or 35 nucleotides in length. In another embodiment, the DsiRNA duplexes of the invention independently comprise about 25 to about 30 base pairs (e.g., about 25, 26, 27, 28, 29, or 30). In another embodiment, one or more strands of the DsiRNA molecule of the invention independently comprises about 19 to about 35 nucleotides (e.g., about 19, 20, 21, 22, 23, 24, 25, 26, 27, 28, 29, 30, 31, 32, 33, 34 or 35) that are complementary to a target nucleic acid molecule of interest. Exemplary DsiRNA molecules of the invention are shown in FIG. 1, and below.

As used herein “cell” is used in its usual biological sense, and does not refer to an entire multicellular organism, e.g., specifically does not refer to a human. The cell can be present in an organism, e.g., birds, plants and mammals such as humans, cows, sheep, apes, monkeys, swine, dogs, and cats. The cell can be prokaryotic (e.g., bacterial cell) or eukaryotic (e.g., mammalian or plant cell). The cell can be of somatic or germ line origin, totipotent or pluripotent, dividing or non-dividing. The cell can also be derived from or can comprise a gamete or embryo, a stem cell, or a fully differentiated cell. Within certain aspects, the term “cell” refers specifically to mammalian cells, such as human cells, that contain one or more isolated dsNA molecules of the present disclosure. In particular aspects, a cell processes dsRNAs or dsRNA-containing molecules resulting in RNA interference of target nucleic acids, and contains proteins and protein complexes required for RNAi, e.g., Dicer and RISC.

The DsiRNA molecules of the invention are added directly, or can be complexed with cationic lipids, packaged within liposomes, or otherwise delivered to target cells or tissues. The nucleic acid or nucleic acid complexes can be locally administered to relevant tissues ex vivo, or in vivo through direct dermal application, transdermal application, or injection, with or without their incorporation in biopolymers. In certain aspects of the invention, the dsRNAs of the exemplary structures of dsRNA-peptides presented in FIG. 1 are modified in accordance with the below description of modification patterning of DsiRNA agents. Chemically modified forms of constructs described in FIG. 1, and the below exemplary structures can be used in any and all uses described for the DsiRNA agents described herein.

In another aspect, the invention provides mammalian cells containing one or more DsiRNA molecules of this invention. The one or more DsiRNA molecules can independently be targeted to the same or different sites.

By “RNA” is meant a molecule comprising at least one ribonucleotide residue. By “ribonucleotide” is meant a nucleotide with a hydroxyl group at the 2′ position of a β-D-ribofuranose moiety. The terms include double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution and/or alteration of one or more nucleotides. Such alterations can include addition of non-nucleotide material, such as to the end(s) of the DsiRNA or internally, for example at one or more nucleotides of the RNA. Nucleotides in the RNA molecules of the instant invention can also comprise non-standard nucleotides, such as non-naturally occurring nucleotides or chemically synthesized nucleotides or deoxynucleotides. These altered RNAs can be referred to as analogs or analogs of naturally-occurring RNA.

By “subject” is meant an organism, which is a donor or recipient of explanted cells or the cells themselves. “Subject” also refers to an organism to which the DsiRNA agents of the invention can be administered. A subject can be a mammal or mammalian cells, including a human or human cells.

The phrase “pharmaceutically acceptable carrier” refers to a carrier for the administration of a therapeutic agent. Exemplary carriers include saline, buffered saline, dextrose, water, glycerol, ethanol, and combinations thereof. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract. The pharmaceutically acceptable carrier of the disclosed dsRNA compositions may be micellar structures, such as a liposomes, capsids, capsoids, polymeric nanocapsules, or polymeric microcapsules.

Polymeric nanocapsules or microcapsules facilitate transport and release of the encapsulated or bound dsRNA into the cell. They include polymeric and monomeric materials, especially including polybutylcyanoacrylate. A summary of materials and fabrication methods has been published (see Kreuter, 1991). The polymeric materials which are formed from monomeric and/or oligomeric precursors in the polymerization/nanoparticle generation step, are per se known from the prior art, as are the molecular weights and molecular weight distribution of the polymeric material which a person skilled in the field of manufacturing nanoparticles may suitably select in accordance with the usual skill.

Various methodologies of the instant invention include step that involves comparing a value, level, feature, characteristic, property, etc. to a “suitable control”, referred to interchangeably herein as an “appropriate control”. A “suitable control” or “appropriate control” is any control or standard familiar to one of ordinary skill in the art useful for comparison purposes. In one embodiment, a “suitable control” or “appropriate control” is a value, level, feature, characteristic, property, etc. determined prior to performing an RNAi methodology, as described herein. For example, a transcription rate, mRNA level, translation rate, protein level, biological activity, cellular characteristic or property, genotype, phenotype, etc. can be determined prior to introducing an RNA silencing agent (e.g., DsiRNA) of the invention into a cell or organism. In another embodiment, a “suitable control” or “appropriate control” is a value, level, feature, characteristic, property, etc. determined in a cell or organism, e.g., a control or normal cell or organism, exhibiting, for example, normal traits. In yet another embodiment, a “suitable control” or “appropriate control” is a predefined value, level, feature, characteristic, property, etc.

The term “in vitro” has its art recognized meaning, e.g., involving purified reagents or extracts, e.g., cell extracts. The term “in vivo” also has its art recognized meaning, e.g., involving living cells, e.g., immortalized cells, primary cells, cell lines, and/or cells in an organism.

“Treatment”, or “treating” as used herein, is defined as the application or administration of a therapeutic agent (e.g., a DsiRNA agent or a vector or transgene encoding same) to a patient, or application or administration of a therapeutic agent to an isolated tissue or cell line from a patient, who has a disorder with the purpose to cure, heal, alleviate, relieve, alter, remedy, ameliorate, improve or affect the disease or disorder, or symptoms of the disease or disorder. The term “treatment” or “treating” is also used herein in the context of administering agents prophylactically. The term “effective dose” or “effective dosage” is defined as an amount sufficient to achieve or at least partially achieve the desired effect. The term “therapeutically effective dose” is defined as an amount sufficient to cure or at least partially arrest the disease and its complications in a patient already suffering from the disease. The term “patient” includes human and other mammalian subjects that receive either prophylactic or therapeutic treatment.

dsRNA-Peptide Design/Synthesis

It has been found empirically that longer dsRNA species of from 25 to about 35 nucleotides (DsiRNAs) and especially from 25 to about 30 nucleotides give unexpectedly effective results in terms of potency and duration of action, as compared to 19-23mer siRNA agents. Without wishing to be bound by the underlying theory of the dsRNA processing mechanism, it is thought that the longer dsRNA species serve as a substrate for the Dicer enzyme in the cytoplasm of a cell. In addition to cleaving the dsRNA of the invention into shorter segments, Dicer is thought to facilitate the incorporation of a single-stranded cleavage product derived from the cleaved dsRNA into the RISC complex that is responsible for the destruction of a target RNA of interest. Prior studies (Rossi et al., U.S. Patent Application No. 2007/0265220) have shown that the cleavability of a dsRNA species (specifically, a DsiRNA agent) by Dicer corresponds with increased potency and duration of action of the dsRNA species.

The invention encompasses dsRNAs comprising double stranded RNAs comprising a first strand and a second strand wherein the first strand and the second strand have a length which is at least 16 and at most 50 nucleotides in length (for example 16-50, 19-35, 19-24, 25-30, 25, 35, 19-23, and 21-23 nucleotides in length).

A. dsRNAs

Design of dsRNAs, including DsiRNAs can optionally involve use of predictive scoring algorithms that perform in silico assessments of the projected activity/efficacy of a number of possible DsiRNA agents spanning a region of sequence. Information regarding the design of such scoring algorithms can be found, e.g., in Gong et al. (BMC Bioinformatics 2006, 7:516), though a more recent “v3” algorithm represents a theoretically improved algorithm relative to siRNA scoring algorithms previously available in the art. (The “v3” scoring algorithm is a machine learning algorithm that is not reliant upon any biases in human sequence. In addition, the “v3” algorithm derives from a data set that is approximately three-fold larger than that from which an older “v2” algorithm such as that described in Gong et al. derives.)

The first and second oligonucleotides of the DsiRNA agents of the instant invention are not required to be completely complementary. In fact, in one embodiment, the 3′-terminus of the sense strand contains one or more mismatches. In one aspect, about two mismatches are incorporated at the 3′ terminus of the sense strand. In another embodiment, the DsiRNA of the invention is a double stranded RNA molecule containing two RNA oligonucleotides each of which is 27 nucleotides in length and, when annealed to each other, have blunt ends and a two nucleotide mismatch on the 3′-terminus of the sense strand (the 5′-terminus of the antisense strand). The use of mismatches or decreased thermodynamic stability (specifically at the 3′-sense/5′-antisense position) has been proposed to facilitate or favor entry of the antisense strand into RISC (Schwarz et al., 2003, Cell 115: 199-208; Khvorova et al., 2003, Cell 115: 209-216), presumably by affecting some rate-limiting unwinding steps that occur with entry of the siRNA into RISC. Thus, terminal base composition has been included in design algorithms for selecting active 21mer siRNA duplexes (Ui-Tei et al., 2004, Nucleic Acids Res 32: 936-948; Reynolds et al., 2004, Nat Biotechnol 22: 326-330). With Dicer cleavage of the dsRNA of this embodiment, the small end-terminal sequence which contains the mismatches will either be left unpaired with the antisense strand (become part of a 3′-overhang) or be cleaved entirely off the final 21-mer siRNA. These “mismatches”, therefore, do not persist as mismatches in the final RNA component of RISC. The finding that base mismatches or destabilization of segments at the 3′-end of the sense strand of Dicer substrate improved the potency of synthetic duplexes in RNAi, presumably by facilitating processing by Dicer, was a surprising finding of past works describing the design and use of 25-30mer dsRNAs (also termed “DsiRNAs” herein; Rossi et al., U.S. Patent Application Nos. 2005/0277610, 2005/0244858 and 2007/0265220).

B. Peptides

The invention provides for compositions comprising a dsRNA of the invention conjugated to a peptide.

Delivery Peptides

In certain embodiments the peptide of interest is a delivery peptide as defined hereinabove.

Delivery Peptide Sequences Useful According to the invention

A delivery peptide useful according to the invention increases at least one of onset of action of a dsRNA, duration of action by the delivered dsRNA or speed of delivery of a dsRNA of the invention, as compared to an unconjugated dsRNA. A peptide of the invention decreases, as defined herein, the onset of action such that there is a decrease in the lag time before a dsRNA of interest reaches a target RNA as compared an unconjugated dsRNA. A delivery peptide useful according to the invention increases, as defined herein, the duration of action such that a dsRNA-peptide conjugate inhibits a target RNA for a longer period of time, as compared to an unconjugated dsRNA. A delivery peptide useful according to the invention increases, as defined herein, the speed of delivery of a dsRNA such that a dsRNA-peptide conjugate reaches a target RNA faster than an unconjugated dsRNA.

According to the invention, an amino acid sequence of a delivery peptide is determined and optimized for the dsRNA to be delivered. Peptide sequences useful for delivery peptides according to the invention are described in the literature.

In one embodiment, a delivery peptide according to the invention comprises proline residues, for example, a sequence of x1-P-x2-P-x3, where x1 and x3 are any amino acid or peptide segment comprising 2 to 50 amino acids and x2 is either 0 or 1 amino acids or peptide segments containing 2 to 20 amino acids. In another embodiment, x1=a peptide comprising 5 amino acid residues; x2=a peptide comprising 7 amino acid residues and x3=a peptide comprising 4 amino acid residues. In another embodiment, x1=a peptide comprising 8 amino acid residues; x2=a peptide comprising 7 amino acid residues and x3=a peptide comprising 4 amino acid residues. In yet another embodiment, x1=a peptide comprising 8 amino acid residues; x2=a peptide comprising 8 amino acid residues and x3=a peptide comprising 4 amino acid residues (Deber et al., Arch Biochem Biophys. 1986; 251(1):68-76; and Du et al., J Pept Res. 1998; 51(3):235-43.)

Delivery peptide sequences useful for the invention include, but are not limited to:

| | |
|---|---|
| VRGIITSKTKSLDKGYNKALNDL | (SEQ ID NO: 1) |
| VRGIIPFKTKSLDEGYNKALNDL | (SEQ ID NO: 2) |
| KSVKAPGI | (SEQ ID NO: 3) |
| HKAIDGRSLYNKTLD | (SEQ ID NO: 4) |
| LRLTKNSRDDST | (SEQ ID NO: 5) |
| KNIVSVKGIRKSI | (SEQ ID NO: 6) |
| KSVIPRKGTKAPPRL | (SEQ ID NO: 7) |
| KPVMYKNTGKSEQ | (SEQ ID NO: 8) |
| EFVMNPANAQGHTPGTRL | (SEQ ID NO: 9) |
| EFVMNPANAQGHTAGTRL | (SEQ ID NO: 10) |
| EFVMNAANAQGHTPGTRL | (SEQ ID NO: 11) |
| EFVMNPANAQGRHTPGTRL | (SEQ ID NO: 12) |
| NPKEFVMNPANAQGHTPGTRL | (SEQ ID NO: 13) |
| NPKEFVMNPANAQGRHTPGTRL | (SEQ ID NO: 14) |
| KKIIPPTNIRENLYNRTASLTDLGGEL | (SEQ ID NO: 15) |
| CVRGIITSKTKSLDKGYNKALNDL | (SEQ ID NO: 16) |
| CVRGIIPFKTKSLDEGYNKALNDL | (SEQ ID NO: 17) |
| CKSVKAPGI | (SEQ ID NO: 18) |
| CHKAIDGRSLYNKTLD | (SEQ ID NO: 19) |
| CLRLTKNSRDDST | (SEQ ID NO: 20) |
| CKNIVSVKGIRKSI | (SEQ ID NO: 21) |
| CKSVIPRKGTKAPPRL | (SEQ ID NO: 22) |
| CKPVMYKNTGKSEQ | (SEQ ID NO: 23) |
| CEFVMNPANAQGHTPGTRL | (SEQ ID NO: 24) |
| CEFVMNPANAQGHTAGTRL | (SEQ ID NO: 25) |
| CEFVMNAANAQGHTPGTRL | (SEQ ID NO: 26) |
| CEFVMNPANAQGRHTPGTRL | (SEQ ID NO: 27) |
| CNPKEFVMNPANAQGHTPGTRL | (SEQ ID NO: 28) |
| CNPKEFVMNPANAQGRHTPGTRL | (SEQ ID NO: 29) |
| CKKIIPPTNIRENLYNRTASLTDLGGEL | (SEQ ID NO: 30) |
| GVRGIITSKTKSLDKGYNKALNDL | (SEQ ID NO: 31) |
| GVRGIIPFKTKSLDEGYNKALNDL | (SEQ ID NO: 32) |
| GKSVKAPGI | (SEQ ID NO: 33) |
| GHKAIDGRSLYNKTLD | (SEQ ID NO: 34) |
| GLRLTKNSRDDST | (SEQ ID NO: 35) |
| GKNIVSVKGIRKSI | (SEQ ID NO: 36) |
| GKSVIPRKGTKAPPRL | (SEQ ID NO: 37) |
| GKPVMYKNTGKSEQ | (SEQ ID NO: 38) |
| GEFVMNPANAQGHTPGTRL | (SEQ ID NO: 39) |
| GEFVMNPANAQGHTAGTRL | (SEQ ID NO: 40) |
| GEFVMNAANAQGHTPGTRL | (SEQ ID NO: 41) |
| GEFVMNPANAQGRHTPGTRL | (SEQ ID NO: 42) |
| GNPKEFVMNPANAQGHTPGTRL | (SEQ ID NO: 43) |
| GNPKEFVMNPANAQGRHTPGTRL | (SEQ ID NO: 44) |
| GKKIIPPTNIRENLYNRTASLTDLGGEL | (SEQ ID NO: 45) |
| VRGIITSKTKSLDKGYNKALNDLC | (SEQ ID NO: 46) |
| VRGIIPFKTKSLDEGYNKALNDLC | (SEQ ID NO: 47) |
| KSVKAPGIC | (SEQ ID NO: 48) |
| HKAIDGRSLYNKTLDC | (SEQ ID NO: 49) |
| LRLTKNSRDDSTC | (SEQ ID NO: 50) |
| KNIVSVKGIRKSIC | (SEQ ID NO: 51) |
| KSVIPRKGTKAPPRLC | (SEQ ID NO: 52) |
| KPVMYKNTGKSEQC | (SEQ ID NO: 53) |
| EFVMNPANAQGHTPGTRLC | (SEQ ID NO: 54) |
| EFVMNPANAQGHTAGTRLC | (SEQ ID NO: 55) |
| EFVMNAANAQGHTPGTRLC | (SEQ ID NO: 56) |
| EFVMNPANAQGRHTPGTRLC | (SEQ ID NO: 57) |
| NPKEFVMNPANAQGHTPGTRLC | (SEQ ID NO: 58) |
| NPKEFVMNPANAQGRHTPGTRLC | (SEQ ID NO: 59) |
| KKIIPPTNIRENLYNRTASLTDLGGELC | (SEQ ID NO: 60) |
| KSVKAPGIGGKSVKAPGI | (SEQ ID NO: 61) |
| KSVKAPGIGGKSVKAPGIGGKSVKAPGI | (SEQ ID NO: 62) |
| KSVKAPGIGG(KSVKAPGI)₂ | (SEQ ID NO: 63) |
| CKSVKAPGIGGKSVKAPGI | (SEQ ID NO: 64) |
| CKSVKAPGIGGKSVKAPGIGGKSVKAPGI | (SEQ ID NO: 65) |
| GLFGAIAGFIENGWEGMIDGWYG | (SEQ ID NO: 66) |
| CGLFGAIAGFIENGWEGMIDGWYG | (SEQ ID NO: 67) |
| GLFGAIAGFIENGWEGMIDGWYGC | (SEQ ID NO: 68) |
| GRGDGG | (SEQ ID NO: 69) |
| CRGDGG | (SEQ ID NO: 70) |
| GRGDGC | (SEQ ID NO: 71) |
| THALWHT | (SEQ ID NO: 72) |
| GTHALWHT | (SEQ ID NO: 73) |
| THALWHTG | (SEQ ID NO: 74) |
| CTHALWHT | (SEQ ID NO: 75) |
| THALWHTC | (SEQ ID NO: 76) |
| QPFMQCLCLIYDASC | (SEQ ID NO: 77) |
| GQPFMQCLCLIYDASC | (SEQ ID NO: 78) |
| QPFMQCLCLIYDASCG | (SEQ ID NO: 79) |
| RNVPPIFNDVYWIAF | (SEQ ID NO: 80) |
| GRNVPPIFNDVYWIAF | (SEQ ID NO: 81) |
| RNVPPIFNDVYWIAFG | (SEQ ID NO: 82) |
| CRNVPPIFNDVYWIAF | (SEQ ID NO: 83) |
| RNVPPIFNDVYWIAFC | (SEQ ID NO: 84) |
| VFRVRPWYQSTSQS | (SEQ ID NO: 85) |
| GVFRVRPWYQSTSQS | (SEQ ID NO: 86) |
| VFRVRPWYQSTSQSG | (SEQ ID NO: 87) |
| CVFRVRPWYQSTSQS | (SEQ ID NO: 88) |
| VFRVRPWYQSTSQSC | (SEQ ID NO: 89) |

or portions thereof.

Targeting Peptides

In other embodiments, the peptide of interest is a targeting peptide as defined hereinabove.

According to the invention, an amino acid sequence of a targeting peptide is determined and optimized for the dsRNA that is conjugated to the peptide for delivery. Peptide sequences useful for targeting peptides according to the invention are described in the literature.

For each ligand family distinct peptide sequence patterns are appropriate. In one embodiment, a peptide useful for targeting the LDL-receptor according to the current invention may contain a sequence of x1-F-x2-YGG-x3, where x1 and x3 are any amino acid or peptide segment containing 2 to 40 amino acids, and x2 is any amino acid. Hussain, Strickland and Bakillah, Annu Rev Nutr. 1999; 19:141-172. Hussain, Front Biosci. 2001; 6:D417-D428.

Targeting Peptides Useful According to the Invention

Targeting peptides useful according to the invention include but are not limited to an amino acid sequence from any of the following ligands:

1. Parathyroid Hormone (PTH) and PTH-Related Protein
P01270; PTHY_HUMAN
P22858; PTHR_MOUSE
Q811S6; Q811S6_MOUSE
P01270; PTHY_HUMAN

(SEQ ID NO: 90)

MIPAKDMAKV MIVMLAICFL TKSDGKSVKK RSVSEIQLMH
NLGKHLNSME RVEWLRKKLQ DVHNFVALGA PLAPRDAGSQ
RPRKKEDNVL VESHEKSLGE ADKADVNVLT KAKSQ

2. Thyroid Stimulating Hormone (TSH)
P01222; TSHB_HUMAN
P12656; TSHB_MOUSE
P01222; TSHB_HUMAN

(SEQ ID NO: 91)

MTALFLMSML FGLACGQAMS FCIPTEYTMH IERRECAYCL
TINTTICAGY CMTRDINGKL FLPKYALSQD VCTYRDFIYR
TVEIPGCPLH VAPYFSYPVA LSCKCGKCNT DYSDCIHEAI
KTNYCTKPQK SYLVGFSV

3. TSH Releasing Hormone
B2R8R1; B2R8R1_HUMAN
B2R8R1; B2R8R1_HUMAN

(SEQ ID NO: 92)

MPGPWLLLAL ALTLNLTGVP GGRAQPEAAQ QEAVTAAEHP
GLDDFLRQVE RLLFLRENIQ RLQGDQGEHS ASQIFQSDWL
SKRQHPGKRE EEEEEGVEEE EEEEGGAVGP HKRQHPGRRE
DEASWSVDVT QHKRQHPGRR SPWLAYAVPK RQHPGRRLAD
PKAQRSWEEE EEEEEREEDL MPEKRQHPGK RALGGPCGPQ
GAYGQAGLLL GLLDDLSRSQ GAEEKRQHPG RRAAWVREPL
EE

4. FSH/LH Releasing Hormone
P01148; GON1_HUMAN
O43555; GON2_HUMAN
P13562; GON1_MOUSE
P01148; GONLHUMAN

(SEQ ID NO: 93)

MKPIQKLLAG LILLTWCVEG CSSQHWSYGL RPGGKRDAEN
LIDSFQEIVK EVGQLAETQR FECTTHQPRS PLRDLKGALE
SLIEEETGQK KI

O43555; GON2_HUMAN

(SEQ ID NO: 94)

MASSRRGLLL LLLLTAHLGP SEAQHWSHGW YPGGKRALSS
AQDPQNALRP PGRALDTAAG SPVQTAHGLP SDALAPLDDS
MPWEGRTTAQ WSLHRKRHLA RTLLTAAREP RPAPPSSNKV

5. Corticotropin Releasing Hormone (CRH)
P06850; CRF_HUMAN
Q8CIT0; CRF_MOUSE
P06850; CRF_HUMAN

(SEQ ID NO: 95)

MRLPLLVSAG VLLVALLPCP PCRALLSRGP VPGARQAPQH
PQPLDFFQPP PQSEQPQQPQ ARPVLLRMGE EYFLRLGNLN
KSPAAPLSPA SSLLAGGSGS RPSPEQATAN FFRVLLQQLL
LPRRSLDSPA ALAERGARNA LGGHQEAPER ERRSEEPPIS
LDLTFHLLRE VLEMARAEQL AQQAHSNRKL MEIIGK

6. Adrenocorticotropic Hormone (ACTH)
P01189; COLI_HUMAN
P01193; COLI_MOUSE

P01189; COLI_HUMAN

(SEQ ID NO: 96)

MPRSCCSRSG ALLLALLLQA SMEVRGWCLE SSQCQDLTTE
SNLLECIRAC KPDLSAETPM FPGNGDEQPL TENPRKYVMG
HFRWDRFGRR NSSSSGSSGA GQKREDVSAG EDCGPLPEGG
PEPRSDGAKP GPREGKRSYS MEHFRWGKPV GKKRRPVKVY
PNGAEDESAE AFPLEFKREL TGQRLREGDG PDGPADDGAG
AQADLEHSLL VAAEKKDEGP YRMEHFRWGS PPKDKRYGGF
MTSEKSQTPL VTLFKNAIIK NAYKKGE

7. Proteinase Activated Receptor (PAR) Ligands and Thrombin Receptor Agonists
P00734; THRB_HUMAN
P19221; THRB_MOUSE
P00734; THRB_HUMAN

(SEQ ID NO: 97)

MAHVRGLQLP GCLALAALCS LVHSQHVFLA PQQARSLLQR
VRRANTFLEE VRKGNLEREC VEETCSYEEA FEALESSTAT
DVFWAKYTAC ETARTPRDKL AACLEGNCAE GLGTNYRGHV
NITRSGIECQ LWRSRYPHKP EINSTTHPGA DLQENFCRNP
DSSTTGPWCY TTDPTVRRQE CSIPVCGQDQ VTVAMTPRSE
GSSVNLSPPL EQCVPDRGQQ YQGRLAVTTH GLPCLAWASA
QAKALSKHQD FNSAVQLVEN FCRNPDGDEE GVWCYVAGKP
GDFGYCDLNY CEEAVEEETG DGLDEDSDRA IEGRTATSEY
QTFFNPRTFG SGEADCGLRP LFEKKSLEDK TERELLESYI
DGRIVEGSDA EIGMSPWQVM LFRKSPQELL CGASLISDRW
VLTAAHCLLY PPWDKNFTEN DLLVRIGKHS RTRYERNIEK
ISMLEKIYIH PRYNWRENLD RDIALMKLKK PVAFSDYIHP
VCLPDRETAA SLLQAGYKGR VTGWGNLKET WTANVGKGQP
SVLQVVNLPI VERPVCKDST RIRITDNMFC AGYKPDEGKR
GDACEGDSGG PFVMKSPFNN RWYQMGIVSW GEGCDRDGKY
GFYTHVFRLK KWIQKVIDQF GE

8. Complement Receptor Ligands
P01024; CO3_HUMAN
P01027; CO3_MOUSE
P01024; CO3_HUMAN

(SEQ ID NO: 98)

MGPTSGPSLL LLLLTHLPLA LGSPMYSIIT PNILRLESEE
TMVLEAHDAQ GDVPVTVTVH DFPGKKLVLS SEKTVLTPAT
NHMGNVTFTI PANREFKSEK GRNKFVTVQA TFGTQVVEKV
VLVSLQSGYL FIQTDKTIYT PGSTVLYRIF TVNHKLLPVG
RTVMVNIENP EGIPVKQDSL SSQNQLGVLP LSWDIPELVN
MGQWKIRAYY ENSPQQVFST EFEVKEYVLP SFEVIVEPTE
KFYYIYNEKG LEVTITARFL YGKKVEGTAF VIFGIQDGEQ
RISLPESLKR IPIEDGSGEV VLSRKVLLDG VQNPRAEDLV
GKSLYVSATV ILHSGSDMVQ AERSGIPIVT SPYQIHFTKT
PKYFKPGMPF DLMVFVTNPD GSPAYRVPVA VQGEDTVQSL
TQGDGVAKLS INTHPSQKPL SITVRTKKQE LSEAEQATRT
NSNNYLHLSV LRTELRPGET LNVNFLLRMD RAHEAKIRYY
TYLIMNKGRL LKAGRQVREP GQDLVVLPLS ITTDFIPSFR
LVAYYTLIGA SGQREVVADS VWVDVKDSCV GSLVVKSGQS
EDRQPVPGQQ MTLKIEGDHG ARVVLVAVDK GVFVLNKKNK
LTQSKIWDVV EKADIGCTPG SGKDYAGVFS DAGLTFTSSS
GQQTAQRAEL QCPQPAARRR RSVQLTEKRM DKVGKYPKEL
RKCCEDGMRE NPMRFSCQRR TRFISLGEAC KKVFLDCCNY
ITELRRQHAR ASHLGLARSN LDEDIIAEEN IVSRSEFPES
WLWNVEDLKE PPKNGISTKL MNIFLKDSIT TWEILAVSMS
DKKGICVADP FEVTVMQDFF IDLRLPYSVV RNEQVEIRAV
LYNYRQNQEL KVRVELLHNP AFCSLATTKR RHQQTVTIPP
KSSLSVPYVI VPLKTGLQEV EVKAAVYHHF ISDGVRKSLK
VVPEGIRMNK TVAVRTLDPE RLGREGVQKE DIPPADLSDQ
VPDTESETRI LLQGTPVAQM TEDAVDAERL KHLIVTPSGC
GEQNMIGMTP TVIAVHYLDE TEQWEKFGLE KRQGALELIK
KGYTQQLAFR QPSSAFAAFV KRAPSTWLTA YVVKVFSLAV
NLIAIDSQVL CGAVKWLILE KQKPDGVFQE DAPVIHQEMI
GGLRNNNEKD MALTAFVLIS LQEAKDICEE QVNSLPGSIT
KAGDFLEANY MNLQRSYTVA IAGYALAQMG RLKGPLLNKF
LTTAKDKNRW EDPGKQLYNV EATSYALLAL LQLKDFDFVP
PVVRWLNEQR YYGGGYGSTQ ATFMVFQALA QYQKDAPDHQ
ELNLDVSLQL PSRSSKITHR IHWESASLLR SEETKENEGF
TVTAEGKGQG TLSVVTMYHA KAKDQLTCNK FDLKVTIKPA
PETEKRPQDA KNTMILEICT RYRGDQDATM SILDISMMTG
FAPDTDDLKQ LANGVDRYIS KYELDKAFSD RNTLIIYLDK
VSHSEDDCLA FKVHQYFNVE LIQPGAVKVY AYYNLEESCT
RFYHPEKEDG KLNKLCRDEL CRCAEENCFI QKSDDKVTLE
ERLDKACEPG VDYVYKTRLV KVQLSNDFDE YIMAIEQTIK
SGSDEVQVGQ QRTFISPIKC REALKLEEKK HYLMWGLSSD
FWGEKPNLSY IIGKDTWVEH WPEEDECQDE ENQKQCQDLG
AFTESMVVFG CPN

P0C0L5; CO4B_HUMAN

(SEQ ID NO: 99)

MRLLWGLIWA SSFFTLSLQK PRLLLFSPSV VHLGVPLSVG
VQLQDVPRGQ VVKGSVFLRN PSRNNVPCSP KVDFTLSSER
DFALLSLQVP LKDAKSCGLH QLLRGPEVQL VAHSPWLKDS
LSRTTNIQGI NLLFSSRRGH LFLQTDQPIY NPGQRVRYRV
FALDQKMRPS TDTITVMVEN SHGLRVRKKE VYMPSSIFQD
DFVIPDISEP GTWKISARFS DGLESNSSTQ FEVKKYVLPN
FEVKITPGKP YILTVPGHLD EMQLDIQARY IYGKPVQGVA
YVRFGLLDED GKKTFFRGLE SQTKLVNGQS HISLSKAEFQ
DALEKLNMGI TDLQGLRLYV AAAIIESPGG EMEEAELTSW
YFVSSPFSLD LSKTKRHLVP GAPFLLQALV REMSGSPASG
IPVKVSATVS SPGSVPEVQD IQQNTDGSGQ VSIPIIIPQT
ISELQLSVSA GSPHPAIARL TVAAPPSGGP GFLSIERPDS
RPPRVGDTLN LNLRAVGSGA TFSHYYYMIL SRGQIVFMNR
EPKRTLTSVS VFVDHHLAPS FYFVAFYYHG DHPVANSLRV
DVQAGACEGK LELSVDGAKQ YRNGESVKLH LETDSLALVA
LGALDTALYA AGSKSHKPLN MGKVFEAMNS YDLGCGPGGG
DSALQVFQAA GLAFSDGDQW TLSRKRLSCP KEKTTRKKRN
VNFQKAINEK LGQYASPTAK RCCQDGVTRL PMMRSCEQRA
ARVQQPDCRE PFLSCCQFAE SLRKKSRDKG QAGLQRALEI
LQEEDLIDED DIPVRSFFPE NWLWRVETVD RFQILTLWLP
DSLTTWEIHG LSLSKTKGLC VATPVQLRVF REFHLHLRLP
MSVRRFEQLE LRPVLYNYLD KNLTVSVHVS PVEGLCLAGG
GGLAQQVLVP AGSARPVAFS VVPTAAAAVS LKVVARGSFE
FPVGDAVSKV LQIEKEGAIH REELVYELNP LDHRGRTLEI
PGNSDPNMIP DGDFNSYVRV TASDPLDTLG SEGALSPGGV
ASLLRLPRGC GEQTMIYLAP TLAASRYLDK TEQWSTLPPE
TKDHAVDLIQ KGYMRIQQFR KADGSYAAWL SRDSSTWLTA
FVLKVLSLAQ EQVGGSPEKL QETSNWLLSQ QQADGSFQDL
SPVIHRSMQG GLVGNDETVA LTAFVTIALH HGLAVFQDEG
AEPLKQRVEA SISKANSFLG EKASAGLLGA HAAAITAYAL
SLTKAPVDLL GVAHNNLMAM AQETGDNLYW GSVTGSQSNA
VSPTPAPRNP SDPMPQAPAL WIETTAYALL HLLLHEGKAE
MADQASAWLT RQGSFQGGFR STQDTVIALD ALSAYWIASH
TTEERGLNVT LSSTGRNGFK SHALQLNNRQ IRGLEEELQF
SLGSKINVKV GGNSKGTLKV LRTYNVLDMK NTTCQDLQIE
VTVKGHVEYT MEANEDYEDY EYDELPAKDD PDAPLQPVTP
LQLFEGRRNR RRREAPKVVE EQESRVHYTV CIWRNGKVGL
SGMAIADVTL LSGFHALRAD LEKLTSLSDR YVSHFETEGP
HVLLYFDSVP TSRECVGFEA VQEVPVGLVQ PASATLYDYY
NPERRCSVFY GAPSKSRLLA TLCSAEVCQC AEGKCPRQRR
ALERGLQDED GYRMKFACYY PRVEYGFQVK VLREDSRAAF
RLFETKITQV LHFTKDVKAA ANQMRNFLVR ASCRLRLEPG
KEYLIMGLDG ATYDLEGHPQ YLLDSNSWIE EMPSERLCRS
TRQRAACAQL NDFLQEYGTQ GCQV

9. Ligands for LDL Receptor Family
P05067; A4_HUMAN
P12023; A4_MOUSE
P05067; A4_HUMAN

(SEQ ID NO: 100)

MLPGLALLLL AAWTARALEV PTDGNAGLLA EPQIAMFCGR
LNMHMNVQNG KWDSDPSGTK TCIDTKEGIL QYCQEVYPEL
QITNVVEANQ PVTIQNWCKR GRKQCKTHPH FVIPYRCLVG
EFVSDALLVP DKCKFLHQER MDVCETHLHW HTVAKETCSE
KSTNLHDYGM LLPCGIDKFR GVEFVCCPLA EESDNVDSAD
AEEDDSDVWW GGADTDYADG SEDKVVEVAE EEEVAEVEEE
EADDDEDDED GDEVEEEAEE PYEEATERTT SIATTTTTTT
ESVEEVVREV CSEQAETGPC RAMISRWYFD VTEGKCAPFF
YGGCGGNRNN FDTEEYCMAV CGSAMSQSLL KTTQEPLARD
PVKLPTTAAS TPDAVDKYLE TPGDENEHAH FQKAKERLEA
KHRERMSQVM REWEEAERQA KNLPKADKKA VIQHFQEKVE
SLEQEAANER QQLVETHMAR VEAMLNDRRR LALENYITAL
QAVPPRPRHV FNMLKKYVRA EQKDRQHTLK HFEHVRMVDP
KKAAQIRSQV MTHLRVIYER MNQSLSLLYN VPAVAEEIQD
EVDELLQKEQ NYSDDVLANM ISEPRISYGN DALMPSLTET
KTTVELLPVN GEFSLDDLQP WHSFGADSVP ANTENEVEPV
DARPAADRGL TTRPGSGLTN IKTEEISEVK MDAEFRHDSG
YEVHHQKLVF FAEDVGSNKG AIIGLMVGGV VIATVIVITL
VMLKKKQYTS IHHGVVEVDA AVTPEERHLS KMQQNGYENP
TYKFFEQMQN

10. Endocrine and Exocrine Receptor Ligands
P01241; SOMA_HUMAN
P06880; SOMA_MOUSE
Q9UBU3; GHRL_HUMAN
Q9EQX0; GHRL_MOUSE
P01241; SOMA_HUMAN

(SEQ ID NO: 101)

MATGSRTSLL LAFGLLCLPW LQEGSAFPTI PLSRLFDNAM
LRAHRLHQLA FDTYQEFEEA YIPKEQKYSF LQNPQTSLCF
SESIPTPSNR EETQQKSNLE LLRISLLLIQ SWLEPVQFLR
SVFANSLVYG ASDSNVYDLL KDLEEGIQTL MGRLEDGSPR
TGQIFKQTYS KFDTNSHNDD ALLKNYGLLY CFRKDMDKVE
TFLRIVQCRS VEGSCGF

Q9UBU3; GHRL_HUMAN

(SEQ ID NO: 102)

MPSPGTVCSL LLLGMLWLDL AMAGSSFLSP EHQRVQQRKE
SKKPPAKLQP RALAGWLRPE DGGQAEGAED ELEVRFNAPF
DVGIKLSGVQ YQQHSQALGK FLQDILWEEA KEAPADK

11. Transforming Growth Factor Ligands
P01137; TGFB1_HUMAN
P04202; TGFB1_MOUSE
P01137; TGFB1_HUMAN

(SEQ ID NO: 103)

MPPSGLRLLL LLLPLLWLLV LTPGRPAAGL STCKTIDMEL
VKRKRIEAIR GQILSKLRLA SPPSQGEVPP GPLPEAVLAL
YNSTRDRVAG ESAEPEPEPE ADYYAKEVTR VLMVETHNEI
YDKFKQSTHS IYMFFNTSEL REAVPEPVLL SRAELRLLRL
KLKVEQHVEL YQKYSNNSWR YLSNRLLAPS DSPEWLSFDV
TGVVRQWLSR GGEIEGFRLS AHCSCDSRDN TLQVDINGFT
TGRRGDLATI HGMNRPFLLL MATPLERAQH LQSSRHRRAL
DTNYCFSSTE KNCCVRQLYI DFRKDLGWKW IHEPKGYHAN
FCLGPCPYIW SLDTQYSKVL ALYNQHNPGA SAAPCCVPQA
LEPLPIVYYV GRKPKVEQLS NMIVRSCKCS

12. Chemokine Receptor Ligands
P13500; CCL2_HUMAN
P10148; CCL2_MOUSE
P13500; CCL2_HUMAN

(SEQ ID NO: 104)

MKVSAALLCL LLIAATFIPQ GLAQPDAINA PVTCCYNFTN
RKISVQRLAS YRRITSSKCP KEAVIFKTIV AKEICADPKQ
KWVQDSMDHL DKQTQTPKT

13. Integrins
P05556; ITB1_HUMAN
P09055; ITB1_MOUSE
P05556; ITB1_HUMAN

(SEQ ID NO: 105)

MNLQPIFWIG LISSVCCVFA QTDENRCLKA NAKSCGECIQ
AGPNCGWCTN STFLQEGMPT SARCDDLEAL KKKGCPPDDI
ENPRGSKDIK KNKNVTNRSK GTAEKLKPED ITQIQPQQLV
LRLRSGEPQT FTLKFKRAED YPIDLYYLMD LSYSMKDDLE
NVKSLGTDLM NEMRRITSDF RIGFGSFVEK TVMPYISTTP
AKLRNPCTSE QNCTSPFSYK NVLSLTNKGE VFNELVGKQR
ISGNLDSPEG GFDAIMQVAV CGSLIGWRNV TRLLVFSTDA
GFHFAGDGKL GGIVLPNDGQ CHLENNMYTM SHYYDYPSIA
HLVQKLSENN IQTIFAVTEE FQPVYKELKN LIPKSAVGTL
SANSSNVIQL IIDAYNSLSS EVILENGKLS EGVTISYKSY
CKNGVNGTGE NGRKCSNISI GDEVQFEISI TSNKCPKKDS
DSFKIRPLGF TEEVEVILQY ICECECQSEG IPESPKCHEG
NGTFECGACR CNEGRVGRHC ECSTDEVNSE DMDAYCRKEN
SSEICSNNGE CVCGQCVCRK RDNTNEIYSG KFCECDNFNC
DRSNGLICGG NGVCKCRVCE CNPNYTGSAC DCSLDTSTCE
ASNGQICNGR GICECGVCKC TDPKFQGQTC EMCQTCLGVC
AEHKECVQCR AFNKGEKKDT CTQECSYFNI TKVESRDKLP
QPVQPDPVSH CKEKDVDDCW FYFTYSVNGN NEVMVHVVEN
PECPTGPDII PIVAGVVAGI VLIGLALLLI WKLLMIIHDR
REFAKFEKEK MNAKWDTGEN PIYKSAVTTV VNPKYEGK

14. Interleukins
Q13169; Q13169_HUMAN
Q0PGS4; Q0PGS4_MOUSE
Q13169; Q13169_HUMAN

(SEQ ID NO: 106)

MYRMQLLSCI ALILALVTNS APTSSSTKKT KKTQLQLEHL
LLDLQMILNG INNYKNPKLT RMLTFKFYMP KKATELKQLQ
CLEEELKPLE EVLNLAQSKN FHLRPRDLIS NINVIVLELK
GSETTFMCEY ADETATIVEF LNRWITFCQS IISTLT

15. Differentiation Factors Like Bone Differentiation Factors
P13497; BMP1_HUMAN
P98063; BMP1_MOUSE
P13497; BMP1_HUMAN

(SEQ ID NO: 107)

MPGVARLPLL LGLLLLPRPG RPLDLADYTY DLAEEDDSEP
LNYKDPCKAA AFLGDIALDE EDLRAFQVQQ AVDLRRHTAR
KSSIKAAVPG NTSTPSCQST NGQPQRGACG RWRGRSRSRR
AATSRPERVW PDGVIPFVIG GNFTGSQRAV FRQAMRHWEK
HTCVTFLERT DEDSYIVFTY RPCGCCSYVG RRGGGPQAIS
IGKNCDKFGI VVHELGHVVG FWHEHTRPDR DRHVSIVREN
IQPGQEYNFL KMEPQEVESL GETYDFDSIM HYARNTFSRG
IFLDTIVPKY EVNGVKPPIG QRTRLSKGDI AQARKLYKCP
ACGETLQDST GNFSSPEYPN GYSAHMHCVW RISVTPGEKI
ILNFTSLDLY RSRLCWYDYV EVRDGFWRKA PLRGRFCGSK
LPEPIVSTDS RLWVEFRSSS NWVGKGFFAV YEAICGGDVK
KDYGHIQSPN YPDDYRPSKV CIWRIQVSEG FHVGLTFQSF
EIERHDSCAY DYLEVRDGHS ESSTLIGRYC GYEKPDDIKS
TSSRLWLKFV SDGSINKAGF AVNFFKEVDE CSRPNRGGCE
QRCLNTLGSY KCSCDPGYEL APDKRRCEAA CGGFLTKLNG
SITSPGWPKE YPPNKNCIWQ LVAPTQYRIS LQFDFFETEG
NDVCKYDFVE VRSGLTADSK LHGKFCGSEK PEVITSQYNN
MRVEFKSDNT VSKKGFKAHF FSDKDECSKD NGGCQQDCVN
TFGSYECQCR SGFVLHDNKH DCKEAGCDHK VTSTSGTITS
PNWPDKYPSK KECTWAISST PGHRVKLTFM EMDIESQPEC
AYDHLEVFDG RDAKAPVLGR FCGSKKPEPV LATGSRMFLR
FYSDNSVQRK GFQASHATEC GGQVRADVKT KDLYSHAQFG
DNNYPGGVDC EWVIVAEEGY GVELVFQTFE VEEETDCGYD
YMELFDGYDS TAPRLGRYCG SGPPEEVYSA GDSVLVKFHS
DDTITKKGFH LRYTSTKFQD TLHSRK

16. Gastrin-Releasing Peptide
P07492; GRP_HUMAN
Q8R112; GRP_MOUSE
P07492; GRP_HUMAN

(SEQ ID NO: 108)

MRGSELPLVL LALVLCLAPR GRAVPLPAGG GTVLTKMYPR
GNHWAVGHLM GKKSTGESSS VSERGSLKQQ LREYIRWEEA
ARNLLGLIEA KENRNHQPPQ PKALGNQQPS WDSEDSSNFK
DVGSKGKVGR LSAPGSQREG RNPQLNQQ

17. Vasoactive Intestinal Peptide (VIP)
P01282; VIP_HUMAN
P32648; VIP_MOUSE
P01282; VIP_HUMAN

(SEQ ID NO: 109)

MDTRNKAQLL VLLTLLSVLF SQTSAWPLYR APSALRLGDR
IPFEGANEPD QVSLKEDIDM LQNALAENDT PYYDVSRNAR
HADGVFTSDF SKLLGQLSAK KYLESLMGKR VSSNISEDPV
PVKRHSDAVF TDNYTRLRKQ MAVKKYLNSI LNGKRSSEGE
SPDFPEELEK

18. Insulin and Insulin-Like Growth Factor
P01308; INS_HUMAN
P01343; IGF1A_HUMAN
P05019; IGF1B_HUMAN
P05017; IGF1_MOUSE
P01308; INS_HUMAN

(SEQ ID NO: 110)

MALWMRLLPL LALLALWGPD PAAAFVNQHL CGSHLVEALY
LVCGERGFFY TPKTRREAED LQVGQVELGG GPGAGSLQPL
ALEGSLQKRG IVEQCCTSIC SLYQLENYCN

P01343; IGF1A_HUMAN

(SEQ ID NO: 111)

MGKISSLPTQ LFKCCFCDFL KVKMHTMSSS HLFYLALCLL
TFTSSATAGP ETLCGAELVD ALQFVCGDRG FYFNKPTGYG
SSSRRAPQTG IVDECCFRSC DLRRLEMYCA PLKPAKSARS
VRAQRHTDMP KTQKEVHLKN ASRGSAGNKN YRM

P05019; IGF1B_HUMAN

(SEQ ID NO: 112)

MGKISSLPTQ LFKCCFCDFL KVKMHTMSSS HLFYLALCLL
TFTSSATAGP ETLCGAELVD ALQFVCGDRG FYFNKPTGYG
SSSRRAPQTG IVDECCFRSC DLRRLEMYCA PLKPAKSARS
VRAQRHTDMP KTQKYQPPST NKNTKSQRRK GWPKTHPGGE
QKEGTEASLQ IRGKKKEQRR EIGSRNAECR GKKGK

19. Calcitonin and Calcitonin Gene-Related Peptide
P01258; CALC_HUMAN
P70160; CALC_MOUSE
P01258; CALC_HUMAN

(SEQ ID NO: 113)

MGFQKFSPFL ALSILVLLQA GSLHAAPFRS ALESSPADPA
TLSEDEARLL LAALVQDYVQ MKASELEQEQ EREGSSLDSP
RSKRCGNLST CMLGTYTQDF NKFHTFPQTA IGVGAPGKKR
DMSSDLERDH RPHVSMPQNA N

20. Ligands to Inflammatory Cells Like Mast Cells, Eosinophils, Macrophage, Monocytes, and Neutrophils
P09603; CSF1_HUMAN
P07141; CSF1_MOUSE
P0C0L5; CO4B_HUMAN
P01029; CO4B_MOUSE
P09603; CSF1_HUMAN

(SEQ ID NO: 114)

MTAPGAAGRC PPTTWLGSLL LLVCLLASRS ITEEVSEYCS
HMIGSGHLQS LQRLIDSQME TSCQITFEFV DQEQLKDPVC
YLKKAFLLVQ DIMEDTMRFR DNTPNAIAIV QLQELSLRLK
SCFTKDYEEH DKACVRTFYE TPLQLLEKVK NVFNETKNLL
DKDWNIFSKN CNNSFAECSS QDVVTKPDCN CLYPKAIPSS
DPASVSPHQP LAPSMAPVAG LTWEDSEGTE GSSLLPGEQP
LHTVDPGSAK QRPPRSTCQS FEPPETPVVK DSTIGGSPQP
RPSVGAFNPG MEDILDSAMG TNWVPEEASG EASEIPVPQG
TELSPSRPGG GSMQTEPARP SNFLSASSPL PASAKGQQPA
DVTGTALPRV GPVRPTGQDW NHTPQKTDHP SALLRDPPEP
GSPRISSLRP QGLSNPSTLS AQPQLSRSHS SGSVLPLGEL
EGRRSTRDRR SPAEPEGGPA SEGAARPLPR FNSVPLTDTG
HERQSEGSSS PQLQESVFHL LVPSVILVLL AVGGLLFYRW
RRRSHQEPQR ADSPLEQPEG SPLTQDDRQV ELPV

Additional targeting peptides useful according to the invention include but are not limited to the following:

(SEQ ID NO: 115)

GTFVYGGCRAKRNNFKSAED

(SEQ ID NO: 116)

GPFFYGGCGGNRNNFDTEEY

(SEQ ID NO: 117)

GTFFYGGCRGKRNNFKTEEY

(SEQ ID NO: 118)

GTFFYGGSRGKRNNFKTEEY

(SEQ ID NO: 119)

GRFFYGGSRGKRNNFRTEEY

(SEQ ID NO: 120)

GTFFYGGSRGRRNNFRTEEY

(SEQ ID NO: 121)

CTFVYGGCRAKRNNFKSAED

(SEQ ID NO: 122)

CPFFYGGCGGNRNNFDTEEY

(SEQ ID NO: 123)

CTFFYGGCRGKRNNFKTEEY

(SEQ ID NO: 124)

CTFFYGGSRGKRNNFKTEEY

(SEQ ID NO: 125)

CRFFYGGSRGKRNNFRTEEY

(SEQ ID NO: 126)

CTFFYGGSRGRRNNFRTEEY

(SEQ ID NO: 127)

TFVYGGCRAKRNNFKSAEDG

(SEQ ID NO: 128)

PFFYGGCGGNRNNFDTEEYG

(SEQ ID NO: 129)

TFFYGGCRGKRNNFKTEEYG

(SEQ ID NO: 130)

TFFYGGSRGKRNNFKTEEYG

(SEQ ID NO: 131)

RFFYGGSRGKRNNFRTEEYG

(SEQ ID NO: 132)

TFFYGGSRGRRNNFRTEEYG

(SEQ ID NO: 133)

TFVYGGCRAKRNNFKSAEDC

(SEQ ID NO: 134)

PFFYGGCGGNRNNFDTEEYC

(SEQ ID NO: 135)

TFFYGGCRGKRNNFKTEEYC

(SEQ ID NO: 136)

TFFYGGSRGKRNNFKTEEYC

(SEQ ID NO: 137)

RFFYGGSRGKRNNFRTEEYC

(SEQ ID NO: 138)

TFFYGGSRGRRNNFRTEEYC

(SEQ ID NO: 149)

STEELRVRLASHLRKLRKRLL

(SEQ ID NO: 150)

SSVIDALQYKLEGTTRLTRKRGLKLATALSLSNKFVEGS

(SEQ ID NO: 154)

EELRVRLASHLRKLRKRLLRDADDLQK

(SEQ ID NO: 155)

GQSTEELRARLASHLRKLRKR

(SEQ ID NO: 156)

RLASHLRKLRKRLLRD

(SEQ ID NO: 151)

H2N-c[D(Cys-Ser-Lys-Cys)]Gly-Peg12-Lys

(SEQ ID NO: 152)

H2N-c[Cys-Phe-Thr-Lys-D-Trp-Phe-Phe-Cys]-Peg12-Lys

(SEQ ID NO: 153)

H2N-Thr-Phe-Thr-Lys-D-Trp-Phe-Phe-D-Phe-Peg12-Lys

In one embodiment, a peptide of the invention is conjugated to a translocation domain, for example a translocation domain of a neurotoxin. Neurotoxin translocation domain peptide sequences that are useful according to the invention include but are not limited the following. Peptides sequences are chosen from any subunit within the sequence. Peptide segments based on the sequences that meet the specifications of the invention are chosen.

1. Botulinum Neurotoxin Type A (BoNT/A) (EC 3.4.24.69) (Bontoxilysin-A)
P10845; BXA1_CLOBO

(SEQ ID NO: 139)

MPFVNKQFNY KDPVNGVDIA YIKIPNVGQM QPVKAFKIHN
KIWVIPERDT FTNPEEGDLN PPPEAKQVPV SYYDSTYLST
DNEKDNYLKG VTKLFERIYS TDLGRMLLTS IVRGIPFWGG
STIDTELKVI DTNCINVIQP DGSYRSEELN LVIIGPSADI
IQFECKSFGH EVLNLTRNGY GSTQYIRFSP DFTFGFEESL
EVDTNPLLGA GKFATDPAVT LAHELIHAGH RLYGIAINPN
RVFKVNTNAY YEMSGLEVSF EELRTFGGHD AKFIDSLQEN
EFRLYYYNKF KDIASTLNKA KSIVGTTASL QYMKNVFKEK
YLLSEDTSGK FSVDKLKFDK LYKMLTEIYT EDNFVKFFKV
LNRKTYLNFD KAVFKINIVP KVNYTIYDGF NLRNTNLAAN
FNGQNTEINN MNFTKLKNFT GLFEFYKLLC VRGIITSKTK
SLDKGYNKAL NDLCIKVNNW DLFFSPSEDN FTNDLNKGEE
ITSDTNIEAA EENISLDLIQ QYYLTFNFDN EPENISIENL
SSDIIGQLEL MPNIERFPNG KKYELDKYTM FHYLRAQEFE
HGKSRIALTN SVNEALLNPS RVYTFFSSDY VKKVNKATEA
AMFLGWVEQL VYDFTDETSE VSTTDKIADI TIIIPYIGPA
LNIGNMLYKD DFVGALIFSG AVILLEFIPE IAIPVLGTFA
LVSYIANKVL TVQTIDNALS KRNEKWDEVY KYIVTNWLAK
VNTQIDLIRK KMKEALENQA EATKAIINYQ YNQYTEEEKN
NINFNIDDLS SKLNESINKA MININKFLNQ CSVSYLMNSM
IPYGVKRLED FDASLKDALL KYIYDNRGTL IGQVDRLKDK
VNNTLSTDIP FQLSKYVDNQ RLLSTFTEYI KNIINTSILN
LRYESNHLID LSRYASKINI GSKVNFDPID KNQIQLFNLE
SSKIEVILKN AIVYNSMYEN FSTSFWIRIP KYFNSISLNN
EYTIINCMEN NSGWKVSLNY GEIIWTLQDT QEIKQRVVFK
YSQMINISDY INRWIFVTIT NNRLNNSKIY INGRLIDQKP
ISNLGNIHAS NNIMFKLDGC RDTHRYIWIK YFNLFDKELN
EKEIKDLYDN QSNSGILKDF WGDYLQYDKP YYMLNLYDPN
KYVDVNNVGI RGYMYLKGPR GSVMTTNIYL NSSLYRGTKF
IIKKYASGNK DNIVRNNDRV YINVVVKNKE YRLATNASQA
GVEKILSALE IPDVGNLSQV VVMKSKNDQG ITNKCKMNLQ
DNNGNDIGFI GFHQFNNIAK LVASNWYNRQ IERSSRTLGC
SWEFIPVDDG WGERPL

2. Botulinum Neurotoxin Type B (BoNT/B) (EC 3.4.24.69) (Bontoxilysin-B)
B11NP5; BXB_CLOBK

(SEQ ID NO: 140)

MPVTINNFNY NDPIDNNNII MMEPPFARGT GRYYKAFKIT
DRIWIIPERY TFGYKPEDFN KSSGIFNRDV CEYYDPDYLN
TNDKKNIFLQ TMIKLFNRIK SKPLGEKLLE MIINGIPYLG
DRRVPLEEFN TNIASVTVNK LISNPGEVER KKGIFANLII
FGPGPVLNEN ETIDIGIQNH FASREGFGGI MQMKFCPEYV
SVFNNVQENK GASIFNRRGY FSDPALILMH ELIHVLHGLY
GIKVDDLPIV PNEKKFFMQS TDAIQAEELY TFGGQDPSII
TPSTDKSIYD KVLQNFRGIV DRLNKVLVCI SDPNININIY
KNKFKDKYKF VEDSEGKYSI DVESFDKLYK SLMFGFTETN
IAENYKIKTR ASYFSDSLPP VKIKNLLDNE IYTIEEGFNI
SDKDMEKEYR GQNKAINKQA YEEISKEHLA VYKIQMCKSV
KAPGICIDVD NEDLFFIADK NSFSDDLSKN ERIEYNTQSN
YIENDFPINE LILDTDLISK IELPSENTES LTDFNVDVPV
YEKQPAIKKI FTDENTIFQY LYSQTFPLDI RDISLTSSFD
DALLFSNKVY SFFSMDYIKT ANKVVEAGLF AGWVKQIVND
FVIEANKSNT MDKIADISLI VPYIGLALNV GNETAKGNFE
NAFEIAGASI LLEFIPELLI PVVGAFLLES YIDNKNKIIK
TIDNALTKRN EKWSDMYGLI VAQWLSTVNT QFYTIKEGMY
KALNYQAQAL EEIIKYRYNI YSEKEKSNINI DFNDINSKL
NEGINQAIDN INNFINGCSV SYLMKKMIPL AVEKLLDFDN
TLKKNLLNYI DENKLYLIGS AEYEKSKVNK YLKTIMPFDL
SIYTNDTILI EMFNKYNSEI LNNIILNLRY KDNNLIDLSG
YGAKVEVYDG VELNDKNQFK LTSSANSKIR VTQNQNIIFN
SVFLDFSVSF WIRIPKYKND GIQNYIHNEY TIINCMKNNS
GWKISIRGNR IIWTLIDING KTKSVFFEYN IREDISEYIN
RWFFVTITNN LNNAKIYING KLESNTDIKD IREVIANGEI
IFKLDGDIDR TQFIWMKYFS IFNTELSQSN IEERYKIQSY
SEYLKDFWGN PLMYNKEYYM FNAGNKNSYI KLKKDSPVGE
ILTRSKYNQN SKYINYRDLY IGEKFIIRRK SNSQSINDDI
VRKEDYIYLD FFNLNQEWRV YTYKYFKKEE EKLFLAPISD
SDEFYNTIQI KEYDEQPTYS CQLLFKKDEE STDEIGLIGI
HRFYESGIVF EEYKDYFCIS KWYLKEVKRK
PYNLKLGCNW QFIPKDEGWT E

3. Botulinum Neurotoxin Type C1 (BoNT/C1) (EC 3.4.24.69) (Bontoxilysin-C1)
P18640; BXC1_CLOBO

(SEQ ID NO: 141)

MPITINNFNY SDPVDNKNIL YLDTHLNTLA NEPEKAFRIT
GNIWVIPDRF SRNSNPNLNK PPRVTSPKSG YYDPNYLSTD
SDKDPFLKEI IKLFKRINSR EIGEELIYRL STDIPFPGNN
NTPINTFDFD VDFNSVDVKT RQGNNWVKTG SINPSVIITG
PRENIIDPET STFKLTNNTF AAQEGFGALS IISISPRFML
TYSNATNDVG EGRFSKSEFC MDPILILMHE LNHAMHNLYG
IAIPNDQTIS SVTSNIFYSQ YNVKLEYAEI YAFGGPTIDL
IPKSARKYFE EKALDYYRSI AKRLNSITTA NPSSFNKYIG
EYKQKLIRKY RFVVESSGEV TVNRNKFVEL YNELTQIFTE
FNYAKIYNVQ NRKIYLSNVY TPVTANILDD NVYDIQNGFN
IPKSNLNVLF MGQNLSRNPA LRKVNPENML YLFTKFCHKA
IDGRSLYNKT LDCRELLVKN TDLPFIGDIS DVKTDIFLRK
DINEETEVIY YPDNVSVDQV ILSKNTSEHG QLDLLYPSID
SESEILPGEN QVFYDNRTQN VDYLNSYYYL ESQKLSDNVE
DFTFTRSIEE ALDNSAKVYT YFPTLANKVN AGVQGGLFLM
WANDVVEDFT TNILRKDTLD KISDVSAIIP YIGPALNISN
SVRRGNFTEA FAVTGVTILL EAFPEFTIPA LGAFVIYSKV
QERNEIIKTI DNCLEQRIKR WKDSYEWMMG TWLSRIITQF
NNISYQMYDS LNYQAGAIKA KIDLEYKKYS GSDKENIKSQ
VENLKNSLDV KISEAMNNIN KFIRECSVTY LFKNMLPKVI
DELNEFDRNT KAKLINLIDS HNIILVGEVD KLKAKVNNSF
QNTIPFNIFS YTNNSLLKDI INEYFNNIND SKILSLQNRK
NTLVDTSGYN AEVSEEGDVQ LNPIFPFDFK LGSSGEDRGK
VIVTQNENIV YNSMYESFSI SFWIRINKWV SNLPGYTIID
SVKNNSGWSI GIISNFLVFT LKQNEDSEQS INFSYDISNN
APGYNKWFFV TVTNNMMGNM KIYINGKLID TIKVKELTGI
NFSKTITFEI NKIPDTGLIT SDSDNINMWI RDFYIFAKEL
DGKDINILFN SLQYTNVVKD YWGNDLRYNK EYYMVNIDYL
NRYMYANSRQ IVFNTRRNNN DFNEGYKIII KRIRGNTNDT
RVRGGDILYF DMTINNKAYN LFMKNETMYA DNHSTEDIYA
IGLREQTKDI NDNIIFQIQP MNNTYYYASQ IFKSNFNGEN
ISGICSIGTY RFRLGGDWYR HNYLVPTVKQ GNYASLLEST
STHWGFVPVS E

4. Botulinum Neurotoxin Type D (BoNT/D) (EC 3.4.24.69) (Bontoxilysin-D)
P19321; BXD_CLOBO

(SEQ ID NO: 142)

MTWPVKDFNY SDPVNDNDIL YLRIPQNKLI TTPVKAFMIT
QNIWVIPERF SSDTNPSLSK PPRPTSKYQS YYDPSYLSTD
EQKDTFLKGI IKLFKRINER DIGKKLINYL VVGSPFMGDS
STPEDTFDFT RHTTNIAVEK FENGSWKVTNI ITPSVLIFG
PLPNILDYTA SLTLQGQQSN PSFEGFGTLS ILKVAPEFLL
TFSDVTSNQS SAVLGKSIFC MDPVIALMHE LTHSLHQLYG
INIPSDKRIR PQVSEGFFSQ DGPNVQFEEL YTFGGLDVEI
IPQIERSQLR EKALGHYKDI AKRLNNINKT IPSSWISNID
KYKKIFSEKY NFDKDNTGNF VVNIDKFNSL YSDLTNVMSE
VVYSSQYNVK NRTHYFSRHY LPVFANILDD NIYTIRDGFN
LTNKGFNIEN SGQNIERNPA LQKLSSESVV DLFTKVCLRL
TKNSRDDSTC IKVKNNRLPY VADKDSISQE IFENKIITDE
TNVQNYSDKF SLDESILDGQ VPINPEIVDP LLPNVNMEPL
NLPGEEIVFY DDITKYVDYL NSYYYLESQK LSNNVENITL
TTSVEEALGY SNKIYTFLPS LAEKVNKGVQ AGLFLNWANE
VVEDFTTNIM KKDTLDKISD VSVIIPYIGP ALNIGNSALR
GNFNQAFATA GVAFLLEGFP EFTIPALGVF TFYSSIQERE
KIIKTIENCL EQRVKRWKDS YQWMVSNWLS RITTQFNHIN
YQMYDSLSYQ ADAIKAKIDL EYKKYSGSDK ENIKSQVENL
KNSLDVKISE AMNNINKFIR ECSVTYLFKN MLPKVIDELN
KFDLRTKTEL INLIDSHNII LVGEVDRLKA KVNESFENTM
PFNIFSYTNN SLLKDIINEY FNSINDSKIL SLQNKKNALV
DTSGYNAEVR VGDNVQLNTI YTNDFKLSSS GDKIIVNLNN
NILYSAIYEN SSVSFWIKIS KDLTNSHNEY TIINSIEQNS
GWKLCIRNGN IEWILQDVNR KYKSLIFDYS ESLSHTGYTN
KWFFVTITNN IMGYMKLYIN GELKQSQKIE DLDEVKLDKT
IVFGIDENID ENQMLWIRDF NIFSKELSNE DINIVYEGQI
LRNVIKDYWG NPLKFDTEYY IINDNYIDRY IAPESNVLVL
VQYPDRSKLY TGNPITIKSV SDKNPYSRIL NGDNIILHML
YNSRKYMIIR DTDTIYATQG GECSQNCVYA LKLQSNLGNY
GIGIFSIKNI VSKNKYCSQI FSSFRENTML LADIYKPWRF
SFKNAYTPVA VTNYETKLLS TSSFWKFISR DPGWVE

5. Botulinum Neurotoxin Type E (BoNT/E) (EC 3.4.24.69) (Bontoxilysin-E)
Q00496; BXE_CLOBO

(SEQ ID NO: 143)

MPKINSFNYN DPVNDRTILY IKPGGCQEFY KSFNIMKNIW
IIPERNVIGT TPQDFHPPTS LKNGDSSYYD PNYLQSDEEK
DRFLKIVTKI FNRINNNLSG GILLEELSKA NPYLGNDNTP
DNQFHIGDAS AVEIKFSNGS QDILLPNVII MGAEPDLFET
NSSNISLRNN YMPSNHRFGS IAIVTFSPEY SFRFNDNCMN
EFIQDPALTL MHELIHSLHG LYGAKGITTK YTITQKQNPL
ITNIRGTNIE EFLTFGGTDL NIITSAQSND IYTNLLADYK
KIASKLSKVQ VSNPLLNPYK DVFEAKYGLD KDASGIYSVN
INKFNDIFKK LYSFTEFDLR TKFQVKCRQT YIGQYKYFKL
SNLLNDSIYN ISEGYNINNL KVNFRGQNAN LNPRIITPIT
GRGLVKKIIR FCKNIVSVKG IRKSICIEIN NGELFFVASE
NSYNDDNINT PKEIDDTVTS NNNYENDLDQ VILNFNSESA
PGLSDEKLNL TIQNDAYIPK YDSNGTSDIE QHDVNELNVF
FYLDAQKVPE GENNVNLTSS IDTALLEQPK IYTFFSSEFI
NNVNKPVQAA LFVSWIQQVL VDFTTEANQK STVDKIADIS
IVVPYIGLAL NIGNEAQKGN FKDALELLGA GILLEFEPEL
LIPTILVFTI KSFLGSSDNK NKVIKAINNA LKERDEKWKE
VYSFIVSNWM TKINTQFNKR KEQMYQALQN QVNAIKTIIE
SKYNSYTLEE KNELTNKYDI KQIENELNQK VSIAMNNIDR
FLTESSISYL MKIINEVKIN KLREYDENVK TYLLNYIIQH
GSILGESQQE LNSMVTDTLN NSIPFKLSSY TDDKILISYF
NKFFKRIKSS SVLNMRYKND KYVDTSGYDS NININGDVYK
YPTNKNQFGI YNDKLSEVNI SQNDYIIYDN KYKNFSISFW
VRIPNYDNKI VNVNNEYTII NCMRDNNSGW KVSLNHNEII
WTFEDNRGIN QKLAFNYGNA NGISDYINKW IFVTITNDRL
GDSKLYINGN LIDQKSILNL GNIHVSDNIL FKIVNCSYTR
YIGIRYFNIF DKELDETEIQ TLYSNEPNTN ILKDFWGNYL
LYDKEYYLLN VLKPNNFIDR RKDSTLSINN IRSTILLANR
LYSGIKVKIQ RVNNSSTNDN LVRKNDQVYI NFVASKTHLF
PLYADTATTN KEKTIKISSS GNRFNQVVVM NSVGNCTMNF
KNNNGNNIGL LGFKADTVVA STWYYTHMRD HTNSNGCFWN
FISEEHGWQE K

6. Botulinum Neurotoxin Type F (BoNT/F) (EC 3.4.24.69) (Bontoxilysin-F)
P30996; BXF_CLOBO

(SEQ ID NO: 144)

MPVAINSFNY NDPVNDDTIL YMQIPYEEKS KKYYKAFEIM
RNVWIIPERN TIGTNPSDFD PPASLKNGSS AYYDPNYLTT
DAEKDRYLKT TIKLFKRINS NPAGKVLLQE ISYAKPYLGN
DHTPIDEFSP VTRTTSVNIK LSTNVESSML LNLLVLGAGP
DIFESCCYPV RKLIDPDVVY DPSNYGFGSI NIVTFSPEYE
YTFNDISGGH NSSTESFIAD PAISLAHELI HALHGLYGAR
GVTYEETIEV KQAPLMIAEK PIRLEEFLTF GGQDLNIITS
AMKEKIYNNL LANYEKIATR LSEVNSAPPE YDINEYKDYF
QWKYGLDKNA DGSYTVNENK FNEIYKKLYS FTESDLANKF
KVKCRNTYFI KYEFLKVPNL LDDDIYTVSE GFNIGNLAVN
NRGQSIKLNP KIIDSIPDKG LVEKIVKFCK SVIPRKGTKA
PPRLCIRVNN SELFFVASES SYNENDINTP KEIDDTTNLN
NNYRNNLDEV ILDYNSQTIP QISNRTLNTL VQDNSYVPRY
DSNGTSEIEE YDVVDFNVFF YLHAQKVPEG ETNISLTSSI
DTALLEESKD IFFSSEFIDT INKPVNAALF IDWISKVIRD
FTTEATQKST VDKIADISLI VPYVGLALNI IIEAEKGNFE
EAFELLGVGI LLEFVPELTI PVILVFTIKS YIDSYENKNK
AIKAINNSLI EREAKWKEIY SWIVSNWLTR INTQFNKRKE
QMYQALQNQV DAIKTAIEYK YNNYTSDEKN RLESEYNINN
IEEELNKKVS LAMKNIERFM TESSISYLMK LINEAKVGKL
KKYDNHVKSD LLNYILDHRS ILGEQTNELS DLVTSTLNSS
IPFELSSYTN DKILIIYFNR LYKKIKDSSI LDMRYENNKF
IDISGYGSNI SINGNVYIYS TNRNQFGIYN SRLSEVNIAQ
NNDIIYNSRY QNFSISFWVR IPKHYKPMNH NREYTIINCM
GNNNSGWKIS LRTVRDCEII WTLQDTSGNK ENLIFRYEEL
NRISNYINKW IFVTITNNRL GNSRIYINGN LIVEKSISNL
GDIHVSDNIL FKIVGCDDET YVGIRYFKVF NTELDKTEIE
TLYSNEPDPS ILKNYWGNYL LYNKKYYLFN LLRKDKYITL
NSGILNINQQ RGVTEGSVFL NYKLYEGVEV IIRKNGPIDI
SNTDNFVRKN DLAYINVVDR GVEYRLYADT KSEKEKIIRT
SNLNDSLGQI IVMDSIGNNC TMNFQNNNGS NIGLLGFHSN
NLVASSWYYN NIRRNTSSNG CFWSSISKEN GWKE

7. Botulinum Neurotoxin Type G (BoNT/G) (EC 3.4.24.69) (Bontoxilysin-G)
Q60393; BXG_CLOBO

(SEQ ID NO: 145)

MPVNIKXFNY NDPINNDDII MMEPFNDPGP GTYYKAFRII
DRIWIVPERF TYGFQPDQFN ASTGVFSKDV YEYYDPTYLK
TDAEKDKFLK TMIKLFNRIN SKPSGQRLLD MIVDAIPYLG
NASTPPDKFA ANVANVSINK KIIQPGAEDQ IKGLMTNLII
FGPGPVLSDN FTDSMIMNGH SPISEGFGAR MMIRFCPSCL
NVFNNVQENK DTSIFSRRAY FADPALTLMH ELIHVLHGLY
GIKISNLPIT PNTKEFFMQH SDPVQAEELY TFGGHDPSVI
SPSTDMNIYN KALQNFQDIA NRLNIVSSAQ GSGIDISLYK
QIYKNKYDFV EDPNGKYSVD KDKFDKLYKA LMFGFTETNL
AGEYGIKTRY SYFSEYLPPI KTEKLLDNTI YTQNEGFNIA
SKNLKTEFNG QNKAVNKEAY EEISLEHLVI YRIAMCKPVM
YKNTGKSEQC IIVNNEDLFF IANKDSFSKD LAKAETIAYN
TQNNTIENNF SIDQLILDND LSSGIDLPNE NTEPFTNFDD
IDIPVYIKQS ALKKIFVDGD SLFEYLHAQT FPSNIENLQL
TNSLNDALRN NNKVYTFFST NLVEKANTVV GASLFVNWVK
GVIDDFTSES TQKSTIDKVS DVSIIIPYIG PALNVGNETA
KENFKNAFEI GGAAILMEFI PELIVPIVGF FTLESYVGNK
GHIIMTISNA LKKRDQKWTD MYGLIVSQWL STVNTQFYTI
KERMYNALNN QSQAIEKIIE DQYNRYSEED KMNINIDFND
IDFKLNQSIN LAINNIDDFI NQCSISYLMN RMIPLAVKKL
KDFDDNLKRD LLEYIDTNEL YLLDEVNILK SKVNRHLKDS
IPFDLSLYTK DTILIQVFNN YISNISSNAI LSLSYRGGRL
IDSSGYGATM NVGSDVIFND IGNGQFKLNN SENSNITAHQ
SKFVVYDSMF DNFSINFWVR TPKYNNNDIQ TYLQNEYTII
SCIKNDSGWK VSIKGNRIIW TLIDVNAKSK SIFFEYSIKD
NISDYINKWF SITITNDRLG NANIYINGSL KKSEKILNLD
RINSSNDIDF KLINCTDTTK FVWIKDFNIF GRELNATEVS
SLYWIQSSTN TLKDFWGNPL RYDTQYYLFN QGMQNIYIKY
FSKASMGETA PRTNFNNAAI NYQNLYLGLR FIIKKASNSR
NINNDNIVRE GDYIYLNIDN ISDESYRVYV LVNSKEIQTQ
LFLAPINDDP TFYDVLQIKK YYEKTTYNCQ ILCEKDTKTF
GLFGIGKFVK DYGYVWDTYD NYFCISQWYL RRISENINKL
RLGCNWQFIP VDEGWTE

8. Tetanus Toxin (EC 3.4.24.68) (Tentoxylysin)
P04958; TETX_CLOTE

(SEQ ID NO: 146)

MPITINNFRY SDPVNNDTII MMEPPYCKGL DIYYKAFKIT
DRIWIVPERY EFGTKPEDFN PPSSLIEGAS EYYDPNYLRT
DSDKDRFLQT MVKLFNRIKN NVAGEALLDK IINAIPYLGN
SYSLLDKFDT NSNSVSFNLL EQDPSGATTK SAMLTNLIIF
GPGPVLNKNE VRGIVLRVDN KNYFPCRDGF GSIMQMAFCP
EYVPTFDNVI ENITSLTIGK SKYFQDPALL LMHELIHVLH
GLYGMQVSSH EIIPSKQEIY MQHTYPISAE ELFTFGGQDA
NLISIDIKND LYEKTLNDYK AIANKLSQVT SCNDPNIDID
SYKQIYQQKY QFDKDSNGQY IVNEDKFQIL YNSIMYGFTE
IELGKKFNIK TRLSYFSMNH DPVKIPNLLD DTIYNDTEGF
NIESKDLKSE YKGQNMRVNT NAFRNVDGSG LVSKLIGLCK
KIIPPTNIRE NLYNRTASLT DLGGELCIKI KNEDLTFIAE
KNSFSEEPFQ DEIVSYNTKN KPLNFNYSLD KIIVDYNLQS
KITLPNDRTT PVTKGIPYAP EYKSNAASTI EIHNIDDNTI
YQYLYAQKSP TTLQRITMTN SVDDALINST KIYSYFPSVI
SKVNQGAQGI LFLQWVRDII DDFTNESSQK TTIDKISDVS
TIVPYIGPAL NIVKQGYEGN FIGALETTGV VLLLEYIPEI
TLPVIAALSI AESSTQKEKI IKTIDNFLEK RYEKWIEVYK
LVKAKWLGTV NTQFQKRSYQ MYRSLEYQVD AIKKIIDYEY
KIYSGPDKEQ IADEINNLKN KLEEKANKAM ININIFMRES
SRSFLVNQMI NEAKKQLLEF DTQSKNILMQ YIKANSKFIG
ITELKKLESK INKVFSTPIP FSYSKNLDCW VDNEEDIDVI
LKKSTILNLD INNDIISDIS GFNSSVITYP DAQLVPGING
KAIHLVNNES SEVIVHKAMD IEYNDMFNNF TVSFWLRVPK
VSASHLEQYG TNEYSIISSM KKHSLSIGSG WSVSLKGNNL
IWTLKDSAGE VRQITFRDLP DKFNAYLANK WVFITITNDR
LSSANLYING VLMGSAEITG LGAIREDNNI TLKLDRCNNN
NQYVSIDKFR IFCKALNPKE IEKLYTSYLS ITFLRDFWGN
PLRYDTEYYL IPVASSSKDV QLKNITDYMY LTNAPSYTNG
KLNIYYRRLY NGLKFIIKRY TPNNEIDSFV KSGDFIKLYV
SYNNNEHIVG YPKDGNAFNN LDRILRVGYN APGIPLYKKM
EAVKLRDLKT YSVQLKLYDD KNASLGLVGT HNGQIGNDPN
RDILIASNWY FNHLKDKILG CDWYFVPTDE GWTND

9. Diphtheria Toxin (DT) (NAD(+)-diphthamide ADP-ribosyltransferase) (EC 2.4.2.36)
P00588; DTX_CORBE

(SEQ ID NO: 147)

MLVRGYVVSR KLFASILIGA LLGIGAPPSA HAGADDVVDS
SKSFVMENFS SYHGTKPGYV DSIQKGIQKP KSGTQGNYDD
DWKGFYSTDN KYDAAGYSVD NENPLSGKAG GVVKVTYPGL
TKVLALKVDN AETIKKELGL SLTEPLMEQV GTEEFIKRFG
DGASRVVLSL PFAEGSSSVE YINNWEQAKA LSVELEINFE
TRGKRGQDAM YEYMAQACAG NRVRRSVGSS LSCINLDWDV
IRDKTKTKIE SLKEHGPIKN KMSESPNKTV SEEKAKQYLE
EFHQTALEHP ELSELKTVTG TNPVFAGANY AAWAVNVAQV
IDSETADNLE KTTAALSILP GIGSVMGIAD GAVHHNTEEI
VAQSIALSSL MVAQAIPLVG ELVDIGFAAY NFVESIINLF
QVVHNSYNRP AYSPGHKTQP FLHDGYAVSW NTVEDSIIRT
GFQGESGHDI KITAENTPLP IAGVLLPTIP GKLDVNKSKT
HISVNGRKIR MRCRAIDGDV TFCRPKSPVY VGNGVHANLH
VAFHRSSSEK IHSNEISSDS IGVLGYQKTV DHTKVNSKLS
LFFEIKS

10. Pseudomonas Exotoxin
P11439; TOXA_PSEAE

(SEQ ID NO: 148)

MHLTPHWIPL VASLGLLAGG SFASAAEEAF DLWNECAKAC
VLDLKDGVRS SRMSVDPAIA DTNGQGVLHY SMVLEGGNDA
LKLAIDNALS ITSDGLTIRL EGGVEPNKPV RYSYTRQARG
SWSLNWLVPI GHEKPSNIKV FIHELNAGNQ LSHMSPIYTI
EMGDELLAKL ARDATFFVRA HESNEMQPTL AISHAGVSVV
MAQAQPRREK RWSEWASGKV LCLLDPLDGV YNYLAQQRCN
LDDTWEGKIY RVLAGNPAKH DLDIKPTVIS HRLHFPEGGS
LAALTAHQAC HLPLETFTRH RQPRGWEQLE QCGYPVQRLV
ALYLAARLSW NQVDQVIRNA LASPGSGGDL GEAIREQPEQ
ARLALTLAAA ESERFVRQGT GNDEAGAASA DVVSLTCPVA
AGECAGPADS GDALLERNYP TGAEFLGDGG DISFSTRGTQ
NWTVERLLQA HRQLEERGYV FVGYHGTFLE AAQSIVFGGV
RARSQDLDAI WRGFYIAGDP ALAYGYAQDQ EPDARGRIRN
GALLRVYVPR SSLPGFYRTG LTLAAPEAAG EVERLIGHPL
PLRLDAITGP EEEGGRLETI LGWPLAERTV VIPSAIPTDP
RNVGGDLDPS SIPDKEQAIS ALPDYASQPG KPPREDLK

Peptide Synthesis

There are at least four ways to obtain a peptide: (1) purification from a biological system (e.g., tissue, serum, urine, etc.); (Donini P et al., Acta Endocrinol (Copenh). 1966; 52(2):169-85 and Donini P et al., Acta Endocrinol (Copenh). 1966; 52(2):186-98. (2) purification of a peptide fragment after digestion of a protein; (Schulz-Knappe P et al., Eur J Med. Res. 1996; 1(5):223-36. and Kilara A, and Panyam D. Crit. Rev Food Sci Nutr. 2003; 43(6):607-33 (3) genetic engineering and recombinant technologies well known in the art (Martial J A et al., Science. 1979; 205(4406):602-7) and (4) direct chemical synthesis Peptide Synthesis and Applications, 1984. Edited by John Howl (Methods in Molecular Biology, Vol. 298), Humana Press, Totowa, N.J. Chemistry of Peptide Synthesis, 2005. N. Leo Benoiton, CRC Press, Boca Raton, Fla.

The first two approaches are often impractical due to a lack of control over the peptide sequences. The first approach is also problematic due to a low concentration of peptide in biological samples that requires significant concentrating steps prior to purification. Typically, therefore, for shorter peptides direct chemical synthesis is an attractive option, whereas, for larger peptides, recombinant technology is preferred.

Traditional synthetic approaches of organic chemistry are generally impractical for peptides with more than four or five amino acid residues due to the complexities of amino acids and peptides. The problems include the presence of multiple reactive groups in the peptide which lead to multiple sites of conjugation on a peptide thereby leading to peptide mixtures that are impure with respect to the peptide of interest and therefore require purification after each step. (Reference: Lehninger Principles of Biochemistry, 3^{rd} Ed., 2000. Edited by David L. Nelson and Michael M. Cox, Worth Publishers, New York, N.Y.)

The advent of solid phase peptide synthesis (Merrifield, 1962) in which a peptide is synthesized while keeping it immobilized at one end to a solid support provided the major breakthrough in the direct chemical synthesis of peptides. Today, most solid phase peptide syntheses involve FMOC chemistry. Briefly, chemical synthesis proceeds from the carboxyl terminus (C terminus) to the amino terminus (N terminus). The solid phase support is an insoluble polymer or resin. The 9-fluorenyl-methoxycarbonyl (FMOC) group prevents unwanted reactions at the α-amino group of the amino acid residue. The peptide is built on a resin support one amino acid at a time using a standard set of reactions in a repeating cycle. First, the C-terminal amino acid with the α-amino group protected by an FMOC group is attached to the reactive group on the resin. The protecting group on the α-amino group of the amino acid attached to the resin is removed, generally with a mild organic base. Now, the resin with the C-terminal amino acid is ready to receive the second amino acid of the peptide. Each amino acid is received, protected with different chemistries at the α-amino group (FMOC) and carboxyl group (generally, Dicyclohexylcarbodiimide, DCC). The carboxyl group of the second amino acid is activated by removing DCC and reacted with the deprotected α-amino group of the first amino acid on the solid support to form the peptide bond (Peptide Synthesis and Applications, 1984. Edited by John Howl (Methods in Molecular Biology, Vol. 298), Humana Press, Totowa, N.J. Chemistry of Peptide Synthesis, 2005. N. Leo Benoiton, CRC Press, Boca Raton, Fla.) (Reference: Lehninger Principles of Biochemistry, 3^{rd} Ed., 2000. Edited by David L. Nelson and Michael M. Cox, Worth Publishers, New York, N.Y.).

At each successive step in the cycle, protective chemical groups block unwanted reactions and the sequence of (i) deprotection of the α-amino group on the nascent peptide; (ii) activation of the carboxyl group on the next amino acid and (iii) reaction to form peptide bond continues until the entire peptide sequence is synthesized. When the peptide synthesis is complete, the linkage between the resin and the peptide is cleaved off to obtain the final peptide. The state-of-the-art solid phase peptide synthesis technology is automated, and several kinds of commercial instruments are now available and well known in the art. (Peptide Synthesis and Applications, 1984. Edited by John Howl (Methods in Molecular Biology, Vol. 298), Humana Press, Totowa, N.J. Chemistry of Peptide Synthesis, 2005. N. Leo Benoiton, CRC Press, Boca Raton, Fla.; Lehninger Principles of Biochemistry, 3^{rd} Ed., 2000. Edited by David L. Nelson and Michael M. Cox, Worth Publishers, New York, N.Y.)

Since the solid phase synthesis is a stepwise process for longer peptides it has the important limitation of lower overall yield and therefore increased cost. For example, with a 96% stepwise yield, the overall yield for 21mer, 51mer and 100mer peptides are 44%, 13% and 1.7%, respectively. Similarly, with a 99.8% stepwise yield, the overall yield for 21mer, 51mer and 100mer peptides are 96%, 90% and 82%, respectively. Therefore, for longer peptides it is more cost- and time-effective to genetically engineer A sequence in an expression cassette and express the sequence in an appropriate expression system (e.g., microbial expression system such as E. coli or yeast) or mammalian expression system (cell culture). For smaller peptides, however, the cost of genetically engineer the sequence and expressing and purifying the peptides are generally cost- and time-effective compared to the solid phase peptide synthesis.

Peptides useful for the current invention are synthesized, expressed or purified using the methods described above or other methods of synthesis, expression or purification known in the art.

Formation of dsRNA-Peptide Conjugate

At least one peptide is conjugated to a dsRNA either to the first or second strand or both and either on the 3′ end or 5′ end or both or internally. A peptide of the invention can be conjugated to a dsRNA of the invention via any amino acid residue in the peptide, e.g., the C-terminal amino acid of the C-terminus via the carboxyl group of the C-terminal amino acid or the N-terminal amino acid of the N-termimus via the α-amino group of the N-terminal amino acid or to a specific functional group on the amino acid residue (e.g., —SH group on Cys or amino group of Lys).

A dsRNA is conjugated to a peptide of the invention using any conjugation chemistry known in the art for peptide or proteins (References: Bioconjugate Techniques, 1996. Greg T. Hermanson, Academic Press, San Diego, Calif.; Chemistry of Protein Conjugation and Cross-linking, 1991. Shan S. Wong, CRC Press, Boca Raton, Fla.).

In one embodiment the 5′ end of the first or second strand is synthesized with a (CH₂)₆—NH₃ linker and conjugated to the —SH group of Cys of a peptide using maleimide chemistry to form a stable conjugate.

In another embodiment the 3′ end of the first or second strand is synthesized with a (CH₂)₆—SH linker and conjugated to the —SH group of Cys or a peptide via disulfide exchange to form a cleavable conjugate.

Following conjugation, dsRNA-peptide conjugates are purified by methods well known in the art (Oehlke J et al., Eur J. Biochem. 2002; 269(16):4025-32, Hamma T and Miller P S. Bioconjug Chem. 2003; 14(2):320-30, Zatsepin T S et al., Bioconjug Chem. 2005; 16(3):471-89, Ferenc G et al. Nucleosides Nucleotides Nucleic Acids. 2005; 24(5-7):1059-61) and characterized for identity and purity with standard analytical methods.

Determining the Function of dsRNA-Delivery Peptide Conjugates

A dsRNA-peptide conjugate of the invention is assayed to determine the ability of the dsRNA to be delivered to the appropriate target and to mediate RNAi cleavage (as described in the section entitled “RNAi In Vitro Assay to Assess DsiRNA Activity”, hereinbelow). A dsRNA peptide conjugate of the invention is also assayed to determine the ability of the peptide to be delivered to the appropriate target.

In one embodiment, a dsRNA-peptide or a peptide alone attaches to or interacts with a cell surface. The dsRNA-peptide conjugates or the peptide alone is taken up by a cell by directly penetrating the cell membrane, by an endocytic pathway, by both or by other methods known in the art.

The functionality of a dsRNA-peptide conjugate of the invention can be determined by quantitation of dsRNA Oligonucleotide according to the following method.

The technology employed to quantitate the DsiRNA oligonucleotides from plasma or tissue samples consists of solid phase extraction to isolate the analyte from the matrix followed by reversed phase ion pairing ultraperformance liquid chromatography (UPLC) separation and detection by electrospray ionization tandem mass spectrometry (ESI-MS/MS). The analytical instrumentation consists of a Waters Acquity UPLC chromatograph with a photodiode array detector connected in series to a Waters Quattro Premiere triple quadrupole mass spectrometer.

The solid phase extraction is accomplished using Phenomenex's Clarity extraction media and protocol. A “load/lysis” buffer is added to the plasma sample containing the oligo to remove any bound proteins. The oligo is preferentially adsorbed onto the solid phase media. Then a series of buffers are used to wash the oligo to remove contaminants and salts which will inhibit separation and ionization. Finally, the oligo is eluted from the media, concentrated and resuspended in a buffer which is amenable to the downstream analysis.

The chromatographic separation is accomplished using a mobile phase of hexafluoroisopropanol (HFIP) and triethylamine (TEA) and a C₁₈ stationary phase. The mass spectrometric detection is accomplished using electrospray ionization followed by a tandem MS (MS/MS) analysis. LC/MS system is developed to determine the characteristic transitions for that particular oligonucleotide molecule. Quantitation of the DsiRNA content in the samples is accomplished by comparing the MS response of the samples to a standard curve of the same DsiRNA in the test sample at varied concentrations (Lin et al. J Pharm Biomed Anal. 2007 Jun. 28: 44(2):330-341). The final data is expressed as a concentration of DsiRNA oligo mass per unit volume of sample (e.g., ng/mL).

### Modification of DsiRNAs

dsRNAs and dsRNA-peptide conjugates are transfected in vitro in cell culture models to establish comparative uptake or delivery of the dsRNAs and dsRNA-peptide conjugates. Appropriate cell culture models are utilized and end point measurements include, but are not limited to, one or more of the following: (i) mRNA quantification using qPCR; (ii) protein quantification using Western blot; (iii) labelled cell internalization of dsRNAs and dsRNA-peptide conjugates. Comparative uptake or delivery of the dsRNAs and dsRNA-peptide conjugates are assessed for the amount of delivered dsRNA, the speed of delivery of dsRNA and the stability of delivered dsRNA, for example, using the above-recited end point measurements.

In one example, transfection is performed in 24- or 48-well plates for transfecting dsRNAs or dsRNA-peptide conjugates into HeLa cells. Prior to application, dsRNAs and dsRNA-peptide conjugates are diluted into the cell culture media and incubated at room temperature for about 30 min. For dose-response experiments, the final concentration of dsRNAs and dsRNA-peptide conjugates applied are varied within a range of 0 to 50 nM. For the time-course experiments, to determine the speed with which a dsRNA is delivered as defined herein, an optimum concentration of dsRNA-peptide conjugate determine from the dose response experiment is studied for various incubation times, e.g., 30 min to 7 days.

The functionality of peptide, dsRNA and dsRNA-peptide conjugates are also tested by differentially labelling the peptide and the dsRNA with fluorescent tags and performing fluorescent co localization studies. A peptide is tagged with a green fluorescent dye and the dsRNAs are tagged with red florescent dye. Using this methodology, and a comparison with the localization of free (i.e., unconjugated) dsRNA confirms the ability of a peptide to internalize both the peptide alone and dsRNA-peptide conjugates. The following references describe how to conduct fluorescent localization and cellular trafficking studies—Moschos et al., Bioconjug Chem. 2007; 18(5):1450-1459; Moschos et al., Biochemical Society Transactions 2007; 35(4):807-810; Lord-Fontaine et al., J. Neurotrauma 2008; 25:1309-1322; Winton et al., J. Biol. Chem. 2002; 36(6):32820-32829; Lu, Langer and Chen. Mol. Pharm. 2009 Mar. 30. [Epub ahead of print]; McNaughton et al., Proc Natl Acad Sci USA. 2009 Apr. 14; 106(15):6111-6116.

Modification of dsRNAs

One major factor that inhibits the effect of double stranded RNAs (“dsRNAs”) is the degradation of dsRNAs (e.g., double-stranded RNA, siRNAs and DsiRNAs) by nucleases. A 3′-exonuclease is the primary nuclease activity present in serum and modification of the 3′-ends of antisense DNA oligonucleotides is crucial to prevent degradation (Eder et al., 1991, Antisense Res Dev, 1: 141-151). An RNase-T family nuclease has been identified called ERI-1 which has 3′ to 5′ exonuclease activity that is involved in regulation and degradation of siRNAs (Kennedy et al., 2004, Nature 427: 645-649; Hong et al., 2005, Biochem J, 390: 675-679). This gene is also known as Thex1 (NM_{—}02067) in mice or THEX1 (NM_{—}153332) in humans and is involved in degradation of histone mRNA; it also mediates degradation of 3′-overhangs in siRNAs, but does not degrade duplex RNA (Yang et al., 2006, J Biol Chem, 281: 30447-30454). It is therefore reasonable to expect that 3′-end-stabilization of dsRNAs, including the DsiRNAs of the instant invention, will improve stability.

XRN1 (NM_{—}019001) is a 5′ to 3′ exonuclease that resides in P-bodies and has been implicated in degradation of mRNA targeted by miRNA (Rehwinkel et al., 2005, RNA 11: 1640-1647) and may also be responsible for completing degradation initiated by internal cleavage as directed by a siRNA. XRN2 (NM_{—}012255) is a distinct 5′ to 3′ exonuclease that is involved in nuclear RNA processing. Although not currently implicated in degradation or processing of siRNAs and miRNAs, these both are known nucleases that can degrade RNAs and may also be important to consider.

RNase A is a major endonuclease activity in mammals that degrades RNAs. It is specific for ssRNA and cleaves at the 3′-end of pyrimidine bases. SiRNA degradation products consistent with RNase A cleavage can be detected by mass spectrometry after incubation in serum (Turner et al., 2007, Mol Biosyst 3: 43-50). The 3′-overhangs enhance the susceptibility of siRNAs to RNase degradation. Depletion of RNase A from serum reduces degradation of siRNAs; this degradation does show some sequence preference and is worse for sequences having poly A/U sequence on the ends (Haupenthal et al., 2006 Biochem Pharmacol 71: 702-710). This suggests the possibility that lower stability regions of the duplex may “breathe” and offer transient single-stranded species available for degradation by RNase A. RNase A inhibitors can be added to serum and improve siRNA longevity and potency (Haupenthal et al., 2007, Int J. Cancer 121: 206-210).

In 21mers, phosphorothioate or boranophosphate modifications directly stabilize the internucleoside phosphate linkage. Boranophosphate modified RNAs are highly nuclease resistant, potent as silencing agents, and are relatively non-toxic. Boranophosphate modified RNAs cannot be manufactured using standard chemical synthesis methods and instead are made by in vitro transcription (IVT) (Hall et al., 2004, Nucleic Acids Res 32: 5991-6000; Hall et al., 2006, Nucleic Acids Res 34: 2773-2781). Phosphorothioate (PS) modifications can be easily placed in the RNA duplex at any desired position and can be made using standard chemical synthesis methods. The PS modification shows dose-dependent toxicity, so most investigators have recommended limited incorporation in siRNAs, favoring the 3′-ends where protection from nucleases is most important (Harborth et al., 2003, Antisense Nucleic Acid Drug Dev 13: 83-105; Chiu and Rana, 2003, Mol Cell 10: 549-561; Braasch et al., 2003, Biochemistry 42: 7967-7975; Amarzguioui et al., 2003, Nucleic Acids Research 31: 589-595). More extensive PS modification can be compatible with potent RNAi activity; however, use of sugar modifications (such as 2′-O-methyl RNA) may be superior (Choung et al., 2006, Biochem Biophys Res Commun 342: 919-927).

A variety of substitutions can be placed at the 2′-position of the ribose which generally increases duplex stability (Tₘ) and can greatly improve nuclease resistance. 2′-O-methyl RNA is a naturally occurring modification found in mammalian ribosomal RNAs and transfer RNAs. 2′-O-methyl modification in siRNAs is known, but the precise position of modified bases within the duplex is important to retain potency and complete substitution of 2′-O-methyl RNA for RNA will inactivate the siRNA. For example, a pattern that employs alternating 2′-O-methyl bases can have potency equivalent to unmodified RNA and is quite stable in serum (Choung et al., 2006, Biochem Biophys Res Commun 342: 919-927; Czauderna et al., 2003, Nucleic Acids Research 31: 2705-2716).

The 2′-fluoro (2′-F) modification is also compatible with dsRNA (e.g., siRNA and DsiRNA) function; it is most commonly placed at pyrimidine sites (due to reagent cost and availability) and can be combined with 2′-O-methyl modification at purine positions; 2′-F purines are available and can also be used. Heavily modified duplexes of this kind can be potent triggers of RNAi in vitro (Allerson et al., 2005, J Med Chem 48: 901-904; Prakash et al., 2005, J Med Chem 48: 4247-4253; Kraynack and Baker, 2006, RNA 12: 163-176) and can improve performance and extend duration of action when used in vivo (Morrissey et al., 2005, Hepatology 41: 1349-1356; Morrissey et al., 2005, Nat Biotechnol 23: 1002-1007). A highly potent, nuclease stable, blunt 19mer duplex containing alternative 2′-F and 2′-O-Me bases is taught by Allerson. In this design, alternating 2′-O-Me residues are positioned in an identical pattern to that employed by Czauderna, however the remaining RNA residues are converted to 2′-F modified bases. A highly potent, nuclease resistant siRNA employed by Morrissey employed a highly potent, nuclease resistant siRNA in vivo. In addition to 2′-O-Me RNA and 2′-F RNA, this duplex includes DNA, RNA, inverted abasic residues, and a 3′-terminal PS internucleoside linkage. While extensive modification has certain benefits, more limited modification of the duplex can also improve in vivo performance and is both simpler and less costly to manufacture. Soutschek et al. (2004, Nature 432: 173-178) employed a duplex in vivo and was mostly RNA with two 2′-O-Me RNA bases and limited 3′-terminal PS internucleoside linkages.

Locked nucleic acids (LNAs) are a different class of 2′-modification that can be used to stabilize dsRNA (e.g., siRNA and DsiRNA). Patterns of LNA incorporation that retain potency are more restricted than 2′-O-methyl or 2′-F bases, so limited modification is preferred (Braasch et al., 2003, Biochemistry 42: 7967-7975; Grunweller et al., 2003, Nucleic Acids Res 31: 3185-3193; Elmen et al., 2005, Nucleic Acids Res 33: 439-447). Even with limited incorporation, the use of LNA modifications can improve dsRNA performance in vivo and may also alter or improve off target effect profiles (Mook et al., 2007, Mol Cancer Ther 6: 833-843).

Synthetic nucleic acids introduced into cells or live animals can be recognized as “foreign” and trigger an immune response. Immune stimulation constitutes a major class of off-target effects which can dramatically change experimental results and even lead to cell death. The innate immune system includes a collection of receptor molecules that specifically interact with DNA and RNA that mediate these responses, some of which are located in the cytoplasm and some of which reside in endosomes (Marques and Williams, 2005, Nat Biotechnol 23: 1399-1405; Schlee et al., 2006, Mol Ther 14: 463-470). Delivery of siRNAs by cationic lipids or liposomes exposes the siRNA to both cytoplasmic and endosomal compartments, maximizing the risk for triggering a type 1 interferon (IFN) response both in vitro and in vivo (Morrissey et al., 2005, Nat Biotechnol 23: 1002-1007; Sioud and Sorensen, 2003, Biochem Biophys Res Commun 312: 1220-1225; Sioud, 2005, J Mol Biol 348: 1079-1090; Ma et al., 2005, Biochem Biophys Res Commun 330: 755-759). RNAs transcribed within the cell are less immunogenic (Robbins et al., 2006, Nat Biotechnol 24: 566-571) and synthetic RNAs that are immunogenic when delivered using lipid-based methods can evade immune stimulation when introduced unto cells by mechanical means, even in vivo (Heidel et al., 2004, Nat Biotechnol 22: 1579-1582). However, lipid based delivery methods are convenient, effective, and widely used. Some general strategy to prevent immune responses is needed, especially for in vivo application where all cell types are present and the risk of generating an immune response is highest. Use of chemically modified RNAs may solve most or even all of these problems.

Although certain sequence motifs are clearly more immunogenic than others, it appears that the receptors of the innate immune system in general distinguish the presence or absence of certain base modifications which are more commonly found in mammalian RNAs than in prokaryotic RNAs. For example, pseudouridine, N6-methyl-A, and 2′-O-methyl modified bases are recognized as “self” and inclusion of these residues in a synthetic RNA can help evade immune detection (Kariko et al., 2005, Immunity 23: 165-175). Extensive 2′-modification of a sequence that is strongly immunostimulatory as unmodified RNA can block an immune response when administered to mice intravenously (Morrissey et al., 2005, Nat Biotechnol 23: 1002-1007). However, extensive modification is not needed to escape immune detection and substitution of as few as two 2′-O-methyl bases in a single strand of a siRNA duplex can be sufficient to block a type 1 IFN response both in vitro and in vivo; modified U and G bases are most effective (Judge et al., 2006, Mol Ther 13: 494-505). As an added benefit, selective incorporation of 2′-O-methyl bases can reduce the magnitude of off-target effects (Jackson et al., 2006, RNA 12: 1197-1205). Use of 2′-O-methyl bases should therefore be considered for all dsRNAs intended for in vivo applications as a means of blocking immune responses and has the added benefit of improving nuclease stability and reducing the likelihood of off-target effects.

Although cell death can result from immune stimulation, assessing cell viability is not an adequate method to monitor induction of IFN responses. IFN responses can be present without cell death, and cell death can result from target knockdown in the absence of IFN triggering (for example, if the targeted gene is essential for cell viability). Relevant cytokines can be directly measured in culture medium and a variety of commercial kits exist which make performing such assays routine. While a large number of different immune effector molecules can be measured, testing levels of IFN-α, TNF-α, and IL-6 at 4 and 24 hours post transfection is usually sufficient for screening purposes. It is important to include a “transfection reagent only control” as cationic lipids can trigger immune responses in certain cells in the absence of any nucleic acid cargo. Including controls for IFN pathway induction should be considered for cell culture work. It is essential to test for immune stimulation whenever administering nucleic acids in vivo, where the risk of triggering IFN responses is highest.

Modifications can be included in the DsiRNA agents of the present invention so long as the modification does not prevent the DsiRNA agent from serving as a substrate for Dicer. In one embodiment, one or more modifications are made that enhance Dicer processing of the DsiRNA agent. In a second embodiment, one or more modifications are made that result in more effective RNAi generation. In a third embodiment, one or more modifications are made that support a greater RNAi effect. In a fourth embodiment, one or more modifications are made that result in greater potency per each DsiRNA agent molecule to be delivered to the cell. Modifications can be incorporated in the 3′-terminal region, the 5′-terminal region, in both the 3′-terminal and 5′-terminal region or in some instances in various positions within the sequence. With the restrictions noted above in mind, any number and combination of modifications can be incorporated into the DsiRNA agent. Where multiple modifications are present, they may be the same or different. Modifications to bases, sugar moieties, the phosphate backbone, and their combinations are contemplated. Either 5′-terminus can be phosphorylated.

Examples of modifications contemplated for the phosphate backbone include phosphonates, including methylphosphonate, phosphorothioate, and phosphotriester modifications such as alkylphosphotriesters, and the like. Examples of modifications contemplated for the sugar moiety include 2′-alkyl pyrimidine, such as 2′-O-methyl, 2′-fluoro, amino, and deoxy modifications and the like (see, e.g., Amarzguioui et al., 2003, Nucleic Acids Research 31: 589-595). Examples of modifications contemplated for the base groups include abasic sugars, 2-O-alkyl modified pyrimidines, 4-thiouracil, 5-bromouracil, 5-iodouracil, and 5-(3-aminoallyl)-uracil and the like. Locked nucleic acids, or LNA's, could also be incorporated. Many other modifications are known and can be used so long as the above criteria are satisfied. Examples of modifications are also disclosed in U.S. Pat. Nos. 5,684,143, 5,858,988 and 6,291,438 and in U.S. published patent application No. 2004/0203145 A1. Other modifications are disclosed in Herdewijn (2000, Antisense Nucleic Acid Drug Dev 10: 297-310), Eckstein (2000, Antisense Nucleic Acid Drug Dev 10: 117-21), Rusckowski et al. (2000, Antisense Nucleic Acid Drug Dev 10: 333-345), Stein et al. (2001, Antisense Nucleic Acid Drug Dev 11: 317-25); Vorobjev et al. (2001, Antisense Nucleic Acid Drug Dev 11: 77-85).

One or more modifications contemplated can be incorporated into either strand. The placement of the modifications in the DsiRNA agent can greatly affect the characteristics of the DsiRNA agent, including conferring greater potency and stability, reducing toxicity, enhance Dicer processing, and minimizing an immune response. In one embodiment, the antisense strand or the sense strand or both strands have one or more 2′-O-methyl modified nucleotides. In another embodiment, the antisense strand contains 2′-O-methyl modified nucleotides. In another embodiment, the antisense stand contains a 3′ overhang that is comprised of 2′-O-methyl modified nucleotides. The antisense strand could also include additional 2′-O-methyl modified nucleotides.

In certain embodiments of the present invention, the DsiRNA agent has one or more properties which enhance its processing by Dicer. According to these embodiments, the DsiRNA agent has a length sufficient such that it is processed by Dicer to produce an active siRNA and at least one of the following properties: (i) the DsiRNA agent is asymmetric, e.g., has a 3′ overhang on the antisense strand and (ii) the DsiRNA agent has a modified 3′ end on the sense strand to direct orientation of Dicer binding and processing of the dsRNA to an active siRNA. According to this embodiment, the longest strand in the dsRNA comprises 25-35 nucleotides. In one embodiment, the DsiRNA agent is asymmetric such that the sense strand comprises 25-28 nucleotides and the antisense strand comprises 25-30 nucleotides. Thus, the resulting dsRNA has an overhang on the 3′ end of the antisense strand. The overhang is 1-4 nucleotides, for example 2 nucleotides. The sense strand may also have a 5′ phosphate.

In other embodiments, the sense strand of the DsiRNA agent is modified for Dicer processing by suitable modifiers located at the 3′ end of the sense strand, i.e., the DsiRNA agent is designed to direct orientation of Dicer binding and processing. Suitable modifiers include nucleotides such as deoxyribonucleotides, dideoxyribonucleotides, acyclonucleotides and the like and sterically hindered molecules, such as fluorescent molecules and the like. Acyclonucleotides substitute a 2-hydroxyethoxymethyl group for the 2′-deoxyribofuranosyl sugar normally present in dNMPs. Other nucleotides modifiers could include 3′-deoxyadenosine (cordycepin), 3′-azido-3′-deoxythymidine (AZT), 2′,3′-dideoxyinosine (ddI), 2′,3′-dideoxy-3′-thiacytidine (3TC), 2′,3′-didehydro-2′,3′-dideoxythymidine (d4T) and the monophosphate nucleotides of 3′-azido-3′-deoxythymidine (AZT), 2′,3′-dideoxy-3′-thiacytidine (3TC) and 2′,3′-didehydro-2′,3′-dideoxythymidine (d4T). In one embodiment, deoxynucleotides are used as the modifiers. When nucleotide modifiers are utilized, 1-3 nucleotide modifiers, or 2 nucleotide modifiers are substituted for the ribonucleotides on the 3′ end of the sense strand. When sterically hindered molecules are utilized, they are attached to the ribonucleotide at the 3′ end of the antisense strand. Thus, the length of the strand does not change with the incorporation of the modifiers. In another embodiment, the invention contemplates substituting two DNA bases in the DsiRNA agent to direct the orientation of Dicer processing of the antisense strand. In a further embodiment of the present invention, two terminal DNA bases are substituted for two ribonucleotides on the 3′-end of the sense strand forming a blunt end of the duplex on the 3′ end of the sense strand and the 5′ end of the antisense strand, and a two-nucleotide RNA overhang is located on the 3′-end of the antisense strand. This is an asymmetric composition with DNA on the blunt end and RNA bases on the overhanging end.

The sense and antisense strands of a DsiRNA agent of the instant invention anneal under biological conditions, such as the conditions found in the cytoplasm of a cell. In addition, a region of one of the sequences, particularly of the antisense strand, of the DsiRNA agent has a sequence length of at least 19 nucleotides, wherein these nucleotides are in the 21-nucleotide region adjacent to the 3′ end of the antisense strand and are sufficiently complementary to a nucleotide sequence of the RNA produced from the target gene.

The DsiRNA agent may also have one or more of the following additional properties: (a) the antisense strand has a right shift from the typical 21mer, (b) the strands may not be completely complementary, i.e., the strands may contain simple mismatch pairings and (c) base modifications such as locked nucleic acid(s) may be included in the 5′ end of the sense strand. A “typical” 21mer siRNA is designed using conventional techniques. In one technique, a variety of sites are commonly tested in parallel or pools containing several distinct siRNA duplexes specific to the same target with the hope that one of the reagents will be effective (Ji et al., 2003, FEBS Lett 552: 247-252). Other techniques use design rules and algorithms to increase the likelihood of obtaining active RNAi effector molecules (Schwarz et al., 2003, Cell 115: 199-208; Khvorova et al., 2003, Cell 115: 209-216; Ui-Tei et al., 2004, Nucleic Acids Res 32: 936-948; Reynolds et al., 2004, Nat Biotechnol 22: 326-330; Krol et al., 2004, J Biol Chem 279: 42230-42239; Yuan et al., 2004, Nucl Acids Res 32(Webserver issue):W130-134; Boese et al., 2005, Methods Enzymol 392: 73-96). High throughput selection of siRNA has also been developed (U.S. published patent application No. 2005/0042641 A1). Potential target sites can also be analyzed by secondary structure predictions (Heale et al., 2005, Nucleic Acids Res 33(3): e30). This 21mer is then used to design a right shift to include 3-9 additional nucleotides on the 5′ end of the 21mer. The sequence of these additional nucleotides may have any sequence. In one embodiment, the added ribonucleotides are based on the sequence of the target gene. Even in this embodiment, full complementarity between the target sequence and the antisense siRNA is not required.

The first and second oligonucleotides of a DsiRNA agent of the instant invention are not required to be completely complementary. They only need to be substantially complementary to anneal under biological conditions and to provide a substrate for Dicer that produces a siRNA sufficiently complementary to the target sequence. Locked nucleic acids, or LNA's, are well known to a skilled artisan (Elmen et al., 2005, Nucleic Acids Res 33: 439-447; Kurreck et al., 2002, Nucleic Acids Res 30: 1911-1918; Crinelli et al., 2002, Nucleic Acids Res 30: 2435-2443; Braasch and Corey, 2001, Chem Biol 8: 1-7; Bondensgaard et al., 2000, Chemistry 6: 2687-2695; Wahlestedt et al., 2000, Proc Natl Acad Sci USA 97: 5633-5638). In one embodiment, an LNA is incorporated at the 5′ terminus of the sense strand. In another embodiment, an LNA is incorporated at the 5′ terminus of the sense strand in duplexes designed to include a 3′ overhang on the antisense strand.

In certain embodiments, the DsiRNA agent of the instant invention has an asymmetric structure, with the sense strand having a 25-base pair length, and the antisense strand having a 27-base pair length with a 2 base 3′-overhang. In other embodiments, this DsiRNA agent having an asymmetric structure further contains 2 deoxynucleotides at the 3′ end of the sense strand in place of two of the ribonucleotides.

Certain DsiRNA agent compositions containing two separate oligonucleotides can be linked by a third structure. The third structure will not block Dicer activity on the DsiRNA agent and will not interfere with the directed destruction of the RNA transcribed from the target gene. In one embodiment, the third structure may be a chemical linking group. Many suitable chemical linking groups are known in the art and can be used. Alternatively, the third structure may be an oligonucleotide that links the two oligonucleotides of the DsiRNA agent in a manner such that a hairpin structure is produced upon annealing of the two oligonucleotides making up the dsRNA composition. The hairpin structure will not block Dicer activity on the DsiRNA agent and will not interfere with the directed destruction of the target RNA.

In certain embodiments, the DsiRNA agents of the invention have several properties which enhance its processing by Dicer. According to such embodiments, the DsiRNA agent has a length sufficient such that it is processed by Dicer to produce an siRNA and at least one of the following properties: (i) the DsiRNA agent is asymmetric, e.g., has a 3′ overhang on the sense strand and (ii) the DsiRNA agent has a modified 3′ end on the antisense strand to direct orientation of Dicer binding and processing of the dsRNA to an active siRNA. According to these embodiments, the longest strand in the DsiRNA agent comprises 25-30 nucleotides. In one embodiment, the sense strand comprises 25-30 nucleotides and the antisense strand comprises 25-28 nucleotides. Thus, the resulting dsRNA has an overhang on the 3′ end of the sense strand. The overhang is 1-4 nucleotides, such as 2 nucleotides. The antisense strand may also have a 5′ phosphate.

In certain embodiments, the sense strand of a DsiRNA agent is modified for Dicer processing by suitable modifiers located at the 3′ end of the sense strand, i.e., the DsiRNA agent is designed to direct orientation of Dicer binding and processing. Suitable modifiers include nucleotides such as deoxyribonucleotides, dideoxyribonucleotides, acyclonucleotides and the like and sterically hindered molecules, such as fluorescent molecules and the like. Acyclonucleotides substitute a 2-hydroxyethoxymethyl group for the 2′-deoxyribofuranosyl sugar normally present in dNMPs. Other nucleotide modifiers could include 3′-deoxyadenosine (cordycepin), 3′-azido-3′-deoxythymidine (AZT), 2′,3′-dideoxyinosine (ddI), 2′,3′-dideoxy-3′-thiacytidine (3TC), 2′,3′-didehydro-2′,3′-dideoxythymidine (d4T) and the monophosphate nucleotides of 3′-azido-3′-deoxythymidine (AZT), 2′,3′-dideoxy-3′-thiacytidine (3TC) and 2′,3′-didehydro-2′,3′-dideoxythymidine (d4T). In one embodiment, deoxynucleotides are used as the modifiers. When nucleotide modifiers are utilized, 1-3 nucleotide modifiers, or 2 nucleotide modifiers are substituted for the ribonucleotides on the 3′ end of the sense strand. When sterically hindered molecules are utilized, they are attached to the ribonucleotide at the 3′ end of the antisense strand. Thus, the length of the strand does not change with the incorporation of the modifiers. In another embodiment, the invention contemplates substituting two DNA bases in the dsRNA to direct the orientation of Dicer processing. In a further invention, two terminal DNA bases are located on the 3′ end of the sense strand in place of two ribonucleotides forming a blunt end of the duplex on the 5′ end of the antisense strand and the 3′ end of the sense strand, and a two-nucleotide RNA overhang is located on the 3′-end of the antisense strand. This is an asymmetric composition with DNA on the blunt end and RNA bases on the overhanging end.

In certain other embodiments, the antisense strand of a DsiRNA agent is modified for Dicer processing by suitable modifiers located at the 3′ end of the antisense strand, i.e., the DsiRNA agent is designed to direct orientation of Dicer binding and processing. Suitable modifiers include nucleotides such as deoxyribonucleotides, dideoxyribonucleotides, acyclonucleotides and the like and sterically hindered molecules, such as fluorescent molecules and the like. Acyclonucleotides substitute a 2-hydroxyethoxymethyl group for the 2′-deoxyribofuranosyl sugar normally present in dNMPs. Other nucleotide modifiers could include 3′-deoxyadenosine (cordycepin), 3′-azido-3′-deoxythymidine (AZT), 2′,3′-dideoxyinosine (ddI), 2′,3′-dideoxy-3′-thiacytidine (3TC), 2′,3′-didehydro-2′,3′-dideoxythymidine (d4T) and the monophosphate nucleotides of 3′-azido-3′-deoxythymidine (AZT), 2′,3′-dideoxy-3′-thiacytidine (3TC) and 2′,3′-didehydro-2′,3′-dideoxythymidine (d4T). In one embodiment, deoxynucleotides are used as the modifiers. When nucleotide modifiers are utilized, 1-3 nucleotide modifiers, or 2 nucleotide modifiers are substituted for the ribonucleotides on the 3′ end of the antisense strand. When sterically hindered molecules are utilized, they are attached to the ribonucleotide at the 3′ end of the antisense strand. Thus, the length of the strand does not change with the incorporation of the modifiers. In another embodiment, the invention contemplates substituting two DNA bases in the dsRNA to direct the orientation of Dicer processing. In a further invention, two terminal DNA bases are located on the 3′ end of the antisense strand in place of two ribonucleotides forming a blunt end of the duplex on the 5′ end of the sense strand and the 3′ end of the antisense strand, and a two-nucleotide RNA overhang is located on the 3′-end of the sense strand. This is also an asymmetric composition with DNA on the blunt end and RNA bases on the overhanging end.

The sense and antisense strands anneal under biological conditions, such as the conditions found in the cytoplasm of a cell. In addition, a region of one of the sequences, particularly of the antisense strand, of the dsRNA has a sequence length of at least 19 nucleotides, wherein these nucleotides are adjacent to the 3′ end of antisense strand and are sufficiently complementary to a nucleotide sequence of the target RNA.

Additionally, the DsiRNA agent structure can be optimized to ensure that the oligonucleotide segment generated from Dicer's cleavage will be the portion of the oligonucleotide that is most effective in inhibiting gene expression. For example, in one embodiment of the invention, a 27-bp oligonucleotide of the DsiRNA agent structure is synthesized wherein the anticipated 21 to 22-bp segment that will inhibit gene expression is located on the 3′-end of the antisense strand. The remaining bases located on the 5′-end of the antisense strand will be cleaved by Dicer and will be discarded. This cleaved portion can be homologous (i.e., based on the sequence of the target sequence) or non-homologous and added to extend the nucleic acid strand.

US 2007/0265220 discloses that 27mer DsiRNAs show improved stability in serum over comparable 21mer siRNA compositions, even absent chemical modification. Modifications of DsiRNA agents, such as inclusion of 2′-O-methyl RNA in the antisense strand, in patterns such as detailed above, when coupled with addition of a 5′ Phosphate, can improve stability of DsiRNA agents. Addition of 5′-phosphate to all strands in synthetic RNA duplexes may be an inexpensive and physiological method to confer some limited degree of nuclease stability. The chemical modification patterns of the DsiRNA agents of the instant invention are designed to enhance the efficacy of such agents. Accordingly, such modifications are designed to avoid reducing potency of DsiRNA agents; to avoid interfering with Dicer processing of DsiRNA agents; to improve stability in biological fluids (reduce nuclease sensitivity) of DsiRNA agents; or to block or evade detection by the innate immune system. Such modifications are also designed to avoid being toxic and to avoid increasing the cost or impact the ease of manufacturing the instant DsiRNA agents of the invention.

### RNAi In Vitro Assay to Assess DsiRNA Activity

An in vitro assay that recapitulates RNAi in a cell-free system can be used to evaluate DsiRNA constructs targeting an RNA sequence(s) of interest. The assay comprises the system described by Tuschl et al., 1999, Genes and Development, 13, 3191-3197 and Zamore et al., 2000, Cell, 101, 25-33 adapted for use with DsiRNA agents directed against a target RNA. A Drosophila extract derived from syncytial blastoderm is used to reconstitute RNAi activity in vitro. Target RNA is generated via in vitro transcription from an appropriate target RNA expressing plasmid using T7 RNA polymerase or via chemical synthesis. Sense and antisense DsiRNA strands (for example 20 uM each) are annealed by incubation in buffer (such as 100 mM potassium acetate, 30 mM HEPES-KOH, pH 7.4, 2 mM magnesium acetate) for 1 minute at 90° C. followed by 1 hour at 37° C., then diluted in lysis buffer (for example 100 mM potassium acetate, 30 mM HEPES-KOH at pH 7.4, 2 mM magnesium acetate). Annealing can be monitored by gel electrophoresis on an agarose gel in TBE buffer and stained with ethidium bromide. The Drosophila lysate is prepared using zero to two-hour-old embryos from Oregon R flies collected on yeasted molasses agar that are dechorionated and lysed. The lysate is centrifuged and the supernatant isolated. The assay comprises a reaction mixture containing 50% lysate [vol/vol], RNA (10-50 μM final concentration), and 10% [vol/vol] lysis buffer containing DsiRNA (10 nM final concentration). The reaction mixture also contains 10 mM creatine phosphate, 10 ug/ml creatine phosphokinase, 100 um GTP, 100 uM UTP, 100 uM CTP, 500 uM ATP, 5 mM DTT, 0.1 U/uL RNasin (Promega), and 100 uM of each amino acid. The final concentration of potassium acetate is adjusted to 100 mM. The reactions are pre-assembled on ice and preincubated at 25° C. for 10 minutes before adding RNA, then incubated at 25° C. for an additional 60 minutes. Reactions are quenched with 4 volumes of 1.25×Passive Lysis Buffer (Promega). Target RNA cleavage is assayed by RT-PCR analysis or other methods known in the art and are compared to control reactions in which DsiRNA is omitted from the reaction.

Alternately, internally-labeled target RNA for the assay is prepared by in vitro transcription in the presence of [alpha-³²P] CTP, passed over a G50 Sephadex column by spin chromatography and used as target RNA without further purification. Optionally, target RNA is 5′-³²P-end labeled using T4 polynucleotide kinase enzyme. Assays are performed as described above and target RNA and the specific RNA cleavage products generated by RNAi are visualized on an autoradiograph of a gel. The percentage of cleavage is determined by PHOSPHOR IMAGER® (autoradiography) quantitation of bands representing intact control RNA or RNA from control reactions without DsiRNA and the cleavage products generated by the assay.

In one embodiment, this assay is used to determine target sites in the RNA target of interest for DsiRNA mediated RNAi cleavage, wherein a plurality of DsiRNA constructs are screened for RNAi mediated cleavage of the RNA target of interest, for example, by analyzing the assay reaction by electrophoresis of labeled target RNA, or by northern blotting, as well as by other methodology well known in the art.

Structures of dsiRNA-Peptide Agents

In certain embodiments, the dsRNA agents of the invention can have any of the following structures:

In one such embodiment, the dsRNA comprises:

5′-XXXXXXXXXXXXXXXXXXXXXXXDDP-3′
3′-YXXXXXXXXXXXXXXXXXXXXXXXXX-5′

wherein “X”=RNA, “Y” is an overhang domain comprised of 1-4 RNA monomers that are optionally 2′-O-methyl RNA monomers, “D”=DNA and “P”=peptide. The top strand is the sense strand, and the bottom strand is the antisense strand.

In another such embodiment, the dsRNA comprises:

5′-PXXXXXXXXXXXXXXXXXXXXXXXDD-3′
3′-YXXXXXXXXXXXXXXXXXXXXXXXXX-5′

wherein “X”=RNA, “Y” is an overhang domain comprised of 1-4 RNA monomers that are optionally 2′-O-methyl RNA monomers, “D”=DNA and “P”=peptide. The top strand is the sense strand, and the bottom strand is the antisense strand.

In another such embodiment, the dsRNA comprises:

5′-XXXXXXXXXXXXXXXXXXXXXXXDD-3′
3′-YXXXXXXXXXXXXXXXXXXXXXXXXXP-5′

wherein “X”=RNA, “Y” is an overhang domain comprised of 1-4 RNA monomers that are optionally 2′-O-methyl RNA monomers, “D”=DNA and “P”=peptide. The top strand is the sense strand, and the bottom strand is the antisense strand.

In another such embodiment, the dsRNA comprises:

5′-XXXXXXXXXXXXXXXXXXXXXXXDD-3′
3′-PYXXXXXXXXXXXXXXXXXXXXXXXXX-5′

wherein “X”=RNA, “Y” is an overhang domain comprised of 1-4 RNA monomers that are optionally 2′-O-methyl RNA monomers, “D”=DNA and “P”=peptide. The top strand is the sense strand, and the bottom strand is the antisense strand.

In another such embodiment, the dsRNA comprises:

5′-XXXXXXXXXXXXXXXXXXXXXXXDD-3′
3′-PXXXXXXXXXXXXXXXXXXXXXXXXX-5′

wherein “X”=RNA, “Y” is an overhang domain comprised of 1-4 RNA monomers that are optionally 2′-O-methyl RNA monomers, “D”=DNA and “P”=peptide. The top strand is the sense strand, and the bottom strand is the antisense strand.

In another such embodiment, the dsRNA comprises:

5′-PXXXXXXXXXXXXXXXXXXXXXXXDDP-3′
3′-YXXXXXXXXXXXXXXXXXXXXXXXXX-5′

wherein “X”=RNA, “Y” is an overhang domain comprised of 1-4 RNA monomers that are optionally 2′-O-methyl RNA monomers, “D”=DNA and “P”=peptide. The top strand is the sense strand, and the bottom strand is the antisense strand.

In another such embodiment, the dsRNA comprises:

5′-XXXXXXXXXXXXXXXXXXXXXXXDD-3′
3′-PYXXXXXXXXXXXXXXXXXXXXXXXXXP-5′

wherein “X”=RNA, “Y” is an overhang domain comprised of 1-4 RNA monomers that are optionally 2′-O-methyl RNA monomers, “D”=DNA and “P”=peptide. The top strand is the sense strand, and the bottom strand is the antisense strand.

In another such embodiment, the dsRNA comprises:

5′-XXXXXXXXXXXXXXXXXXXXXXXDD-3′
3′-PXXXXXXXXXXXXXXXXXXXXXXXXXP-5′

wherein “X”=RNA, “Y” is an overhang domain comprised of 1-4 RNA monomers that are optionally 2′-O-methyl RNA monomers, “D”=DNA and “P”=peptide. The top strand is the sense strand, and the bottom strand is the antisense strand.

In another such embodiment, the dsRNA comprises:

5′-PXXXXXXXXXXXXXXXXXXXXXXXDDP-3′
3′-PYXXXXXXXXXXXXXXXXXXXXXXXXXP-5′

wherein “X”=RNA, “Y” is an overhang domain comprised of 1-4 RNA monomers that are optionally 2′-O-methyl RNA monomers, “D”=DNA and “P”=peptide. The top strand is the sense strand, and the bottom strand is the antisense strand.

In another such embodiment, the dsRNA comprises:

5′-PXXXXXXXXXXXXXXXXXXXXXXXDDP-3′
3′-PXXXXXXXXXXXXXXXXXXXXXXXXXP-5′

wherein “X”=RNA, “Y” is an overhang domain comprised of 1-4 RNA monomers that are optionally 2′-O-methyl RNA monomers, “D”=DNA and “P”=peptide. The top strand is the sense strand, and the bottom strand is the antisense strand.

In other embodiments, the DsiRNA comprises:

5′-XXXXXXXXXXXXXXXXXXXXXXXXXP-3′
3′-YXXXXXXXXXXXXXXXXXXXXXXXXX-5′
or
5′-PXXXXXXXXXXXXXXXXXXXXXXXXX-3′
3′-YXXXXXXXXXXXXXXXXXXXXXXXXX-5′
or
5′-XXXXXXXXXXXXXXXXXXXXXXXXX-3′
3′-YXXXXXXXXXXXXXXXXXXXXXXXXXP-5′
or
5′-XXXXXXXXXXXXXXXXXXXXXXXXX-3′
3′-PYXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXX-5′
or
5′-PXXXXXXXXXXXXXXXXXXXXXXXXXP-3′
3′-YXXXXXXXXXXXXXXXXXXXXXXXXX-5′
or
5′-XXXXXXXXXXXXXXXXXXXXXXXXX-3′
3′-PYXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXP-5′
or
5′-PXXXXXXXXXXXXXXXXXXXXXXXXXP-3′
3′-PYXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXDDP-3′
3′-YXXXXXXXXXXXXXXXXXXXXXXXXX-5′
or
5′-PXXXXXXXXXXXXXXXXXXXXXXXDD-3′
3′-YXXXXXXXXXXXXXXXXXXXXXXXXX-5′
or
5′-XXXXXXXXXXXXXXXXXXXXXXXDD-3′
3′-YXXXXXXXXXXXXXXXXXXXXXXXXXP-5′
or
5′-XXXXXXXXXXXXXXXXXXXXXXXDD-3′
3′-PYXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXX-5′
or
5′-PXXXXXXXXXXXXXXXXXXXXXXXDDP-3′
3′-YXXXXXXXXXXXXXXXXXXXXXXXXX-5′
or
5′-XXXXXXXXXXXXXXXXXXXXXXXDD-3′
3′-PYXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXP-5′
or
5′-PXXXXXXXXXXXXXXXXXXXXXXXDDP-3′
3′-PYXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXXXP-3′
3′-YXXXXXXXXXXXXXXXXXXXXXXXXX-5′
or
5′-PXXXXXXXXXXXXXXXXXXXXXXXXX-3′
3′-YXXXXXXXXXXXXXXXXXXXXXXXXX-5′
or
5′-XXXXXXXXXXXXXXXXXXXXXXXXX-3′
3′-YXXXXXXXXXXXXXXXXXXXXXXXXXP-5′
or
5′-XXXXXXXXXXXXXXXXXXXXXXXXX-3′
3′-PYXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXX-5′
or
5′-PXXXXXXXXXXXXXXXXXXXXXXXXXP-3′
3′-YXXXXXXXXXXXXXXXXXXXXXXXXX-5′
or
5′-XXXXXXXXXXXXXXXXXXXXXXXXX-3′
3′-PYXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXP-5′
or
5′-PXXXXXXXXXXXXXXXXXXXXXXXXXP-3′
3′-PYXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXDDP-3′
3′-YXXXXXXXXXXXXXXXXXXXXXXXXX-5′
or
5′-PXXXXXXXXXXXXXXXXXXXXXXXDD-3′
3′-YXXXXXXXXXXXXXXXXXXXXXXXXX-5′
or
5′-XXXXXXXXXXXXXXXXXXXXXXXDD-3′
3′-YXXXXXXXXXXXXXXXXXXXXXXXXXP-5′
or
5′-XXXXXXXXXXXXXXXXXXXXXXXDD-3′
3′-PYXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXX-5′
or
5′-PXXXXXXXXXXXXXXXXXXXXXXXDDP-3′
3′-YXXXXXXXXXXXXXXXXXXXXXXXXX-5′
or
5′-XXXXXXXXXXXXXXXXXXXXXXXDD-3′
3′-PYXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXP-5′
or
5′-PXXXXXXXXXXXXXXXXXXXXXXXDDP-3′
3′-PYXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXXXP-3′
3′-YXXXXXXXXXXXXXXXXXXXXXXXXX-5′
or
5′-PXXXXXXXXXXXXXXXXXXXXXXXXX-3′
3′-YXXXXXXXXXXXXXXXXXXXXXXXXX-5′
or
5′-XXXXXXXXXXXXXXXXXXXXXXXXX-3′
3′-YXXXXXXXXXXXXXXXXXXXXXXXXXP-5′
or
5′-XXXXXXXXXXXXXXXXXXXXXXXXX-3′
3′-PYXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXX-5′
or
5′-PXXXXXXXXXXXXXXXXXXXXXXXXXP-3′
3′-YXXXXXXXXXXXXXXXXXXXXXXXXX-5′
or
5′-XXXXXXXXXXXXXXXXXXXXXXXXX-3′
3′-PYXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXP-5′
or
5′-PXXXXXXXXXXXXXXXXXXXXXXXXXP-3′
3′-PYXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXDDP-3′
3′-YXXXXXXXXXXXXXXXXXXXXXXXXX-5′
or
5′-PXXXXXXXXXXXXXXXXXXXXXXXDD-3′
3′-YXXXXXXXXXXXXXXXXXXXXXXXXX-5′
or
5′-XXXXXXXXXXXXXXXXXXXXXXXDD-3′
3′-YXXXXXXXXXXXXXXXXXXXXXXXXXP-5′
or
5′-XXXXXXXXXXXXXXXXXXXXXXXDD-3′
3′-PYXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXX-5′
or
5′-PXXXXXXXXXXXXXXXXXXXXXXXDDP-3′
3′-YXXXXXXXXXXXXXXXXXXXXXXXXX-5′
or
5′-XXXXXXXXXXXXXXXXXXXXXXXDD-3′
3′-PYXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXP-5′
or
5′-PXXXXXXXXXXXXXXXXXXXXXXXDDP-3′
3′-PYXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXXXP-3′
3′-YXXXXXXXXXXXXXXXXXXXXXXXXX-5′
or
5′-PXXXXXXXXXXXXXXXXXXXXXXXXX-3′
3′-YXXXXXXXXXXXXXXXXXXXXXXXXX-5′
or
5′-XXXXXXXXXXXXXXXXXXXXXXXXX-3′
3′-YXXXXXXXXXXXXXXXXXXXXXXXXXP-5′
or
5′-XXXXXXXXXXXXXXXXXXXXXXXXX-3′
3′-PYXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXX-5′
or
5′-PXXXXXXXXXXXXXXXXXXXXXXXXXP-3′
3′-YXXXXXXXXXXXXXXXXXXXXXXXXX-5′
or
5′-XXXXXXXXXXXXXXXXXXXXXXXXX-3′
3′-PYXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXP-5′
or
5′-PXXXXXXXXXXXXXXXXXXXXXXXXXP-3′
3′-PYXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXDDP-3′
3′-YXXXXXXXXXXXXXXXXXXXXXXXXX-5′
or
5′-PXXXXXXXXXXXXXXXXXXXXXXXDD-3′
3′-YXXXXXXXXXXXXXXXXXXXXXXXXX-5′
or
5′-XXXXXXXXXXXXXXXXXXXXXXXDD-3′
3′-YXXXXXXXXXXXXXXXXXXXXXXXXXP-5′
or
5′-XXXXXXXXXXXXXXXXXXXXXXXDD-3′
3′-PYXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXX-5′
or
5′-PXXXXXXXXXXXXXXXXXXXXXXXDDP-3′
3′-YXXXXXXXXXXXXXXXXXXXXXXXXX-5′
or
5′-XXXXXXXXXXXXXXXXXXXXXXXDD-3′
3′-PYXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXP-5′
or
5′-PXXXXXXXXXXXXXXXXXXXXXXXDDP-3′
3′-PYXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXXXP-3′
3′-YXXXXXXXXXXXXXXXXXXXXXXXXX-5′
or
5′-PXXXXXXXXXXXXXXXXXXXXXXXXX-3′
3′-YXXXXXXXXXXXXXXXXXXXXXXXXX-5′
or
5′-XXXXXXXXXXXXXXXXXXXXXXXXX-3′
3′-YXXXXXXXXXXXXXXXXXXXXXXXXXP-5′
or
5′-XXXXXXXXXXXXXXXXXXXXXXXXX-3′
3′-PYXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXX-5′
or
5′-PXXXXXXXXXXXXXXXXXXXXXXXXXP-3′
3′-YXXXXXXXXXXXXXXXXXXXXXXXXX-5′
or
5′-XXXXXXXXXXXXXXXXXXXXXXXXX-3′
3′-PYXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXP-5′
or
5′-PXXXXXXXXXXXXXXXXXXXXXXXXXP-3′
3′-PYXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXDDP-3′
3′-YXXXXXXXXXXXXXXXXXXXXXXXXX-5′
or
5′-PXXXXXXXXXXXXXXXXXXXXXXXDD-3′
3′-YXXXXXXXXXXXXXXXXXXXXXXXXX-5′
or
5′-XXXXXXXXXXXXXXXXXXXXXXXDD-3′
3′-YXXXXXXXXXXXXXXXXXXXXXXXXXP-5′
or
5′-XXXXXXXXXXXXXXXXXXXXXXXDD-3′
3′-PYXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXX-5′
or
5′-PXXXXXXXXXXXXXXXXXXXXXXXDDP-3′
3′-YXXXXXXXXXXXXXXXXXXXXXXXXX-5′
or
5′-XXXXXXXXXXXXXXXXXXXXXXXDD-3′
3′-PYXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXP-5′
or
5′-PXXXXXXXXXXXXXXXXXXXXXXXDDP-3′
3′-PYXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXXXP-3′
3′-YXXXXXXXXXXXXXXXXXXXXXXXXX-5′
or
5′-PXXXXXXXXXXXXXXXXXXXXXXXXX-3′
3′-YXXXXXXXXXXXXXXXXXXXXXXXXX-5′
or
5′-XXXXXXXXXXXXXXXXXXXXXXXXX-3′
3′-YXXXXXXXXXXXXXXXXXXXXXXXXXP-5′
or
5′-XXXXXXXXXXXXXXXXXXXXXXXXX-3′
3′-PYXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXX-5′
or
5′-PXXXXXXXXXXXXXXXXXXXXXXXXXP-3′
3′-YXXXXXXXXXXXXXXXXXXXXXXXXX-5′
or
5′-XXXXXXXXXXXXXXXXXXXXXXXXX-3′
3′-PYXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXP-5′
or
5′-PXXXXXXXXXXXXXXXXXXXXXXXXXP-3′
3′-PYXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXDDP-3′
3′-YXXXXXXXXXXXXXXXXXXXXXXXXX-5′
or
5′-PXXXXXXXXXXXXXXXXXXXXXXXDD-3′
3′-YXXXXXXXXXXXXXXXXXXXXXXXXX-5′
or
5′-XXXXXXXXXXXXXXXXXXXXXXXDD-3′
3′-YXXXXXXXXXXXXXXXXXXXXXXXXXP-5′
or
5′-XXXXXXXXXXXXXXXXXXXXXXXDD-3′
3′-PYXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXX-5′
or
5′-PXXXXXXXXXXXXXXXXXXXXXXXDDP-3′
3′-YXXXXXXXXXXXXXXXXXXXXXXXXX-5′
or
5′-XXXXXXXXXXXXXXXXXXXXXXXDD-3′
3′-PYXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXP-5′
or
5′-PXXXXXXXXXXXXXXXXXXXXXXXDDP-3′
3′-PYXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXXXP-3′
3′-YXXXXXXXXXXXXXXXXXXXXXXXXX-5′
or
5′-PXXXXXXXXXXXXXXXXXXXXXXXXX-3′
3′-YXXXXXXXXXXXXXXXXXXXXXXXXX-5′
or
5′-XXXXXXXXXXXXXXXXXXXXXXXXX-3′
3′-YXXXXXXXXXXXXXXXXXXXXXXXXXP-5′
or
5′-XXXXXXXXXXXXXXXXXXXXXXXXX-3′
3′-PYXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXX-5′
or
5′-PXXXXXXXXXXXXXXXXXXXXXXXXXP-3′
3′-YXXXXXXXXXXXXXXXXXXXXXXXXX-5′
or
5′-XXXXXXXXXXXXXXXXXXXXXXXXX-3′
3′-PYXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXP-5′
or
5′-PXXXXXXXXXXXXXXXXXXXXXXXXXP-3′
3′-PYXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXDDP-3′
3′-YXXXXXXXXXXXXXXXXXXXXXXXXX-5′
or
5′-PXXXXXXXXXXXXXXXXXXXXXXXDD-3′
3′-YXXXXXXXXXXXXXXXXXXXXXXXXX-5′
or
5′-XXXXXXXXXXXXXXXXXXXXXXXDD-3′
3′-YXXXXXXXXXXXXXXXXXXXXXXXXXP-5′
or
5′-XXXXXXXXXXXXXXXXXXXXXXXDD-3′
3′-PYXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXX-5′
or
5′-PXXXXXXXXXXXXXXXXXXXXXXXDDP-3′
3′-YXXXXXXXXXXXXXXXXXXXXXXXXX-5′
or
5′-XXXXXXXXXXXXXXXXXXXXXXXDD-3′
3′-PYXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXP-5′
or
5′-PXXXXXXXXXXXXXXXXXXXXXXXDDP-3′
3′-PYXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXXXP-3′
3′-YXXXXXXXXXXXXXXXXXXXXXXXXX-5′
or
5′-PXXXXXXXXXXXXXXXXXXXXXXXXX-3′
3′-YXXXXXXXXXXXXXXXXXXXXXXXXX-5′
or
5′-XXXXXXXXXXXXXXXXXXXXXXXXX-3′
3′-YXXXXXXXXXXXXXXXXXXXXXXXXXP-5′
or
5′-XXXXXXXXXXXXXXXXXXXXXXXXX-3′
3′-PYXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXX-5′
or
5′-PXXXXXXXXXXXXXXXXXXXXXXXXXP-3′
3′-YXXXXXXXXXXXXXXXXXXXXXXXXX-5′
or
5′-XXXXXXXXXXXXXXXXXXXXXXXXX-3′
3′-PYXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXP-5′
or
5′-PXXXXXXXXXXXXXXXXXXXXXXXXXP-3′
3′-PYXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXDDP-3′
3′-YXXXXXXXXXXXXXXXXXXXXXXXXX-5′
or
5′-PXXXXXXXXXXXXXXXXXXXXXXXDD-3′
3′-YXXXXXXXXXXXXXXXXXXXXXXXXX-5′
or
5′-XXXXXXXXXXXXXXXXXXXXXXXDD-3′
3′-YXXXXXXXXXXXXXXXXXXXXXXXXXP-5′
or
5′-XXXXXXXXXXXXXXXXXXXXXXXDD-3′
3′-PYXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXX-5′
or
5′-PXXXXXXXXXXXXXXXXXXXXXXXDDP-3′
3′-YXXXXXXXXXXXXXXXXXXXXXXXXX-5′
or
5′-XXXXXXXXXXXXXXXXXXXXXXXDD-3′
3′-PYXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXP-5′
or
5′-PXXXXXXXXXXXXXXXXXXXXXXXDDP-3′
3′-PYXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXXXP-3′
3′-YXXXXXXXXXXXXXXXXXXXXXXXXXXX-5′
or
5′-PXXXXXXXXXXXXXXXXXXXXXXXXX-3′
3′-YXXXXXXXXXXXXXXXXXXXXXXXXXXX-5′
or
5′-XXXXXXXXXXXXXXXXXXXXXXXXX-3′
3′-YXXXXXXXXXXXXXXXXXXXXXXXXXXXP-5′
or
5′-XXXXXXXXXXXXXXXXXXXXXXXXX-3′
3′-PYXXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-YXXXXXXXXXXXXXXXXXXXXXXXXXXX-5′
or
5′-XXXXXXXXXXXXXXXXXXXXXXXXX-3′
3′-PYXXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PYXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXDDP-3′
3′-YXXXXXXXXXXXXXXXXXXXXXXXXX-5′
or
5′-PXXXXXXXXXXXXXXXXXXXXXXXDD-3′
3′-YXXXXXXXXXXXXXXXXXXXXXXXXX-5′
or
5′-XXXXXXXXXXXXXXXXXXXXXXXDD-3′
3′-YXXXXXXXXXXXXXXXXXXXXXXXXXP-5′
or
5′-XXXXXXXXXXXXXXXXXXXXXXXDD-3′
3′-PYXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXX-5′
or
5′-PXXXXXXXXXXXXXXXXXXXXXXXDDP-3′
3′-YXXXXXXXXXXXXXXXXXXXXXXXXX-5′
or
5′-XXXXXXXXXXXXXXXXXXXXXXXDD-3′
3′-PYXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXP-5′
or
5′-PXXXXXXXXXXXXXXXXXXXXXXXDDP-3′
3′-PYXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXXXP-3′
3′-YXXXXXXXXXXXXXXXXXXXXXXXXX-5′
or
5′-PXXXXXXXXXXXXXXXXXXXXXXXXX-3′
3′-YXXXXXXXXXXXXXXXXXXXXXXXXX-5′
or
5′-XXXXXXXXXXXXXXXXXXXXXXXXX-3′
3′-YXXXXXXXXXXXXXXXXXXXXXXXXXP-5′
or
5′-XXXXXXXXXXXXXXXXXXXXXXXXX-3′
3′-PYXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXX-5′
or
5′-PXXXXXXXXXXXXXXXXXXXXXXXXXP-3′
3′-YXXXXXXXXXXXXXXXXXXXXXXXXX-5′
or
5′-XXXXXXXXXXXXXXXXXXXXXXXXX-3′
3′-PYXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXP-5′
or
5′-PXXXXXXXXXXXXXXXXXXXXXXXXXP-3′
3′-PYXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXDDP-3′
3′-YXXXXXXXXXXXXXXXXXXXXXXXXX-5′
or
5′-PXXXXXXXXXXXXXXXXXXXXXXXDD-3′
3′-YXXXXXXXXXXXXXXXXXXXXXXXXX-5′
or
5′-XXXXXXXXXXXXXXXXXXXXXXXDD-3′
3′-YXXXXXXXXXXXXXXXXXXXXXXXXXP-5′
or
5′-XXXXXXXXXXXXXXXXXXXXXXXDD-3′
3′-PYXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXX-5′
or
5′-PXXXXXXXXXXXXXXXXXXXXXXXDDP-3′
3′-YXXXXXXXXXXXXXXXXXXXXXXXXX-5′
or
5′-XXXXXXXXXXXXXXXXXXXXXXXDD-3′
3′-PYXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXP-5′
or
5′-PXXXXXXXXXXXXXXXXXXXXXXXDDP-3′
3′-PYXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXXXP-3′
3′-YXXXXXXXXXXXXXXXXXXXXXXXXX-5′
or
5′-PXXXXXXXXXXXXXXXXXXXXXXXXX-3′
3′-YXXXXXXXXXXXXXXXXXXXXXXXXX-5′
or
5′-XXXXXXXXXXXXXXXXXXXXXXXXX-3′
3′-YXXXXXXXXXXXXXXXXXXXXXXXXXP-5′
or
5′-XXXXXXXXXXXXXXXXXXXXXXXXX-3′
3′-PYXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXX-5′
or
5′-PXXXXXXXXXXXXXXXXXXXXXXXXXP-3′
3′-YXXXXXXXXXXXXXXXXXXXXXXXXX-5′
or
5′-XXXXXXXXXXXXXXXXXXXXXXXXX-3′
3′-PYXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXP-5′
or
5′-PXXXXXXXXXXXXXXXXXXXXXXXXXP-3′
3′-PYXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXDDP-3′
3′-YXXXXXXXXXXXXXXXXXXXXXXXXX-5′
or
5′-PXXXXXXXXXXXXXXXXXXXXXXXDD-3′
3′-YXXXXXXXXXXXXXXXXXXXXXXXXX-5′
or
5′-XXXXXXXXXXXXXXXXXXXXXXXDD-3′
3′-YXXXXXXXXXXXXXXXXXXXXXXXXXP-5′
or
5′-XXXXXXXXXXXXXXXXXXXXXXXDD-3′
3′-PYXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXX-5′
or
5′-PXXXXXXXXXXXXXXXXXXXXXXXDDP-3′
3′-YXXXXXXXXXXXXXXXXXXXXXXXXX-5′
or
5′-XXXXXXXXXXXXXXXXXXXXXXXDD-3′
3′-PYXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXP-5′
or
5′-PXXXXXXXXXXXXXXXXXXXXXXXDDP-3′
3′-PYXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXXXP-3′
3′-YXXXXXXXXXXXXXXXXXXXXXXXXX-5′
or
5′-PXXXXXXXXXXXXXXXXXXXXXXXXX-3′
3′-YXXXXXXXXXXXXXXXXXXXXXXXXX-5′
or
5′-XXXXXXXXXXXXXXXXXXXXXXXXX-3′
3′-YXXXXXXXXXXXXXXXXXXXXXXXXXP-5′
or
5′-XXXXXXXXXXXXXXXXXXXXXXXXX-3′
3′-PYXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXX-5′
or
5′-PXXXXXXXXXXXXXXXXXXXXXXXXXP-3′
3′-YXXXXXXXXXXXXXXXXXXXXXXXXX-5′
or
5′-XXXXXXXXXXXXXXXXXXXXXXXXX-3′
3′-PYXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXP-5′
or
5′-PXXXXXXXXXXXXXXXXXXXXXXXXXP-3′
3′-PYXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXDDP-3′
3′-YXXXXXXXXXXXXXXXXXXXXXXXXX-5′
or
5′-PXXXXXXXXXXXXXXXXXXXXXXXDD-3′
3′-YXXXXXXXXXXXXXXXXXXXXXXXXX-5′
or
5′-XXXXXXXXXXXXXXXXXXXXXXXDD-3′
3′-YXXXXXXXXXXXXXXXXXXXXXXXXXP-5′
or
5′-XXXXXXXXXXXXXXXXXXXXXXXDD-3′
3′-PYXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXX-5′
or
5′-PXXXXXXXXXXXXXXXXXXXXXXXDDP-3′
3′-YXXXXXXXXXXXXXXXXXXXXXXXXX-5′
or
5′-XXXXXXXXXXXXXXXXXXXXXXXDD-3′
3′-PYXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXP-5′
or
5′-PXXXXXXXXXXXXXXXXXXXXXXXDDP-3′
3′-PYXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXXXP-3′
3′-YXXXXXXXXXXXXXXXXXXXXXXXXX-5′
or
5′-PXXXXXXXXXXXXXXXXXXXXXXXXX-3′
3′-YXXXXXXXXXXXXXXXXXXXXXXXXX-5′
or
5′-XXXXXXXXXXXXXXXXXXXXXXXXX-3′
3′-YXXXXXXXXXXXXXXXXXXXXXXXXXP-5′
or
5′-XXXXXXXXXXXXXXXXXXXXXXXXX-3′
3′-PYXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXX-5′
or
5′-PXXXXXXXXXXXXXXXXXXXXXXXXXP-3′
3′-YXXXXXXXXXXXXXXXXXXXXXXXXX-5′
or
5′-XXXXXXXXXXXXXXXXXXXXXXXXX-3′
3′-PYXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXP-5′
or
5′-PXXXXXXXXXXXXXXXXXXXXXXXXXP-3′
3′-PYXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXDDP-3′
3′-YXXXXXXXXXXXXXXXXXXXXXXXXX-5′
or
5′-PXXXXXXXXXXXXXXXXXXXXXXXDD-3′
3′-YXXXXXXXXXXXXXXXXXXXXXXXXX-5′
or
5′-XXXXXXXXXXXXXXXXXXXXXXXDD-3′
3′-YXXXXXXXXXXXXXXXXXXXXXXXXXP-5′
or
5′-XXXXXXXXXXXXXXXXXXXXXXXDD-3′
3′-PYXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXX-5′
or
5′-PXXXXXXXXXXXXXXXXXXXXXXXDDP-3′
3′-YXXXXXXXXXXXXXXXXXXXXXXXXX-5′
or
5′-XXXXXXXXXXXXXXXXXXXXXXXDD-3′
3′-PYXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXP-5′
or
5′-PXXXXXXXXXXXXXXXXXXXXXXXDDP-3′
3′-PYXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXXXP-3′
3′-YXXXXXXXXXXXXXXXXXXXXXXXXX-5′
or
5′-PXXXXXXXXXXXXXXXXXXXXXXXXX-3′
3′-YXXXXXXXXXXXXXXXXXXXXXXXXX-5′
or
5′-XXXXXXXXXXXXXXXXXXXXXXXXX-3′
3′-YXXXXXXXXXXXXXXXXXXXXXXXXXP-5′
or
5′-XXXXXXXXXXXXXXXXXXXXXXXXX-3′
3′-PYXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXX-5′
or
5′-PXXXXXXXXXXXXXXXXXXXXXXXXXP-3′
3′-YXXXXXXXXXXXXXXXXXXXXXXXXX-5′
or
5′-XXXXXXXXXXXXXXXXXXXXXXXXX-3′
3′-PYXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXP-5′
or
5′-PXXXXXXXXXXXXXXXXXXXXXXXXXP-3′
3′-PYXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXDDP-3′
3′-YXXXXXXXXXXXXXXXXXXXXXXXXX-5′
or
5′-PXXXXXXXXXXXXXXXXXXXXXXXDD-3′
3′-YXXXXXXXXXXXXXXXXXXXXXXXXX-5′
or
5′-XXXXXXXXXXXXXXXXXXXXXXXDD-3′
3′-YXXXXXXXXXXXXXXXXXXXXXXXXXP-5′
or
5′-XXXXXXXXXXXXXXXXXXXXXXXDD-3′
3′-PYXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXX-5′
or
5′-PXXXXXXXXXXXXXXXXXXXXXXXDDP-3′
3′-YXXXXXXXXXXXXXXXXXXXXXXXXX-5′
or
5′-XXXXXXXXXXXXXXXXXXXXXXXDD-3′
3′-PYXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXP-5′
or
5′-PXXXXXXXXXXXXXXXXXXXXXXXDDP-3′
3′-PYXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXXXP-3′
3′-YXXXXXXXXXXXXXXXXXXXXXXXXX-5′
or
5′-PXXXXXXXXXXXXXXXXXXXXXXXXX-3′
3′-YXXXXXXXXXXXXXXXXXXXXXXXXX-5′
or
5′-XXXXXXXXXXXXXXXXXXXXXXXXX-3′
3′-YXXXXXXXXXXXXXXXXXXXXXXXXXP-5′
or
5′-XXXXXXXXXXXXXXXXXXXXXXXXX-3′
3′-PYXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXX-5′
or
5′-PXXXXXXXXXXXXXXXXXXXXXXXXXP-3′
3′-YXXXXXXXXXXXXXXXXXXXXXXXXX-5′
or
5′-XXXXXXXXXXXXXXXXXXXXXXXXX-3′
3′-PYXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXP-5′
or
5′-PXXXXXXXXXXXXXXXXXXXXXXXXXP-3′
3′-PYXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXDDP-3′
3′-YXXXXXXXXXXXXXXXXXXXXXXXXX-5′
or
5′-PXXXXXXXXXXXXXXXXXXXXXXXDD-3′
3′-YXXXXXXXXXXXXXXXXXXXXXXXXX-5′
or
5′-XXXXXXXXXXXXXXXXXXXXXXXDD-3′
3′-YXXXXXXXXXXXXXXXXXXXXXXXXXP-5′
or
5′-XXXXXXXXXXXXXXXXXXXXXXXDD-3′
3′-PYXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXX-5′
or
5′-PXXXXXXXXXXXXXXXXXXXXXXXDDP-3′
3′-YXXXXXXXXXXXXXXXXXXXXXXXXX-5′
or
5′-XXXXXXXXXXXXXXXXXXXXXXXDD-3′
3′-PYXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXP-5′
or
5′-PXXXXXXXXXXXXXXXXXXXXXXXDDP-3′
3′-PYXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXXXP-3′
3′-YXXXXXXXXXXXXXXXXXXXXXXXXX-5′
or
5′-PXXXXXXXXXXXXXXXXXXXXXXXXX-3′
3′-YXXXXXXXXXXXXXXXXXXXXXXXXX-5′
or
5′-XXXXXXXXXXXXXXXXXXXXXXXXX-3′
3′-YXXXXXXXXXXXXXXXXXXXXXXXXXP-5′
or
5′-XXXXXXXXXXXXXXXXXXXXXXXXX-3′
3′-PYXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXX-5′
or
5′-PXXXXXXXXXXXXXXXXXXXXXXXXXP-3′
3′-YXXXXXXXXXXXXXXXXXXXXXXXXX-5′
or
5′-XXXXXXXXXXXXXXXXXXXXXXXXX-3′
3′-PYXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXP-5′
or
5′-PXXXXXXXXXXXXXXXXXXXXXXXXXP-3′
3′-PYXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXDDP-3′
3′-YXXXXXXXXXXXXXXXXXXXXXXXXX-5′
or
5′-PXXXXXXXXXXXXXXXXXXXXXXXDD-3′
3′-YXXXXXXXXXXXXXXXXXXXXXXXXX-5′
or
5′-XXXXXXXXXXXXXXXXXXXXXXXDD-3′
3′-YXXXXXXXXXXXXXXXXXXXXXXXXXP-5′
or
5′-XXXXXXXXXXXXXXXXXXXXXXXDD-3′
3′-PYXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXX-5′
or
5′-PXXXXXXXXXXXXXXXXXXXXXXXDDP-3′
3′-YXXXXXXXXXXXXXXXXXXXXXXXXX-5′
or
5′-XXXXXXXXXXXXXXXXXXXXXXXDD-3′
3′-PYXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXP-5′
or
5′-PXXXXXXXXXXXXXXXXXXXXXXXDDP-3′
3′-PYXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXXXP-3′
3′-YXXXXXXXXXXXXXXXXXXXXXXXXX-5′
or
5′-PXXXXXXXXXXXXXXXXXXXXXXXXX-3′
3′-YXXXXXXXXXXXXXXXXXXXXXXXXX-5′
or
5′-XXXXXXXXXXXXXXXXXXXXXXXXX-3′
3′-YXXXXXXXXXXXXXXXXXXXXXXXXXP-5′
or
5′-XXXXXXXXXXXXXXXXXXXXXXXXX-3′
3′-PYXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXX-5′
or
5′-PXXXXXXXXXXXXXXXXXXXXXXXXXP-3′
3′-YXXXXXXXXXXXXXXXXXXXXXXXXX-5′
or
5′-XXXXXXXXXXXXXXXXXXXXXXXXX-3′
3′-PYXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXP-5′
or
5′-PXXXXXXXXXXXXXXXXXXXXXXXXXP-3′
3′-PYXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXDDP-3′
3′-YXXXXXXXXXXXXXXXXXXXXXXXXX-5′
or
5′-PXXXXXXXXXXXXXXXXXXXXXXXDD-3′
3′-YXXXXXXXXXXXXXXXXXXXXXXXXX-5′
or
5′-XXXXXXXXXXXXXXXXXXXXXXXDD-3′
3′-YXXXXXXXXXXXXXXXXXXXXXXXXXP-5′
or
5′-XXXXXXXXXXXXXXXXXXXXXXXDD-3′
3′-PYXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXX-5′
or
5′-PXXXXXXXXXXXXXXXXXXXXXXXDDP-3′
3′-YXXXXXXXXXXXXXXXXXXXXXXXXX-5′
or
5′-XXXXXXXXXXXXXXXXXXXXXXXDD-3′
3′-PYXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXP-5′
or
5′-PXXXXXXXXXXXXXXXXXXXXXXXDDP-3′
3′-PYXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXXXP-3′
3′-YXXXXXXXXXXXXXXXXXXXXXXXXX-5′
or
5′-PXXXXXXXXXXXXXXXXXXXXXXXXX-3′
3′-YXXXXXXXXXXXXXXXXXXXXXXXXX-5′
or
5′-XXXXXXXXXXXXXXXXXXXXXXXXX-3′
3′-YXXXXXXXXXXXXXXXXXXXXXXXXXP-5′
or
5′-XXXXXXXXXXXXXXXXXXXXXXXXX-3′
3′-PYXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXX-5′
or
5′-PXXXXXXXXXXXXXXXXXXXXXXXXXP-3′
3′-YXXXXXXXXXXXXXXXXXXXXXXXXX-5′
or
5′-XXXXXXXXXXXXXXXXXXXXXXXXX-3′
3′-PYXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXP-5′
or
5′-PXXXXXXXXXXXXXXXXXXXXXXXXXP-3′
3′-PYXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXDDP-3′
3′-YXXXXXXXXXXXXXXXXXXXXXXXXX-5′
or
5′-PXXXXXXXXXXXXXXXXXXXXXXXDD-3′
3′-YXXXXXXXXXXXXXXXXXXXXXXXXX-5′
or
5′-XXXXXXXXXXXXXXXXXXXXXXXDD-3′
3′-YXXXXXXXXXXXXXXXXXXXXXXXXXP-5′
or
5′-XXXXXXXXXXXXXXXXXXXXXXXDD-3′
3′-PYXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXX-5′
or
5′-PXXXXXXXXXXXXXXXXXXXXXXXDDP-3′
3′-YXXXXXXXXXXXXXXXXXXXXXXXXX-5′
or
5′-XXXXXXXXXXXXXXXXXXXXXXXDD-3′
3′-PYXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXP-5′
or
5′-PXXXXXXXXXXXXXXXXXXXXXXXDDP-3′
3′-PYXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXXXP-3′
3′-YXXXXXXXXXXXXXXXXXXXXXXXXX-5′
or
5′-PXXXXXXXXXXXXXXXXXXXXXXXXX-3′
3′-YXXXXXXXXXXXXXXXXXXXXXXXXX-5′
or
5′-XXXXXXXXXXXXXXXXXXXXXXXXX-3′
3′-YXXXXXXXXXXXXXXXXXXXXXXXXXP-5′
or
5′-XXXXXXXXXXXXXXXXXXXXXXXXX-3′
3′-PYXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXX-5′
or
5′-PXXXXXXXXXXXXXXXXXXXXXXXXXP-3′
3′-YXXXXXXXXXXXXXXXXXXXXXXXXX-5′
or
5′-XXXXXXXXXXXXXXXXXXXXXXXXX-3′
3′-PYXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXP-5′
or
5′-PXXXXXXXXXXXXXXXXXXXXXXXXXP-3′
3′-PYXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXDDP-3′
3′-YXXXXXXXXXXXXXXXXXXXXXXXXX-5′
or
5′-PXXXXXXXXXXXXXXXXXXXXXXXDD-3′
3′-YXXXXXXXXXXXXXXXXXXXXXXXXX-5′
or
5′-XXXXXXXXXXXXXXXXXXXXXXXDD-3′
3′-YXXXXXXXXXXXXXXXXXXXXXXXXXP-5′
or
5′-XXXXXXXXXXXXXXXXXXXXXXXDD-3′
3′-PYXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXX-5′
or
5′-PXXXXXXXXXXXXXXXXXXXXXXXDDP-3′
3′-YXXXXXXXXXXXXXXXXXXXXXXXXX-5′
or
5′-XXXXXXXXXXXXXXXXXXXXXXXDD-3′
3′-PYXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXP-5′
or
5′-PXXXXXXXXXXXXXXXXXXXXXXXDDP-3′
3′-PYXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXXXP-3′
3′-YXXXXXXXXXXXXXXXXXXXXXXXXX-5′
or
5′-PXXXXXXXXXXXXXXXXXXXXXXXXX-3′
3′-YXXXXXXXXXXXXXXXXXXXXXXXXX-5′
or
5′-XXXXXXXXXXXXXXXXXXXXXXXXX-3′
3′-YXXXXXXXXXXXXXXXXXXXXXXXXXP-5′
or
5′-XXXXXXXXXXXXXXXXXXXXXXXXX-3′
3′-PYXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXX-5′
or
5′-PXXXXXXXXXXXXXXXXXXXXXXXXXP-3′
3′-YXXXXXXXXXXXXXXXXXXXXXXXXX-5′
or
5′-XXXXXXXXXXXXXXXXXXXXXXXXX-3′
3′-PYXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXP-5′
or
5′-PXXXXXXXXXXXXXXXXXXXXXXXXXP-3′
3′-PYXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXDDP-3′
3′-YXXXXXXXXXXXXXXXXXXXXXXXXX-5′
or
5′-PXXXXXXXXXXXXXXXXXXXXXXXDD-3′
3′-YXXXXXXXXXXXXXXXXXXXXXXXXX-5′
or
5′-XXXXXXXXXXXXXXXXXXXXXXXDD-3′
3′-YXXXXXXXXXXXXXXXXXXXXXXXXXP-5′
or
5′-XXXXXXXXXXXXXXXXXXXXXXXDD-3′
3′-PYXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXX-5′
or
5′-PXXXXXXXXXXXXXXXXXXXXXXXDDP-3′
3′-YXXXXXXXXXXXXXXXXXXXXXXXXX-5′
or
5′-XXXXXXXXXXXXXXXXXXXXXXXDD-3′
3′-PYXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXP-5′
or
5′-PXXXXXXXXXXXXXXXXXXXXXXXDDP-3′
3′-PYXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXXXP-3′
3′-YXXXXXXXXXXXXXXXXXXXXXXXXX-5′
or
5′-PXXXXXXXXXXXXXXXXXXXXXXXXX-3′
3′-YXXXXXXXXXXXXXXXXXXXXXXXXX-5′
or
5′-XXXXXXXXXXXXXXXXXXXXXXXXX-3′
3′-YXXXXXXXXXXXXXXXXXXXXXXXXXP-5′
or
5′-XXXXXXXXXXXXXXXXXXXXXXXXX-3′
3′-PYXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXX-5′
or
5′-PXXXXXXXXXXXXXXXXXXXXXXXXXP-3′
3′-YXXXXXXXXXXXXXXXXXXXXXXXXX-5′
or
5′-XXXXXXXXXXXXXXXXXXXXXXXXX-3′
3′-PYXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXP-5′
or
5′-PXXXXXXXXXXXXXXXXXXXXXXXXXP-3′
3′-PYXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXDDP-3′
3′-YXXXXXXXXXXXXXXXXXXXXXXXXX-5′
or
5′-PXXXXXXXXXXXXXXXXXXXXXXXDD-3′
3′-YXXXXXXXXXXXXXXXXXXXXXXXXX-5′
or
5′-XXXXXXXXXXXXXXXXXXXXXXXDD-3′
3′-YXXXXXXXXXXXXXXXXXXXXXXXXXP-5′
or
5′-XXXXXXXXXXXXXXXXXXXXXXXDD-3′
3′-PYXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXX-5′
or
5′-PXXXXXXXXXXXXXXXXXXXXXXXDDP-3′
3′-YXXXXXXXXXXXXXXXXXXXXXXXXX-5′
or
5′-XXXXXXXXXXXXXXXXXXXXXXXDD-3′
3′-PYXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXP-5′
or
5′-PXXXXXXXXXXXXXXXXXXXXXXXDDP-3′
3′-PYXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXXXP-3′
3′-YXXXXXXXXXXXXXXXXXXXXXXXXX-5′
or
5′-PXXXXXXXXXXXXXXXXXXXXXXXXX-3′
3′-YXXXXXXXXXXXXXXXXXXXXXXXXX-5′
or
5′-XXXXXXXXXXXXXXXXXXXXXXXXX-3′
3′-YXXXXXXXXXXXXXXXXXXXXXXXXXP-5′
or
5′-XXXXXXXXXXXXXXXXXXXXXXXXX-3′
3′-PYXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXX-5′
or
5′-PXXXXXXXXXXXXXXXXXXXXXXXXXP-3′
3′-YXXXXXXXXXXXXXXXXXXXXXXXXX-5′
or
5′-XXXXXXXXXXXXXXXXXXXXXXXXX-3′
3′-PYXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXP-5′
or
5′-PXXXXXXXXXXXXXXXXXXXXXXXXXP-3′
3′-PYXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXDDP-3′
3′-YXXXXXXXXXXXXXXXXXXXXXXXXX-5′
or
5′-PXXXXXXXXXXXXXXXXXXXXXXXDD-3′
3′-YXXXXXXXXXXXXXXXXXXXXXXXXX-5′
or
5′-XXXXXXXXXXXXXXXXXXXXXXXDD-3′
3′-YXXXXXXXXXXXXXXXXXXXXXXXXXP-5′
or
5′-XXXXXXXXXXXXXXXXXXXXXXXDD-3′
3′-PYXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXX-5′
or
5′-PXXXXXXXXXXXXXXXXXXXXXXXDDP-3′
3′-YXXXXXXXXXXXXXXXXXXXXXXXXX-5′
or
5′-XXXXXXXXXXXXXXXXXXXXXXXDD-3′
3′-PYXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXP-5′
or
5′-PXXXXXXXXXXXXXXXXXXXXXXXDDP-3′
3′-PYXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXXXP-3′
3′-YXXXXXXXXXXXXXXXXXXXXXXXXX-5′
or
5′-PXXXXXXXXXXXXXXXXXXXXXXXXX-3′
3′-YXXXXXXXXXXXXXXXXXXXXXXXXX-5′
or
5′-XXXXXXXXXXXXXXXXXXXXXXXXX-3′
3′-YXXXXXXXXXXXXXXXXXXXXXXXXXP-5′
or
5′-XXXXXXXXXXXXXXXXXXXXXXXXX-3′
3′-PYXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXX-5′
or
5′-PXXXXXXXXXXXXXXXXXXXXXXXXXP-3′
3′-YXXXXXXXXXXXXXXXXXXXXXXXXX-5′
or
5′-XXXXXXXXXXXXXXXXXXXXXXXXX-3′
3′-PYXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXP-5′
or
5′-PXXXXXXXXXXXXXXXXXXXXXXXXXP-3′
3′-PYXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXDDP-3′
3′-YXXXXXXXXXXXXXXXXXXXXXXXXX-5′
or
5′-PXXXXXXXXXXXXXXXXXXXXXXXDD-3′
3′-YXXXXXXXXXXXXXXXXXXXXXXXXX-5′
or
5′-XXXXXXXXXXXXXXXXXXXXXXXDD-3′
3′-YXXXXXXXXXXXXXXXXXXXXXXXXXP-5′
or
5′-XXXXXXXXXXXXXXXXXXXXXXXDD-3′
3′-PYXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXX-5′
or
5′-PXXXXXXXXXXXXXXXXXXXXXXXDDP-3′
3′-YXXXXXXXXXXXXXXXXXXXXXXXXX-5′
or
5′-XXXXXXXXXXXXXXXXXXXXXXXDD-3′
3′-PYXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXP-5′
or
5′-PXXXXXXXXXXXXXXXXXXXXXXXDDP-3′
3′-PYXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXDDP-3′
3′-XXXXXXXXXXXXXXXXXXXXXXXXXXX-5′
or
5′-PXXXXXXXXXXXXXXXXXXXXXXXDD-3′
3′-XXXXXXXXXXXXXXXXXXXXXXXXXXX-5′
or
5′-XXXXXXXXXXXXXXXXXXXXXXXDD-3′
3′-XXXXXXXXXXXXXXXXXXXXXXXXXXXP-5′
or
5′-XXXXXXXXXXXXXXXXXXXXXXXDD-3′
3′-PXXXXXXXXXXXXXXXXXXXXXXXXXXX-5′
or
5′-PXXXXXXXXXXXXXXXXXXXXXXXDDP-3′
3′-XXXXXXXXXXXXXXXXXXXXXXXXXXX-5′
or
5′-XXXXXXXXXXXXXXXXXXXXXXXDD-3′
3′-PXXXXXXXXXXXXXXXXXXXXXXXXXXXP-5′
or
5′-PXXXXXXXXXXXXXXXXXXXXXXXDDP-3′
3′-PXXXXXXXXXXXXXXXXXXXXXXXXXXXP-5′
or
5′-XXXXXXXXXXXXXXXXXXXXXXXXXP-3′
3′-YXXXXXXXXXXXXXXXXXXXXXXXXX-5′
or
5′-PXXXXXXXXXXXXXXXXXXXXXXXXX-3′
3′-YXXXXXXXXXXXXXXXXXXXXXXXXX-5′
or
5′-XXXXXXXXXXXXXXXXXXXXXXXXX-3′
3′-YXXXXXXXXXXXXXXXXXXXXXXXXXP-5′
or
5′-XXXXXXXXXXXXXXXXXXXXXXXXX-3′
3′-PYXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXX-5′
or
5′-PXXXXXXXXXXXXXXXXXXXXXXXXXP-3′
3′-YXXXXXXXXXXXXXXXXXXXXXXXXX-5′
or
5′-XXXXXXXXXXXXXXXXXXXXXXXXX-3′
3′-PYXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXP-5′
or
5′-PXXXXXXXXXXXXXXXXXXXXXXXXXP-3′
3′-PYXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′
or
5′-XXXXXXXXXXXXXXXXXXXXXXXXXP-3′
3′-XXXXXXXXXXXXXXXXXXXXXXXXXXX-5′
or
5′-PXXXXXXXXXXXXXXXXXXXXXXXXX-3′
3′-XXXXXXXXXXXXXXXXXXXXXXXXXXX-5′
or
5′-XXXXXXXXXXXXXXXXXXXXXXXXX-3′
3′-XXXXXXXXXXXXXXXXXXXXXXXXXXXP-5′
or
5′-XXXXXXXXXXXXXXXXXXXXXXXXX-3′
3′-PXXXXXXXXXXXXXXXXXXXXXXXXXXX-5′
or
5′-PXXXXXXXXXXXXXXXXXXXXXXXXXP-3′
3′-XXXXXXXXXXXXXXXXXXXXXXXXXXX-5′
or
5′-XXXXXXXXXXXXXXXXXXXXXXXXX-3′
3′-PXXXXXXXXXXXXXXXXXXXXXXXXXXXP-5′
or
5′-PXXXXXXXXXXXXXXXXXXXXXXXXXP-3′
3′-PXXXXXXXXXXXXXXXXXXXXXXXXXXXP-5′

wherein “X”=RNA, “X”=2′-O-methyl RNA, “Y” is an overhang domain comprised of 1-4 RNA monomers that are optionally 2′-O-methyl RNA monomers, underlined residues are 2′-O-methyl RNA monomers, “D”=DNA and “P”=peptide. The top strand is the sense strand, and the bottom strand is the antisense strand.

In another embodiment, the dsRNA comprises strands having equal lengths.

In one such embodiment, the dsRNA comprises:

5′-XXXXXXXXXXXXXXXXXXXXXXXXXMMP-3′
3′-XXXXXXXXXXXXXXXXXXXXXXXXXMM-5′

wherein “X”=RNA, “M” is Nucleic acid residues (RNA, DNA or non-natural or modified nucleic acids) and “P”=a peptide. Any such residues of such agents can optionally be 2′-O-methyl RNA monomers-alternating positioning of 2′-O-methyl RNA monomers that commences from the 3′-terminal residue of the bottom (second) strand, as shown for above asymmetric agents, can also be used in the above blunt-blunt dsRNA agents.

In one such embodiment, the dsRNA comprises:

5′-PXXXXXXXXXXXXXXXXXXXXXXXXXMM-3′
3′-XXXXXXXXXXXXXXXXXXXXXXXXXMM-5′

wherein “X”=RNA, “M” is Nucleic acid residues (RNA, DNA or non-natural or modified nucleic acids) and “P”=a peptide. Any such residues of such agents can optionally be 2′-O-methyl RNA monomers-alternating positioning of 2′-O-methyl RNA monomers that commences from the 3′-terminal residue of the bottom (second) strand, as shown for above asymmetric agents, can also be used in the above blunt-blunt dsRNA agents.

In one such embodiment, the dsRNA comprises:

5′-XXXXXXXXXXXXXXXXXXXXXXXXXMM-3′
3′-XXXXXXXXXXXXXXXXXXXXXXXXXMMP-5′

wherein “X”=RNA, “M” is Nucleic acid residues (RNA, DNA or non-natural or modified nucleic acids) and “P”=a peptide. Any such residues of such agents can optionally be 2′-O-methyl RNA monomers-alternating positioning of 2′-O-methyl RNA monomers that commences from the 3′-terminal residue of the bottom (second) strand, as shown for above asymmetric agents, can also be used in the above blunt-blunt dsRNA agents.

In one such embodiment, the dsRNA comprises:

5′-XXXXXXXXXXXXXXXXXXXXXXXXXMM-3′
3′-PXXXXXXXXXXXXXXXXXXXXXXXXXMM-5′

wherein “X”=RNA, “M” is Nucleic acid residues (RNA, DNA or non-natural or modified nucleic acids) and “P”=a peptide. Any such residues of such agents can optionally be 2′-O-methyl RNA monomers-alternating positioning of 2′-O-methyl RNA monomers that commences from the 3′-terminal residue of the bottom (second) strand, as shown for above asymmetric agents, can also be used in the above blunt-blunt dsRNA agents.

In one such embodiment, the dsRNA comprises:

5′-PXXXXXXXXXXXXXXXXXXXXXXXXXMMP-3′
3′-XXXXXXXXXXXXXXXXXXXXXXXXXMM-5′

wherein “X”=RNA, “M” is Nucleic acid residues (RNA, DNA or non-natural or modified nucleic acids) and “P”=a peptide. Any such residues of such agents can optionally be 2′-O-methyl RNA monomers-alternating positioning of 2′-O-methyl RNA monomers that commences from the 3′-terminal residue of the bottom (second) strand, as shown for above asymmetric agents, can also be used in the above blunt-blunt dsRNA agents.

In one such embodiment, the dsRNA comprises:

5′-XXXXXXXXXXXXXXXXXXXXXXXXXMM-3′
3′-PXXXXXXXXXXXXXXXXXXXXXXXXXMMP-5′

wherein “X”=RNA, “M” is Nucleic acid residues (RNA, DNA or non-natural or modified nucleic acids) and “P”=a peptide. Any such residues of such agents can optionally be 2′-O-methyl RNA monomers-alternating positioning of 2′-O-methyl RNA monomers that commences from the 3′-terminal residue of the bottom (second) strand, as shown for above asymmetric agents, can also be used in the above blunt-blunt dsRNA agents.

In one such embodiment, the dsRNA comprises:

5′-PXXXXXXXXXXXXXXXXXXXXXXXXXMMP-3′
3′-PXXXXXXXXXXXXXXXXXXXXXXXXXMMP-5′

wherein “X”=RNA, “M” is Nucleic acid residues (RNA, DNA or non-natural or modified nucleic acids) and “P”=a peptide. Any such residues of such agents can optionally be 2′-O-methyl RNA monomers-alternating positioning of 2′-O-methyl RNA monomers that commences from the 3′-terminal residue of the bottom (second) strand, as shown for above asymmetric agents, can also be used in the above blunt-blunt dsRNA agents.

The invention also contemplates any of the exemplary structures recited below, wherein at least one peptide of the invention is conjugated to at least one end of at least one of the first or second strand or internally to at least one of the first or second strand of the dsRNA of the invention.

In another embodiment, the DsiRNA comprises strands having equal lengths possessing 1-3 mismatched residues that serve to orient Dicer cleavage (specifically, one or more of positions 1, 2 or 3 on the first strand of the DsiRNA, when numbering from the 3′-terminal residue, are mismatched with corresponding residues of the 5′-terminal region on the second strand when first and second strands are annealed to one another). An exemplary 27mer DsiRNA agent with two terminal mismatched residues is shown:

5′-XXXXXXXXXXXXXXXXXXXXXXXXX^{M}^{^{M-3′}}
3′-XXXXXXXXXXXXXXXXXXXXXXXXX_{M}_{_{M-5′}}

wherein “X”=RNA, “M”=Nucleic acid residues (RNA, DNA or non-natural or modified nucleic acids) that do not base pair (hydrogen bond) with corresponding “M” residues of otherwise complementary strand when strands are annealed. Any of the residues of such agents can optionally be 2′-O-methyl RNA monomers—alternating positioning of 2′-O-methyl RNA monomers that commences from the 3′-terminal residue of the bottom (second) strand, as shown for above asymmetric agents, can also be used in the above “blunt/fray” DsiRNA agent. The top strand (first strand) is the sense strand, and the bottom strand (second strand) is the antisense strand.

In certain additional embodiments, the present invention provides compositions for RNA interference (RNAi) that possess one or more base paired deoxyribonucleotides within a region of a double stranded nucleic acid (dsNA) that is positioned 3′ of a projected sense strand Dicer cleavage site and correspondingly 5′ of a projected antisense strand Dicer cleavage site. The compositions of the invention comprise a dsNA which is a precursor molecule, i.e., the dsNA of the present invention is processed in vivo to produce an active small interfering nucleic acid (siRNA). The dsNA is processed by Dicer to an active siRNA which is incorporated into RISC.

In certain embodiments, the DsiRNA agents of the invention can have any of the following exemplary structures:

In one such embodiment, the DsiRNA comprises:

5′-XXXXXXXXXXXXXXXXXXXXXXXX_{N*}D_{N}DD-3′
3′-YXXXXXXXXXXXXXXXXXXXXXXXX_{N*}D_{N}XX-5′

wherein “X”=RNA, “Y” is an optional overhang domain comprised of 0-10 RNA monomers that are optionally 2′-O-methyl RNA monomers—in certain embodiments, “Y” is an overhang domain comprised of 1-4 RNA monomers that are optionally 2′-O-methyl RNA monomers, “D”=DNA, and “N”=1 to 50 or more, but is optionally 1-8. “N*”=0 to 15 or more, but is optionally 0, 1, 2, 3, 4, 5 or 6. In one embodiment, the top strand is the sense strand, and the bottom strand is the antisense strand. Alternatively, the bottom strand is the sense strand and the top strand is the antisense strand.

In a related embodiment, the DsiRNA comprises:

5′-XXXXXXXXXXXXXXXXXXXXXXXX_{N*}D_{N}DD-3′
3′-YXXXXXXXXXXXXXXXXXXXXXXXX_{N*}D_{N}DD-5′

wherein “X”=RNA, “Y” is an optional overhang domain comprised of 0-10 RNA monomers that are optionally 2′-O-methyl RNA monomers—in certain embodiments, “Y” is an overhang domain comprised of 1-4 RNA monomers that are optionally 2′-O-methyl RNA monomers, “D”=DNA, and “N”=1 to 50 or more, but is optionally 1-8. “N*”=0 to 15 or more, but is optionally 0, 1, 2, 3, 4, 5 or 6. In one embodiment, the top strand is the sense strand, and the bottom strand is the antisense strand. Alternatively, the bottom strand is the sense strand and the top strand is the antisense strand.

In another such embodiment, the DsiRNA comprises:

5′-XXXXXXXXXXXXXXXXXXXXXXXX_{N*}D_{N}DD-3′
3′-YXXXXXXXXXXXXXXXXXXXXXXXX_{N*}D_{N}ZZ-5′

wherein “X”=RNA, “X”=2′-O-methyl RNA, “Y” is an optional overhang domain comprised of 0-10 RNA monomers that are optionally 2′-O-methyl RNA monomers—in certain embodiments, “Y” is an overhang domain comprised of 1-4 RNA monomers that are optionally 2′-O-methyl RNA monomers, “D”=DNA, “Z”=DNA or RNA, and “N”=1 to 50 or more, but is optionally 1-8. “N*”=0 to 15 or more, but is optionally 0, 1, 2, 3, 4, 5 or 6. In one embodiment, the top strand is the sense strand, and the bottom strand is the antisense strand. Alternatively, the bottom strand is the sense strand and the top strand is the antisense strand, with 2′-O-methyl RNA monomers located at alternating residues along the top strand, rather than the bottom strand presently depicted in the above schematic.

In another such embodiment, the DsiRNA comprises:

5′-XXXXXXXXXXXXXXXXXXXXXXXX_{N*}D_{N}DD-3′
3′-YXXXXXXXXXXXXXXXXXXXXXXXX_{N*}D_{N}ZZ-5′

wherein “X”=RNA, “X”=2′-O-methyl RNA, “Y” is an optional overhang domain comprised of 0-10 RNA monomers that are optionally 2′-O-methyl RNA monomers—in certain embodiments, “Y” is an overhang domain comprised of 1-4 RNA monomers that are optionally 2′-O-methyl RNA monomers, “D”=DNA, “Z”=DNA or RNA, and “N”=1 to 50 or more, but is optionally 1-8. “N*”=0 to 15 or more, but is optionally 0, 1, 2, 3, 4, 5 or 6. In one embodiment, the top strand is the sense strand, and the bottom strand is the antisense strand. Alternatively, the bottom strand is the sense strand and the top strand is the antisense strand, with 2′-O-methyl RNA monomers located at alternating residues along the top strand, rather than the bottom strand presently depicted in the above schematic.

In another embodiment, the DsiRNA comprises:

5′-XXXXXXXXXXXXXXXXXXXXXXXX_{N*}[X1/D1]_{N}DD-3′
3′-YXXXXXXXXXXXXXXXXXXXXXXXX_{N*}[X2/D2]_{N}ZZ-5′

wherein “X”=RNA, “Y” is an optional overhang domain comprised of 0-10 RNA monomers that are optionally 2′-O-methyl RNA monomers—in certain embodiments, “Y” is an overhang domain comprised of 1-4 RNA monomers that are optionally 2′-O-methyl RNA monomers, “D”=DNA, “Z”=DNA or RNA, and “N”=1 to 50 or more, but is optionally 1-8, where at least one D1_{N} is present in the top strand and is base paired with a corresponding D2_{N} in the bottom strand. Optionally, D1_{N} and D1_{N+1} are base paired with corresponding D2_{N} and D2_{N+1}; D1_{N}, D1_{N+1} and D1_{N+2} are base paired with corresponding D2_{N}, D1_{N+1} and D1_{N+2}, etc. “N*”=0 to 15 or more, but is optionally 0, 1, 2, 3, 4, 5 or 6. In one embodiment, the top strand is the sense strand, and the bottom strand is the antisense strand. Alternatively, the bottom strand is the sense strand and the top strand is the antisense strand, with 2′-O-methyl RNA monomers located at alternating residues along the top strand, rather than the bottom strand presently depicted in the above schematic.

In any of the above-depicted structures, the 5′ end of either the sense strand or antisense strand optionally comprises a phosphate group.

In another embodiment, the DNA:DNA-extended DsiRNA comprises strands having equal lengths possessing 1-3 mismatched residues that serve to orient Dicer cleavage (specifically, one or more of positions 1, 2 or 3 on the first strand of the DsiRNA, when numbering from the 3′-terminal residue, are mismatched with corresponding residues of the 5′-terminal region on the second strand when first and second strands are annealed to one another). An exemplary DNA:DNA-extended DsiRNA agent with two terminal mismatched residues is shown:

5′-XXXXXXXXXXXXXXXXXXXXXXXXXX_{N*}D_{N}^{M}^{^{M-3′}}
3′-XXXXXXXXXXXXXXXXXXXXXXXXXX_{N*}D_{N}_{M}_{_{M-5′}}

wherein “X”=RNA, “M”=Nucleic acid residues (RNA, DNA or non-natural or modified nucleic acids) that do not base pair (hydrogen bond) with corresponding “M” residues of otherwise complementary strand when strands are annealed, “D”=DNA and “N”=1 to 50 or more, but is optionally 1-8. “N*”=0 to 15 or more, but is optionally 0, 1, 2, 3, 4, 5 or 6. Any of the residues of such agents can optionally be 2′-O-methyl RNA monomers—alternating positioning of 2′-O-methyl RNA monomers that commences from the 3′-terminal residue of the bottom (second) strand, as shown for above asymmetric agents, can also be used in the above “blunt/fray” DsiRNA agent. In one embodiment, the top strand (first strand) is the sense strand, and the bottom strand (second strand) is the antisense strand. Alternatively, the bottom strand is the sense strand and the top strand is the antisense strand. Modification and DNA:DNA extension patterns paralleling those shown above for asymmetric/overhang agents can also be incorporated into such “blunt/frayed” agents.

In one embodiment, a length-extended DsiRNA agent is provided that comprises deoxyribonucleotides positioned at sites modeled to function via specific direction of Dicer cleavage, yet which does not require the presence of a base-paired deoxyribonucleotide in the dsNA structure. An exemplary structure for such a molecule is shown:

5′-XXXXXXXXXXXXXXXXXXXDDXX-3′
3′-YXXXXXXXXXXXXXXXXXDDXXXX-5′

wherein “X”=RNA, “Y” is an optional overhang domain comprised of 0-10 RNA monomers that are optionally 2′-O-methyl RNA monomers—in certain embodiments, “Y” is an overhang domain comprised of 1-4 RNA monomers that are optionally 2′-O-methyl RNA monomers, and “D”=DNA. In one embodiment, the top strand is the sense strand, and the bottom strand is the antisense strand. Alternatively, the bottom strand is the sense strand and the top strand is the antisense strand. The above structure is modeled to force Dicer to cleave a minimum of a 21mer duplex as its primary post-processing form. In embodiments where the bottom strand of the above structure is the antisense strand, the positioning of two deoxyribonucleotide residues at the ultimate and penultimate residues of the 5′ end of the antisense strand is likely to reduce off-target effects (as prior studies have shown a 2′-O-methyl modification of at least the penultimate position from the 5′ terminus of the antisense strand to reduce off-target effects; see, e.g., US 2007/0223427).

In one embodiment, the DsiRNA comprises:

5′-D_{N}XXXXXXXXXXXXXXXXXXXXXXXX_{N*}Y-3′
3′-D_{N}XXXXXXXXXXXXXXXXXXXXXXXX_{N*}-5′

wherein “X”=RNA, “Y” is an optional overhang domain comprised of 0-10 RNA monomers that are optionally 2′-O-methyl RNA monomers—in certain embodiments, “Y” is an overhang domain comprised of 1-4 RNA monomers that are optionally 2′-O-methyl RNA monomers, “D”=DNA, and “N”=1 to 50 or more, but is optionally 1-8. “N*”=0 to 15 or more, but is optionally 0, 1, 2, 3, 4, 5 or 6. In one embodiment, the top strand is the sense strand, and the bottom strand is the antisense strand. Alternatively, the bottom strand is the sense strand and the top strand is the antisense strand.

In a related embodiment, the DsiRNA comprises:

5′-D_{N}XXXXXXXXXXXXXXXXXXXXXXXX_{N*}DD-3′
3′-D_{N}XXXXXXXXXXXXXXXXXXXXXXXX_{N*}XX-5′

wherein “X”=RNA, optionally a 2′-O-methyl RNA monomers “D”=DNA, “N”=1 to 50 or more, but is optionally 1-8. “N*”=0 to 15 or more, but is optionally 0, 1, 2, 3, 4, 5 or 6. In one embodiment, the top strand is the sense strand, and the bottom strand is the antisense strand. Alternatively, the bottom strand is the sense strand and the top strand is the antisense strand.

In another such embodiment, the DsiRNA comprises:

5′-D_{N}XXXXXXXXXXXXXXXXXXXXXXXX_{N*}DD-3′
3′-D_{N}XXXXXXXXXXXXXXXXXXXXXXXX_{N*}ZZ-5′

wherein “X”=RNA, optionally a 2′-O-methyl RNA monomers “D”=DNA, “N”=1 to 50 or more, but is optionally 1-8. “N*”=0 to 15 or more, but is optionally 0, 1, 2, 3, 4, 5 or 6. “Z”=DNA or RNA. In one embodiment, the top strand is the sense strand, and the bottom strand is the antisense strand. Alternatively, the bottom strand is the sense strand and the top strand is the antisense strand, with 2′-O-methyl RNA monomers located at alternating residues along the top strand, rather than the bottom strand presently depicted in the above schematic.

In another such embodiment, the DsiRNA comprises:

5′-D_{N}ZZXXXXXXXXXXXXXXXXXXXXXXXX_{N*}DD-3′
3′-D_{N}XXXXXXXXXXXXXXXXXXXXXXXXXX_{N*}ZZ-5′

wherein “X”=RNA, “X”=2′-O-methyl RNA, “D”=DNA, “Z”=DNA or RNA, and “N”=1 to 50 or more, but is optionally 1-8. “N*”=0 to 15 or more, but is optionally 0, 1, 2, 3, 4, 5 or 6. In one embodiment, the top strand is the sense strand, and the bottom strand is the antisense strand. Alternatively, the bottom strand is the sense strand and the top strand is the antisense strand, with 2′-O-methyl RNA monomers located at alternating residues along the top strand, rather than the bottom strand presently depicted in the above schematic.

In another such embodiment, the DsiRNA comprises:

5′-D_{N}ZZXXXXXXXXXXXXXXXXXXXXXXXX_{N*}Y-3′
3′-D_{N}XXXXXXXXXXXXXXXXXXXXXXXXXX_{N*}-5′

wherein “X”=RNA, “X”=2′-O-methyl RNA, “D”=DNA, “Z”=DNA or RNA, and “N”=1 to 50 or more, but is optionally 1-8. “N*”=0 to 15 or more, but is optionally 0, 1, 2, 3, 4, 5 or 6. “Y” is an optional overhang domain comprised of 0-10 RNA monomers that are optionally 2′-O-methyl RNA monomers—in certain embodiments, “Y” is an overhang domain comprised of 1-4 RNA monomers that are optionally 2′-O-methyl RNA monomers. In one embodiment, the top strand is the sense strand, and the bottom strand is the antisense strand. Alternatively, the bottom strand is the sense strand and the top strand is the antisense strand, with 2′-O-methyl RNA monomers located at alternating residues along the top strand, rather than the bottom strand presently depicted in the above schematic.

In another embodiment, the DsiRNA comprises:

5′-[X1/D1]_{N}XXXXXXXXXXXXXXXXXXXXXXXX_{N*}DD-3′
3′-[X2/D2]_{N}XXXXXXXXXXXXXXXXXXXXXXXX_{N*}ZZ-5′

wherein “X”=RNA, “D”=DNA, “Z”=DNA or RNA, and “N”=1 to 50 or more, but is optionally 1-8, where at least one D1_{N} is present in the top strand and is base paired with a corresponding D2_{N} in the bottom strand. Optionally, D1_{N} and D1_{N+1} are base paired with corresponding D2_{N} and D2_{N+1}; D1_{N}, D1_{N+1} and D1_{N+2} are base paired with corresponding D2_{N}, D1_{N+1} and D1_{N+2}, etc. “N*”=0 to 15 or more, but is optionally 0, 1, 2, 3, 4, 5 or 6. In one embodiment, the top strand is the sense strand, and the bottom strand is the antisense strand. Alternatively, the bottom strand is the sense strand and the top strand is the antisense strand, with 2′-O-methyl RNA monomers located at alternating residues along the top strand, rather than the bottom strand presently depicted in the above schematic.

In a related embodiment, the DsiRNA comprises:

5′-[X1/D1]_{N}XXXXXXXXXXXXXXXXXXXXXXXX_{N*}Y-3′
3′-[X2/D2]_{N}XXXXXXXXXXXXXXXXXXXXXXXX_{N*}-5′

wherein “X”=RNA, “D”=DNA, “Y” is an optional overhang domain comprised of 0-10 RNA monomers that are optionally 2′-O-methyl RNA monomers—in certain embodiments, “Y” is an overhang domain comprised of 1-4 RNA monomers that are optionally 2′-O-methyl RNA monomers, and “N”=1 to 50 or more, but is optionally 1-8, where at least one D1_{N} is present in the top strand and is base paired with a corresponding D2_{N} in the bottom strand. Optionally, D1_{N} and D1_{N+1} are base paired with corresponding D2_{N} and D2_{N+1}; D1_{N}, D1_{N+1} and D1_{N+2} are base paired with corresponding D2_{N}, D1_{N+1} and D1_{N+2}, etc. “N*”=0 to 15 or more, but is optionally 0, 1, 2, 3, 4, 5 or 6. In one embodiment, the top strand is the sense strand, and the bottom strand is the antisense strand. Alternatively, the bottom strand is the sense strand and the top strand is the antisense strand, with 2′-O-methyl RNA monomers located at alternating residues along the top strand, rather than the bottom strand presently depicted in the above schematic.

In any of the above-depicted structures, the 5′ end of either the sense strand or antisense strand optionally comprises a phosphate group.

In another embodiment, the DNA:DNA-extended DsiRNA comprises strands having equal lengths possessing 1-3 mismatched residues that serve to orient Dicer cleavage (specifically, one or more of positions 1, 2 or 3 on the first strand of the DsiRNA, when numbering from the 3′-terminal residue, are mismatched with corresponding residues of the 5′-terminal region on the second strand when first and second strands are annealed to one another). An exemplary DNA:DNA-extended DsiRNA agent with two terminal mismatched residues is shown:

5′-D_{N}XXXXXXXXXXXXXXXXXXXXXXXXXX_{N*}^{M}^{^{M-3′}}
3′-D_{N}XXXXXXXXXXXXXXXXXXXXXXXXXX_{N*}_{M}_{_{M-5′}}

wherein “X”=RNA, “M”=Nucleic acid residues (RNA, DNA or non-natural or modified nucleic acids) that do not base pair (hydrogen bond) with corresponding “M” residues of otherwise complementary strand when strands are annealed, “D”=DNA and “N”=1 to 50 or more, but is optionally 1-8. “N*”=0 to 15 or more, but is optionally 0, 1, 2, 3, 4, 5 or 6. Any of the residues of such agents can optionally be 2′-O-methyl RNA monomers—alternating positioning of 2′-O-methyl RNA monomers that commences from the 3′-terminal residue of the bottom (second) strand, as shown for above asymmetric agents, can also be used in the above “blunt/fray” DsiRNA agent. In one embodiment, the top strand (first strand) is the sense strand, and the bottom strand (second strand) is the antisense strand. Alternatively, the bottom strand is the sense strand and the top strand is the antisense strand. Modification and DNA:DNA extension patterns paralleling those shown above for asymmetric/overhang agents can also be incorporated into such “blunt/frayed” agents.

In another embodiment, a length-extended DsiRNA agent is provided that comprises deoxyribonucleotides positioned at sites modeled to function via specific direction of Dicer cleavage, yet which does not require the presence of a base-paired deoxyribonucleotide in the dsNA structure. An exemplary structure for such a molecule is shown:

5′-XXDDXXXXXXXXXXXXXXXXXXXX_{N*}Y-3′
3′-DDXXXXXXXXXXXXXXXXXXXXXX_{N*}-5′

wherein “X”=RNA, “Y” is an optional overhang domain comprised of 0-10 RNA monomers that are optionally 2′-O-methyl RNA monomers—in certain embodiments, “Y” is an overhang domain comprised of 1-4 RNA monomers that are optionally 2′-O-methyl RNA monomers, and “D”=DNA. “N*”=0 to 15 or more, but is optionally 0, 1, 2, 3, 4, 5 or 6. In one embodiment, the top strand is the sense strand, and the bottom strand is the antisense strand. Alternatively, the bottom strand is the sense strand and the top strand is the antisense strand. The above structure is modeled to force Dicer to cleave a minimum of a 21mer duplex as its primary post-processing form. In embodiments where the bottom strand of the above structure is the antisense strand, the positioning of two deoxyribonucleotide residues at the ultimate and penultimate residues of the 5′ end of the antisense strand is likely to reduce off-target effects (as prior studies have shown a 2′-O-methyl modification of at least the penultimate position from the 5′ terminus of the antisense strand to reduce off-target effects; see, e.g., US 2007/0223427).

In certain embodiments, the “D” residues of any of the above structures include at least one PS-DNA or PS-RNA. Optionally, the “D” residues of any of the above structures include at least one modified nucleotide that inhibits Dicer cleavage.

While the above-described “DNA-extended” DsiRNA agents can be categorized as either “left extended” or “right extended”, DsiRNA agents comprising both left- and right-extended DNA-containing sequences within a single agent (e.g., both flanks surrounding a core dsRNA structure are dsDNA extensions) can also be generated and used in similar manner to those described herein for “right-extended” and “left-extended” agents.

In some embodiments, the DsiRNA of the instant invention further comprises a linking moiety or domain that joins the sense and antisense strands of a DNA:DNA-extended DsiRNA agent. Optionally, such a linking moiety domain joins the 3′ end of the sense strand and the 5′ end of the antisense strand. The linking moiety may be a chemical (non-nucleotide) linker, such as an oligomethylenediol linker, oligoethylene glycol linker, or other art-recognized linker moiety. Alternatively, the linker can be a nucleotide linker, optionally including an extended loop and/or tetraloop.

In one embodiment, the DsiRNA agent has an asymmetric structure, with the sense strand having a 25-base pair length, and the antisense strand having a 27-base pair length with a 1-4 base 3′-overhang (e.g., a one base 3′-overhang, a two base 3′-overhang, a three base 3′-overhang or a four base 3′-overhang). In another embodiment, this DsiRNA agent has an asymmetric structure further containing 2 deoxynucleotides at the 3′ end of the sense strand.

In another embodiment, the DsiRNA agent has an asymmetric structure, with the antisense strand having a 25-base pair length, and the sense strand having a 27-base pair length with a 1-4 base 3′-overhang (e.g., a one base 3′-overhang, a two base 3′-overhang, a three base 3′-overhang or a four base 3′-overhang). In another embodiment, this DsiRNA agent has an asymmetric structure further containing 2 deoxynucleotides at the 3′ end of the antisense strand.

For the above blunt/fray agents of the invention, it is recognized that the precise sequence of a frayed end structure is not critical to efficacy, e.g., one or two of the 3′-terminal residues of the first strand only need to be non-complementary to the corresponding 5′-terminal residues of the second strand. In certain embodiments, the DsiRNA agents of the invention require, e.g., at least 19, at least 20, at least 21, at least 22, at least 23, at least 24, at least 25 or at least 26 residues of the first strand to be complementary to corresponding residues of the second strand. In certain related embodiments, these first strand residues complementary to corresponding residues of the second strand are optionally consecutive residues. Additionally and/or alternatively, certain mismatch residues can also be positioned within, e.g., the 5′ half of the sense strand, meaning that in certain embodiments, perfect complementarity between first and second strands does not hold across the entirety of the first and second strands even exclusive of a fray structure at the 3′ terminus of the first strand/5′ terminus of the second strand—in such embodiments, the first strand can be an effective first strand while the extent of complementarity between first and second strands across residues—optionally exclusive of the two 3′-terminal residues of the first strand/two 5′-terminal residues of the second strand for frayed agents—is at least 80% of residues, at least 85% of residues, at least 90% of residues, at least 90% of residues, at least 96% of residues. In certain embodiments, the extent of complementarity of the second strand of a DsiRNA of the invention relative to the first strand or relative to the target RNA sequence can be a level of complementarity with the first strand or with the target RNA sequence equivalent to that described above for the first strand of such DsiRNAs.

### RNA Processing

siRNA

The process of siRNA-mediated RNAi is triggered by the presence of long, dsRNA molecules in a cell. During the initiation step of RNAi, these dsRNA molecules are cleaved into 21-23 nucleotide (nt) small-interfering RNA duplexes (siRNAs) by Dicer, a conserved family of enzymes containing two RNase III-like domains (Bernstein et al. 2001; Elbashir et al. 2001). The siRNAs are characterized by a 19-21 base pair duplex region and 2 nucleotide 3′ overhangs on each strand. During the effector step of RNAi, the siRNAs become incorporated into a multimeric protein complex called RNA-induced silencing complex (RISC), where they serve as guides to select fully complementary mRNA substrates for degradation. Degradation is initiated by endonucleolytic cleavage of the mRNA within the region complementary to the siRNA. More precisely, the mRNA is cleaved at a position 10 nucleotides from the 5′ end of the guiding siRNA (Elbashir et al. 2001 Genes & Dev. 15: 188-200; Nykanen et al. 2001 Cell 107: 309-321; Martinez et al. 2002 Cell 110: 563-574). An endonuclease responsible for this cleavage was identified as Argonaute2 (Ago2; Liu et al. Science, 305: 1437-41).

miRNA

The majority of human miRNAs (70%)—and presumably the majority of miRNAs of other mammals—are transcribed from introns and/or exons, and approximately 30% are located in intergenic regions (Rodriguez et al., Genome Res. 2004, 14(10A), 1902-1910). In human and animal, miRNAs are usually transcribed by RNA polymerase II (Farh et al. Science 2005, 310(5755), 1817-1821), and in some cases by pol III (Borchert et al. Nat. Struct. Mol. Biol. 2006, 13(12), 1097-1101). Certain viral encoded miRNAs are transcribed by RNA polymerase III (Pfeffer et al. Nat. Methods 2005, 2(4), 269-276; Andersson et al. J. Virol. 2005, 79(15), 9556-9565), and some are located in the open reading frame of viral gene (Pfeffer et al. Nat. Methods 2005, 2(4), 269-276; Samols et al. J. Virol. 2005, 79(14), 9301-9305). miRNA transcription results in the production of large monocistronic, bicistronic or polycistronic primary transcripts (pri-miRNAs). A single pri-miRNA may range from approximately 200 nucleotides (nt) to several kilobases (kb) in length and have both a 5′ 7-methylguanosine (m7) caps and a 3′ poly (A) tail. Characteristically, the mature miRNA sequences are localized to regions of imperfect stem-loop sequences within the pri-miRNAs (Cullen, Mol. Cell. 2004, 16(6), 861-865).

The first step of miRNA maturation in the nucleus is the recognition and cleavage of the pri-miRNAs by the RNase III Drosha-DGCR8 nuclear microprocessor complex, which releases a ˜70 nt hairpin-containing precursor molecule called pre-miRNAs, with a monophosphate at the 5′ terminus and a 2-nt overhang with a hydroxyl group at the 3′ terminus (Cai et al. RNA 2004, 10(12), 1957-1966; Lee et al. Nature 2003, 425(6956), 415-419; Kim Nat. Rev. Mol. Cell. Biol. 2005, 6(5), 376-385). The next step is the nuclear transport of the pre-miRNAs out of the nucleus into the cytoplasm by Exportin-5, a carrier protein (Yi et al. Genes. Dev. 2003, 17(24), 3011-3016, Bohnsack et al. RNA 2004, 10(2), 185-191). Exportin-5 and the GTP-bound form of its cofactor Ran together recognize and bind the 2 nucleotide 3′ overhang and the adjacent stem that are characteristics of pre-miRNA (Basyuk et al. Nucl. Acids Res. 2003, 31(22), 6593-6597, Zamore Mol. Cell. 2001, 8(6), 1158-1160). In the cytoplasm, GTP hydrolysis results in release of the pre-miRNA, which is then processed by a cellular endonuclease III enzyme Dicer (Bohnsack et al.). Dicer was first recognized for its role in generating siRNAs that mediate RNA interference (RNAi). Dicer acts in concert with its cofactors TRBP (Transactivating region binding protein; Chendrimata et al. Nature 2005, 436(7051), 740-744) and PACT (interferon-inducible double strand-RNA-dependant protein kinase activator; Lee et al. EMBO J. 2006, 25(3), 522-532). These enzymes bind at the 3′ 2 nucleotide overhang at the base of the pre-miRNA hairpin and remove the terminal loop, yielding an approximately 21-nt miRNA duplex intermediate with both termini having 5′ monophosphates, 3′ 2 nucleotide overhangs and 3′ hydroxyl groups. The miRNA guide strand, the 5′ terminus of which is energetically less stable, is then selected for incorporation into the RISC(RNA-induced silencing complex), while the ‘passenger’ strand is released and degraded (Maniataki et al. Genes. Dev. 2005, 19(24), 2979-2990; Hammond et al. Nature 2000, 404(6775), 293-296). The composition of RISC remains incompletely defined, but a key component is a member of the Argonaute (Ago) protein family (Maniataki et al.; Meister et al. Mol. Cell. 2004, 15(2), 185-197).

The mature miRNA then directs RISC to complementary mRNA species. If the target mRNA has perfect complementarity to the miRNA-armed RISC, the mRNA will be cleaved and degraded (Zeng et al. Proc. Natl. Acad. Sci. USA 2003, 100(17), 9779-9784; Hutvagner et al. Science 2002, 297(55 89), 2056-2060). But as the most common situation in mammalian cells, the miRNAs targets mRNAs with imperfect complementarity and suppress their translation, resulting in reduced expression of the corresponding proteins (Yekta et al. Science 2004, 304(5670), 594-596; Olsen et al. Dev. Biol. 1999, 216(2), 671-680). The 5′ region of the miRNA, especially the match between miRNA and target sequence at nucleotides 2-7 or 8 of miRNA (starting from position 1 at the 5′ terminus), which is called the seed region, is essentially important for miRNA targeting, and this seed match has also become a key principle widely used in computer prediction of the miRNA targeting (Lewis et al. Cell 2005, 120(1), 15-20; Brennecke et al. PLoS Biol. 2005, 3(3), e85). miRNA regulation of the miRNA-mRNA duplexes is mediated mainly through multiple complementary sites in the 3′ UTRs, but there are many exceptions. miRNAs may also bind the 5′ UTR and/or the coding region of mRNAs, resulting in a similar outcome (Lytle et al. Proc. Natl. Acad. Sci. USA 2007, 104(23), 9667-9672).

### RNase H

RNase H is a ribonuclease that cleaves the 3′-O—P bond of RNA in a DNA/RNA duplex to produce 3′-hydroxyl and 5′-phosphate terminated products. RNase H is a non-specific endonuclease and catalyzes cleavage of RNA via a hydrolytic mechanism, aided by an enzyme-bound divalent metal ion. Members of the RNase H family are found in nearly all organisms, from archaea and prokaryotes to eukaryotes. During DNA replication, RNase H is believed to cut the RNA primers responsible for priming generation of Okazaki fragments; however, the RNase H enzyme may be more generally employed to cleave any DNA:RNA hybrid sequence of sufficient length (e.g., typically DNA:RNA hybrid sequences of 4 or more base pairs in length in mammals).

### MicroRNA and MicroRNA-Like Therapeutics

MicroRNAs (miRNAs) have been described to act by binding to the 3′ UTR of a template transcript, thereby inhibiting expression of a protein encoded by the template transcript by a mechanism related to but distinct from classic RNA interference. Specifically, miRNAs are believed to act by reducing translation of the target transcript, rather than by decreasing its stability. Naturally-occurring miRNAs are typically approximately 22 nt in length. It is believed that they are derived from larger precursors known as small temporal RNAs (stRNAs) approximately 70 nt long.

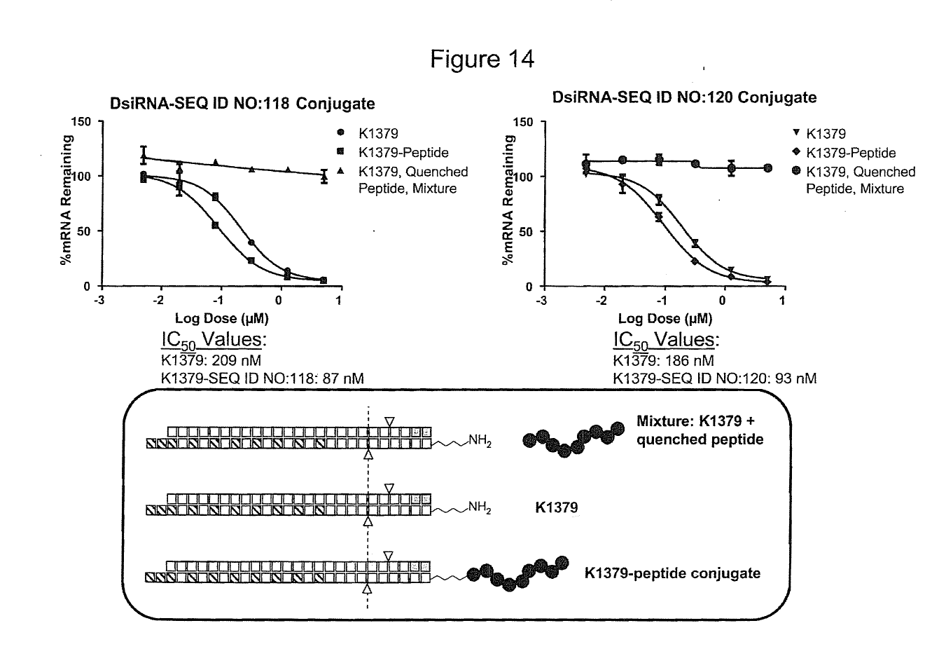

Interference agents such as siRNAs, and more specifically such as miRNAs, that bind within the 3′ UTR (or elsewhere in a target transcript, e.g., in repeated elements of, e.g., Notch and/or transcripts of the Notch family) and inhibit translation may tolerate a larger number of mismatches in the siRNA/template (miRNA/template) duplex, and particularly may tolerate mismatches within the central region of the duplex. In fact, there is evidence that some mismatches may be desirable or required, as naturally occurring stRNAs frequently exhibit such mismatches, as do miRNAs that have been shown to inhibit translation in vitro (Zeng et al., Molecular Cell, 9: 1-20). For example, when hybridized with the target transcript, such miRNAs frequently include two stretches of perfect complementarity separated by a region of mismatch. Such a hybridized complex commonly includes two regions of perfect complementarily (duplex portions) comprising nucleotide pairs, and at least a single mismatched base pair, which may be, e.g., G:A, G:U, G:G, A:A, A:C, U:U, U:C, C:C, G:-, A:-, U:-, C:-, etc. Such mismatched nucleotides, especially if present in tandem (e.g., a two, three or four nucleotide area of mismatch) can form a bulge that separates duplex portions which are located on either flank of such a bulge. A variety of structures are possible. For example, the miRNA may include multiple areas of nonidentity (mismatch). The areas of nonidentity (mismatch) need not be symmetrical in the sense that both the target and the miRNA include nonpaired nucleotides. For example, structures have been described in which only one strand includes nonpaired nucleotides (Zeng et al., FIG. 14). Typically the stretches of perfect complementarily within a miRNA agent are at least 5 nucleotides in length, e.g., 6, 7, or more nucleotides in length, while the regions of mismatch may be, for example, 1, 2, 3, or 4 nucleotides in length.

In general, any particular siRNA could function to inhibit gene expression both via (i) the “classical” siRNA pathway, in which stability of a target transcript is reduced and in which perfect complementarily between the siRNA and the target is frequently preferred, and also by (ii) the “alternative” pathway (generally characterized as the miRNA pathway in animals), in which translation of a target transcript is inhibited. Generally, the transcripts targeted by a particular siRNA via mechanism (i) would be distinct from the transcript targeted via mechanism (ii), although it is possible that a single transcript could contain regions that could serve as targets for both the classical and alternative pathways. (Note that the terms “classical” and “alternative” are used merely for convenience and generally are believed to reflect historical timing of discovery of such mechanisms in animal cells, but do not reflect the importance, effectiveness, or other features of either mechanism.) One common goal of siRNA design has been to target a single transcript with great specificity, via mechanism (i), while minimizing off-target effects, including those effects potentially elicited via mechansim (ii). However, it is among the goals of the instant invention to provide RNA interference agents that possess mismatch residues by design, either for purpose of mimicking the activities of naturally-occurring miRNAs, or to create agents directed against target RNAs for which no corresponding miRNA is presently known, with the inhibitory and/or therapeutic efficacies/potencies of such “DmiRNA” agents tolerant of, and indeed possibly enhanced by, such mismatches.

The tolerance of miRNA agents for mismatched nucleotides (and, indeed the existence and natural use of mechanism (ii) above in the cell) suggests the use of miRNAs in manners that are advantageous to and/or expand upon the “classical” use of perfectly complementary siRNAs that act via mechanism (i). Because niiRNAs are naturally occurring molecules, there are likely to be distinct advantages in applying miRNAs as therapeutic agents. miRNAs benefit from hundreds of millions of years of evolutionary “fine tuning” of their function, Thus, sequence-specific “off target” effects should not be an issue with naturally occurring miRNAs, nor, by extension, with synthetic DmiRNAs of the invention designed to directly mimic naturally occurring miRNAs. In addition, miRNAs have evolved to modulate the expression of groups of genes, driving both up and down regulation (in certain instances, performing both functions concurrently within a cell with a single miRNA acting promiscuously upon multiple target RNAs), with the result that complex cell functions can be precisely modulated. Such replacement of naturally occurring miRNAs can involve introducing synthetic niiRNAs or miRNA mimetics DmiRNAs) into diseased tissues in an effort to restore normal proliferation, apoptosis, cell cycle, and other cellular functions that have been affected by down-regulation of one or more miRNAs. In certain instances, reactivation of these miRNA-regulated pathways has produced a significant therapeutic response (e.g., In one study on cardiac hypertrophy, overexpression of miR-133 by adenovirus-mediated delivery of a miRNA expression cassette protected animals from agonist-induced cardiac hypertrophy, whereas reciprocally reduction of miR-133 in wild-type mice by antagomirs caused an increase in hypertrophic markers (Care et al. Nat. Med. 13: 613-618)).

To date, more than 600 miRNAs have been identified as encoded within the human genome, with such miRNAs expressed and processed by a combination of proteins in the nucleus and cytoplasm. miRNAs are highly conserved among vertebrates and comprise approximately 2% of all mammalian genes. Since each miRNA appears to regulate the expression of multiple, e.g., two, three, four, five, six, seven, eight, nine or even tens to hundreds of different genes, miRNAs can function as “master-switches”, efficiently regulating and coordinating multiple cellular pathways and processes. By coordinating the expression of multiple genes, miRNAs play key roles in embryonic development, immunity, inflammation, as well as cellular growth and proliferation.

Expression and functional studies suggest that the altered expression of specific miRNAs is critical to a variety of human diseases. Mounting evidence indicates that the introduction of specific miRNAs into disease cells and tissues can induce favorable therapeutic responses (Pappas et al., Expert Opin Ther Targets. 12: 115-27), The promise of miRNA therapy is perhaps greatest in cancer due to the apparent role of certain miRNAs as tumor suppressors. The rationale for miRNA-based therapeutics for, e.g., cancer is supported, at least in part, by the following observations:
(1) miRNAs are frequently mis-regulated and expressed at altered levels in diseased tissues when compared to normal tissues. A number of studies have shown altered levels of miRNAs in cancerous tissues relative to their corresponding normal tissues. Often, altered expression is the consequence of genetic mutations that lead to increased or reduced expression of particular miRNAs, Diseases that possess unique miRNA expression signatures can be exploited as diagnostic and prognostic markers, and can be targeted with the DsiRNA (DmiRNA) agents of the invention.
(2) Mis-regulated miRNAs contribute to cancer development by functioning as oncogenes or tumor suppressors, Oncogenes are defined as genes whose over-expression or inappropriate activation leads to oncogenesis, Tumor suppressors are genes that are required to keep cells from being cancerous; the down-regulation or inactivation of tumor suppressors is a common inducer of cancer. Both types of genes represent preferred drug targets, as such targeting can specifically act upon the molecular basis for a particular cancer, Examples of oncogenic miRNAs are miR-155 and miR-17-92; let-7 is an example of a tumor suppressive miRNA.
(3) Administration of miRNA induces a therapeutic response by blocking or reducing tumor growth in pre-clinical animal studies. The scientific literature, provides proof-of-concept studies demonstrating that restoring miRNA function can prevent or reduce the growth of cancer cells in vitro and also in animal models. A well-characterized example is the anti-tumor activity of let-7 in models for breast and lung cancer. DsiRNAs (DmiRNAs) of the invention which are designed to mimic let-7 can be used to target such cancers, and it is also possible to use the DsiRNA design parameters described herein to generate new DsiRNA (DmiRNA) agents directed against target RNAs for which no counterpart naturally occurring miRNA is known (e.g., repeats within Notch or other transcripts), to screen for therapeutic lead compounds, e.g., agents that are capable of reducing tumor burden in pre-clinical animal models.
(4) A given miRNA controls multiple cellular pathways and therefore may have superior therapeutic activity. Based on their biology, miRNAs can function as “master switches” of the genome, regulating multiple gene products and coordinating multiple pathways. Genes regulated by miRNAs include genes that encode conventional oncogenes and tumor suppressors, many of which are individually pursued as drug targets by the pharmaceutical industry. Thus, miRNA therapeutics could possess activity superior to siRNAs and other forms of lead compounds by targeting multiple disease and/or cancer-associated genes. Given the observation that mis-regulation of miRNAs is frequently an early event in the process of tumorigenesis, miRNA therapeutics, which replace missing miRNAs, may be the most appropriate therapy.
(5) miRNAs are natural molecules and are therefore less prone to induce non-specific side-effects. Millions of years of evolution helped to develop the regulatory network of miRNAs, fine-tuning the interaction of miRNA with target messenger RNAs. Therefore, miRNAs and miRNA derivatives (e.g., DmiRNAs designed to mimic naturally occurring miRNAs) will have few if any sequence-specific “off-target” effects when applied in the proper context.

The physical characteristics of siRNAs and miRNAs are similar. Accordingly, technologies that are effective in delivering siRNAs (e.g., DsiRNAs of the invention) are likewise effective in delivering synthetic miRNAs (e.g., DmiRNAs of the invention).

### Conjugation and Delivery of DsiRNA Agents

In certain embodiments the present invention relates to a method for treating a subject having a disease or disorder, or at risk of developing a disease or disorder. In such embodiments, the DsiRNA can act as novel therapeutic agents for controlling the disease or disorder. The method comprises administering a pharmaceutical composition of the invention to the patient (e.g., human), such that the expression, level and/or activity of a target RNA is reduced. The expression, level and/or activity of a polypeptide encoded by an RNA of interest might also be reduced by a DsiRNA of the instant invention, even where said DsiRNA is directed against a non-coding region of the transcript (e.g., a targeted 5′ UTR or 3′ UTR sequence). Because of their high specificity, the DsiRNAs of the present invention can specifically target a sequence of interest of cells and tissues, optionally in an allele-specific manner where polymorphic alleles exist within an individual and/or population.

In the treatment of a disease or disorder, the DsiRNA can be brought into contact with the cells or tissue of a subject, e.g., the cells or tissue of a subject exhibiting disregulation of a protein and/or otherwise targeted for reduction of protein levels. For example, DsiRNA substantially identical to all or part of an RNA sequence of interest, may be brought into contact with or introduced into such a cell, either in vivo or in vitro. Similarly, DsiRNA substantially identical to all or part of an RNA sequence of interest may be administered directly to a subject having or at risk of developing a disease or disorder.

Therapeutic use of the DsiRNA agents of the instant invention can involve use of formulations of DsiRNA agents comprising multiple different DsiRNA agent sequences. For example, two or more, three or more, four or more, five or more, etc. of the presently described agents can be combined to produce a formulation that, e.g., targets multiple different regions of a target RNA, or that not only target an RNA of interest but also target, e.g., cellular target genes associated with a disease or disorder associated with a target RNA of interest. A DsiRNA agent of the instant invention may also be constructed such that either strand of the DsiRNA agent independently targets two or more regions of an RNA target, or such that one of the strands of the DsiRNA agent targets a cellular target gene of a target mRNA known in the art.

Use of multifunctional DsiRNA molecules that target more then one region of a target nucleic acid molecule can also provide potent inhibition of RNA levels and expression. For example, a single multifunctional DsiRNA construct of the invention can target both.

Thus, the DsiRNA agents of the instant invention, individually, or in combination or in conjunction with other drugs, can be used to treat, inhibit, reduce, or prevent a disease or disorder-associated with a target RNA. For example, the DsiRNA molecules can be administered to a subject or can be administered to other appropriate cells evident to those skilled in the art, individually or in combination with one or more drugs under conditions suitable for the treatment.

The DsiRNA molecules also can be used in combination with other known treatments to treat, inhibit, reduce, or prevent a disease or disorder associated with a target RNA in a subject or organism. For example, the described molecules could be used in combination with one or more known compounds, treatments, or procedures to treat, inhibit, reduce, or prevent a disease or disorder associated with a target RNA in a subject or organism as are known in the art.

A DsiRNA agent of the invention can be conjugated (e.g., at its 5′ or 3′ terminus of its sense or antisense strand) or unconjugated to another moiety (e.g. a non-nucleic acid moiety such as a peptide), an organic compound (e.g., a dye, cholesterol, or the like). Modifying DsiRNA agents in this way may improve cellular uptake or enhance cellular targeting activities of the resulting DsiRNA agent derivative as compared to the corresponding unconjugated DsiRNA agent, are useful for tracing the DsiRNA agent derivative in the cell, or improve the stability of the DsiRNA agent derivative compared to the corresponding unconjugated DsiRNA agent.

The invention also contemplates dsRNA-peptide conjugates further conjugated to a therapeutic agent, for example an agent that treats or ameliorates the symptoms and/or progression of a disease, for example cancer.

### Methods of Introducing Nucleic Acids, Vectors, and Host Cells

DsiRNA agents of the invention may be directly introduced into a cell (i.e., intracellularly); or introduced extracellularly into a cavity, interstitial space, into the circulation of an organism, introduced orally, or may be introduced by bathing a cell or organism in a solution containing the nucleic acid. Vascular or extravascular circulation, the blood or lymph system, and the cerebrospinal fluid are sites where the nucleic acid may be introduced.

The DsiRNA agents of the invention can be introduced using nucleic acid delivery methods known in art including injection of a solution containing the nucleic acid, bombardment by particles covered by the nucleic acid, soaking the cell or organism in a solution of the nucleic acid, or electroporation of cell membranes in the presence of the nucleic acid. Other methods known in the art for introducing nucleic acids to cells may be used, such as lipid-mediated carrier transport, chemical-mediated transport, and cationic liposome transfection such as calcium phosphate, and the like. The nucleic acid may be introduced along with other components that perform one or more of the following activities: enhance nucleic acid uptake by the cell or other-wise increase inhibition of the target RNA.

A cell having a target RNA may be from the germ line or somatic, totipotent or pluripotent, dividing or non-dividing, parenchyma or epithelium, immortalized or transformed, or the like. The cell may be a stem cell or a differentiated cell. Cell types that are differentiated include adipocytes, fibroblasts, myocytes, cardiomyocytes, endothelium, neurons, glia, blood cells, megakaryocytes, lymphocytes, macrophages, neutrophils, eosinophils, basophils, mast cells, leukocytes, granulocytes, keratinocytes, chondrocytes, osteoblasts, osteoclasts, hepatocytes, and cells of the endocrine or exocrine glands.

Depending on the particular target RNA sequence and the dose of DsiRNA agent material delivered, this process may provide partial or complete loss of function for the RNA. A reduction or loss of RNA levels or expression (either RNA expression or encoded polypeptide expression) in at least 50%, 60%, 70%, 80%, 90%, 95% or 99% or more of targeted cells is exemplary. Inhibition of RNA levels or expression refers to the absence (or observable decrease) in the level of RNA or RNA-encoded protein. Specificity refers to the ability to inhibit the RNA without manifest effects on other genes of the cell. The consequences of inhibition can be confirmed by examination of the outward properties of the cell or organism or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). Inhibition of target RNA sequence(s) by the DsiRNA agents of the invention also can be measured based upon the effect of administration of such DsiRNA agents upon development/progression of a disease or disorder associated with a target RNA of interest, e.g., tumor formation, growth, metastasis, etc., either in vivo or in vitro. Treatment and/or reductions in tumor or cancer cell levels can include halting or reduction of growth of tumor or cancer cell levels or reductions of, e.g., 10%, 20%, 30%, 40%, 50%, 60%, 70%, 80%, 90%, 95% or 99% or more, and can also be measured in logarithmic terms, e.g., 10-fold, 100-fold, 1000-fold, 10⁵-fold, 10⁶-fold, 10⁷-fold reduction in cancer cell levels could be achieved via administration of the DsiRNA agents of the invention to cells, a tissue, or a subject.

For RNA-mediated inhibition in a cell line or whole organism, expression a reporter or drug resistance gene whose protein product is easily assayed can be measured. Such reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentamycin, hygromycin, kanamycin, lincomycin, methotrexate, phosphinothricin, puromycin, and tetracyclin. Depending on the assay, quantitation of the amount of gene expression allows one to determine a degree of inhibition which is greater than 10%, 33%, 50%, 90%, 95% or 99% as compared to a cell not treated according to the present invention.

Lower doses of injected material and longer times after administration of RNA silencing agent may result in inhibition in a smaller fraction of cells (e.g., at least 10%, 20%, 50%, 75%, 90%, or 95% of targeted cells). Quantitation of gene expression in a cell may show similar amounts of inhibition at the level of accumulation of target RNA or translation of target protein. As an example, the efficiency of inhibition may be determined by assessing the amount of gene product in the cell; RNA may be detected with a hybridization probe having a nucleotide sequence outside the region used for the inhibitory DsiRNA, or translated polypeptide may be detected with an antibody raised against the polypeptide sequence of that region.

The DsiRNA agent may be introduced in an amount which allows delivery of at least one copy per cell. Higher doses (e.g., at least 5, 10, 100, 500 or 1000 copies per cell) of material may yield more effective inhibition; lower doses may also be useful for specific applications.

### Pharmaceutical Compositions

In certain embodiments, the present invention provides for a pharmaceutical composition comprising the dsRNA-peptide agents of the present invention. The dsRNA-peptide agent sample can be suitably formulated and introduced into the environment of the cell by any means that allows for a sufficient portion of the sample to enter the cell to induce gene silencing, if it is to occur. Many formulations for dsRNA are known in the art and can be used so long as the dsRNA gains entry to the target cells so that it can act. See, e.g., U.S. published patent application Nos. 2004/0203145 A1 and 2005/0054598 A1. For example, the dsRNA-peptide agent of the instant invention can be formulated in buffer solutions such as phosphate buffered saline solutions, liposomes, micellar structures, and capsids. Formulations of DsiRNA agent with cationic lipids can be used to facilitate transfection of the DsiRNA agent into cells. For example, cationic lipids, such as lipofectin (U.S. Pat. No. 5,705,188), cationic glycerol derivatives, and polycationic molecules, such as polylysine (published PCT International Application WO 97/30731), can be used. Suitable lipids include Oligofectamine, Lipofectamine (Life Technologies), NC388 (Ribozyme Pharmaceuticals, Inc., Boulder, Colo.), or FuGene 6 (Roche) all of which can be used according to the manufacturer's instructions.

Such compositions typically include the nucleic acid molecule and a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer. Such methods include those described in U.S. Pat. No. 6,468,798.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

The compounds can also be administered by transfection or infection using methods known in the art, including but not limited to the methods described in McCaffrey et al. (2002), Nature, 418(6893), 38-9 (hydrodynamic transfection); Xia et al. (2002), Nature Biotechnol., 20(10), 1006-10 (viral-mediated delivery); or Putnam (1996), Am. J. Health Syst. Pharm. 53(2), 151-160, erratum at Am. J. Health Syst. Pharm. 53(3), 325 (1996).

The compounds can also be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al. (1998), Clin. Immunol. Immunopathol., 88(2), 205-10. Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of a nucleic acid molecule (i.e., an effective dosage) depends on the nucleic acid selected. For instance, if a plasmid encoding a DsiRNA agent is selected, single dose amounts in the range of approximately 1 pg to 1000 mg may be administered; in some embodiments, 10, 30, 100, or 1000 pg, or 10, 30, 100, or 1000 ng, or 10, 30, 100, or 1000 μg, or 10, 30, 100, or 1000 mg may be administered. In some embodiments, 1-5 g of the compositions can be administered. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

As therapeutically useful peptide according to the invention “increases” targeting, as defined hereinabove, of a peptide-dsRNA conjugate such that less dsRNA (a lower dose of dsRNA) as compared to the amount or dose of an identical dsRNA that is not conjugated to a peptide and that is required to achieve an equivalent level of binding, association or internalization, as determined by the IC₅₀, in the assays described hereinbelow is required. For example, the IC₅₀ for a dsRNA-peptide conjugate that is required to achieve a 50% reduction in RNA/gene expression is decreased as compared to the IC₅₀ for an identical dsRNA that is not conjugated to a peptide, as measured in vivo or in vitro (see for example Hefner et al. J Biomol Tech. 2008 September: 19(4) 231-237; Zimmermann et al. Nature. 2006 May 4: 441(7089):111-114; Durcan et al. Mol. Pharm. 2008 July-August; 5(4):559-566; Heidel et al. Proc Natl Acad Sci USA. 2007 Apr. 3: 104(14):5715-5721.).

A useful dose of dsRNA-peptide as defined herein is on the order of 0.1 mg/kg-100 mg/kg, for example, 0.2 kg/mg-50 kg/mg, 0.5 kg/mg-30 kg/mg or 0.5 mg/kg-20 mg/kg (including 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20 kg/mg or more).

The nucleic acid molecules of the invention can be inserted into expression constructs, e.g., viral vectors, retroviral vectors, expression cassettes, or plasmid viral vectors, e.g., using methods known in the art, including but not limited to those described in Xia et al., (2002), supra. Expression constructs can be delivered to a subject by, for example, inhalation, orally, intravenous injection, local administration (see U.S. Pat. No. 5,328,470) or by stereotactic injection (see e.g., Chen et al. (1994), Proc. Natl. Acad. Sci. USA, 91, 3054-3057). The pharmaceutical preparation of the delivery vector can include the vector in an acceptable diluent, or can comprise a slow release matrix in which the delivery vehicle is imbedded. Alternatively, where the complete delivery vector can be produced intact from recombinant cells, e.g., retroviral vectors, the pharmaceutical preparation can include one or more cells which produce the gene delivery system.

The expression constructs may be any construct suitable for use in the appropriate expression system and include, but are not limited to retroviral vectors, linear expression cassettes, plasmids and viral or virally-derived vectors, as known in the art. Such expression constructs may include one or more inducible promoters, RNA Pol III promoter systems such as U6 snRNA promoters or H1 RNA polymerase III promoters, or other promoters known in the art. The constructs can include one or both strands of the siRNA. Expression constructs expressing both strands can also include loop structures linking both strands, or each strand can be separately transcribed from separate promoters within the same construct. Each strand can also be transcribed from a separate expression construct, e.g., Tuschl (2002, Nature Biotechnol 20: 500-505).

It can be appreciated that the method of introducing DsiRNA agents into the environment of the cell will depend on the type of cell and the make up of its environment. For example, when the cells are found within a liquid, one preferable formulation is with a lipid formulation such as in lipofectamine and the DsiRNA agents can be added directly to the liquid environment of the cells. Lipid formulations can also be administered to animals such as by intravenous, intramuscular, or intraperitoneal injection, or orally or by inhalation or other methods as are known in the art. When the formulation is suitable for administration into animals such as mammals and more specifically humans, the formulation is also pharmaceutically acceptable. Pharmaceutically acceptable formulations for administering oligonucleotides are known and can be used. In some instances, it may be preferable to formulate DsiRNA agents in a buffer or saline solution and directly inject the formulated DsiRNA agents into cells, as in studies with oocytes. The direct injection of DsiRNA agents duplexes may also be done. For suitable methods of introducing dsRNA (e.g., DsiRNA agents), see U.S. published patent application No. 2004/0203145 A1.

Suitable amounts of a DsiRNA agent must be introduced and these amounts can be empirically determined using standard methods. Typically, effective concentrations of individual DsiRNA agent species in the environment of a cell will be about 50 nanomolar or less, 10 nanomolar or less, or compositions in which concentrations of about 1 nanomolar or less can be used. In another embodiment, methods utilizing a concentration of about 200 picomolar or less, 100 picomolar or less, 50 picomolar or less, 20 picomolar or less and even a concentration of about 10 picomolar or less, 5 picomolar or less, 2 picomolar or less or 1 picomolar or less can be used in many circumstances.

The method can be carried out by addition of the DsiRNA agent compositions to any extracellular matrix in which cells can live provided that the DsiRNA agent composition is formulated so that a sufficient amount of the DsiRNA agent can enter the cell to exert its effect. For example, the method is amenable for use with cells present in a liquid such as a liquid culture or cell growth media, in tissue explants, or in whole organisms, including animals, such as mammals and especially humans.

The level or activity of an RNA can be determined by any suitable method now known in the art or that is later developed. It can be appreciated that the method used to measure a target RNA and/or the expression of a target RNA can depend upon the nature of the target RNA. For example, where the target RNA sequence encodes a protein, the term “expression” can refer to a protein or the RNA/transcript derived from the gene of interest (either genomic or of exogenous origin). In such instances the expression of the target RNA can be determined by measuring the amount of target RNA/transcript directly or by measuring the amount of the protein product of the RNA of interest. Protein can be measured in protein assays such as by staining or immunoblotting or, if the protein catalyzes a reaction that can be measured, by measuring reaction rates. All such methods are known in the art and can be used. Where target RNA levels are to be measured, any art-recognized methods for detecting RNA levels can be used (e.g., RT-PCR, Northern Blotting, etc.). In targeting RNAs with the DsiRNA agents of the instant invention, it is also anticipated that measurement of the efficacy of a DsiRNA agent in reducing levels of RNA or protein in a subject, tissue, in cells, either in vitro or in vivo, or in cell extracts can also be used to determine the extent of reduction of phenotypes associated with a particular RNA of interest (e.g., disease or disorders, e.g., cancer or tumor formation, growth, metastasis, spread, etc.). Any of the above measurements can be made on cells, cell extracts, tissues, tissue extracts or any other suitable source material.

The determination of whether the expression of a target RNA has been reduced can be by any suitable method that can reliably detect changes in RNA levels. Typically, the determination is made by introducing into the environment of a cell undigested DsiRNA such that at least a portion of that DsiRNA agent enters the cytoplasm, and then measuring the level of the target RNA. The same measurement is made on identical untreated cells and the results obtained from each measurement are compared.

The DsiRNA agent can be formulated as a pharmaceutical composition which comprises a pharmacologically effective amount of a DsiRNA agent and pharmaceutically acceptable carrier. A pharmacologically or therapeutically effective amount refers to that amount of a DsiRNA agent effective to produce the intended pharmacological, therapeutic or preventive result. The phrases “pharmacologically effective amount” and “therapeutically effective amount” or simply “effective amount” refer to that amount of an RNA effective to produce the intended pharmacological, therapeutic or preventive result. For example, if a given clinical treatment is considered effective when there is at least a 20% reduction in a measurable parameter associated with a disease or disorder, a therapeutically effective amount of a drug for the treatment of that disease or disorder is the amount necessary to effect at least a 20% reduction in that parameter.

Suitably formulated pharmaceutical compositions of this invention can be administered by any means known in the art such as by parenteral routes, including intravenous, intramuscular, intraperitoneal, subcutaneous, transdermal, airway (aerosol), rectal, vaginal and topical (including buccal and sublingual) administration. In some embodiments, the pharmaceutical compositions are administered by intravenous or intraparenteral infusion or injection.

In general, a suitable dosage unit of dsRNA will be in the range of 0.001 to 0.25 milligrams per kilogram body weight of the recipient per day, or in the range of 0.01 to 20 micrograms per kilogram body weight per day, or in the range of 0.01 to 10 micrograms per kilogram body weight per day, or in the range of 0.10 to 5 micrograms per kilogram body weight per day, or in the range of 0.1 to 2.5 micrograms per kilogram body weight per day. Pharmaceutical composition comprising the dsRNA can be administered once daily. However, the therapeutic agent may also be dosed in dosage units containing two, three, four, five, six or more sub-doses administered at appropriate intervals throughout the day. In that case, the dsRNA contained in each sub-dose must be correspondingly smaller in order to achieve the total daily dosage unit. The dosage unit can also be compounded for a single dose over several days, e.g., using a conventional sustained release formulation which provides sustained and consistent release of the dsRNA over a several day period. Sustained release formulations are well known in the art. In this embodiment, the dosage unit contains a corresponding multiple of the daily dose. Regardless of the formulation, the pharmaceutical composition must contain dsRNA in a quantity sufficient to inhibit expression of the target gene in the animal or human being treated. The composition can be compounded in such a way that the sum of the multiple units of dsRNA together contain a sufficient dose.

Data can be obtained from cell culture assays and animal studies to formulate a suitable dosage range for humans. The dosage of compositions of the invention lies within a range of circulating concentrations that include the ED₅₀ (as determined by known methods) with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range of the compound that includes the IC₅₀ (i.e., the concentration of the test compound which achieves a half-maximal inhibition of symptoms) as determined in cell culture. Such information can be used to more accurately determine useful doses in humans. Levels of dsRNA in plasma may be measured by standard methods, for example, by high performance liquid chromatography.

The pharmaceutical compositions can be included in a kit, container, pack, or dispenser together with instructions for administration.

### Methods of Treatment

The present invention provides for both prophylactic and therapeutic methods of treating a subject at risk of (or susceptible to) a disease or disorder caused, in whole or in part, by the an RNA of interest (e.g., misregulation and/or elevation of transcript and/or protein levels), or treatable via selective targeting of an RNA of interest.

“Treatment”, or “treating” as used herein, is defined as the application or administration of a therapeutic agent (e.g., a DsiRNA agent or vector or transgene encoding same) to a patient, or application or administration of a therapeutic agent to an isolated tissue or cell line from a patient, who has the disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease.

In one aspect, the invention provides a method for preventing in a subject, a disease or disorder as described above (including, e.g., prevention of the commencement of transforming events within a subject via inhibition of expression of an RNA of interest), by administering to the subject a therapeutic agent (e.g., a DsiRNA agent or vector or transgene encoding same). Subjects at risk for the disease can be identified by, for example, any or a combination of diagnostic or prognostic assays as described herein. Administration of a prophylactic agent can occur prior to the detection of, e.g., cancer in a subject, or the manifestation of symptoms characteristic of the disease or disorder, such that the disease or disorder is prevented or, alternatively, delayed in its progression.

Another aspect of the invention pertains to methods of treating subjects therapeutically, i.e., altering the onset of symptoms of the disease or disorder. These methods can be performed in vitro (e.g., by culturing the cell with the DsiRNA agent) or, alternatively, in vivo (e.g., by administering the DsiRNA agent to a subject).

With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”). Thus, another aspect of the invention provides methods for tailoring an individual's prophylactic or therapeutic treatment with either the target RNA molecules of the present invention or target RNA modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

Therapeutic agents can be tested in an appropriate animal model. For example, a DsiRNA agent (or expression vector or transgene encoding same) as described herein can be used in an animal model to determine the efficacy, toxicity, or side effects of treatment with said agent. Alternatively, an agent (e.g., a therapeutic agent) can be used in an animal model to determine the mechanism of action of such an agent.

Models Useful to Evaluate the Down-Regulation of mRNA Levels and Expression

### Cell Culture

The dsRNA-peptide agents of the invention can be tested for cleavage activity in vivo, for example, using the following procedure.

The dsRNA-peptide reagents of the invention can be tested in cell culture using HeLa or other mammalian cells to determine the extent of target RNA and target protein inhibition. dsRNA-peptide reagents (e.g., see FIG. 1 and above-recited structures) are selected against the target as described herein. Target RNA inhibition is measured after delivery of these reagents by a suitable transfection agent to, for example, cultured HeLa cells or other transformed or non-transformed mammalian cells in culture. Relative amounts of target RNA are measured versus actin or other appropriate control using real-time PCR monitoring of amplification (e.g., ABI 7700 TAQMAN®). A comparison is made to a mixture of oligonucleotide sequences made to unrelated targets or to a randomized DsiRNA control with the same overall length and chemistry, but randomly substituted at each position, or simply to appropriate vehicle-treated or untreated controls. Primary and secondary lead reagents are chosen for the target and optimization performed. After an optimal transfection agent concentration is chosen, a RNA time-course of inhibition is performed with the lead DsiRNA molecule.

TAQMAN® (Real-Time PCR Monitoring of Amplification) and Lightcycler Quantification of mRNA

Total RNA is prepared from cells following dsRNA delivery, for example, using Ambion Rnaqueous 4-PCR purification kit for large scale extractions, or Ambion Rnaqueous-96 purification kit for 96-well assays. For Taqman analysis, dual-labeled probes are synthesized with, for example, the reporter dyes FAM or VIC covalently linked at the 5′-end and the quencher dye TAMARA conjugated to the 3′-end. One-step RT-PCR amplifications are performed on, for example, an ABI PRISM 7700 Sequence detector using 50 uL reactions consisting of 10 uL total RNA, 100 nM forward primer, 100 mM reverse primer, 100 nM probe, 1×TaqMan PCR reaction buffer (PE-Applied Biosystems), 5.5 mM MgCl2, 100 uM each dATP, dCTP, dGTP and dTTP, 0.2 U RNase Inhibitor (Promega), 0.025 U AmpliTaq Gold (PE-Applied Biosystems) and 0.2 U M-MLV Reverse Transcriptase (Promega). The thermal cycling conditions can consist of 30 minutes at 48° C., 10 minutes at 95° C., followed by 40 cycles of 15 seconds at 95° C. and 1 minute at 60° C. Quantitation of target KRAS mRNA level is determined relative to standards generated from serially diluted total cellular RNA (300, 100, 30, 10 ng/r×n) and normalizing to, for example, 36B4 mRNA in either parallel or same tube TaqMan reactions.

Western Blotting

Nuclear extracts can be prepared using a standard micro preparation technique (see for example Andrews and Faller, 1991, Nucleic Acids Research, 19, 2499). Protein extracts from supernatants are prepared, for example using TCA precipitation. An equal volume of 20% TCA is added to the cell supernatant, incubated on ice for 1 hour and pelleted by centrifugation for 5 minutes. Pellets are washed in acetone, dried and resuspended in water. Cellular protein extracts are run on a 10% Bis-Tris NuPage (nuclear extracts) or 4-12% Tris-Glycine (supernatant extracts) polyacrylamide gel and transferred onto nitro-cellulose membranes. Non-specific binding can be blocked by incubation, for example, with 5% non-fat milk for 1 hour followed by primary antibody for 16 hours at 4° C. Following washes, the secondary antibody is applied, for example (1:10,000 dilution) for 1 hour at room temperature and the signal detected with SuperSignal reagent (Pierce).

In several cell culture systems, cationic lipids have been shown to enhance the bioavailability of oligonucleotides to cells in culture (Bennet, et al., 1992, Mol. Pharmacology, 41, 1023-1033). In one embodiment, DsiRNA molecules of the invention are complexed with cationic lipids for cell culture experiments. DsiRNA and cationic lipid mixtures are prepared in serum-free DMEM immediately prior to addition to the cells. DMEM plus additives are warmed to room temperature (about 20-25° C.) and cationic lipid is added to the final desired concentration and the solution is vortexed briefly. DsiRNA molecules are added to the final desired concentration and the solution is again vortexed briefly and incubated for 10 minutes at room temperature. In dose response experiments, the RNA/lipid complex is serially diluted into DMEM following the 10 minute incubation.

### Animal Models

Evaluating the efficacy of dsRNA-peptide agents in animal models is an important prerequisite to human clinical trials. Various animal models of cancer and/or proliferative diseases, conditions, or disorders as are known in the art can be adapted for use for pre-clinical evaluation of the efficacy of DsiRNA compositions of the invention in modulating target gene expression toward therapeutic use.

For example, if the target is KRAS, as in cell culture models, the most Ras sensitive mouse tumor xenografts are those derived from cancer cells that express mutant Ras proteins. Nude mice bearing H-Ras transformed bladder cancer cell xenografts were sensitive to an anti-Ras antisense nucleic acid, resulting in an 80% inhibition of tumor growth after a 31 day treatment period (Wickstrom, 2001, Mol. Biotechnol., 18, 35-35). Zhang et al., 2000, Gene Ther., 7, 2041, describes an anti-KRAS ribozyme adenoviral vector (KRbz-ADV) targeting a KRAS mutant (KRAS codon 12 GGT to GTT; H441 and H1725 cells respectively). Non-small cell lung cancer cells (NSCLC H441 and H1725 cells) that express the mutant KRas protein were used in nude mouse xenografts compared to NSCLC H1650 cells that lack the relevant mutation. Pre-treatment with KRbz-ADV completely abrogated engraftment of both H441 and H1725 cells and compared to 100% engraftment and tumor growth in animals that received untreated tumor cells or a control vector. Additional mouse models of KRAS misregulation/mutation have also been described (e.g., in Kim et al. Cell 121: 823-835, which identified a role of KRAS in promoting lung adenocarcinomas). The above studies provide proof that inhibition of Ras expression (e.g., KRAS expression) by anti-Ras agents causes inhibition of tumor growth in animals.

As such, these models can be used in evaluating the efficacy of DsiRNA molecules of the invention in inhibiting KRAS levels, expression, tumor/cancer formation, growth, spread, development of other KRAS-associated phenotypes, diseases or disorders, etc. These models and others can similarly be used to evaluate the safety/toxicity and efficacy of DsiRNA molecules of the invention in a pre-clinical setting.

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

The present invention is described by reference to the following Examples, which are offered by way of illustration and are not intended to limit the invention in any manner. Standard techniques well known in the art or the techniques specifically described below were utilized.

### Example 1

### Preparation of Double-Stranded RNA Oligonucleotides

Oligonucleotide Synthesis and Purification

DsiRNA molecules can be designed to interact with various sites in the RNA message, for example, target sequences within the RNA sequences described herein. The DsiRNA molecules are chemically synthesized using methods described herein. Generally, DsiRNA constructs are synthesized using solid phase oligonucleotide synthesis methods as described for 19-23mer siRNAs (see for example Usman et al., U.S. Pat. Nos. 5,804,683; 5,831,071; 5,998,203; 6,117,657; 6,353,098; 6,362,323; 6,437,117; 6,469,158; Scaringe et al., U.S. Pat. Nos. 6,111,086; 6,008,400; 6,111,086).

Individual RNA strands are synthesized and HPLC purified according to standard methods (Integrated DNA Technologies, Coralville, Iowa). For example, RNA oligonucleotides are synthesized using solid phase phosphoramidite chemistry, deprotected and desalted on NAP-5 columns (Amersham Pharmacia Biotech, Piscataway, N.J.) using standard techniques (Damha and Olgivie, 1993, Methods Mol Biol 20: 81-114; Wincott et al., 1995, Nucleic Acids Res 23: 2677-84). The oligomers are purified using ion-exchange high performance liquid chromatography (IE-HPLC) on an Amersham Source 15Q column (1.0 cm×25 cm; Amersham Pharmacia Biotech, Piscataway, N.J.) using a 15 min step-linear gradient. The gradient varies from 90:10 Buffers A:B to 52:48 Buffers A:B, where Buffer A is 100 mM Tris pH 8.5 and Buffer B is 100 mM Tris pH 8.5, 1 M NaCl. Samples are monitored at 260 nm and peaks corresponding to the full-length oligonucleotide species are collected, pooled, desalted on NAP-5 columns, and lyophilized.

The purity of each oligomer was determined by capillary electrophoresis (CE) on a Beckman PACE 5000 (Beckman Coulter, Inc., Fullerton, Calif.). The CE capillaries had a 100 μm inner diameter and contains ssDNA 100R Gel (Beckman-Coulter). Typically, about 0.6 nmole of oligonucleotide was injected into a capillary, run in an electric field of 444 V/cm and detected by UV absorbance at 260 nm. Denaturing Tris-Borate-7 M-urea running buffer was purchased from Beckman-Coulter. Oligoribonucleotides were obtained that are at least 90% pure as assessed by CE for use in experiments described below. Compound identity was verified by matrix-assisted laser desorption ionization time-of-flight (MALDI-TOF) mass spectroscopy on a Voyager DE™ Biospectometry Work Station (Applied Biosystems, Foster City, Calif.) following the manufacturer's recommended protocol. Relative molecular masses of all oligomers were obtained, often within 0.2% of expected molecular mass.

Preparation of Duplexes

Single-stranded RNA (ssRNA) oligomers were resuspended, e.g., at 100 μM concentration in duplex buffer consisting of 100 mM potassium acetate, 30 mM HEPES, pH 7.5. Complementary sense and antisense strands were mixed in equal molar amounts to yield a final solution of, e.g., 50 μM duplex. Samples were heated to 100° C. for 5′ in RNA buffer (IDT) and allowed to cool to room temperature before use. Double-stranded RNA (dsRNA) oligomers were stored at −20° C. Single-stranded RNA oligomers were stored lyophilized or in nuclease-free water at −80° C.

Nomenclature

For consistency, the following nomenclature has been employed in the instant specification. Names given to duplexes indicate the length of the oligomers and the presence or absence of overhangs. A “ 25/27” is an asymmetric duplex having a 25 base sense strand and a 27 base antisense strand with a 2-base 3′-overhang. A “ 27/25” is an asymmetric duplex having a 27 base sense strand and a 25 base antisense strand.

Cell Culture and RNA Transfection

HeLa cells were obtained from ATCC and maintained in Dulbecco's modified Eagle medium (HyClone) supplemented with 10% fetal bovine serum (HyClone) at 37° C. under 5% CO₂. For RNA transfections, HeLa cells were transfected with DsiRNAs as indicated at a final concentration of 1 nM or 0.1 nM using Lipofectamine™ RNAiMAX (Invitrogen) and following manufacturer's instructions. Briefly, 2.5 μL of a 0.2 μM or 0.02 μM stock solution of each DsiRNA were mixed with 46.5 μL of Opti-MEM I (Invitrogen) and 1 μL of Lipofectamine™ RNAiMAX. The resulting 50 μL mix was added into individual wells of 12 well plates and incubated for 20 min at RT to allow DsiRNA:Lipofectamine™ RNAiMAX complexes to form. Meanwhile, HeLa cells were trypsinized and resuspended in medium at a final concentration of 367 cells/μL. Finally, 450 μL of the cell suspension were added to each well (final volume 500 μL) and plates were placed into the incubator for 24 hours.

Assessment of Inhibition

Target gene knockdown was determined by qRT-PCR, with values normalized to HPRT expression control treatments, including Lipofectamine™ RNAiMAX alone (Vehicle control) or untreated.

RNA Isolation and Analysis

Cells were washed once with 2 mL of PBS, and total RNA was extracted using RNeasy Mini Kit™ (Qiagen) and eluted in a final volume of 30 μL. 1 μg of total RNA was reverse-transcribed using Transcriptor 1^{st} Strand cDNA Kit™ (Roche) and random hexamers following manufacturer's instructions. One-thirtieth (0.66 μL) of the resulting cDNA was mixed with 5 μL of IQ Multiplex Powermix (Bio-Rad) together with 3.33 μL of H₂O and 1 μL of a 3 μM mix containing primers and probes specific for human genes HPRT-1 (accession number NM_{—}000194) and KRAS target sequences.

Quantitative RT-PCR

A CFX96 Real-time System with a C1000 Thermal cycler (Bio-Rad) was used for the amplification reactions. PCR conditions were: 95° C. for 3 min; and then cycling at 95° C., 10 sec; 55° C., 1 min for 40 cycles. Each sample was tested in triplicate. Relative HPRT mRNA levels were normalized to target mRNA levels and compared with mRNA levels obtained in control samples treated with the transfection reagent alone, or untreated. Data was analyzed using Bio-Rad CFX Manager version 1.0 software.

### Example 2

### Preparation and Use of DsiRNA-Delivery Peptide Conjugates

Oligonucleotide-delivery peptide conjugates of the present invention were synthesized with chemistry based on the conjugation of HyNic (6-Hydrazinonicotinamide)-modified peptides to 4FB (4-Formylbenzamide)-modified oligonucleotides. Other peptide synthesis methods and conjugation procedures known in the art are also applicable.

HyNic moieties were incorporated on a peptide at either N- or C-termini using 6-Boc-HyNic or FMOC-Lys-(ε-6-BocHyNic)OH, respectively. Cleavage from resin was accomplished using TFA/acetone/water/triisopropylsilane (TIS) (92.5/2.5/2.5/2.5) for 2 hours. The presence of the acetone forms a hydrazone with the deprotected hydrazine moiety in situ blocking any trifluoroacetamide formation from the reaction of TFA with the strongly nucleophilic hydrazine. Crude peptides were analyzed by HPLC and ES-MS. Products were isolated by RP-HPLC using a gradient method. For Peg12 peptides, polyethylene glycol synthons were directly added during solid phase peptide synthesis. In some instances, additional polyethylene glycol spacers were also added to the oligonucleotide termini using polyethylene glycol oligonucleotide synthons.

Amino-modified oligonucleotides were converted to 5′-4FB-oligonucleotides. Linking of HyNic-peptides to 4FB-modified oligonucleotides was performed at a 2-5 mole excess of HyNic-peptide and generally produced >80% conjugate yield. Hydrazone bond formation was catalyzed and reaction kinetics improved 10-100-fold via inclusion of aniline, generally leading to conjugation yields >95%. Optimal conjugation kinetics (formation of the hydrazone bond) was achieved between pH 4.5-5.0. However, the reaction also can proceed at higher pH, albeit at a slower rate. The optimum pH for each conjugation was determined empirically, also taking into account the solubility of the different peptide sequences. The degree of conjugation can be monitored spectrophotometrically. Formation of the bis-aryl hydrazone bond was utilized both to trace and to quantify progress of the conjugation reaction, using the known molar extinction coefficient (29,000 @ 354 nm). Diafiltration was used to remove excess peptide, yielding the oligonucleotide-peptide conjugates. To produce HyNic-quenched peptides, HyNic-peptides were reacted with 2-Sulfobenzaldehyde to inactivate the HyNic reactive moiety on the peptide.

Cell Free Dicing Assay

DsiRNA or peptide-conjugated DsiRNA (final concentration at 5 μM) was incubated with recombinant human dicer enzyme mixture (Genlantis, #T52002) at 37° C. for 2 hrs, and the reaction was stopped with stop solution. This final solution was mixed with gel loading buffer (Bio-Rad, #161-0767). Dicer-cleaved dsRNAs and intact DsiRNAs were resolved by 18% native polyacrylamide gel electrophoresis. Gel images were obtained using the Bio-Rad VersaDoc™ imaging system (model#4000 MP).

Serum Stability Assay

DsiRNA or peptide-conjugated DsiRNA (2 μM final concentration) was incubated in 90% (v/v) mouse serum (Sigma #M5905) at 37° C. At different time points (0, 2, 4, 8, 1, 10 & 25 hours), 10 μL sample was mixed with 2 μL H2O and 3 μL gel loading buffer (Bio-Rad #161-0767) and was immediately flash frozen in an alcohol-dry ice bath. Samples were electrophoresed on an 18% native polyacrylamide gel (Bio-Rad #161-1216). Resolved siRNA bands were quantified using the Bio-Rad VersaDoc™ imaging system (Bio-Rad model#4000 MP). The half-life of individual dsRNAs in 90% serum was calculated by plotting the change in dsRNA band intensity over time.

HPRT1- and KRAS-Targeting DsiRNAs

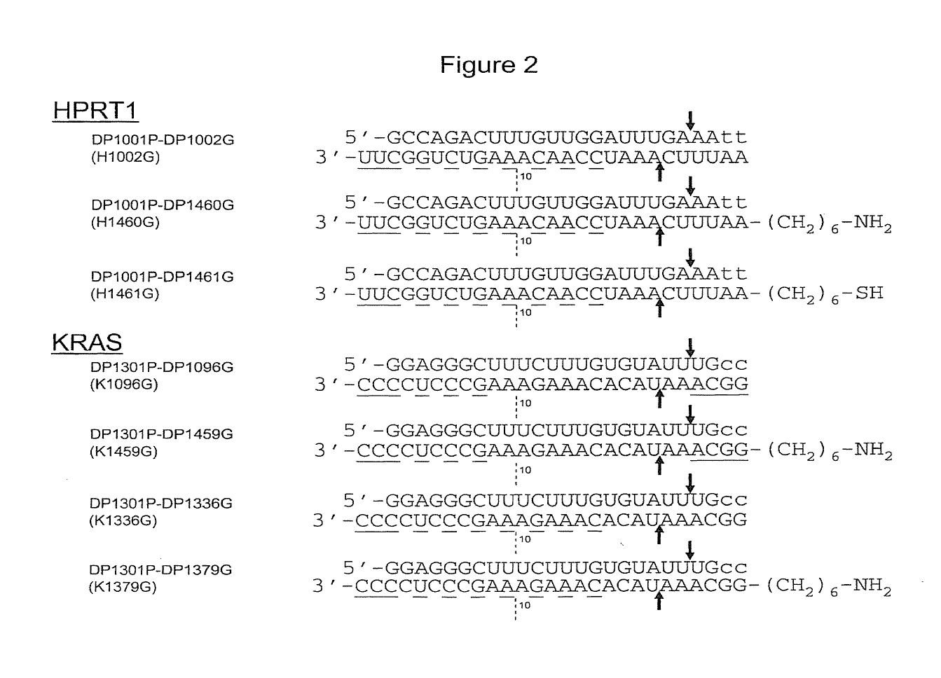

Exemplary DsiRNAs directed against HPRT1 and KRAS target genes were synthesized as described herein, with DsiRNAs possessing the oligonucleotide sequences, 2′-O-methyl and end modifications shown in FIG. 2.

Conjugated Peptides

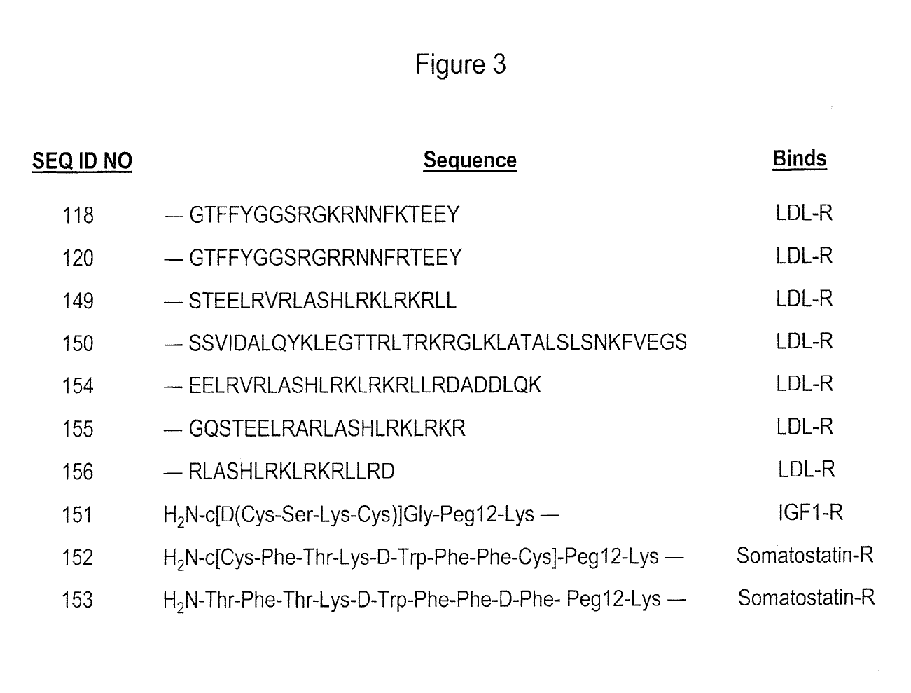

Exemplary delivery peptides used or capable of use in conjugation with the DsiRNAs of the instant invention are listed in FIG. 3, which also indicates the binding target associated with each individual delivery peptide.

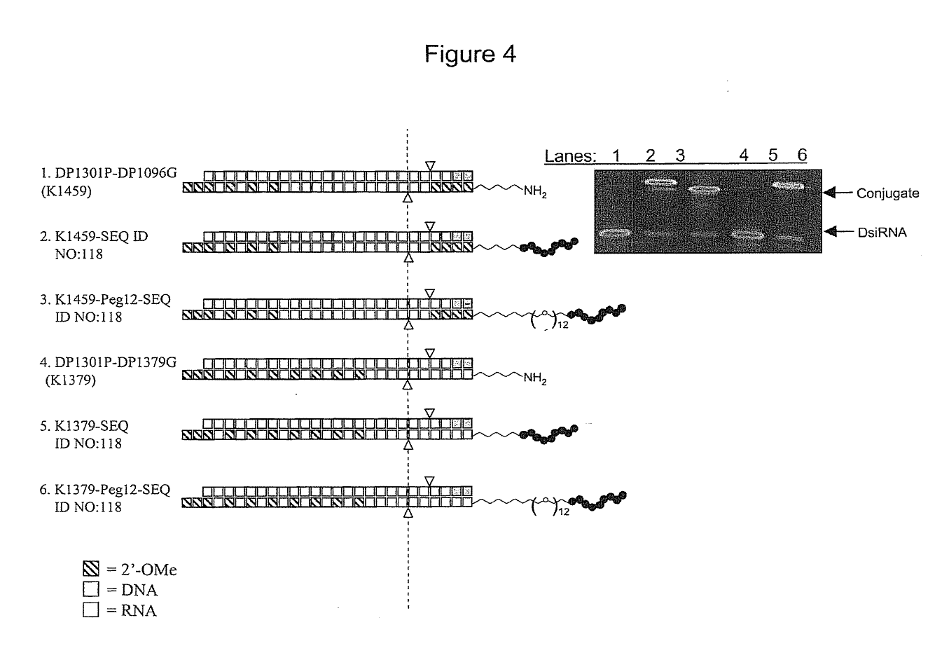

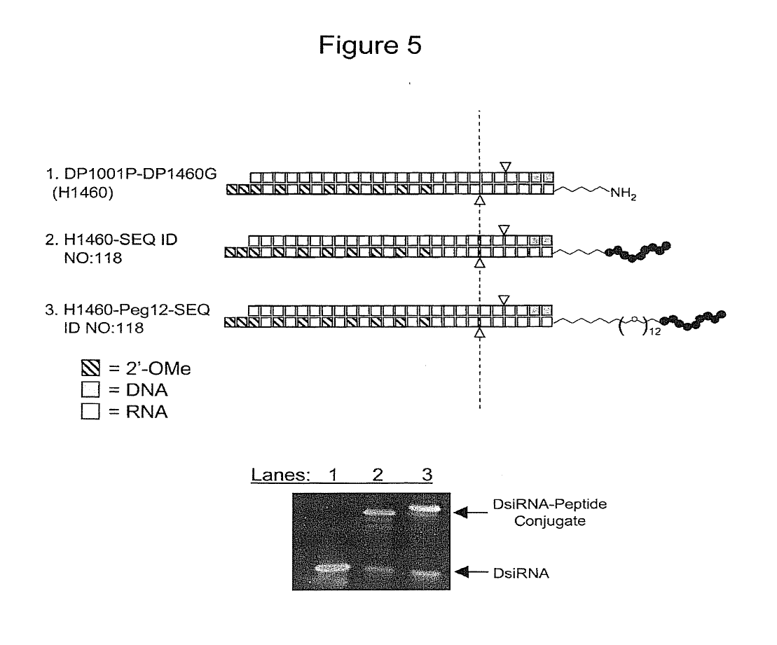

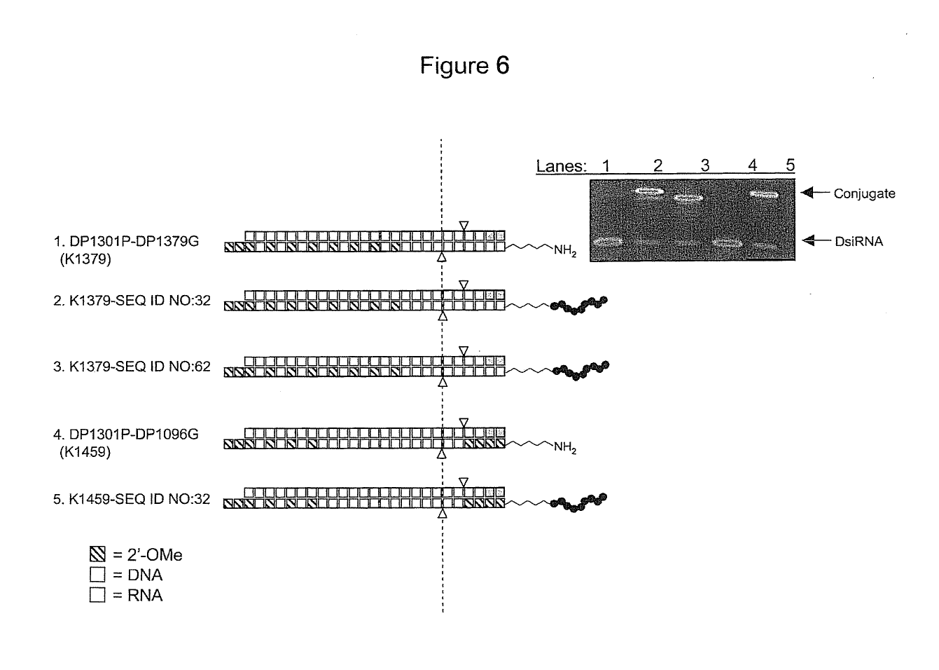

The successful synthesis of various peptide-DsiRNA conjugates was confirmed via observation of the increased size (and, therefore, retarded electrophoretic mobility) associated with a successful conjugation. As shown in FIGS. 4-6, both HPRT1- and KRAS-targeting DsiRNAs were successfully conjugated with a number of peptides, forming the following conjugates: K1459-SEQ ID NO:118; K1459-Peg12-SEQ ID NO:118; K1379-SEQ ID NO:118; K1379-Peg12-SEQ ID NO:118 (FIG. 4); H1460-SEQ ID NO:118; H1460-Peg12-SEQ ID NO:118 (FIG. 5); K1379-SEQ ID NO:149; K1379-SEQ ID NO:154; K1379-SEQ ID NO:155; and K1379-SEQ ID NO:156 (FIG. 6).

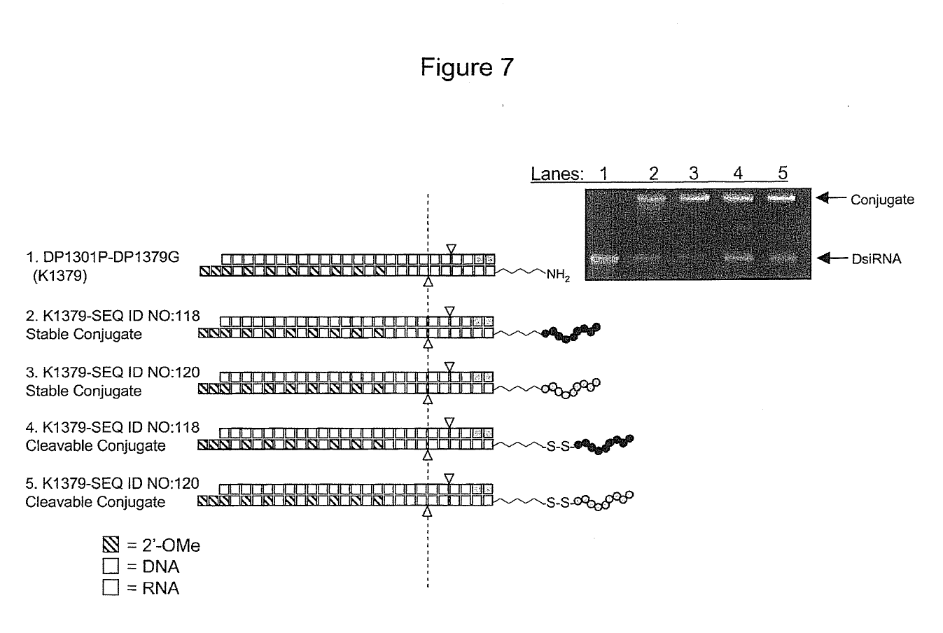

DsiRNA-peptide conjugates possessing cleavable linker moieties (e.g., disulfide groups) were also successfully synthesized. Specifically, FIG. 7 shows that both stable and cleavable conjugates of SEQ ID NOs: 118 and 120 peptides with KRAS-targeting DsiRNA K1379 were produced.

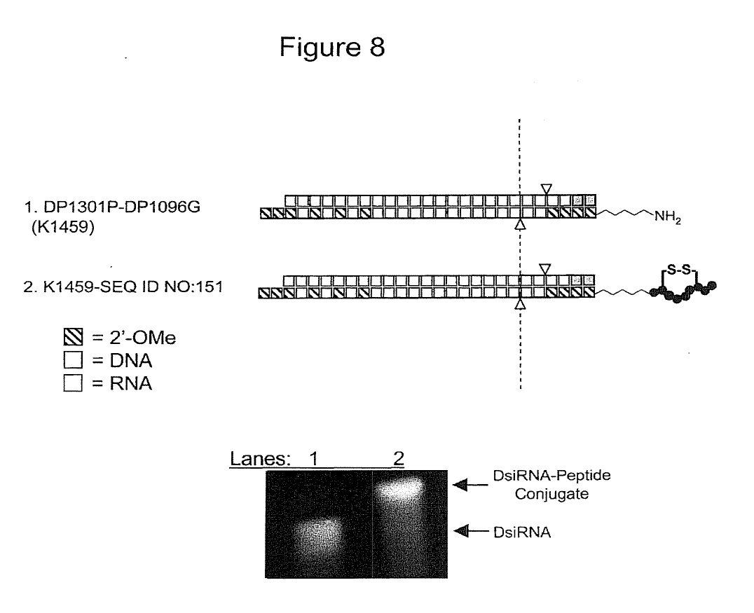

DsiRNAs were also conjugated with cyclic peptides. As shown in FIG. 8, the KRAS-targeting DsiRNA, K1459, was successfully conjugated with cyclic peptide SEQ ID NO:151.

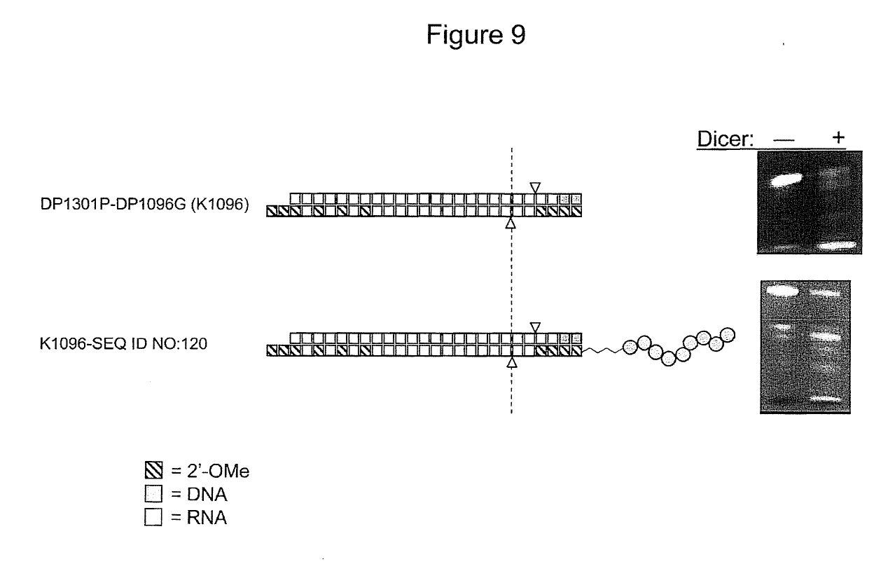

To assess Dicer cleavage of an exemplary peptide-DsiRNA conjugate, a cell-free dicing assay was performed as described above upon the K1096-SEQ ID NO:120 conjugate. As shown in FIG. 9, this conjugate was confirmed to have been cleaved by Dicer.

### Example 3

### Transfected dsRNA-Delivery Peptide Conjugates Reduced Expression of Target Gene Levels in a Cell

Cell Culture and RNA Transfection

HeLa and HepG2 cells were obtained from ATCC and maintained in Dulbecco's modified Eagle medium (HyClone) supplemented with 10% fetal bovine serum (HyClone) at 37° C. under 5% CO₂. For dsRNA and dsRNA-delivery peptide conjugate transfections, HeLa cells were transfected with the unconjugated or conjugated DsiRNAs at indicated final concentrations (e.g., 1 nM or 0.1 nM) in the presence of Lipofectamine™ RNAiMAX (Invitrogen). In certain examples, unconjugated DsiRNAs were also used as positive controls. In certain examples, 2.5 μL of a 0.2 μM or 0.02 μM stock solution of each DsiRNA was mixed with 47.5 μL of Opti-MEM I (Invitrogen). For Lipofectamine™ controls, 2.5 μL of a 0.2 μM or 0.02 μM stock solution of each DsiRNA was mixed with 46.5 μL of Opti-MEM I (Invitrogen) and 1 μL of Lipofectamine™ RNAiMAX. The resulting 50 μL mix was added into individual wells of 12 well plates and incubated for 20 minutes at room temperature to allow DsiRNA:Lipofectamine™ RNAiMAX complexes to form. Meanwhile, HeLa or HepG2 cells were trypsinized and resuspended in medium at a final concentration of about 367 cells/μL. Finally, 450 μL of the cell suspension was added to each well (final volume 500 μL) and plates were placed into the incubator for 24 hours. For dose-response studies, the concentrations of transfected DsiRNAs were varied from initially 1 μM to 1 nM. For dose-response studies involving DsiRNA-peptide conjugates administered to cells in the absence of transfection vehicle, the concentrations of administered DsiRNAs and DsiRNA-peptide conjugates were varied from approximately 5 nM to approximately 5 μM. Time course studies can also be performed, with incubation times of about 4 hours to about 72 hours studied.

Assessment of Inhibition

Target gene knockdown was determined by qRT-PCR, with values normalized to HPRT expression control treatments, including Lipofectamine™ RNAiMAX alone (Vehicle control) or untreated.

RNA Isolation and Analysis

Cells were washed once with 2 mL of PBS, and total RNA was extracted using RNeasy Mini Kit™ (Qiagen) and eluted in a final volume of 30 μL. 1 μg of total RNA was reverse-transcribed using Transcriptor 1^{st} Strand cDNA Kit™ (Roche) and random hexamers following manufacturer's instructions. One-thirtieth (0.66 μL) of the resulting cDNA was mixed with 5 μL of IQ Multiplex Powermix (Bio-Rad) together with 3.33 μL of H₂O and 1 μL of a 3 μM mix containing primers and probes specific for human genes HPRT-1 (accession number NM_{—}000194) and KRAS target sequences.

Quantitative RT-PCR

A CFX96 Real-time System with a C1000 Thermal cycler (Bio-Rad) was used for the amplification reactions. PCR conditions were: 95° C. for 3 min; and then cycling at 95° C., 10 sec; 55° C., 1 min for 40 cycles. Each sample was tested in duplicate (with duplicate experiments performed for each agent for which data is shown in FIGS. 2-19). Relative HPRT mRNA levels were normalized to target mRNA levels and compared with mRNA levels obtained in control samples treated with the transfection reagent alone, or untreated. Data were analyzed using Bio-Rad CFX Manager version 1.0 software. Expression data were presented as a comparison of the expression under the treatment of unconjugated dsRNA versus that of dsRNA-delivery peptide conjugates.

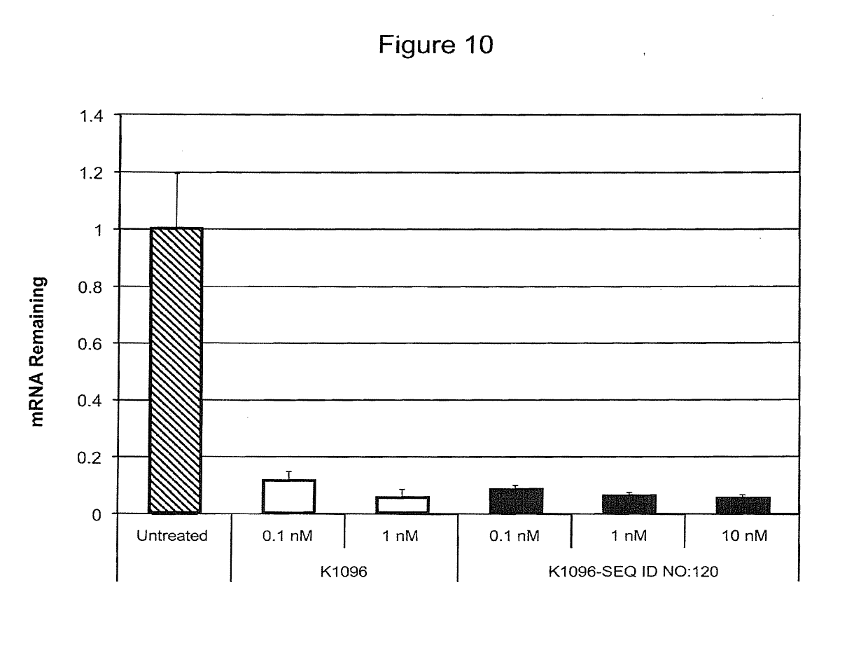

DsiRNA-peptide conjugates were initially examined for the ability to inhibit target mRNA levels in a cell when administered via transfection. As shown in FIG. 10, the KRAS-targeting DsiRNA K1096-SEQ ID NO:120 was observed to inhibit target mRNA levels by at least 80% when administered to HeLa cells via transfection at 0.1 nM and higher concentrations. The levels of target gene inhibition seen for peptide-DsiRNA conjugate were observed to be similar to those seen for the free K1096 DsiRNA. Accordingly, a peptide-DsiRNA conjugate was observed to be an effective inhibitor of target RNA levels when administered to HeLa cells via transfection.

### Example 4

### DsiRNA-Peptide Conjugates Demonstrated Stability in Serum

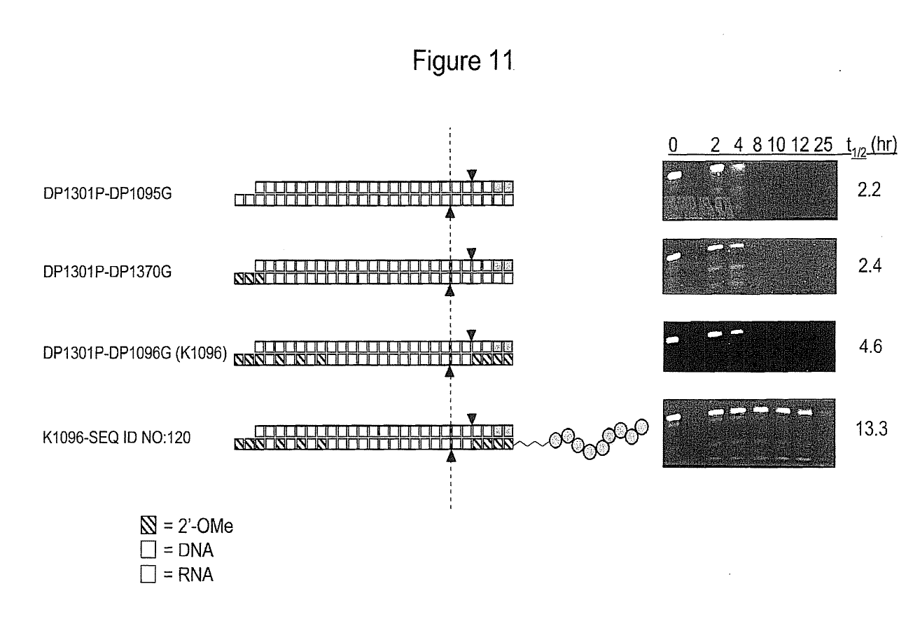

Serum stability of DsiRNA agents was assessed as described herein. As shown in FIG. 11, conjugation of the DsiRNA K1096 with the SEQ ID NO:120 peptide resulted in significantly extended half-lives for the conjugated agent (K1096-SEQ ID NO:120 (half life=13.3 hours)), as compared to another DsiRNA possessing an otherwise identical modification pattern but lacking the SEQ ID NO:120 peptide.

### Example 5

### dsRNA-Peptide Conjugates (e.g., dsRNA-Delivery Peptide Conjugates) Administered without Transfection Vehicle Reduced Expression of Target Gene Levels in a Cell

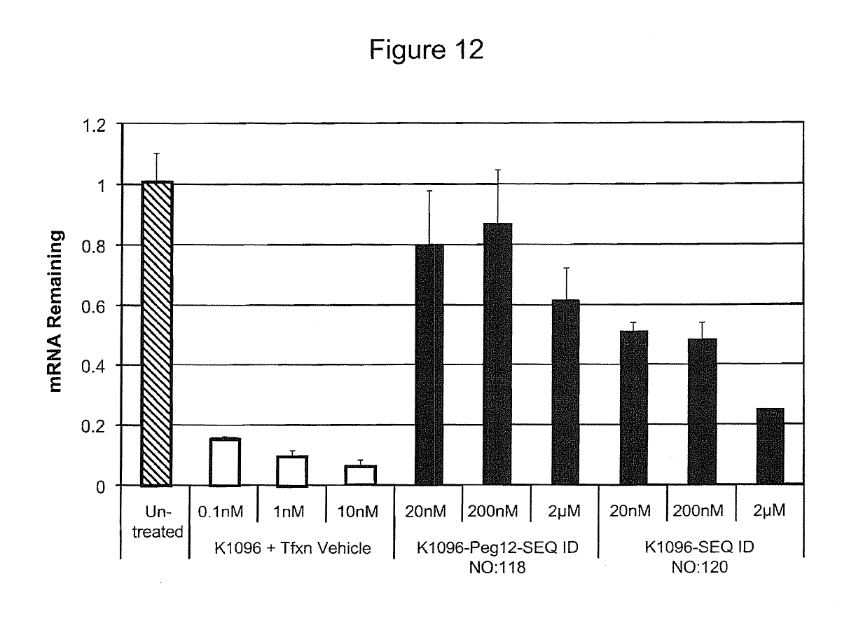

To assess the ability of exemplified DsiRNA-peptide conjugates to promote delivery of such conjugated agents to target cells in the absence of any transfection vehicle, a series of DsiRNA-peptide conjugates were administered to HeLa cells at concentrations ranging from 20 nM to 2 μM. As shown in FIG. 12, elevated concentrations of DsiRNA-peptide conjugates were required to achieve significant inhibition of target mRNA levels, as compared to transfected DsiRNAs. However, the two tested conjugates (K1096-Peg12-SEQ ID NO:118 and K1096-SEQ ID NO:120) behaved in a dose-responsive manner, with significant levels of target gene inhibition observed for both conjugated molecules at 2 μM concentration. Indeed, the K1096-SEQ ID NO:120 conjugate exhibited surprising and significant reduction of target mRNA levels at all tested concentrations (20 nM, 200 nM and 2 μM).

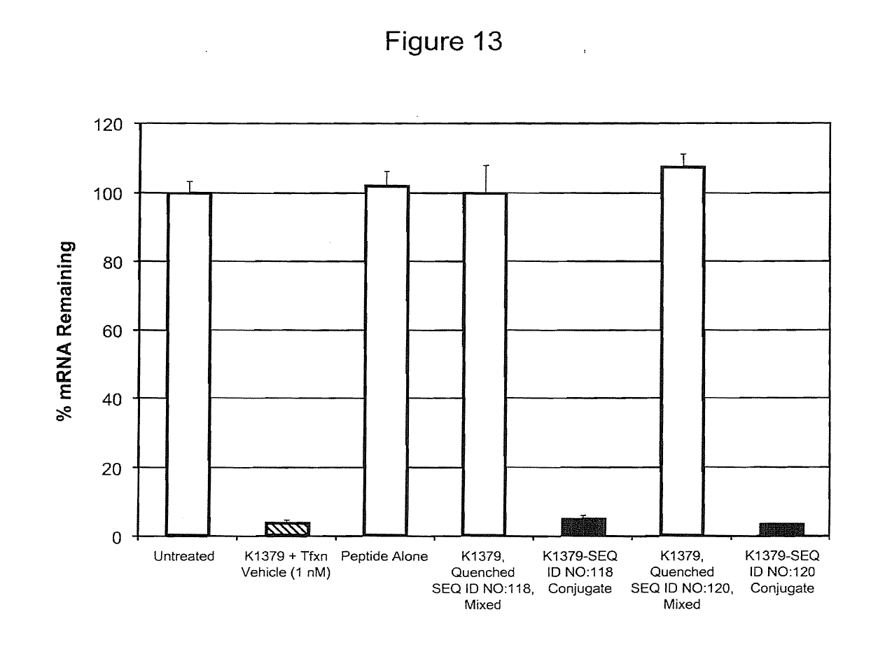

DsiRNA-peptide conjugates K1379-SEQ ID NO:118 and K1379-SEQ ID NO:120 were then assessed for the ability to inhibit the KRAS target gene when administered to HepG2 cells at a concentration of 5 μM in the absence of transfection vehicle. As shown in FIG. 13, remarkably, greater than 90% reduction in target mRNA levels were observed for both conjugates administered to HepG2 cells at a concentration of 5 μM in the absence of transfection vehicle. Such results were similar to results observed for the K1379 DsiRNA alone administered at 1 nM to HepG2 cells. Surprisingly, inclusion of a quenched peptide (either quenched SEQ ID NO:118 or quenched SEQ ID NO:120) with free DsiRNA K1379 in a mixture that was administered to HepG2 cells resulted in a complete loss of any target mRNA inhibition activity.

To examine whether exemplary DsiRNA-peptide conjugates were more efficient inhibitors of target mRNA levels in the absence of delivery vehicle than corresponding free DsiRNAs, dose-response curves were obtained that compared free K1379 DsiRNA with K1379-SEQ ID NO:118 conjugate and also compared free K1379 DsiRNA with the K1379-SEQ ID NO:120 conjugate. As shown in FIG. 14, K1379-peptide conjugates performed significantly better than corresponding free K1379 DsiRNAs across all IC₅₀-informative concentrations. Indeed, measured IC₅₀ values for DsiRNA-peptide conjugates in the absence of transfection vehicle were two- to three-fold higher (and therefore less potent) for free DsiRNA as compared to corresponding DsiRNA-peptide conjugates.

### Example 6

### Preparation of Additional Delivery Peptide-DsRNA and/or Targeting Peptide-dsRNA Conjugates

Additional preferred target DsiRNA agents are selected from a pre-screened population of DsiRNAs. Design of DsiRNAs can optionally involve use of predictive scoring algorithms that perform in silico assessments of the projected activity/efficacy of a number of possible DsiRNAs spanning a region of sequence.

A dsRNA of the invention is conjugated to a delivery peptide or a targeting peptide by any of the methods described herein above. About 20 mg of DsiRNAs (˜1 μmoles) with 5′ amino group are reacted with 3-5 molar excess of peptides with terminal Cys sulfhydryl group using maleimide chemistry (Moschos et al., Bioconjug Chem. 2007; 18(5):1450-9; Nishina et al., Mol. Ther. 2008; 16(4):734-40). The peptide-RNA conjugates are purified by diafiltration to remove excess peptide, desalted and supplied as lyophilized powder. The purity of the final products is determined by analytical anion-exchange HPLC and electrospray mass spectroscopy with deconvolution.

### Example 7

### Additional use of a dsRNA-Targeting Peptide Conjugate to Reduce Expression of a Target Gene in a Cell

Cell Culture and RNA Transfection

HeLa, Hep3B, HepG2, HT29, LS174T, and Neuro2a are obtained from ATCC and maintained in the recommended basal medium with 10% heat-inactivated FBS at 37° C. under 5% CO₂. For dsRNA and dsRNA-targeting peptide conjugate transfections, cells are transfected with the unconjugated or conjugated DsiRNAs as indicated at a final concentration of 1 nM or 0.1 nM. Lipofectamine™ RNAiMAX (Invitrogen). DsiRNAs are used as positive controls. Briefly, 2.5 μL of a 0.2 μM or 0.02 μM stock solution of each DsiRNAs is mixed with 47.5 μL of Opti-MEM I (Invitrogen). For Lipofectamine™ control, 2.5 μL of a 0.2 μM or 0.02 μM stock solution of each DsiRNAs is mixed with 46.5 μL of Opti-MEM I (Invitrogen) and 1 μL of Lipofectamine™ RNAiMAX. The resulting 50 μL mix is added into individual wells of 12 well plates and incubated for 20 min at RT to allow DsiRNA:Lipofectamine™ RNAiMAX complexes to form. Meanwhile, cells are trypsinized and resuspended in medium at a final concentration of about 367 cells/μL. Finally, 450 μL of the cell suspension are added to each well (final volume 500 μL) and plates are placed into the incubator for 24 hours. For dose response study, the concentrations of DsiRNAs are varied from initially 1 pM to 1 nM. For time course studies, incubation times of about 4 hours to about 72 hours are studied.

Assessment of Inhibition

Target gene knockdown is determined by qRT-PCR, with values normalized to HPRT expression control treatments, including Lipofectamine™ RNAiMAX alone (Vehicle control) or untreated.

RNA Isolation and Analysis

Cells are washed once with 2 mL of PBS, and total RNA is extracted using RNeasy Mini Kit™ (Qiagen) and eluted in a final volume of 30 μL. 1 μg of total RNA is reverse-transcribed using Transcriptor 1^{st} Strand cDNA Kit™ (Roche) and random hexamers following manufacturer's instructions. One-thirtieth (0.66 μL) of the resulting cDNA is mixed with 5 μL of IQ Multiplex Powermix (Bio-Rad) together with 3.33 μL of H₂O and 1 μL of a 3 μM mix containing primers and probes specific for human genes HPRT-1 (accession number NM_{—}000194) and KRAS target sequences.

Quantitative RT-PCR

A CFX96 Real-time System with a C1000 Thermal cycler (Bio-Rad) is used for the amplification reactions. PCR conditions are: 95° C. for 3 min; and then cycling at 95° C., 10 sec; 55° C., 1 min for 40 cycles. Each sample is tested in triplicate. Relative HPRT mRNA levels are normalized to target mRNA levels and compared with mRNA levels obtained in control samples treated with the transfection reagent alone, or untreated. Data are analyzed using Bio-Rad CFX Manager version 1.0 software. Expression data are presented as a comparison of the expression under the treatment of unconjugated dsRNA versus that of dsRNA-targeting peptide conjugates.

### Example 8

### Use of a dsRNA-Delivery Peptide Conjugate to Reduce Expression of a Target Gene in an Animal

In order to assess the efficiency of delivery and subsequent functionality of the dsRNAs, peptides and dsRNA-delivery peptide conjugates, subcutaneous (s.c.) tumor models (Judge et al., J Clin Invest. 2009; 119(3):661-73) are utilized. Hep3B tumors are established in female SCID/beige mice by s.c. injection of 3×10⁶ cells in 50 μL PBS into the left-hind flank. Mice are randomized into treatment groups 10-17 days after seeding as tumors became palpable. Formulations of dsRNA, peptide and dsRNA-delivery peptide conjugates or PBS vehicle controls are administered by standard intravenous (i.v.) injection via the lateral tail vein, calculated based on a mg dsRNAs/kg body weight basis according to individual animal weights. Tumors are measured in 2 dimensions (width×length) to assess tumor growth using digital calipers. Tumor volume is calculated using the equation x*y*y/2, where x=largest diameter and y=smallest diameter, and is expressed as group mean±SD. Tumor tissues are also removed from the animals of different treatment groups and gene knockdown is confirmed. Tumor volume, survival and RNA expression data are presented as a comparison between the treatments of unconjugated dsRNA versus dsRNA-delivery peptide conjugates.

### Example 9

### Use of a dsRNA-Targeting Peptide Conjugate to Reduce Expression of a Target Gene in a Cell

In order to assess the efficiency of targeting and subsequent functionality of the dsRNAs, peptide and dsRNA-targeting peptide conjugates, intrahepatic tumor models (Judge et al., J Clin Invest. 2009; 119(3):661-73) are used. Liver tumors are established in mice by direct intrahepatic injection of Hep3B or Neuro2a tumor cells. Female SCID/beige mice and male A/J mice are used as hosts for the Hep3B and Neuro2a tumors, respectively. Maintaining the mice under gas anesthesia, a single 1.5-cm incision across the midline is made below the sternum, and the left lateral hepatic lobe is exteriorized. 1×10⁶ Hep3B cells or 1×10⁵ Neuro2a cells suspended in 25 μL PBS are injected slowly into the lobe at a shallow angle using a Hamilton syringe and a 30-gauge needle. A swab is then applied to the puncture wound to stop any bleeding prior to suturing. Mice are allowed to recover from anesthesia in a sterile cage and monitored closely for 2-4 hours before being returned to conventional housing. Eight to eleven days after tumor implantation, mice are randomized into treatment groups: dsRNA, peptide and dsRNA-peptide conjugate formulations or PBS vehicle controls are administered by standard intravenous (i.v.) injection via the lateral tail vein, calculated based on a mg dsRNAs/kg body weight basis according to individual animal weights. Body weights are monitored throughout the duration of the study as an indicator of developing tumor burden and treatment tolerability. For efficacy studies, defined humane end points are determined as a surrogate for survival. Assessments are made based on a combination of clinical signs, weight loss, and abdominal distension to define the day of euthanization due to tumor burden. Tumor tissues are removed from the animals of different treatment groups and gene knockdown is confirmed.

Functionality of peptide, dsRNA and dsRNA-peptide conjugates for tumor cell targeting are also tested by labeling the peptide and/or dsRNA with fluorescent tags and performing fluorescent biodistribution studies using a live-animal imaging system (Xenogen or BioRad) (Eguchi et al., Nat. Biotechnol. 2009 May 17. [Epub ahead of print]). Using this methodology, and by comparing with the free (i.e., unconjugated) dsRNAs the ability of the peptide to bind to the tumor cell for both the peptide alone and dsRNA-peptide conjugates is confirmed. Unconjugated dsRNAs, used as a control in this study, by contrast, are unable to bind to the same extent as conjugated dsRNAs to the tumor surface. Efficacy end points, RNA expression and biodistribution data are presented as a comparison between the treatments with unconjugated dsRNA versus dsRNA-targeting peptide conjugates.

### Example 10

### Use of Additional Cell Culture Models to Evaluate the Down-Regulation of KRAS Gene Expression

A variety of endpoints have been used in cell culture models to look at Ras-mediated effects after treatment with anti-Ras agents. Phenotypic endpoints include inhibition of cell proliferation, RNA expression, and reduction of Ras protein expression. Because KRAS oncogene mutations are directly associated with increased proliferation of certain tumor cells, a proliferation endpoint for cell culture assays is preferably used as the primary screen. There are several methods by which this endpoint can be measured. Following treatment of cells with DsiRNA-peptide conjugates of the invention, cells are allowed to grow (typically 5 days), after which the cell viability, the incorporation of [³H] thymidine into cellular DNA and/or the cell density are measured. The assay of cell density can be done in a 96-well format using commercially available fluorescent nucleic acid stains (such as Syto® 13 or CyQuant®). As a secondary, confirmatory endpoint, a DsiRNA-peptide-mediated decrease in the level of KRas protein expression can be evaluated using a KRas-specific ELISA.

### Example 11

### Evaluation of Anti-KRAS DsiRNA Efficacy in a Mouse Model of KRAS Misregulation

Anti-KRAS DsiRNA-peptide conjugates chosen from in vitro assays can be further tested in mouse models, including, e.g., xenograft and other animal models as recited above. In one example, mice possessing misregulated (e.g., elevated) KRAS levels are administered a DsiRNA-peptide agent of the present invention via hydrodynamic tail vein injection. 3-4 mice per group (divided based upon specific DsiRNA agent tested) are injected with 50 μg or 200 μg of DsiRNA. Levels of KRAS RNA are evaluated using RT-qPCR. Additionally or alternatively, levels of KRas (e.g., KRas protein levels and/or cancer cell/tumor formation, growth or spread) can be evaluated using an art-recognized method, or phenotypes associated with misregulation of KRAS (e.g., tumor formation, growth, metastasis, etc.) are monitored (optionally as a proxy for measurement of KRAS transcript or KRas protein levels). Active DsiRNA-peptide conjugates in such animal models can also be subsequently tested in combination with standard chemotherapies.

### Example 12

### Diagnostic Uses

The dsRNA-peptide molecules of the invention can be used in a variety of diagnostic applications, such as in the identification of molecular targets (e.g., RNA) in a variety of applications, for example, in clinical, industrial, environmental, agricultural and/or research settings. Such diagnostic use of dsRNA-peptide molecules involves utilizing reconstituted RNAi systems, for example, using cellular lysates or partially purified cellular lysates. dsRNA-peptide molecules of this invention can be used as diagnostic tools to examine genetic drift and mutations within diseased cells. The close relationship between dsRNA-peptide activity and the structure of the target RNA allows the detection of mutations in any region of the target molecule, which alters the base-pairing and three-dimensional structure of the target RNA. By using multiple dsRNA-peptide molecules described in this invention, one can map nucleotide changes, which are important to RNA structure and function in vitro, as well as in cells and tissues. Cleavage of target RNAs with DsiRNA molecules can be used to inhibit gene expression and define the role of specified gene products in the progression of a disease or disorder associated with a particular target. In this manner, other genetic targets can be defined as important mediators of a disease. These experiments will lead to better treatment of the disease progression by affording the possibility of combination therapies (e.g., multiple DsiRNA molecules targeted to different genes, DsiRNA molecules coupled with known small molecule inhibitors, or intermittent treatment with combinations of DsiRNA molecules and/or other chemical or biological molecules). Other in vitro uses of DsiRNA molecules of this invention are well known in the art, and include detection of the presence of RNAs associated with a disease or related condition. Such RNA is detected by determining the presence of a cleavage product after treatment with a DsiRNA using standard methodologies, for example, fluorescence resonance emission transfer (FRET).

In a specific example, DsiRNA molecules that cleave only wild-type or mutant or polymorphic forms of the target KRAS RNA are used for the assay. The first DsiRNA molecules (i.e., those that cleave only wild-type forms of target KRAS RNA) are used to identify wild-type KRAS RNA present in the sample and the second DsiRNA molecules (i.e., those that cleave only mutant or polymorphic forms of target RNA) are used to identify mutant or polymorphic KRAS RNA in the sample. As reaction controls, synthetic substrates of both wild-type and mutant or polymorphic KRAS RNA are cleaved by both DsiRNA molecules to demonstrate the relative DsiRNA efficiencies in the reactions and the absence of cleavage of the “non-targeted” KRAS RNA species. The cleavage products from the synthetic substrates also serve to generate size markers for the analysis of wild-type and mutant KRAS RNAs in the sample population. Thus, each analysis requires two DsiRNA molecules, two substrates and one unknown sample, which is combined into six reactions. The presence of cleavage products is determined using an RNase protection assay so that full-length and cleavage fragments of each KRAS RNA can be analyzed in one lane of a polyacrylamide gel. It is not absolutely required to quantify the results to gain insight into the expression of mutant or polymorphic KRAS RNAs and putative risk of KRAS-associated phenotypic changes in target cells. The expression of KRAS mRNA whose protein product is implicated in the development of the phenotype (i.e., disease related/associated) is adequate to establish risk. If probes of comparable specific activity are used for both transcripts, then a qualitative comparison of KRAS RNA levels is adequate and decreases the cost of the initial diagnosis. Higher mutant or polymorphic form to wild-type ratios are correlated with higher risk whether KRAS RNA levels are compared qualitatively or quantitatively.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications can be made to the invention disclosed herein without departing from the scope and spirit of the invention. Thus, such additional embodiments are within the scope of the present invention and the following claims. The present invention teaches one skilled in the art to test various combinations and/or substitutions of chemical modifications described herein toward generating nucleic acid constructs with improved activity for mediating RNAi activity. Such improved activity can comprise improved stability, improved bioavailability, and/or improved activation of cellular responses mediating RNAi. Therefore, the specific embodiments described herein are not limiting and one skilled in the art can readily appreciate that specific combinations of the modifications described herein can be tested without undue experimentation toward identifying DsiRNA molecules with improved RNAi activity.

The invention illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description.

The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. Those skilled in the art will recognize, or be able to ascertain using no more than routine experimentation, many equivalents to the specific embodiments of the invention described herein. Such equivalents are intended to be encompassed by the following claims.

We claim:

1. An isolated double stranded ribonucleic acid (dsRNA) composition comprising a first oligonucleotide strand having a 5′ terminus and a 3′ terminus and a second oligonucleotide strand having a 5′ terminus and a 3′ terminus, wherein said first strand and said second strand have a length that is at least 16 and at most 50 nucleotides in length, wherein said peptide is conjugated to said dsRNA and wherein said dsRNA-peptide conjugate binds to a target.

2. An isolated double stranded ribonucleic acid (dsRNA) composition comprising a first oligonucleotide strand having a 5′ terminus and a 3′ terminus and a second oligonucleotide strand having a 5′ terminus and a 3′ terminus, wherein said first strand and said second strand have a length that is at least 16 and at most 50 nucleotides in length, wherein said peptide is conjugated to said dsRNA wherein said dsRNA-peptide conjugate is internalized by a target cell.

3. The isolated composition of claim 1 or 2 wherein said first strand and said second strand have a length that is at least 25 and at most 35 nucleotides, at least 19 and at most 35 nucleotides, at least 19 and at most 24 nucleotides, at least 25 and at most 30 nucleotides, at least 26 and at most 30 nucleotides or at least 21 and a most 23 nucleotides.

4. The isolated composition of claim 1 or 2 wherein said second strand comprises an overhang at the 3′ terminus.

5. The isolated composition of claim 1 or 2 wherein said first strand comprises an overhang at the 3′ terminus.

6. The isolated composition of claim 1 or 2 wherein at least one of said second strand and said first strand comprises an overhang at the 3′ terminus.

7. The isolated composition of claims 6, wherein said nucleotides of said 3′ overhang of said first and/or second strand comprise a modified nucleotide.

8. The isolated composition of claims 6, wherein said 3′ overhang(s) is/are 1-5 nucleotides in length.

9. The isolated composition of claim 1 or 2 wherein each of said first and second strands consists of the same number of nucleotide residues.

10. The isolated composition of claim 9, wherein the ultimate residue of said 5′ terminus of said first strand and the ultimate residue of said 3′ terminus of said second strand form a mismatched base pair.

11. The isolated composition of claim 9, wherein the ultimate residue of said 3′ terminus of said first strand and the ultimate residue of said 5′ terminus of said second strand form a mismatched base pair.

12. The isolated composition of claim 9 wherein the ultimate and penultimate residues of said 5′ terminus of said first strand and the ultimate and penultimate residues of said 3′ terminus of said second strand form two mismatched base pairs.

13. The isolated composition of claim 9 wherein the ultimate and penultimate residues of said 3′ terminus of said first strand and the ultimate and penultimate residues of said 5′ terminus of said second strand form two mismatched base pairs.

14. The isolated composition of claim 1 or 2, wherein said peptide comprises 6-100 amino acids.

15. The isolated composition of claim 1 or 2, wherein said peptide comprises 10-50 amino acids.

16. The isolated composition of claim 1 or 2, wherein said peptide comprises 15-30 amino acids.

17. The isolated composition of claim 1 or 2, wherein said peptide comprises 10 amino acids.

18. The isolated composition of claim 1 or 2, wherein said dsRNA-peptide conjugate binds to a receptor.

19. The isolated composition of claim 1 or 2, wherein said dsRNA-peptide conjugate binds at least one member of the LDL receptor family.

20. The isolated composition of claim 1 or 2, wherein said peptide is a PAR ligand.

21. The isolated composition of claim 1 or 2, wherein said peptide is a PAR1 ligand.

22. The isolated composition of claim 1 or 2, wherein said peptide is a growth factor ligand.

23. The isolated composition of claim 1 or 2, wherein said peptide is an insulin or insulin-like growth factor ligand.

24. The isolated composition of claim 1 or 2, wherein said peptide is an IGF-1 ligand.

25. The isolated composition of claim 1 or 2, wherein said peptide is a hormone ligand.

26. The isolated composition of claim 1 or 2, wherein said peptide is a PTH ligand.

27. The isolated composition of claim 1 or 2, wherein said peptide is a PTH-1 ligand.

28. The isolated composition of claim 1 or 2, wherein said dsRNA-peptide conjugate binds to a receptor binding protein.

29. The isolated composition of claim 1 or 2, wherein said peptide is conjugated to said dsRNA with a stable linker.

30. The isolated composition of claim 29, wherein said stable linker comprises a homobifunctional crosslinker.

31. The isolated composition of claim 29, wherein said stable linker comprises a hetero-bifunctional crosslinker.

32. The isolated composition of claim 29, wherein said stable linker comprises a trifunctional crosslinker.

33. The isolated composition of claim 1 or 2, wherein said peptide is conjugated to said dsRNA with a cleavable linker.

34. The isolated composition of claim 33, wherein said cleavable linker comprises a disulfide linker.

35. The isolated composition of claim 1 or 2, wherein said peptide is conjugated to said dsRNA with a carbon linker.

36. The isolated composition of claim 35, where said carbon linker comprises no more than eighteen carbons

37. The isolated composition of claim 35, wherein said carbon linker comprises 6 carbons.

38. The isolated composition of claim 1 or 2 wherein said peptide and said dsRNA are conjugated without a linker.

39. The isolated composition of claim 1 or 2, wherein said peptide is conjugated to the 3′ end of the first strand of said dsRNA.

40. The isolated composition of claim 1 or 2, wherein said peptide is conjugated to the 3′ end of said second strand of said dsRNA.

41. The isolated composition of claim 1 or 2, wherein said peptide is conjugated to the 5′ end of the first strand of said dsRNA.

42. The isolated composition of claim 1 or 2, wherein said peptide is conjugated to the 5′ end of said second strand of said dsRNA.

43. The isolated composition of claim 1 or 2, wherein said peptide is conjugated to the 5′ end of the first strand and the 5′ end of said second strand of said dsRNA.

44. The isolated composition of claim 1 or 2, wherein said peptide is conjugated to the 5′ end of said first strand and said 3′ end of said second strand of said dsRNA.

45. The isolated composition of claim 1 or 2, wherein said peptide is conjugated to the 3′ end of the first strand and the 3′ end of said second strand of said dsRNA.

46. The isolated composition of claim 1 or 2, wherein said peptide is conjugated to the 3′ end of said first strand and said 5′ end of said second strand of said dsRNA.

47. The isolated composition of claim 1 or 2, wherein at least one peptide is conjugated internally to said first strand of said dsRNA.

48. The isolated composition of claim 1 or 2, wherein at least one peptide is conjugated internally to said second strand of said dsRNA.

49. The isolated composition of claim 1 or 2, wherein at least one peptide is conjugated internally to said first strand and wherein at least one peptide is conjugated internally to said second strand of said dsRNA.

50. The isolated composition of claim 1 or 2, wherein at least two peptides are conjugated to said dsRNA.

51. The isolated composition of claim 50, wherein said at least two peptides are identical.

52. The isolated composition of claim 50, wherein said at least two peptides are not identical.

53. The isolated composition of claim 1 or 2, further comprising at least one dye molecule, and wherein said dye molecule is conjugated to at least one of said dsRNA and said peptide.

54. The isolated composition of claim 53, wherein said dye molecule is polyaromatic.

55. The isolated composition of claim 53, wherein said dye is a fluorescent dye.

56. The isolated composition of claim 1 or 2, further comprising a therapeutic agent.

57. The isolated composition of claim 56, wherein said therapeutic agent is an anticancer drug.

58. The isolated composition of claim 57 wherein said anticancer drug is selected from the group consisting of: paclitaxel, tamoxifen, cisplatin, doxorubicin and vinblastine.

59. The composition of claim 58, wherein said therapeutic agent is a drug to treat a metabolic disease or disorder.

60. The isolated composition of claim 1 or 2, wherein said peptide comprises a portion of a targeting moiety of a toxin.

61. The isolated composition of claim 60, wherein said neurotoxin is a clostridial toxin.

62. The isolated composition of claim 1 or 2 further comprising at least one delivery peptide.

63. The isolated composition of claim 1 or 2, wherein starting from the first nucleotide (position 1) at the 3′ terminus of the first oligonucleotide strand of said dsRNA, position 1, 2 and/or 3 is/are substituted with a modified nucleotide.

64. The isolated dsRNA of claim 60 wherein said modified nucleotide is a deoxyribonucleotide.

65. The isolated dsRNA of claim 1 or 2, wherein one or both of the first and second oligonucleotide strands comprises a 5′ phosphate.

66. The isolated composition of claim 1 or 2, wherein at least one nucleotide of said first or second strand is modified.

67. The isolated composition of claim 66 wherein said modified nucleotide residues are selected from the group consisting of 2′-O-methyl, 2′-methoxyethoxy, 2′-fluoro, 2′-allyl, 2′-O-[2-(methylamino)-2-oxoethyl], 4′-thio, 4′-CH₂—O-2′-bridge, 4′-(CH₂)₂—O-2′-bridge, 2′-LNA, 2′-amino and 2′-O-(N-methlycarbamate).

68. The isolated composition of claim 1 or 2, wherein said dsRNA is cleaved endogenously in said cell by Dicer.

69. The isolated composition of claim 1 or 2, wherein the amount of said isolated double stranded nucleic acid sufficient to reduce expression of the target gene is selected from the group consisting of 1 nanomolar or less, 200 picomolar or less, 100 picomolar or less, 50 picomolar or less, 20 picomolar or less and 10 picomolar or less in the environment of said cell.

70. The isolated composition of claim 1 or 2, wherein the first and second strands are joined by a chemical linker.

71. The isolated composition of claim 1 or 2, wherein said 3′ terminus of said first strand and said 5′ terminus of said second strand are joined by a chemical linker.

72. The isolated composition of claim 1 or 2, wherein a nucleotide of said second or first strand is substituted with a modified nucleotide that directs the orientation of Dicer cleavage.

73. The isolated composition of claim 1 or 2, comprising a modified nucleotide selected from the group consisting of a deoxyribonucleotide, a dideoxyribonucleotide, an acyclonucleotide, a 3′-deoxyadenosine (cordycepin), a 3′-azido-3′-deoxythymidine (AZT), a 2′,3′-dideoxyinosine (ddI), a 2′,3′-dideoxy-3′-thiacytidine (3TC), a 2′,3′-didehydro-2′,3′-dideoxythymidine (d4T), a monophosphate nucleotide of 3′-azido-3′-deoxythymidine (AZT), a 2′,3′-dideoxy-3′-thiacytidine (3TC) and a monophosphate nucleotide of 2′,3′-didehydro-2′,3′-dideoxythymidine (d4T), a 4-thiouracil, a 5-bromouracil, a 5-iodouracil, a 5-(3-aminoallyl)-uracil, a 2′-O-alkyl ribonucleotide, a 2′-O-methyl ribonucleotide, a 2′-amino ribonucleotide, a 2′-fluoro ribonucleotide, and a locked nucleic acid.

74. The isolated composition of claim 1 or 2 comprising a phosphate backbone modification selected from the group consisting of a phosphonate, a phosphorothioate and a phosphotriester.

75. The isolated composition of claim 1 or 2, wherein said modified nucleotide residue of said 3′ terminus of said first strand is selected from the group consisting of a deoxyribonucleotide, an acyclonucleotide and a fluorescent molecule.

76. The isolated composition of claim 1 or 2, wherein at least one of said nucleotides of said first strand and at least one of said nucleotides of said second strand form a mismatched base pair.

77. The isolated composition of claim 62, wherein said delivery peptide has an amino acid sequence selected from the group consisting of SEQ ID NO: 1-89.

78. The isolated composition of claim 1 or 2 wherein said composition is a pharmaceutical composition.

79. A method for reducing expression of a target gene in a cell, comprising:
contacting a cell with said isolated composition as claimed in claim 1 or 2, in an amount effective to reduce expression of a target gene in a cell in comparison to a reference dsRNA.

80. A method for selectively inhibiting the growth of a cell comprising contacting a cell with an amount of said isolated composition of claim 1 or 2 sufficient to inhibit the growth of the cell.

81. A method for reducing expression of a target gene in an animal, comprising: treating an animal with said isolated composition as claimed in claim 1 or 2, in an amount effective to reduce expression of a target gene in a cell of the animal in comparison to a reference dsRNA.

82. The method of claim 81, wherein said isolated composition possesses enhanced pharmacokinetics when compared to an appropriate control dsRNA.

83. The method of claim 81, wherein said dsRNA possesses enhanced pharmacodynamics when compared to an appropriate control dsRNA.

84. The method of claim 81, wherein said dsRNA possesses reduced toxicity when compared to an appropriate control dsRNA.

85. The method of claim 81, wherein said dsRNA possesses enhanced intracellular uptake when compared to an appropriate control dsRNA.

86. A pharmaceutical composition for reducing expression of a target gene in a cell of a subject comprising said isolated composition of claim 1 or 2 in an amount effective to reduce expression of a target gene in a cell in comparison to a reference dsRNA and a pharmaceutically acceptable carrier.

87. A method of synthesizing a dsRNA-peptide conjugate as claimed in any one of claims 1-78, comprising chemically or enzymatically synthesizing said dsNA.

88. A kit comprising the dsRNA-peptide conjugate of any one of claims 1-78 and instructions for its use.
